(19) United States
(12) Patent Application Publication
(10) Pub. No.: US 2025/0205352 A1
(43) Pub. Date: Jun. 26, 2025

(54) COMPOSITIONS AND METHODS FOR MODULATING NaV1.8

(71) Applicants: Eli Lilly and Company, Indianapolis, IN (US); Avidity Biosciences, Inc., San Diego, CA (US)

(72) Inventors: Jeffrey BOYLES, Indianapolis, IN (US); Arvind BHATTACHARYA, San Diego, CA (US); Patricia BROWN-AUGSBURGER, Indianapolis, IN (US); Michael Caramian COCHRAN, San Diego, CA (US); Julian DAVIES, San Diego, CA (US); Wendy Loza HOBBS, San Diego, CA (US); Yu Yan KWAN, San Diego, CA (US); Jonatan MATALONGA BORREL, San Diego, CA (US); Jeff S. McDERMOTT, Indianapolis, IN (US); Thiago OLIVA DETANICO, San Diego, CA (US); Christopher Earle WALL, San Diego, CA (US)

(21) Appl. No.: 18/976,126

(22) Filed: Dec. 10, 2024

Related U.S. Application Data
(60) Provisional application No. 63/609,643, filed on Dec. 13, 2023.

(51) Int. Cl. A61K 47/68; C07K 16/28; C12N 15/113

(52) U.S. Cl. CPC A61K 47/6807; A61K 47/6849; C07K 16/2881; C12N 15/1138; C07K 2317/24; C12N 2310/14; C12N 2310/335

(57) ABSTRACT

The present disclosure is related to oligonucleotides, antibody-siRNA conjugates, or pharmaceutical compositions thereof for inhibiting the expression of Nav1.8 gene and methods of using such oligonucleotides, antibody-siRNA conjugates, or pharmaceutical compositions thereof in reducing/treating pain or diseases/disorders related to Nav1.8.

### TECHNICAL FIELD

This disclosure is related to oligonucleotides and their use in inhibiting/modulating the expression of the Nav 1.8 gene (SCNA10A gene/transcript). The disclosure is also related to a conjugate including the oligonucleotide and anti-TfR antibodies or antigen-binding fragments thereof and their use in treating or reducing pain and/or diseases associated with Nav1.8 channel.

### SEQUENCE LISTING

The present application is being filed along with a Sequence Listing in ST.26 XML format. The Sequence Listing is provided as a file titled “30152_WO” created Nov. 20, 2024 and is 1132 kilobytes in size. The Sequence Listing information in the ST.26 XML format is incorporated herein by reference in its entirety.

### BACKGROUND

Treatment for chronic pain is a significant unmet medical problem: over one-quarter of United States citizens suffer from chronic pain (Nahin R L, “Estimates of Pain Prevalence and Severity in Adults: United States 2012,” J. Pain. 16 (8): 769-80 (2015)). Moreover, pain is one of the most common complaints seen in outpatient clinics. There are multiple categories and types of pain, including neuropathic, nociceptive, musculoskeletal, inflammatory, psychogenic, and mechanical.

There are three kinds of chronic pain: neuropathic, inflammatory, and mixed pain which includes both neuropathic and inflammatory. Neuropathic pain can be classified as peripheral and central neuropathic pain. Peripheral neuropathic pain is caused by injury or infection of peripheral sensory nerves, whereas central neuropathic pain is caused by damage to the CNS and/or the spinal cord. Both peripheral and central neuropathic pain can occur without obvious initial nerve damage. Pain due to diabetic peripheral neuropathy (DPN) is a classic example of peripheral neuropathic pain. A similar definition is given by the International Association for the Study of Pain (IASP, Seattle, Wash., USA): peripheral neuropathic pain is pain initiated or caused by a primary lesion or dysfunction in the peripheral nervous system. Central neuropathic pain is pain initiated or caused by a primary lesion or dysfunction in the central nervous system. Inflammatory pain refers to increased sensitivity due to the inflammatory response associated with tissue damage. Inflammatory pain results from the increased excitability of peripheral nociceptive sensory fibers produced by the action of inflammatory mediators. This excitatory effect, in turn, is a result of the altered activity of ion channels within affected sensory fibers. Conditions that exhibit features of both nociceptive and neuropathic pain, such as chronic low back pain (CLBP), are categorized as mixed pain. Sodium channels are central to the generation of action potentials in all excitable cells such as neurons and myocytes and play a role in disease states such as pain (Waxman et al. “Sodium Channels and Pain” Proc. Natl. Acad. Sci. USA 96 (14): 7635-9 (1999) and Waxman et al. (2000) “Voltage-gated Sodium Channels and the Molecular Pathogenesis of Pain: A Review:” J. Rehabil. Res. Dev. 37 (5): 517-28 (2000)). Voltage-gated sodium channels play a significant role in regulating neuronal excitability in normal and pathological pain states. However, the function of sodium channels (such as Nav1.7 and Nav1.8) in the pathophysiology of chronic pain is not fully understood. Nonselective antagonists of Nav channels can attenuate pain signals and are useful for treating a variety of pain conditions; however, such pain-relieving drugs (such as, analgesics) cause a range of adverse effects and are often not effective in completely relieving pain.

Common analgesics like opioids and non-steroidal anti-inflammatory drugs (NSAIDs) do not sufficiently improve pathological pain, such as, peripheral, and central neuropathic pain due to lack of adequate efficacy and/or dose limiting side effects. Side effects also limit the utility of nonselective Nav antagonists, including dizziness, somnolence, nausea, and vomiting (Tremont-Lukats et al., “Anticonvulsants for Neuropathic Pain Syndromes: Mechanisms of Action and Place in Therapy,” Drugs 60:1029-1052 (2000)) that limit the utility of Nav antagonists for the treatment of pain. These side effects are thought to result at least in part from the block of multiple Nav subtypes.

There remains a need for an agent that can selectively and efficiently modulate the activity of Nav1.8 channels for use in treating or reducing pain. An agent that inhibits the Nav1.8 channel selectively could provide a beneficial therapeutic benefit over non-selective Nav antagonists and reduce adverse effects. Also, there is a need for a pain-relieving agent that has both high biological activity and in vivo stability for treating or reducing pain.

### SUMMARY OF THE INVENTION

The present disclosure provides oligonucleotides, conjugates comprising the oligonucleotides and an anti-transferrin receptor (anti-TfR) antibody or antigen binding fragment thereof, pharmaceutical compositions, and methods for inhibiting the expression of the Nav1.8 gene in a cell or mammal. The invention also provides pharmaceutical compositions and methods for treating chronic pain and other diseases caused by the expression of Nav1.8 gene.

Nav1.8 is a voltage-gated sodium channel expressed almost exclusively in the nociceptor population of primary afferent dorsal root ganglion neurons in humans. It is responsible for the terminal phase of the action potential upstroke in primary afferents. Several Nav1.8 gain of function mutations have been identified in human populations that lead to hyperexcitable primary afferents and a variety of chronic pain phenotypes (Xiao et al., “Increased Resurgent Sodium Currents in Nav1.8 Contribute to Nociceptive Sensory Neuron Hyperexcitability Associated with Peripheral Neuropathies.” J. Neuroscience 39, no. 8:1539-1550 (2019)). In addition, selective small molecules targeting Nav1.8 have shown efficacy in both acute and chronic pain indications (Qin, H., et al., “Discovery of Selective Nav1.8 Inhibitors Based on 5-chloro-2-(4, 4-difluoroazepan-1-yl)-6-methyl Nicotinamide Scaffold for the Treatment of Pain.” European Journal of Medicinal Chemistry, 254, p. 115371 (2023); Jones, et al., “Selective Inhibition of Nav1.8 with VX-548 for Acute Pain.” New England Journal of Medicine 389, no. 5:393-405 (2023)), and multiple small molecule programs are currently in various stages of clinical testing. However, targeting peripheral sodium channels with small molecules has been historically challenging (Waxman. Stephen G., “Targeting a Peripheral Sodium Channel to Treat Pain.” New England Journal of Medicine 389:466-469 (2023)). This may be due to several factors including molecular selectivity as well as the variable nature of the blood-nerve-barrier and inconsistent target engagement throughout the multiple compartments of primary afferent neurons. The approach of delivering therapeutic oligonucleotides directly to primary afferent neurons may offer the opportunity for both exquisite molecular selectivity as well as offering consistent suppression of Nav1.8 protein expression throughout the multiple compartments of primary afferent neurons (skin terminals, nerve axons, neuronal somas, and proximal dorsal horn synapses).

In some embodiments, the conjugates, described herein, have superior benefit in that they can deliver an oligonucleotide to dorsal root ganglion (DRG) neurons. In some aspects, the present disclosure provides conjugates that have superior benefit in that they can deliver an oligonucleotide to dorsal root ganglion (DRG) neurons with reduced immunogenicity risk as compared to other conjugates having anti-TfR antibodies. In some embodiments, the conjugates, as described herein, cause reduction of pain, and can be used in treatment of chronic pain in human subjects.

In some embodiments, the TfR antibody of the present disclosure is an anti-human/cynomolgus macaque reactive transferrin receptor bivalent antibody attached to a siRNA targeting Nav1.8 (SCN10A gene/transcript) via a linker to form a conjugate antibody-oligo nucleotide (AOC) that has been engineered for uptake into peripheral tissues with the transferrin receptor. In some embodiments, when administered, the AOC binds to the apical domain of the transferrin receptor expressed on peripheral tissues including dorsal root ganglia, whereby it undergoes receptor-mediated internalization and enters lysosomal degradative compartments. The siRNA targeting Nav1.8 then is presumably released to the cytoplasm to elicit RNAi targeting mechanism.

One aspect of the present disclosure is related to oligonucleotides for inhibiting the expression of a human Nav1.8 gene (SCN10A) in a cell comprising a single stranded oligonucleotide or double stranded oligonucleotide that comprises a region of complementarity to Nav 1.8 mRNA.

Another aspect of the disclosure is related to an oligonucleotide for inhibiting the expression of a human Nav1.8 gene (SCN10A) in a cell, the oligonucleotide comprising: a sense strand and an antisense strand, wherein the sense strand and/or the antisense strand form a duplex region and the antisense strand comprises a region of complementarity to Nav1.8 mRNA (i.e., SCN10A mRNA), wherein said region of complementarity is less than 30 nucleotides in length.

Another aspect of the disclosure is related to a cell comprising one of the oligonucleotides of the disclosure. The cell is preferably a mammalian cell, such as a human cell.

In some embodiments, the oligonucleotide of the present disclosure is a single stranded (ss) oligonucleotide that includes an antisense sense strand that includes a region of complementarity to Nav1.8 mRNA (i.e., SCN10A mRNA), wherein said region of complementarity is less than 30 nucleotides in length. In some embodiments, the antisense strand is 15 to 30 nucleotides in length, 18 to 25 nucleotides in length, 19 nucleotides in length, or 21 nucleotides in length. In some embodiments, the antisense strand comprises a region of complementarity to a target sequence of any one of SEQ ID NOs: 1 to 141. In some embodiments, the region of complementarity of the ss oligonucleotide is at least 15 contiguous nucleotides in length, at least 16 contiguous nucleotides in length, at least 17 contiguous nucleotides in length, at least 18 contiguous nucleotides in length, at least 19 contiguous nucleotides in length, or at least 20 contiguous nucleotides in length. In some embodiments, the antisense strand comprises a 3′ sequence of 1 or more nucleotides in length. In some embodiments, the antisense strand includes at least 1 modified nucleotide. In some embodiments, all nucleotides of the antisense strand are modified. In some embodiments, the antisense strand has modified nucleotide, and the modified nucleotide comprises a 2′-modification on the sugar. In some embodiments, the 2′-modification is 2′-fluoro, 2′-O-methyl, or 2′-O-methoxyethyl. In some embodiments, the modification is 2′-fluoro, vinyl phosphonate uridine (VpUq), or 2′-O-methyl. In some embodiments, the 5′ end of the antisense strand comprises at least one vinyl phosphonate uridine (VpUq). In some embodiments, one or more nucleotides at positions 1-21 of the antisense strand are modified with 2′-fluoro. In some embodiments, one or more nucleotides at positions 1-21 of the antisense strand are modified with 2′-O-methyl. In some embodiments, the antisense strand has at least one modified internucleotide linkage. In some embodiments, at least one modified internucleotide linkage is a phosphorothioate linkage or a phosphorodithioate linkage. In some embodiments, at least two terminal nucleotides on 5′ end or 3′ end of the antisense strand have a phosphorothioate linkage or a phosphorodithioate linkage. In some embodiments, at least three terminal nucleotides on 5′ end or 3′ end of the antisense strand have phosphorothioate linkages or a phosphorodithioate linkages. In some embodiments, a linker moiety is attached to one or more termini of the antisense strand. In some embodiments, the linker is succinimidyl 4-(N-maleimidomethyl)cyclohexane-1-carboxylate (SMCC). In some embodiments, at least one nucleotide of the oligonucleotide is conjugated to cholesterol, lipid, polypeptide, or an antibody or an antigen binding fragment thereof. In some embodiments, the antisense strand has the sequence of i) any one of the odd numbers of SEQ ID NOs: 143 to 423 or ii) any one of SEQ ID Nos: 424 to 564. In some embodiments, (i) the antisense strand has a sequence as set forth in Table 2 or (ii) the antisense strand has a sequence as set forth in Table 3.

In another aspect, the disclosure provides a pharmaceutical composition for inhibiting the expression of the Nav1.8 gene in an organism, comprising one or more of the oligonucleotides of the invention and a pharmaceutically acceptable carrier.

In another aspect, the disclosure provides a cell comprising one of the oligonucleotides of the invention. The cell is preferably a mammalian cell, such as a human cell.

In another aspect, the disclosure provides a cell comprising a vector for inhibiting the expression of the Nav1.8 gene in a cell. The vector comprises a regulatory sequence operably linked to a nucleotide sequence that encodes at least one strand of one of the oligonucleotides of the invention.

Another aspect of the disclosure is related to a conjugate comprising the oligonucleotide of the present disclosure and an anti-transferrin receptor antibody or antigen-binding fragment thereof conjugated to the sense strand of the Nav1.8 targeting nucleotide or the antisense strand of the Nav1.8 targeting nucleotide. One embodiment of the disclosure is related to a pharmaceutical composition comprising the conjugate of the present disclosure and a pharmaceutically acceptable carrier.

Another aspect of the present disclosure is related to a method of preventing, treating, or reducing pain in a patient in need thereof, the method comprising administering to the patient an effective amount of the oligonucleotide, the conjugate, or the pharmaceutical composition of the present disclosure.

In another aspect, the invention provides a method for inhibiting the expression of the Nav1.8 gene in a cell, comprising the following steps: a) introducing into the cell the oligonucleotide or a conjugate of the present disclosure; and b) maintaining the cell produced in step a) for a time sufficient to obtain degradation of the mRNA transcript of the Nav1.8 gene, thereby inhibiting expression of the Nav 1.8 gene in the cell.

### BRIEF DESCRIPTION OF THE DRAWINGS

FIGS. 1A and 1B show modification patterns that may be used in the oligonucleotides (e.g., Nav1.8 targeting siRNAs) of the present disclosure. In the Figures, VpUq is vinyl phosphonate uridine, 2′-OMe is 2′-OMethyl modification, 2′F is 2′-Fluoro, and PS is phosphorothioate. The notations shown at the bottom of FIG. 1B are applicable to both FIGS. 1A and 1B.

FIG. 2 shows the comparison between modified siRNA 410 without VpUq (i.e., siRNA No. 285 in Table 4) and modified siRNA 410 with VpUq (i.e., siRNA No. 305 in Table 4).

FIG. 3 shows the comparison between modified siRNA 535 without VpUq (i.e., siRNA No. 291 in Table 4) and modified siRNA 535 with VpUq (i.e., siRNA No. 311 in Table 4).

FIG. 4 shows the potency of various Nav1.8 siRNA sequences in primary human dorsal root ganglia neurons (DRGs). The most potent siRNAs showed equal to or greater than 70% Nav1.8 mRNA reduction in the DRG neurons. The figure identifies the siRNA by the start position of target region on human SCN10A transcript.

FIG. 5 shows the concentration-response of some Nav1.8 siRNAs. The figure identifies the siRNA by the start position of target region on human SCN10A transcript.

FIGS. 6A-C show RT-qPCR analysis of SCN10A expression and ELISA analysis of SCN10A expression for two siRNAs.

FIG. 7 is a representative structure of one embodiment of a Nav1.8 antibody-oligonucleotide conjugate, also referred to herein as “AOC” or “conjugate”. The antibody is shown as a “Y” shape in the figure connected to a siRNA molecule via an SMCC Linker. In some embodiments, two siRNA molecules may be attached to the antibody molecule (e.g., using eCys or nCys to connect each siRNA molecule to each heavy chain of the antibody using a linker).

FIG. 8 shows a representative analytical strong anion exchange column chromatography (SAX) trace of Nav1.8 AOC reaction mixture using SAX method 1.

FIG. 9 shows a representative Fast Protein Liquid Chromatography (FPLC) trace of Nav1.8 AOC reaction mixture using FPLC method 1.

FIG. 10 shows a representative Size Exclusion Chromatography (SEC) trace using SEC Method 1.

FIG. 11 shows a representative Strong Anion Exchange (SAX) trace using SAX Method 1.

FIG. 12 shows SDS-PAGE analysis of AOCs. MWM indicates the molecular weight marker, and the molecular weight (MW) of each band of the marker is annotated. TBP1-410 is shown in the left gel, and TBP2-410 is shown in the right gel.

FIG. 13 shows that the TBP1-si410 AOC reduces Nav1.8 mRNA expression in human DRGs in vitro. The TBP1-si410 AOC includes TBP1 antibody and modified 410 siRNA linked via an SMCC linker.

FIG. 14 shows the reversal of cold-induced nocifensive response following administration of OX26-410 and OX26-535 AOCs in humanized Nav1.8 rats following IV administration of AOCs (6 mg/kg single-dose IV, by oligo weight, 13-day exposure). Rats treated with OX26-410 AOC (grey bars) displayed a statistically significant reduction of cold-induced nocifensive behavior vs PBS controls (white bars) at both temperatures tested, 2° C. and −1° C., (p<0.05, one-way ANOVA, Dunnet's post-hoc). Rats treated with OX26-535 AOC (black bars) showed a decreased response to cold, but this effect did not reach statistical significance.

FIG. 15 shows the reduction of Nav1.8 protein in humanized Nav1.8 rat DRGs following administration of OX26-410 and OX26-535 AOCs. Administration of Nav1.8-targeting AOCs, OX26-410 and OX26-535 (6 mg/kg, IV, 13 days exposure), resulted in a significant decrease in Nav1.8 protein measured in the dorsal root ganglia (DRG) and correlated with a reduction in cold-induced nocifensive behavior in humanized Nav 1.8 rats. AOC OX26-535 treated rats (black square) showed a significant reduction in DRG Nav1.8 protein vs. PBS controls (white circle) but did not cause a significant decrease in cold behavior. Humanized Nav1.8 rats treated with AOC OX26-410 (grey triangle) showed a statistically significant decrease in both Nav1.8 DRG protein levels as well as cold-induced nocifensive behavior (p<0.05, one-way ANOVA, Dunnet's post-hoc).

FIG. 16 shows reduction in Nav1.8 protein in rat glabrous skin following administration of OX26-410 and OX26-535 AOCs. Administration of Nav1.8-targeting AOCs, OX26-410 and OX26-535 (6 mg/kg, IV, 13 days exposure), resulted in a significant decrease in Nav1.8 protein measured in glabrous paw skin and correlated with a reduction in cold-induced nocifensive behavior in humanized Nav1.8 rats. AOC OX26-535 treated rats (black square) showed a significant reduction in glabrous paw skin Nav1.8 protein vs. PBS controls (white circle) but did not cause a significant decrease in cold behavior. Humanized Nav1.8 rats treated with AOC OX26-410 (grey triangle) showed a statistically significant decrease in both Nav1.8 glabrous paw skin protein levels as well as cold-induced nocifensive behavior (one-way ANOVA, Dunnet's post-hoc, p<0.05).

FIG. 17 shows reductions in Nav1.8 protein in glabrous paw skin in cynomolgus monkeys following a single dose of anti-Tfr-410 nCys mAb (dark grey bar: TBP1-siRNA410 AOC), anti-Tfr-410 eCys mAb (light grey bar: TBP2-siRNA410 AOC), anti-Tfr-410 Fab (shaded grey bar′ TBP3-siRNA410 AOC), 2 doses of anti-Tfr-410 nCys mAb, or PBS (white bar). Single-dose groups received either 1.0 or 6.0 mg/kg (by oligo weight) of AOCs. The two-dose group of anti-Tfr-410 nCys mAb was administered AOC twice on day 1 and day 8 at 3.0 mg/kg (by oligo weight) and this resulted in a significant reduction of glabrous paw skin Nav 1.8 protein levels (p<0.05, one-way ANOVA). All exposures were 29 days.

FIG. 18 shows the effect of TBP2-410 eCys AOC on Nav1.8 protein knockdown in NHP glabrous paw skin in cynomolgus monkeys. The Nav 1.8 protein is reduced in glabrous paw skin following treatment with the TBP2-410 eCys AOC. Groups received either PBS (control), 0.3, 3.0, or 30.0 mg/kg (by oligo weight) of the TBP2-410 eCys AOC (N=4/group, except for 30.0 mg/kg group which was N=2). The AOC was administered in four weekly IV doses. Administration of 3.0 and 30.0 mg/kg (by oligo weight) of AOC resulted in a significant reduction of glabrous paw skin Nav1.8 protein levels (52.3% and 42.2% of the PBS control group respectively, *p<0.05, one-way ANOVA). All exposures were 27 days following final dose (49-day total study duration).

### DETAILED DESCRIPTION

One aspect of the present disclosure is related to oligonucleotides, conjugates including such oligonucleotides, pharmaceutical compositions, and methods for inhibiting the expression of the Nav 1.8 gene in a cell or mammal using the oligonucleotides, the conjugates. or the compositions. The invention also provides compositions and methods for treating pathological conditions and diseases in a mammal caused by Nav1.8 gene using the oligonucleotides, conjugates, or compositions thereof. In some embodiments, the oligonucleotide directs the sequence-specific degradation of Nav1.8 mRNA (i.e. SCN104 mRNA) through a process known as RNA interference (RNAi).

The use of these oligonucleotides and the conjugates enables targeted degradation of Nav1.8 mRNA that is implicated in pain response in mammals. The present disclosure has demonstrated that these oligonucleotides or the conjugates can specifically and efficiently mediate RNAi, resulting in significant inhibition of expression of the Nav1.8 gene. The present disclosure has also demonstrated that the oligonucleotides and the conjugates (such as with anti-TfR antibodies) can be effectively delivered to neurons (such as dorsal root ganglion neurons) resulting in significant inhibition of expression of the Nav1.8 gene and in reduction/treatment of pain. Thus, the methods and compositions of the invention comprising these oligonucleotides and conjugates are useful for reducing or treating pain.

The following detailed description discloses how to make and use the oligonucleotides, the conjugates, and related compositions to inhibit the expression of a target Nav 1.8 gene, as well as compositions and methods for treating diseases and disorders caused by the expression of Nav1.8, such as pain.

The conjugates of the present disclosure include an oligonucleotide of the present disclosure having an antisense strand comprising a region of complementarity to at least part of an RNA transcript of the Nav 1.8 gene and an anti-TfR antibody (anti-transferrin receptor antibody) or an antigen binding fragment thereof.

The pharmaceutical compositions of the disclosure comprise an oligonucleotide having an antisense strand comprising a region of complementarity to at least part of an RNA transcript of the Nav1.8 gene, together with a pharmaceutically acceptable carrier. Another embodiment of the pharmaceutical compositions comprises the conjugate of the present disclosure together with a pharmaceutically acceptable carrier. Accordingly, certain aspects of the disclosure provide pharmaceutical compositions comprising the oligonucleotides or the conjugates of the present disclosure together with a pharmaceutically acceptable carrier, methods of using such pharmaceutical compositions to inhibit expression of the Nav1.8 gene, and methods of using the pharmaceutical compositions to treat diseases caused by expression of the Nav1.8 gene (e.g., treat pain or reduce pain).

One aspect of the present disclosure is related to an oligonucleotide (e.g., siRNA) for inhibiting the expression of a human Nav1.8 gene in a cell. This oligonucleotide includes: a sense strand and/or an antisense strand, wherein the sense strand and/or the antisense strand form a duplex region and the antisense strand comprises a region of complementarity to Nav1.8 mRNA, wherein said region of complementarity is less than 30 nucleotides in length.

In some embodiments, the oligonucleotide of the present disclosure is such that the region of complementarity of the antisense strand to the Nav1.8 mRNA target sequence is at least 15 contiguous nucleotides in length, at least 18 contiguous nucleotides in length, at least 19 contiguous nucleotides in length, at least 20 contiguous nucleotides in length, or at least 21 contiguous nucleotides in length. In some embodiments, the region of complementarity of the antisense strand to the Nav1.8 mRNA target sequence is at least 16, 17, 22, 23, 24, 25, 26, 27, 28, 29, or 30 nucleotides in length.

In some embodiments, the sense strand of the oligonucleotide is 15 to 30 nucleotides in length, 18 to 25 nucleotides in length, or 19 nucleotides in length. In some embodiments, the sense strand is 16, 17, 18, 19, 20, 21, 22, 23, 24, or 25 nucleotides in length. In some embodiments, the sense strand is 19 nucleotides in length. In some embodiments, the sense strand is 21 nucleotides in length.

In some embodiments, the antisense strand of the oligonucleotide is 15 to 30 nucleotides in length, 18 to 25 nucleotides in length, 19 nucleotides in length, or 21 nucleotides in length. In some embodiments, the antisense strand is 16, 17, 18, 19, 20, 21, 22, 23, 24, or 25 nucleotides in length. In some embodiments, the antisense strand is 19 nucleotides in length. In some embodiments, the antisense strand is 21 nucleotides in length. In some embodiments, the sense strand is 19 nucleotides in length and the antisense strand is 21 nucleotides in length. In some embodiments, the sense strand is 19 nucleotides in length and the antisense strand is 19 nucleotides in length.

In some embodiments, the oligonucleotide includes an antisense strand comprising a region of complementarity to a target sequence of any one of SEQ ID NOs: 1 to 141. Table 1 shows the target sequences present in Nav 1.8 mRNA.

TABLE 1
Target Sequences (18-mers) for SCNA10A
mRNA Transcript.

| | | Start position |
|---|---|---|
| SEQ | | of target |
| ID | Target Sequence | region on human |
| NO. | (5′ to 3′) | SCN10A transcript |
| 1 | UUACGGUCACUAUUUUGG | 407 |
| 2 | UGAUCUUGGCUGUAGUCA | 1172 |
| 3 | UAAUUGUGUGUGCAUGAC | 426 |
| 4 | CGGUCACUAUUUUGGUUA | 410 |
| 5 | AACCUGAUCAGAAGAACG | 349 |
| 6 | AAAUGAGUUCACGUACCU | 537 |
| 7 | CAACCCGGAUUUUAACUA | 990 |
| 8 | CUGACAACCCGGAUUUUA | 986 |
| 9 | AAUGAGUUCACGUACCUG | 538 |
| 10 | ACAACCCGGAUUUUAACU | 989 |
| 11 | AGAACGGCCAUCAAAGUG | 361 |
| 12 | CUUCUGACAACCCGGAUU | 983 |
| 13 | UGUCUAAAUGAGUUCACG | 532 |
| 14 | AGAAUACUUAGAUGAACC | 1794 |
| 15 | UACCUUCGAAGCCAUGCU | 2073 |
| 16 | GUGCAUGACCCGAACUGA | 435 |
| 17 | UGUGCAUGACCCGAACUG | 434 |
| 18 | CUUACAACCAGCGCAGGA | 1442 |
| 19 | CCUACCUUCGAAGCCAUG | 2071 |
| 20 | GCGUCAUUACCCUGGCAU | 584 |
| 21 | AAUUGUGUGUGCAUGACC | 427 |
| 22 | GGAUUUUAGCGUCAUUAC | 576 |
| 23 | CAGCAGAAUACUUAGAUG | 1790 |
| 24 | GCAUGAGCCCUACCUUCG | 2063 |
| 25 | UGACAACCCGGAUUUUAA | 987 |
| 26 | CUAAAUGAGUUCACGUAC | 535 |
| 27 | UGUCAGCAGAAUACUUAG | 1787 |
| 28 | AACCAACUACUCAUCUCA | 858 |
| 29 | CGCUUUACUCCGGAGUCA | 43 |
| 30 | ACAACCAACUACUCAUCU | 856 |
| 31 | CUUGUACCUUUGUCGAUU | 3958 |
| 32 | CACGUACCUGAGAGAUCC | 546 |
| 33 | CCCAAAACCCAAUCGAAA | 5331 |
| 34 | GGGUGAAGCUCAAGACAA | 1952 |
| 35 | GUUUAUUUAUUACGGUCA | 398 |
| 36 | CAGUUUAUUUAUUACGGU | 396 |
| 37 | ACCAACUACUCAUCUCAC | 859 |
| 38 | ACCUGAUCAGAAGAACGG | 350 |
| 39 | CUUUUUUGUGCUCGUAAU | 1125 |
| 40 | GAAUUCCCCAUUGGAUCC | 4 |
| 41 | GGCAUAUGUUGGCACAGC | 597 |
| 42 | GCCUUAGUGAUAGAGUCA | 5780 |
| 43 | UCAUUACCCUGGCAUAUG | 587 |
| 44 | AAUGAGUGUUGUCAGUAU | 1830 |
| 45 | AGGACAUCUAGCUCAAUA | 5803 |
| 46 | UGAUCCUUACAACCAGCG | 1437 |
| 47 | UUAUUUAUUACGGUCACU | 400 |
| 48 | AAGAACGGCCAUCAAAGU | 360 |
| 49 | GACAACCCGGAUUUUAAC | 988 |
| 50 | UCUAGCUCAAUACAAAAU | 5809 |
| 51 | GAUCUCCGUGGGAUCUCA | 619 |
| 52 | CCGGUUUAGUGCCACUCG | 306 |
| 53 | CUCAUCCUUUUCUUGACG | 2599 |
| 54 | ACCCAAUCGAAAUAUACU | 5337 |
| 55 | UUUAUCUUUGUGUUCGAG | 3583 |
| 56 | ACAGAAGGAUGCAUUCGC | 3346 |
| 57 | GUCACUAGAGGCCUUAGU | 5770 |
| 58 | UCUUUUUUGUGCUCGUAA | 1124 |
| 59 | UUGUCGAUUGUGAAUAAC | 3967 |
| 60 | AGGGCAAUGAGUGUUGUC | 1825 |
| 61 | UUUUUGUGCUCGUAAUCU | 1127 |
| 62 | UUCGAUGCAGGACAAAAG | 1759 |
| 63 | CAACCGAAAAAAUAUCUC | 2445 |
| 64 | ACGGUCACUAUUUUGGUU | 409 |
| 65 | GGAUGCAUUCGCCACUGU | 3352 |
| 66 | GGGCAAUGAGUGUUGUCA | 1826 |
| 67 | UGAUAGAGUCAACAUGAG | 5787 |
| 68 | AUCCUUACAACCAGCGCA | 1439 |
| 69 | UGUACCUUUGUCGAUUGU | 3960 |
| 70 | UUGUACCUUUGUCGAUUG | 3959 |
| 71 | ACUCGAGGAGUCUGAACA | 1869 |
| 72 | CAGUAUCAUAACCUCCGU | 1842 |
| 73 | AAGGGCAAUGAGUGUUGU | 1824 |
| 74 | CAAUGAGUGUUGUCAGUA | 1829 |
| 75 | UGCAUUCGCCACUGUCCC | 3355 |
| 76 | AUCUUUUUUGUGCUCGUA | 1123 |
| 77 | CAGUGACUUCAUCGCUAA | 2952 |
| 78 | GUUUAUGGCAACUAAUCU | 5484 |
| 79 | CUUGUGCAUCGUGGUGAA | 2016 |
| 80 | CUUUGGGCUUGUGACGGA | 1974 |
| 81 | CUUGGGCUUUCCUCUCAC | 1028 |
| 82 | CAUCUAGCUCAAUACAAA | 5807 |
| 83 | GUUGUCAGUAUCAUAACC | 1837 |
| 84 | CUCCGGAGUCACUGGUGG | 50 |
| 85 | AGGUCUUUGGCCAUCGUA | 2753 |
| 86 | GUGUGCAUGACCCGAACU | 433 |
| 87 | CACGGAUCCAGGUCUUUG | 2744 |
| 88 | ACUUAGAUGAACCUUUCC | 1799 |
| 89 | AACAUGCAACCCAAGUGG | 4147 |
| 90 | UAGAGGCCUUAGUGAUAG | 5775 |
| 91 | AUCGAAAUAUACUGAUCC | 5342 |
| 92 | UUUCAGCCACUGUCAUUC | 5558 |
| 93 | AUAACCUCCGUCCUUGAG | 1849 |
| 94 | UUUAUGGCAACUAAUCUU | 5485 |
| 95 | CUAUGAACCAAUAGCAAC | 5514 |
| 96 | CCAACUACUCAUCUCACA | 860 |
| 97 | GUUCAUCGCCCUGCUAUU | 2652 |
| 98 | AUCCUUUUCUUGACGGUG | 2602 |
| 99 | CUAUCGGAGCUAUGUGCU | 5583 |
| 100 | CCAAGAAUGUCCUAGGAG | 5423 |
| 101 | UUCUACAGCACACACCGG | 253 |
| 102 | AGUUUAUUUAUUACGGUC | 397 |
| 103 | AUCAUCCUAUGAACCAAU | 5508 |
| 104 | CAGGUCUUUGGCCAUCGU | 2752 |
| 105 | GCACACACCGGACAUUUA | 260 |
| 106 | CAUCGUGACCAGACAAGC | 4413 |
| 107 | GCAAUGGGUUACCUUGCA | 4060 |
| 108 | UCCUUUUCUUGACGGUGA | 2603 |
| 109 | UCGAAAUAUACUGAUCCA | 5343 |
| 110 | UCGAUGCAGGACAAAAGA | 1760 |
| 111 | CAAAAGGCCUAUCGGAGC | 5575 |
| 112 | GGCGAAUGUGUCAUGAAG | 4561 |
| 113 | GCUAUGUGCUGCACCGCU | 5591 |
| 114 | UCCCAGCAGAACUGAUCG | 209 |
| 115 | CACACCGGACAUUUAUGG | 263 |
| 116 | GUGUCACUAGAGGCCUUA | 5768 |
| 117 | UGUCGAUUGUGAAUAACA | 3968 |
| 118 | AACUUCUGACAACCCGGA | 981 |
| 119 | CUAUGUGCUGCACCGCUC | 5592 |
| 120 | AAGUCUGACUGCAAGAUU | 3985 |
| 121 | CCUUUGUCGAUUGUGAAU | 3964 |
| 122 | GUUUAGUGCCACUCGGGC | 309 |
| 123 | UCCUUACAACCAGCGCAG | 1440 |
| 124 | AACCCAAUCGAAAUAUAC | 5336 |
| 125 | UGUGUGCAUGACCCGAAC | 432 |
| 126 | CAUCGCCCUGCUAUUGAA | 2655 |
| 127 | CCUUACAACCAGCGCAGG | 1441 |
| 128 | AUCUCCGCGCCCCAUGAA | 2458 |
| 129 | CUUUUCUUGACGGUGAUG | 2605 |
| 130 | UCACUAGAGGCCUUAGUG | 5771 |
| 131 | GCGAAUGUGUCAUGAAGA | 4562 |
| 132 | UCUGACAACCCGGAUUUU | 985 |
| 133 | AGUAUCAUAACCUCCGUC | 1843 |
| 134 | GCAUAUGUUGGCACAGCA | 598 |
| 135 | CCCUUACUGUGUGGCAAU | 919 |
| 136 | CUACAGCACACACCGGAC | 255 |
| 137 | CGAAAAAAUAUCUCCGCG | 2449 |
| 138 | CUUGGUGGGGCUGCAACU | 783 |
| 139 | CUGCAAGAUUCAAAACUC | 3993 |
| 140 | CGUCGCUUUACUCCGGAG | 40 |
| 141 | UCAUCCUAUGAACCAAUA | 5509 |

In some embodiments, the oligonucleotide includes an antisense strand comprising a region of complementarity to a Nav1.8 mRNA target sequence wherein the region of complementarity of the antisense strand to the target sequence (e.g., as shown in Table 1) is at least 15 contiguous nucleotides in length, at least 16 contiguous nucleotides in length, at least 17 contiguous nucleotides in length, at least 18 contiguous nucleotides in length, at least 19 contiguous nucleotides in length, or at least 20 contiguous nucleotides in length.

In some embodiments, the oligonucleotide is such that the sense strand has the sequence of anyone of the even numbers of SEQ ID NOs: 142 to 422. In some embodiments, the antisense strand of the oligonucleotide has the sequence of any one of the odd numbers of SEQ ID NOs: 143 to 423. In some embodiments, the antisense strand of the oligonucleotide has the sequence of anyone of SEQ ID Nos: 424 to 564.

Tables 2 and 3 below show some exemplary oligonucleotides of the present disclosure.

TABLE 2
Nucleic Acid Sequences of 19-mer
Nav1.8 siRNAs

| dsRNA | | | | | |
|---|---|---|---|---|---|
| No. | | | | | |
| (or | | | | | Start |
| SiRNA | | | | | posi- |
| No.) | | | | | tion |
| (refers | | | | | of |
| to du- | | | | | target |
| plexed | | | Antisense | | region |
| SS | | | Strand | | on |
| and AS | | | (5′ to 3′) | | human |
| strand | Sense | SEQ | (without | SEQ | SCN10A |
| of | Strand | ID | 3′ UU | ID | trans- |
| siRNA) | (5′ to 3′) | NO. | overhang) | NO. | cript |
| 1 | UUACGGUCACU | 142 | UCCAAAAUAGU | 143 | 407 |
| | AUUUUGGA | | GACCGUAA | | |
| 2 | UGAUCUUGGCU | 144 | UUGACUACAGC | 145 | 1172 |
| | GUAGUCAA | | CAAGAUCA | | |
| 3 | UAAUUGUGUGU | 146 | UGUCAUGCACA | 147 | 426 |
| | GCAUGACA | | CACAAUUA | | |
| 4 | CGGUCACUAUU | 148 | UUAACCAAAAU | 149 | 410 |
| | UUGGUUAA | | AGUGACCG | | |
| 5 | AACCUGAUCAG | 150 | UCGUUCUUCUG | 151 | 349 |
| | AAGAACGA | | AUCAGGUU | | |
| 6 | AAAUGAGUUCA | 152 | UAGGUACGUGA | 153 | 537 |
| | CGUACCUA | | ACUCAUUU | | |
| 7 | CAACCCGGAUU | 154 | UUAGUUAAAAU | 155 | 990 |
| | UUAACUAA | | CCGGGUUG | | |
| 8 | CUGACAACCCG | 156 | UUAAAAUCCGG | 157 | 986 |
| | GAUUUUAA | | GUUGUCAG | | |
| 9 | AAUGAGUUCAC | 158 | UCAGGUACGUG | 159 | 538 |
| | GUACCUGA | | AACUCAUU | | |
| 10 | ACAACCCGGAU | 160 | UAGUUAAAAUC | 161 | 989 |
| | UUUAACUA | | CGGGUUGU | | |
| 11 | AGAACGGCCAU | 162 | UCACUUUGAUG | 163 | 361 |
| | CAAAGUGA | | GCCGUUCU | | |
| 12 | CUUCUGACAAC | 164 | UAAUCCGGGUU | 165 | 983 |
| | CCGGAUUA | | GUCAGAAG | | |
| 13 | UGUCUAAAUGA | 166 | UCGUGAACUCA | 167 | 532 |
| | GUUCACGA | | UUUAGACA | | |
| 14 | AGAAUACUUAG | 168 | UGGUUCAUCUA | 169 | 1794 |
| | AUGAACCA | | AGUAUUCU | | |
| 15 | UACCUUCGAAG | 170 | UAGCAUGGCUU | 171 | 2073 |
| | CCAUGCUA | | CGAAGGUA | | |
| 16 | GUGCAUGACCC | 172 | UUCAGUUCGGG | 173 | 435 |
| | GAACUGAA | | UCAUGCAC | | |
| 17 | UGUGCAUGACC | 174 | UCAGUUCGGGU | 175 | 434 |
| | CGAACUGA | | CAUGCACA | | |
| 18 | CUUACAACCAG | 176 | UUCCUGCGCUG | 177 | 1442 |
| | CGCAGGAA | | GUUGUAAG | | |
| 19 | CCUACCUUCGA | 178 | UCAUGGCUUCG | 179 | 2071 |
| | AGCCAUGA | | AAGGUAGG | | |
| 20 | GCGUCAUUACC | 180 | UAUGCCAGGGU | 181 | 584 |
| | CUGGCAUA | | AAUGACGC | | |
| 21 | AAUUGUGUGUG | 182 | UGGUCAUGCAC | 183 | 427 |
| | CAUGACCA | | ACACAAUU | | |
| 22 | GGAUUUUAGCG | 184 | UGUAAUGACGC | 185 | 576 |
| | UCAUUACA | | UAAAAUCC | | |
| 23 | CAGCAGAAUAC | 186 | UCAUCUAAGUA | 187 | 1790 |
| | UUAGAUGA | | UUCUGCUG | | |
| 24 | GCAUGAGCCCU | 188 | UCGAAGGUAGG | 189 | 2063 |
| | ACCUUCGA | | GCUCAUGC | | |
| 25 | UGACAACCCGG | 190 | UUUAAAAUCCG | 191 | 987 |
| | AUUUUAAA | | GGUUGUCA | | |
| 26 | CUAAAUGAGUU | 192 | UGUACGUGAAC | 193 | 535 |
| | CACGUACA | | UCAUUUAG | | |
| 27 | UGUCAGCAGAA | 194 | UCUAAGUAUUC | 195 | 1787 |
| | UACUUAGA | | UGCUGACA | | |
| 28 | AACCAACUACU | 196 | UUGAGAUGAGU | 197 | 858 |
| | CAUCUCAA | | AGUUGGUU | | |
| 29 | CGCUUUACUCC | 198 | UUGACUCCGGA | 199 | 43 |
| | GGAGUCAA | | GUAAAGCG | | |
| 30 | ACAACCAACUA | 200 | UAGAUGAGUAG | 201 | 856 |
| | CUCAUCUA | | UUGGUUGU | | |
| 31 | CUUGUACCUUU | 202 | UAAUCGACAAA | 203 | 3958 |
| | GUCGAUUA | | GGUACAAG | | |
| 32 | CACGUACCUGA | 204 | UGGAUCUCUCA | 205 | 546 |
| | GAGAUCCA | | GGUACGUG | | |
| 33 | CCCAAAACCCA | 206 | UUUUCGAUUGG | 207 | 5331 |
| | AUCGAAAA | | GUUUUGGG | | |
| 34 | GGGUGAAGCUC | 208 | UUUGUCUUGAG | 209 | 1952 |
| | AAGACAAA | | CUUCACCC | | |
| 35 | GUUUAUUUAUU | 210 | UUGACCGUAAU | 211 | 398 |
| | ACGGUCAA | | AAAUAAAC | | |
| 36 | CAGUUUAUUUA | 212 | UACCGUAAUAA | 213 | 396 |
| | UUACGGUA | | AUAAACUG | | |
| 37 | ACCAACUACUC | 214 | UGUGAGAUGAG | 215 | 859 |
| | AUCUCACA | | UAGUUGGU | | |
| 38 | ACCUGAUCAGA | 216 | UCCGUUCUUCU | 217 | 350 |
| | AGAACGGA | | GAUCAGGU | | |
| 39 | CUUUUUUGUGC | 218 | UAUUACGAGCA | 219 | 1125 |
| | UCGUAAUA | | CAAAAAAG | | |
| 40 | GAAUUCCCCAU | 220 | UGGAUCCAAUG | 221 | 4 |
| | UGGAUCCA | | GGGAAUUC | | |
| 41 | GGCAUAUGUUG | 222 | UGCUGUGCCAA | 223 | 597 |
| | GCACAGCA | | CAUAUGCC | | |
| 42 | GCCUUAGUGAU | 224 | UUGACUCUAUC | 225 | 5780 |
| | AGAGUCAA | | ACUAAGGC | | |
| 43 | UCAUUACCCUG | 226 | UCAUAUGCCAG | 227 | 587 |
| | GCAUAUGA | | GGUAAUGA | | |
| 44 | AAUGAGUGUUG | 228 | UAUACUGACAA | 229 | 1830 |
| | UCAGUAUA | | CACUCAUU | | |
| 45 | AGGACAUCUAG | 230 | UUAUUGAGCUA | 231 | 5803 |
| | CUCAAUAA | | GAUGUCCU | | |
| 46 | UGAUCCUUACA | 232 | UCGCUGGUUGU | 233 | 1437 |
| | ACCAGCGA | | AAGGAUCA | | |
| 47 | UUAUUUAUUAC | 234 | UAGUGACCGUA | 235 | 400 |
| | GGUCACUA | | AUAAAUAA | | |
| 48 | AAGAACGGCCA | 236 | UACUUUGAUGG | 237 | 360 |
| | UCAAAGUA | | CCGUUCUU | | |
| 49 | GACAACCCGGA | 238 | UGUUAAAAUCC | 239 | 988 |
| | UUUUAACA | | GGGUUGUC | | |
| 50 | UCUAGCUCAAU | 240 | UAUUUUGUAUU | 241 | 5809 |
| | ACAAAAUA | | GAGCUAGA | | |
| 51 | GAUCUCCGUGG | 242 | UUGAGAUCCCA | 243 | 619 |
| | GAUCUCAA | | CGGAGAUC | | |
| 52 | CCGGUUUAGUG | 244 | UCGAGUGGCAC | 245 | 306 |
| | CCACUCGA | | UAAACCGG | | |
| 53 | CUCAUCCUUUU | 246 | UCGUCAAGAAA | 247 | 2599 |
| | CUUGACGA | | AGGAUGAG | | |
| 54 | ACCCAAUCGAA | 248 | UAGUAUAUUUC | 249 | 5337 |
| | AUAUACUA | | GAUUGGGU | | |
| 55 | UUUAUCUUUGU | 250 | UCUCGAACACA | 251 | 3583 |
| | GUUCGAGA | | AAGAUAAA | | |
| 56 | ACAGAAGGAUG | 252 | UGCGAAUGCAU | 253 | 3346 |
| | CAUUCGCA | | CCUUCUGU | | |
| 57 | GUCACUAGAGG | 254 | UACUAAGGCCU | 255 | 5770 |
| | CCUUAGUA | | CUAGUGAC | | |
| 58 | UCUUUUUUGUG | 256 | UUUACGAGCAC | 257 | 1124 |
| | CUCGUAAA | | AAAAAAGA | | |
| 59 | UUGUCGAUUGU | 258 | UGUUAUUCACA | 259 | 3967 |
| | GAAUAACA | | AUCGACAA | | |
| 60 | AGGGCAAUGAG | 260 | UGACAACACUC | 261 | 1825 |
| | UGUUGUCA | | AUUGCCCU | | |
| 61 | UUUUUGUGCUC | 262 | UAGAUUACGAG | 263 | 1127 |
| | GUAAUCUA | | CACAAAAA | | |
| 62 | UUCGAUGCAGG | 264 | UCUUUUGUCCU | 265 | 1759 |
| | ACAAAAGA | | GCAUCGAA | | |
| 63 | CAACCGAAAAA | 266 | UGAGAUAUUUU | 267 | 2445 |
| | AUAUCUCA | | UUCGGUUG | | |
| 64 | ACGGUCACUAU | 268 | UAACCAAAAUA | 269 | 409 |
| | UUUGGUUA | | GUGACCGU | | |
| 65 | GGAUGCAUUCG | 270 | UACAGUGGCGA | 271 | 3352 |
| | CCACUGUA | | AUGCAUCC | | |
| 66 | GGGCAAUGAGU | 272 | UUGACAACACU | 273 | 1826 |
| | GUUGUCAA | | CAUUGCCC | | |
| 67 | UGAUAGAGUCA | 274 | UCUCAUGUUGA | 275 | 5787 |
| | ACAUGAGA | | CUCUAUCA | | |
| 68 | AUCCUUACAAC | 276 | UUGCGCUGGUU | 277 | 1439 |
| | CAGCGCAA | | GUAAGGAU | | |
| 69 | UGUACCUUUGU | 278 | UACAAUCGACA | 279 | 3960 |
| | CGAUUGUA | | AAGGUACA | | |
| 70 | UUGUACCUUUG | 280 | UCAAUCGACAA | 281 | 3959 |
| | UCGAUUGA | | AGGUACAA | | |
| 71 | ACUCGAGGAGU | 282 | UUGUUCAGACU | 283 | 1869 |
| | CUGAACAA | | CCUCGAGU | | |
| 72 | CAGUAUCAUAA | 284 | UACGGAGGUUA | 285 | 1842 |
| | CCUCCGUA | | UGAUACUG | | |
| 73 | AAGGGCAAUGA | 286 | UACAACACUCA | 287 | 1824 |
| | GUGUUGUA | | UUGCCCUU | | |
| 74 | CAAUGAGUGUU | 288 | UUACUGACAAC | 289 | 1829 |
| | GUCAGUAA | | ACUCAUUG | | |
| 75 | UGCAUUCGCCA | 290 | UGGGACAGUGG | 291 | 3355 |
| | CUGUCCCA | | CGAAUGCA | | |
| 76 | AUCUUUUUUGU | 292 | UUACGAGCACA | 293 | 1123 |
| | GCUCGUAA | | AAAAAGAU | | |
| 77 | CAGUGACUUCA | 294 | UUUAGCGAUGA | 295 | 2952 |
| | UCGCUAAA | | AGUCACUG | | |
| 78 | GUUUAUGGCAA | 296 | UAGAUUAGUUG | 297 | 5484 |
| | CUAAUCUA | | CCAUAAAC | | |
| 79 | CUUGUGCAUCG | 298 | UUUCACCACGA | 299 | 2016 |
| | UGGUGAAA | | UGCACAAG | | |
| 80 | CUUUGGGCUUG | 300 | UUCCGUCACAA | 301 | 1974 |
| | UGACGGAA | | GCCCAAAG | | |
| 81 | CUUGGGCUUUC | 302 | UGUGAGAGGAA | 303 | 1028 |
| | CUCUCACA | | AGCCCAAG | | |
| 82 | CAUCUAGCUCA | 304 | UUUUGUAUUGA | 305 | 5807 |
| | AUACAAAA | | GCUAGAUG | | |
| 83 | GUUGUCAGUAU | 306 | UGGUUAUGAUA | 307 | 1837 |
| | CAUAACCA | | CUGACAAC | | |
| 84 | CUCCGGAGUCA | 308 | UCCACCAGUGA | 309 | 50 |
| | CUGGUGGA | | CUCCGGAG | | |
| 85 | AGGUCUUUGGC | 310 | UUACGAUGGCC | 311 | 2753 |
| | CAUCGUAA | | AAAGACCU | | |
| 86 | GUGUGCAUGAC | 312 | UAGUUCGGGUC | 313 | 433 |
| | CCGAACUA | | AUGCACAC | | |
| 87 | CACGGAUCCAG | 314 | UCAAAGACCUG | 315 | 2744 |
| | GUCUUUGA | | GAUCCGUG | | |
| 88 | ACUUAGAUGAA | 316 | UGGAAAGGUUC | 317 | 1799 |
| | CCUUUCCA | | AUCUAAGU | | |
| 89 | AACAUGCAACC | 318 | UCCACUUGGGU | 319 | 4147 |
| | CAAGUGGA | | UGCAUGUU | | |
| 90 | UAGAGGCCUUA | 320 | UCUAUCACUAA | 321 | 5775 |
| | GUGAUAGA | | GGCCUCUA | | |
| 91 | AUCGAAAUAUA | 322 | UGGAUCAGUAU | 323 | 5342 |
| | CUGAUCCA | | AUUUCGAU | | |
| 92 | UUUCAGCCACU | 324 | UGAAUGACAGU | 325 | 5558 |
| | GUCAUUCA | | GGCUGAAA | | |
| 93 | AUAACCUCCGU | 326 | UCUCAAGGACG | 327 | 1849 |
| | CCUUGAGA | | GAGGUUAU | | |
| 94 | UUUAUGGCAAC | 328 | UAAGAUUAGUU | 329 | 5485 |
| | UAAUCUUA | | GCCAUAAA | | |
| 95 | CUAUGAACCAA | 330 | UGUUGCUAUUG | 331 | 5514 |
| | UAGCAACA | | GUUCAUAG | | |
| 96 | CCAACUACUCA | 332 | UUGUGAGAUGA | 333 | 860 |
| | UCUCACAA | | GUAGUUGG | | |
| 97 | GUUCAUCGCCC | 334 | UAAUAGCAGGG | 335 | 2652 |
| | UGCUAUUA | | CGAUGAAC | | |
| 98 | AUCCUUUUCUU | 336 | UCACCGUCAAG | 337 | 2602 |
| | GACGGUGA | | AAAAGGAU | | |
| 99 | CUAUCGGAGCU | 338 | UAGCACAUAGC | 339 | 5583 |
| | AUGUGCUA | | UCCGAUAG | | |
| 100 | CCAAGAAUGUC | 340 | UCUCCUAGGAC | 341 | 5423 |
| | CUAGGAGA | | AUUCUUGG | | |
| 101 | UUCUACAGCAC | 342 | UCCGGUGUGUG | 343 | 253 |
| | ACACCGGA | | CUGUAGAA | | |
| 102 | AGUUUAUUUAU | 344 | UGACCGUAAUA | 345 | 397 |
| | UACGGUCA | | AAUAAACU | | |
| 103 | AUCAUCCUAUG | 346 | UAUUGGUUCAU | 347 | 5508 |
| | AACCAAUA | | AGGAUGAU | | |
| 104 | CAGGUCUUUGG | 348 | UACGAUGGCCA | 349 | 2752 |
| | CCAUCGUA | | AAGACCUG | | |
| 105 | GCACACACCGG | 350 | UUAAAUGUCCG | 351 | 260 |
| | ACAUUUAA | | GUGUGUGC | | |
| 106 | CAUCGUGACCA | 352 | UGCUUGUCUGG | 353 | 4413 |
| | GACAAGCA | | UCACGAUG | | |
| 107 | GCAAUGGGUUA | 354 | UUGCAAGGUAA | 355 | 4060 |
| | CCUUGCAA | | CCCAUUGC | | |
| 108 | UCCUUUUCUUG | 356 | UUCACCGUCAA | 357 | 2603 |
| | ACGGUGAA | | GAAAAGGA | | |
| 109 | UCGAAAUAUAC | 358 | UUGGAUCAGUA | 359 | 5343 |
| | UGAUCCAA | | UAUUUCGA | | |
| 110 | UCGAUGCAGGA | 360 | UUCUUUUGUCC | 361 | 1760 |
| | CAAAAGAA | | UGCAUCGA | | |
| 111 | CAAAAGGCCUA | 362 | UGCUCCGAUAG | 363 | 5575 |
| | UCGGAGCA | | GCCUUUUG | | |
| 112 | GGCGAAUGUGU | 364 | UCUUCAUGACA | 365 | 4561 |
| | CAUGAAGA | | CAUUCGCC | | |
| 113 | GCUAUGUGCUG | 366 | UAGCGGUGCAG | 367 | 5591 |
| | CACCGCUA | | CACAUAGC | | |
| 114 | UCCCAGCAGAA | 368 | UCGAUCAGUUC | 369 | 209 |
| | CUGAUCGA | | UGCUGGGA | | |
| 115 | CACACCGGACA | 370 | UCCAUAAAUGU | 371 | 263 |
| | UUUAUGGA | | CCGGUGUG | | |
| 116 | GUGUCACUAGA | 372 | UUAAGGCCUCU | 373 | 5768 |
| | GGCCUUAA | | AGUGACAC | | |
| 117 | UGUCGAUUGUG | 374 | UUGUUAUUCAC | 375 | 3968 |
| | AAUAACAA | | AAUCGACA | | |
| 118 | AACUUCUGACA | 376 | UUCCGGGUUGU | 377 | 981 |
| | ACCCGGAA | | CAGAAGUU | | |
| 119 | CUAUGUGCUGC | 378 | UGAGCGGUGCA | 379 | 5592 |
| | ACCGCUCA | | GCACAUAG | | |
| 120 | AAGUCUGACUG | 380 | UAAUCUUGCAG | 381 | 3985 |
| | CAAGAUUA | | UCAGACUU | | |
| 121 | CCUUUGUCGAU | 382 | UAUUCACAAUC | 383 | 3964 |
| | UGUGAAUA | | GACAAAGG | | |
| 122 | GUUUAGUGCCA | 384 | UGCCCGAGUGG | 385 | 309 |
| | CUCGGGCA | | CACUAAAC | | |
| 123 | UCCUUACAACC | 386 | UCUGCGCUGGU | 387 | 1440 |
| | AGCGCAGA | | UGUAAGGA | | |
| 124 | AACCCAAUCGA | 388 | UGUAUAUUUCG | 389 | 5336 |
| | AAUAUACA | | AUUGGGUU | | |
| 125 | UGUGUGCAUGA | 390 | UGUUCGGGUCA | 391 | 432 |
| | CCCGAACA | | UGCACACA | | |
| 126 | CAUCGCCCUGC | 392 | UUUCAAUAGCA | 393 | 2655 |
| | UAUUGAAA | | GGGCGAUG | | |
| 127 | CCUUACAACCA | 394 | UCCUGCGCUGG | 395 | 1441 |
| | GCGCAGGA | | UUGUAAGG | | |
| 128 | AUCUCCGCGCC | 396 | UUUCAUGGGGC | 397 | 2458 |
| | CCAUGAAA | | GCGGAGAU | | |
| 129 | CUUUUCUUGAC | 398 | UCAUCACCGUC | 399 | 2605 |
| | GGUGAUGA | | AAGAAAAG | | |
| 130 | UCACUAGAGGC | 400 | UCACUAAGGCC | 401 | 5771 |
| | CUUAGUGA | | UCUAGUGA | | |
| 131 | GCGAAUGUGUC | 402 | UUCUUCAUGAC | 403 | 4562 |
| | AUGAAGAA | | ACAUUCGC | | |
| 132 | UCUGACAACCC | 404 | UAAAAUCCGGG | 405 | 985 |
| | GGAUUUUA | | UUGUCAGA | | |
| 133 | AGUAUCAUAAC | 406 | UGACGGAGGUU | 407 | 1843 |
| | CUCCGUCA | | AUGAUACU | | |
| 134 | GCAUAUGUUGG | 408 | UUGCUGUGCCA | 409 | 598 |
| | CACAGCAA | | ACAUAUGC | | |
| 135 | CCCUUACUGUG | 410 | UAUUGCCACAC | 411 | 919 |
| | UGGCAAUA | | AGUAAGGG | | |
| 136 | CUACAGCACAC | 412 | UGUCCGGUGUG | 413 | 255 |
| | ACCGGACA | | UGCUGUAG | | |
| 137 | CGAAAAAAUAU | 414 | UCGCGGAGAUA | 415 | 2449 |
| | CUCCGCGA | | UUUUUUCG | | |
| 138 | CUUGGUGGGGC | 416 | UAGUUGCAGCC | 417 | 783 |
| | UGCAACUA | | CCACCAAG | | |
| 139 | CUGCAAGAUUC | 418 | UGAGUUUUGAA | 419 | 3993 |
| | AAAACUCA | | UCUUGCAG | | |
| 140 | CGUCGCUUUAC | 420 | UCUCCGGAGUA | 421 | 40 |
| | UCCGGAGA | | AAGCGACG | | |
| 141 | UCAUCCUAUGA | 422 | UUAUUGGUUCA | 423 | 5509 |
| | ACCAAUAA | | UAGGAUGA | | |

TABLE 3
Nucleic Acid Sequences of 19/21-mer Nav1.8 siRNAs

| | | | | | Start |
|---|---|---|---|---|---|
| | | | | | position of |
| | | | | | target |
| | | SEQ | Antisense Strand | SEQ | region on |
| siRNA | Sense Strand | ID | (5′ to 3′) | ID | human SCN10A |
| No. | (5′ to 3′) | NO. | (with 3′ UU overhang) | NO. | transcript |
| 142 | UUACGGUCACU | 142 | UCCAAAAUAGUG | 424 | 407 |
| | AUUUUGGA | | ACCGUAAUU | | |
| 143 | UGAUCUUGGCU | 144 | UUGACUACAGCC | 425 | 1172 |
| | GUAGUCAA | | AAGAUCAUU | | |
| 144 | UAAUUGUGUGU | 146 | UGUCAUGCACAC | 426 | 426 |
| | GCAUGACA | | ACAAUUAUU | | |
| 145 | CGGUCACUAUU | 148 | UUAACCAAAAUA | 427 | 410 |
| | UUGGUUAA | | GUGACCGUU | | |
| 146 | AACCUGAUCAG | 150 | UCGUUCUUCUGA | 428 | 349 |
| | AAGAACGA | | UCAGGUUUU | | |
| 147 | AAAUGAGUUCA | 152 | UAGGUACGUGAA | 429 | 537 |
| | CGUACCUA | | CUCAUUUUU | | |
| 148 | CAACCCGGAUU | 154 | UUAGUUAAAAUC | 430 | 990 |
| | UUAACUAA | | CGGGUUGUU | | |
| 149 | CUGACAACCCG | 156 | UUAAAAUCCGGG | 431 | 986 |
| | GAUUUUAA | | UUGUCAGUU | | |
| 150 | AAUGAGUUCAC | 158 | UCAGGUACGUGA | 432 | 538 |
| | GUACCUGA | | ACUCAUUUU | | |
| 151 | ACAACCCGGAU | 160 | UAGUUAAAAUCC | 433 | 989 |
| | UUUAACUA | | GGGUUGUUU | | |
| 152 | AGAACGGCCAU | 162 | UCACUUUGAUGG | 434 | 361 |
| | CAAAGUGA | | CCGUUCUUU | | |
| 153 | CUUCUGACAAC | 164 | UAAUCCGGGUUG | 435 | 983 |
| | CCGGAUUA | | UCAGAAGUU | | |
| 154 | UGUCUAAAUGA | 166 | UCGUGAACUCAU | 436 | 532 |
| | GUUCACGA | | UUAGACAUU | | |
| 155 | AGAAUACUUAG | 168 | UGGUUCAUCUAA | 437 | 1794 |
| | AUGAACCA | | GUAUUCUUU | | |
| 156 | UACCUUCGAAG | 170 | UAGCAUGGCUUC | 438 | 2073 |
| | CCAUGCUA | | GAAGGUAUU | | |
| 157 | GUGCAUGACCC | 172 | UUCAGUUCGGGU | 439 | 435 |
| | GAACUGAA | | CAUGCACUU | | |
| 158 | UGUGCAUGACC | 174 | UCAGUUCGGGUC | 440 | 434 |
| | CGAACUGA | | AUGCACAUU | | |
| 159 | CUUACAACCAG | 176 | UUCCUGCGCUGG | 441 | 1442 |
| | CGCAGGAA | | UUGUAAGUU | | |
| 160 | CCUACCUUCGA | 178 | UCAUGGCUUCGA | 442 | 2071 |
| | AGCCAUGA | | AGGUAGGUU | | |
| 161 | GCGUCAUUACC | 180 | UAUGCCAGGGUA | 443 | 584 |
| | CUGGCAUA | | AUGACGCUU | | |
| 162 | AAUUGUGUGUG | 182 | UGGUCAUGCACA | 444 | 427 |
| | CAUGACCA | | CACAAUUUU | | |
| 163 | GGAUUUUAGCG | 184 | UGUAAUGACGCU | 445 | 576 |
| | UCAUUACA | | AAAAUCCUU | | |
| 164 | CAGCAGAAUAC | 186 | UCAUCUAAGUAU | 446 | 1790 |
| | UUAGAUGA | | UCUGCUGUU | | |
| 165 | GCAUGAGCCCU | 188 | UCGAAGGUAGGG | 447 | 2063 |
| | ACCUUCGA | | CUCAUGCUU | | |
| 166 | UGACAACCCGG | 190 | UUUAAAAUCCGG | 448 | 987 |
| | AUUUUAAA | | GUUGUCAUU | | |
| 167 | CUAAAUGAGUU | 192 | UGUACGUGAACU | 449 | 535 |
| | CACGUACA | | CAUUUAGUU | | |
| 168 | UGUCAGCAGAA | 194 | UCUAAGUAUUCU | 450 | 1787 |
| | UACUUAGA | | GCUGACAUU | | |
| 169 | AACCAACUACU | 196 | UUGAGAUGAGUA | 451 | 858 |
| | CAUCUCAA | | GUUGGUUUU | | |
| 170 | CGCUUUACUCC | 198 | UUGACUCCGGAG | 452 | 43 |
| | GGAGUCAA | | UAAAGCGUU | | |
| 171 | ACAACCAACUA | 200 | UAGAUGAGUAGU | 453 | 856 |
| | CUCAUCUA | | UGGUUGUUU | | |
| 172 | CUUGUACCUUU | 202 | UAAUCGACAAAG | 454 | 3958 |
| | GUCGAUUA | | GUACAAGUU | | |
| 173 | CACGUACCUGA | 204 | UGGAUCUCUCAG | 455 | 546 |
| | GAGAUCCA | | GUACGUGUU | | |
| 174 | CCCAAAACCCA | 206 | UUUUCGAUUGGG | 456 | 5331 |
| | AUCGAAAA | | UUUUGGGUU | | |
| 175 | GGGUGAAGCUC | 208 | UUUGUCUUGAGC | 457 | 1952 |
| | AAGACAAA | | UUCACCCUU | | |
| 176 | GUUUAUUUAUU | 210 | UUGACCGUAAUA | 458 | 398 |
| | ACGGUCAA | | AAUAAACUU | | |
| 177 | CAGUUUAUUUA | 212 | UACCGUAAUAAA | 459 | 396 |
| | UUACGGUA | | UAAACUGUU | | |
| 178 | ACCAACUACUC | 214 | UGUGAGAUGAGU | 460 | 859 |
| | AUCUCACA | | AGUUGGUUU | | |
| 179 | ACCUGAUCAGA | 216 | UCCGUUCUUCUG | 461 | 350 |
| | AGAACGGA | | AUCAGGUUU | | |
| 180 | CUUUUUUGUGC | 218 | UAUUACGAGCAC | 462 | 1125 |
| | UCGUAAUA | | AAAAAAGUU | | |
| 181 | GAAUUCCCCAU | 220 | UGGAUCCAAUGG | 463 | 4 |
| | UGGAUCCA | | GGAAUUCUU | | |
| 182 | GGCAUAUGUUG | 222 | UGCUGUGCCAAC | 464 | 597 |
| | GCACAGCA | | AUAUGCCUU | | |
| 183 | GCCUUAGUGAU | 224 | UUGACUCUAUCA | 465 | 5780 |
| | AGAGUCAA | | CUAAGGCUU | | |
| 184 | UCAUUACCCUG | 226 | UCAUAUGCCAGG | 466 | 587 |
| | GCAUAUGA | | GUAAUGAUU | | |
| 185 | AAUGAGUGUUG | 228 | UAUACUGACAAC | 467 | 1830 |
| | UCAGUAUA | | ACUCAUUUU | | |
| 186 | AGGACAUCUAG | 230 | UUAUUGAGCUAG | 468 | 5803 |
| | CUCAAUAA | | AUGUCCUUU | | |
| 187 | UGAUCCUUACA | 232 | UCGCUGGUUGUA | 469 | 1437 |
| | ACCAGCGA | | AGGAUCAUU | | |
| 188 | UUAUUUAUUAC | 234 | UAGUGACCGUAA | 470 | 400 |
| | GGUCACUA | | UAAAUAAUU | | |
| 189 | AAGAACGGCCA | 236 | UACUUUGAUGGC | 471 | 360 |
| | UCAAAGUA | | CGUUCUUUU | | |
| 190 | GACAACCCGGA | 238 | UGUUAAAAUCCG | 472 | 988 |
| | UUUUAACA | | GGUUGUCUU | | |
| 191 | UCUAGCUCAAU | 240 | UAUUUUGUAUUG | 473 | 5809 |
| | ACAAAAUA | | AGCUAGAUU | | |
| 192 | GAUCUCCGUGG | 242 | UUGAGAUCCCAC | 474 | 619 |
| | GAUCUCAA | | GGAGAUCUU | | |
| 193 | CCGGUUUAGUG | 244 | UCGAGUGGCACU | 475 | 306 |
| | CCACUCGA | | AAACCGGUU | | |
| 194 | CUCAUCCUUUU | 246 | UCGUCAAGAAAA | 476 | 2599 |
| | CUUGACGA | | GGAUGAGUU | | |
| 195 | ACCCAAUCGAA | 248 | UAGUAUAUUUCG | 477 | 5337 |
| | AUAUACUA | | AUUGGGUUU | | |
| 196 | UUUAUCUUUGU | 250 | UCUCGAACACAA | 478 | 3583 |
| | GUUCGAGA | | AGAUAAAUU | | |
| 197 | ACAGAAGGAUG | 252 | UGCGAAUGCAUC | 479 | 3346 |
| | CAUUCGCA | | CUUCUGUUU | | |
| 198 | GUCACUAGAGG | 254 | UACUAAGGCCUC | 480 | 5770 |
| | CCUUAGUA | | UAGUGACUU | | |
| 199 | UCUUUUUUGUG | 256 | UUUACGAGCACA | 481 | 1124 |
| | CUCGUAAA | | AAAAAGAUU | | |
| 200 | UUGUCGAUUGU | 258 | UGUUAUUCACAA | 482 | 3967 |
| | GAAUAACA | | UCGACAAUU | | |
| 201 | AGGGCAAUGAG | 260 | UGACAACACUCA | 483 | 1825 |
| | UGUUGUCA | | UUGCCCUUU | | |
| 202 | UUUUUGUGCUC | 262 | UAGAUUACGAGC | 484 | 1127 |
| | GUAAUCUA | | ACAAAAAUU | | |
| 203 | UUCGAUGCAGG | 264 | UCUUUUGUCCUG | 485 | 1759 |
| | ACAAAAGA | | CAUCGAAUU | | |
| 204 | CAACCGAAAAA | 266 | UGAGAUAUUUUU | 486 | 2445 |
| | AUAUCUCA | | UCGGUUGUU | | |
| 205 | ACGGUCACUAU | 268 | UAACCAAAAUAG | 487 | 409 |
| | UUUGGUUA | | UGACCGUUU | | |
| 206 | GGAUGCAUUCG | 270 | UACAGUGGCGAA | 488 | 3352 |
| | CCACUGUA | | UGCAUCCUU | | |
| 207 | GGGCAAUGAGU | 272 | UUGACAACACUC | 489 | 1826 |
| | GUUGUCAA | | AUUGCCCUU | | |
| 208 | UGAUAGAGUCA | 274 | UCUCAUGUUGAC | 490 | 5787 |
| | ACAUGAGA | | UCUAUCAUU | | |
| 209 | AUCCUUACAAC | 276 | UUGCGCUGGUUG | 491 | 1439 |
| | CAGCGCAA | | UAAGGAUUU | | |
| 210 | UGUACCUUUGU | 278 | UACAAUCGACAA | 492 | 3960 |
| | CGAUUGUA | | AGGUACAUU | | |
| 211 | UUGUACCUUUG | 280 | UCAAUCGACAAA | 493 | 3959 |
| | UCGAUUGA | | GGUACAAUU | | |
| 212 | ACUCGAGGAGU | 282 | UUGUUCAGACUC | 494 | 1869 |
| | CUGAACAA | | CUCGAGUUU | | |
| 213 | CAGUAUCAUAA | 284 | UACGGAGGUUAU | 495 | 1842 |
| | CCUCCGUA | | GAUACUGUU | | |
| 214 | AAGGGCAAUGA | 286 | UACAACACUCAU | 496 | 1824 |
| | GUGUUGUA | | UGCCCUUUU | | |
| 215 | CAAUGAGUGUU | 288 | UUACUGACAACA | 497 | 1829 |
| | GUCAGUAA | | CUCAUUGUU | | |
| 216 | UGCAUUCGCCA | 290 | UGGGACAGUGGC | 498 | 3355 |
| | CUGUCCCA | | GAAUGCAUU | | |
| 217 | AUCUUUUUUGU | 292 | UUACGAGCACAA | 499 | 1123 |
| | GCUCGUAA | | AAAAGAUUU | | |
| 218 | CAGUGACUUCA | 294 | UUUAGCGAUGAA | 500 | 2952 |
| | UCGCUAAA | | GUCACUGUU | | |
| 219 | GUUUAUGGCAA | 296 | UAGAUUAGUUGC | 501 | 5484 |
| | CUAAUCUA | | CAUAAACUU | | |
| 220 | CUUGUGCAUCG | 298 | UUUCACCACGAU | 502 | 2016 |
| | UGGUGAAA | | GCACAAGUU | | |
| 221 | CUUUGGGCUUG | 300 | UUCCGUCACAAG | 503 | 1974 |
| | UGACGGAA | | CCCAAAGUU | | |
| 222 | CUUGGGCUUUC | 302 | UGUGAGAGGAAA | 504 | 1028 |
| | CUCUCACA | | GCCCAAGUU | | |
| 223 | CAUCUAGCUCA | 304 | UUUUGUAUUGAG | 505 | 5807 |
| | AUACAAAA | | CUAGAUGUU | | |
| 224 | GUUGUCAGUAU | 306 | UGGUUAUGAUAC | 506 | 1837 |
| | CAUAACCA | | UGACAACUU | | |
| 225 | CUCCGGAGUCA | 308 | UCCACCAGUGAC | 507 | 50 |
| | CUGGUGGA | | UCCGGAGUU | | |
| 226 | AGGUCUUUGGC | 310 | UUACGAUGGCCA | 508 | 2753 |
| | CAUCGUAA | | AAGACCUUU | | |
| 227 | GUGUGCAUGAC | 312 | UAGUUCGGGUCA | 509 | 433 |
| | CCGAACUA | | UGCACACUU | | |
| 228 | CACGGAUCCAG | 314 | UCAAAGACCUGG | 510 | 2744 |
| | GUCUUUGA | | AUCCGUGUU | | |
| 229 | ACUUAGAUGAA | 316 | UGGAAAGGUUCA | 511 | 1799 |
| | CCUUUCCA | | UCUAAGUUU | | |
| 230 | AACAUGCAACC | 318 | UCCACUUGGGUU | 512 | 4147 |
| | CAAGUGGA | | GCAUGUUUU | | |
| 231 | UAGAGGCCUUA | 320 | UCUAUCACUAAG | 513 | 5775 |
| | GUGAUAGA | | GCCUCUAUU | | |
| 232 | AUCGAAAUAUA | 322 | UGGAUCAGUAUA | 514 | 5342 |
| | CUGAUCCA | | UUUCGAUUU | | |
| 233 | UUUCAGCCACU | 324 | UGAAUGACAGUG | 515 | 5558 |
| | GUCAUUCA | | GCUGAAAUU | | |
| 234 | AUAACCUCCGU | 326 | UCUCAAGGACGG | 516 | 1849 |
| | CCUUGAGA | | AGGUUAUUU | | |
| 235 | UUUAUGGCAAC | 328 | UAAGAUUAGUUG | 517 | 5485 |
| | UAAUCUUA | | CCAUAAAUU | | |
| 236 | CUAUGAACCAA | 330 | UGUUGCUAUUGG | 518 | 5514 |
| | UAGCAACA | | UUCAUAGUU | | |
| 237 | CCAACUACUCA | 332 | UUGUGAGAUGAG | 519 | 860 |
| | UCUCACAA | | UAGUUGGUU | | |
| 238 | GUUCAUCGCCC | 334 | UAAUAGCAGGGC | 520 | 2652 |
| | UGCUAUUA | | GAUGAACUU | | |
| 239 | AUCCUUUUCUU | 336 | UCACCGUCAAGA | 521 | 2602 |
| | GACGGUGA | | AAAGGAUUU | | |
| 240 | CUAUCGGAGCU | 338 | UAGCACAUAGCU | 522 | 5583 |
| | AUGUGCUA | | CCGAUAGUU | | |
| 24 | CCAAGAAUGUC | 340 | UCUCCUAGGACA | 523 | 5423 |
| | CUAGGAGA | | UUCUUGGUU | | |
| 242 | UUCUACAGCAC | 342 | UCCGGUGUGUGC | 524 | 253 |
| | ACACCGGA | | UGUAGAAUU | | |
| 243 | AGUUUAUUUAU | 344 | UGACCGUAAUAA | 525 | 397 |
| | UACGGUCA | | AUAAACUUU | | |
| 244 | AUCAUCCUAUG | 346 | UAUUGGUUCAUA | 526 | 5508 |
| | AACCAAUA | | GGAUGAUUU | | |
| 245 | CAGGUCUUUGG | 348 | UACGAUGGCCAA | 527 | 2752 |
| | CCAUCGUA | | AGACCUGUU | | |
| 246 | GCACACACCGG | 350 | UUAAAUGUCCGG | 528 | 260 |
| | ACAUUUAA | | UGUGUGCUU | | |
| 247 | CAUCGUGACCA | 352 | UGCUUGUCUGGU | 529 | 4413 |
| | GACAAGCA | | CACGAUGUU | | |
| 248 | GCAAUGGGUUA | 354 | UUGCAAGGUAAC | 530 | 4060 |
| | CCUUGCAA | | CCAUUGCUU | | |
| 249 | UCCUUUUCUUG | 356 | UUCACCGUCAAG | 531 | 2603 |
| | ACGGUGAA | | AAAAGGAUU | | |
| 250 | UCGAAAUAUAC | 358 | UUGGAUCAGUAU | 532 | 5343 |
| | UGAUCCAA | | AUUUCGAUU | | |
| 251 | UCGAUGCAGGA | 360 | UUCUUUUGUCCU | 533 | 1760 |
| | CAAAAGAA | | GCAUCGAUU | | |
| 252 | CAAAAGGCCUA | 362 | UGCUCCGAUAGG | 534 | 5575 |
| | UCGGAGCA | | CCUUUUGUU | | |
| 253 | GGCGAAUGUGU | 364 | UCUUCAUGACAC | 535 | 4561 |
| | CAUGAAGA | | AUUCGCCUU | | |
| 254 | GCUAUGUGCUG | 366 | UAGCGGUGCAGC | 536 | 5591 |
| | CACCGCUA | | ACAUAGCUU | | |
| 255 | UCCCAGCAGAA | 368 | UCGAUCAGUUCU | 537 | 209 |
| | CUGAUCGA | | GCUGGGAUU | | |
| 256 | CACACCGGACA | 370 | UCCAUAAAUGUC | 538 | 263 |
| | UUUAUGGA | | CGGUGUGUU | | |
| 257 | GUGUCACUAGA | 372 | UUAAGGCCUCUA | 539 | 5768 |
| | GGCCUUAA | | GUGACACUU | | |
| 258 | UGUCGAUUGUG | 374 | UUGUUAUUCACA | 540 | 3968 |
| | AAUAACAA | | AUCGACAUU | | |
| 259 | AACUUCUGACA | 376 | UUCCGGGUUGUC | 541 | 981 |
| | ACCCGGAA | | AGAAGUUUU | | |
| 260 | CUAUGUGCUGC | 378 | UGAGCGGUGCAG | 542 | 5592 |
| | ACCGCUCA | | CACAUAGUU | | |
| 261 | AAGUCUGACUG | 380 | UAAUCUUGCAGU | 543 | 3985 |
| | CAAGAUUA | | CAGACUUUU | | |
| 262 | CCUUUGUCGAU | 382 | UAUUCACAAUCG | 544 | 3964 |
| | UGUGAAUA | | ACAAAGGUU | | |
| 263 | GUUUAGUGCCA | 384 | UGCCCGAGUGGC | 545 | 309 |
| | CUCGGGCA | | ACUAAACUU | | |
| 264 | UCCUUACAACC | 386 | UCUGCGCUGGUU | 546 | 1440 |
| | AGCGCAGA | | GUAAGGAUU | | |
| 265 | AACCCAAUCGA | 388 | UGUAUAUUUCGA | 547 | 5336 |
| | AAUAUACA | | UUGGGUUUU | | |
| 266 | UGUGUGCAUGA | 390 | UGUUCGGGUCAU | 548 | 432 |
| | CCCGAACA | | GCACACAUU | | |
| 267 | CAUCGCCCUGC | 392 | UUUCAAUAGCAG | 549 | 2655 |
| | UAUUGAAA | | GGCGAUGUU | | |
| 268 | CCUUACAACCA | 394 | UCCUGCGCUGGU | 550 | 1441 |
| | GCGCAGGA | | UGUAAGGUU | | |
| 269 | AUCUCCGCGCC | 396 | UUUCAUGGGGCG | 551 | 2458 |
| | CCAUGAAA | | CGGAGAUUU | | |
| 270 | CUUUUCUUGAC | 398 | UCAUCACCGUCA | 552 | 2605 |
| | GGUGAUGA | | AGAAAAGUU | | |
| 271 | UCACUAGAGGC | 400 | UCACUAAGGCCU | 553 | 5771 |
| | CUUAGUGA | | CUAGUGAUU | | |
| 272 | GCGAAUGUGUC | 402 | UUCUUCAUGACA | 554 | 4562 |
| | AUGAAGAA | | CAUUCGCUU | | |
| 273 | UCUGACAACCC | 404 | UAAAAUCCGGGU | 555 | 985 |
| | GGAUUUUA | | UGUCAGAUU | | |
| 274 | AGUAUCAUAAC | 406 | UGACGGAGGUUA | 556 | 1843 |
| | CUCCGUCA | | UGAUACUUU | | |
| 275 | GCAUAUGUUGG | 408 | UUGCUGUGCCAA | 557 | 598 |
| | CACAGCAA | | CAUAUGCUU | | |
| 276 | CCCUUACUGUG | 410 | UAUUGCCACACA | 558 | 919 |
| | UGGCAAUA | | GUAAGGGUU | | |
| 277 | CUACAGCACAC | 412 | UGUCCGGUGUGU | 559 | 255 |
| | ACCGGACA | | GCUGUAGUU | | |
| 278 | CGAAAAAAUAU | 414 | UCGCGGAGAUAU | 560 | 2449 |
| | CUCCGCGA | | UUUUUCGUU | | |
| 279 | CUUGGUGGGGC | 416 | UAGUUGCAGCCC | 561 | 783 |
| | UGCAACUA | | CACCAAGUU | | |
| 280 | CUGCAAGAUUC | 418 | UGAGUUUUGAAU | 562 | 3993 |
| | AAAACUCA | | CUUGCAGUU | | |
| 281 | CGUCGCUUUAC | 420 | UCUCCGGAGUAA | 563 | 40 |
| | UCCGGAGA | | AGCGACGUU | | |
| 282 | UCAUCCUAUGA | 422 | UUAUUGGUUCAU | 564 | 5509 |
| | ACCAAUAA | | AGGAUGAUU | | |

In some embodiments, the oligonucleotide of the present disclosure is such that the sense strand and the antisense strands comprise nucleotide sequences selected from the group consisting of:
(a) SEQ ID NOs: 565 and 566, respectively;
(b) SEQ ID NOs: 567 and 568, respectively;
(c) SEQ ID NOs: 569 and 570, respectively;
(d) SEQ ID NOs: 571 and 572, respectively;
(e) SEQ ID NOs: 573 and 574, respectively;
(f) SEQ ID NOs: 575 and 576, respectively;
(g) SEQ ID NOs: 577 and 578 respectively;
(h) SEQ ID NOs: 579 and 580, respectively;
(i) SEQ ID NOs: 581 and 582, respectively;
(j) SEQ ID NOs: 583 and 584, respectively;
(k) SEQ ID NOs: 585 and 586, respectively;
(l) SEQ ID NOs: 587 and 588, respectively;
(m) SEQ ID NOs: 589 and 590 respectively;
(n) SEQ ID NOs: 591 and 592, respectively;
(o) SEQ ID NOs: 593 and 594, respectively;
(p) SEQ ID NOs: 595 and 596, respectively;
(q) SEQ ID NOs: 597 and 598, respectively;
(r) SEQ ID NOs: 599 and 600, respectively;
(s) SEQ ID NOs: 601 and 602 respectively; and
(t) SEQ ID NOs: 603 and 604 respectively.

In some embodiments, the oligonucleotide of the present disclosure is such that the sense strand and the antisense strands comprise nucleotide sequences selected from the group consisting of:
(a) SEQ ID NOs: 565 and 605, respectively;
(b) SEQ ID NOs: 567 and 606, respectively;
(c) SEQ ID NOs: 569 and 607, respectively;
(d) SEQ ID NOs: 571 and 608, respectively;
(e) SEQ ID NOs: 573 and 609, respectively;
(f) SEQ ID NOs: 575 and 610, respectively;
(g) SEQ ID NOs: 577 and 611 respectively;
(h) SEQ ID NOs: 579 and 612, respectively;
(i) SEQ ID NOs: 581 and 613, respectively;
(j) SEQ ID NOs: 583 and 614, respectively;
(k) SEQ ID NOs: 585 and 615, respectively;
(l) SEQ ID NOs: 587 and 616, respectively;
(m) SEQ ID NOs: 589 and 617 respectively;
(n) SEQ ID NOs: 591 and 618, respectively;
(o) SEQ ID NOs: 593 and 619, respectively;
(p) SEQ ID NOs: 595 and 620, respectively;
(q) SEQ ID NOs: 597 and 621, respectively;
(r) SEQ ID NOs: 599 and 622, respectively;
(s) SEQ ID NOs: 601 and 623 respectively; and
(t) SEQ ID NOs: 603 and 624 respectively.

In some embodiments, the oligonucleotide of the present disclosure is such that i) the sense strand has a sequence as set forth in Table 2 and antisense strand has a sequence as set forth in Table 2: ii) the sense strand has a sequence set forth in Table 3 and the antisense strand has a sequence set forth in Table 3; iii) the oligonucleotide has a sequence set forth by any one of siRNA No. 1 to 141; iv) the oligonucleotide has a sequence set forth by any one of siRNA No. 142 to 282: or v) the oligonucleotide has a sequence set forth by any one of siRNA No. 283 to 322.

### RNA Modifications

A variety of oligonucleotide types and/or structures are useful for targeting SCN10A mRNA including, but not limited to, oligonucleotides, antisense oligonucleotides (ASOs), miRNAs, etc. Any of the oligonucleotide types described herein or elsewhere are contemplated for use as a framework to incorporate a targeting sequence herein for the purposes of inhibiting SCN10A activity. In some embodiments, the oligonucleotide herein inhibits SCN10A activity by engaging with RNAi pathways upstream or downstream of Dicer involvement. For example, oligonucleotides have been developed with each strand having sizes of about 19-25 nucleotides with at least one 3′ overhang of 1 to 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended RNAi oligonucleotides where at least one end of at least one strand is extended beyond a duplex targeting region, including structures where one of the strands includes a thermodynamically stabilizing tetraloop (see, e.g., U.S. Pat. Nos. 8,513,207 and 8,927,705, as well as Intl. Patent Application Publication No. WO 2010/033225). Such structures include single strand extensions (on one or both sides of the molecule) as well as double strand extensions.

In some embodiments, the oligonucleotides herein engage with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide (e.g., siRNA) includes a 21-nucleotide antisense strand that is antisense to a target mRNA (e.g., SCN10A mRNA) and a complementary sense strand, in which both strands anneal to form a 19-bp duplex and 2 nucleotide overhangs at either or both 3′ ends. Longer oligonucleotide designs also are contemplated, including oligonucleotides having an antisense strand of 23 nucleotides and a sense strand of 21 nucleotides, where there is a blunt end on the right side of the molecule (3′ end of sense strand/5′ end of antisense strand) and a two nucleotide 3′ antisense strand overhang on the left side of the molecule (5′ end of the sense strand/3′ end of the antisense strand). In such molecules, there is a 21 bp duplex region. See, e.g., U.S. Pat. Nos. 9,012,138; 9,012,621 and 9,193,753.

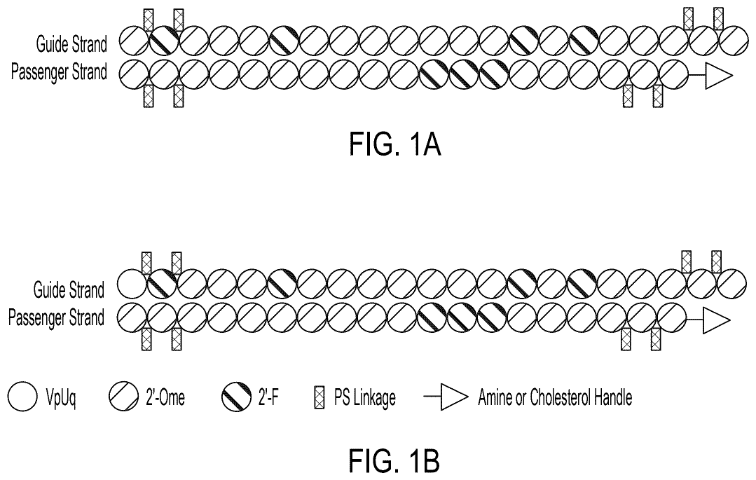

In some embodiments, the oligonucleotide of the present disclosure has a modification pattern as shown in FIG. 1A, optionally without the amine or cholesterol handle. In some embodiments, the oligonucleotide has a modification pattern as shown in FIG. 1B, optionally without the amine or cholesterol handle.

In some embodiments, the oligonucleotide of the present disclosure has an antisense strand that includes a 3′ overhang sequence of 1 or more nucleotides in length. In some embodiments, the 3′ overhang sequence is 2 nucleotides in length. In some embodiments, the 3′ overhang sequence is UU. In some embodiments, the 3′ overhang sequence is AA, GG, or CC.

In some embodiments, the oligonucleotide of the present disclosure includes at least 1 modified nucleotide. In some embodiments, all nucleotides of the oligonucleotide are modified.

A modified sugar (also referred to herein as a sugar analog) includes a modified deoxyribose or ribose moiety in which, for example, one or more modifications occur at the 2′, 3′, 4′, and/or 5′ carbon position of the sugar. A modified sugar also includes non-natural, alternative, carbon structures such as those present in locked nucleic acids (“LNA”; see, e.g., Koshkin et al., “LNA (Locked Nucleic Acids): Synthesis of the Adenine, Cytosine, Guanine, 5-methylcytosine, Thymine and Uracil Bicyclonucleoside Monomers, Oligomerisation, and Unprecedented Nucleic Acid Recognition,” Tetrahedron 54, no. 14 (1998): 3607-3630), unlocked nucleic acids (“UNA”; see, e.g., Snead et al., “5′ Unlocked Nucleic Acid Modification Improves siRNA Targeting,” Molecular Therapy—Nucleic Acids 2 (2013)) and bridged nucleic acids (“BNA”; see, e.g., Imanishi, Takeshi, and Satoshi Obika, “BNAs: Novel Nucleic Acid Analogs with a Bridged Sugar Moiety,” Chemical Communications 16 (2002): 1653-1659).

In some embodiments, the nucleotide modification in the sugar is a 2′-modification, for example, 2′-O-propargyl, 2′-O-propylamin, 2′-amino, 2′-ethyl, 2′-F (2′-Fluoro), 2′-aminoethyl (EA), 2′-OMe (2′-OMethyl), 2′-MOE (2′-methoxyethyl), 2′-O-[2-(methylamino)-2-oxoethyl] (2′-O-NMA), or 2′-FANA (2′-Fluoro-arabinonucleic acid). In certain embodiments, the modification is 2′-F, 2′-OMe, or 2′-MOE. In other embodiments, the modification in the sugar is a modification of the sugar ring, which includes modification of one or more carbons of the sugar ring. For example, the modification in the sugar is a 2′-oxygen of the sugar linked to a 1′-carbon or 4′-carbon of the sugar, or a 2′-oxygen linked to the 1′-carbon or 4′-carbon via an ethylene or methylene bridge. In other embodiments, the modification is an acyclic sugar that lacks a 2′-carbon to 3′-carbon bond. In other embodiments, the modification is a thiol group such as, for example, in the 4′ position of the sugar.

The oligonucleotides herein include at least 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand comprises at least 1 modified nucleotide (e.g., at least 1, at least 5, at least 10, at least 15, at least 20, at least 25, at least 30, at least 35, or more). In some embodiments, the antisense strand comprises at least 1 modified nucleotide (e.g., at least 1, at least 5, at least 10, at least 15, at least 20, or more).

In certain embodiments, all nucleotides of the sense strand except the tetraL (also referred to herein as tetraloop) are modified. Likewise, all nucleotides of the antisense strand are modified. In some embodiments, all the nucleotides of the oligonucleotide (i.e., paired nucleotides of the sense strand and the antisense strand) are modified. As above, and in some embodiments, the modified nucleotide is a 2′-modification (e.g., a 2′-F, 2′-OMe, 2′-MOE, and/or 2′-FANA. In certain embodiments, the modified nucleotide is a 2′-modification such as, for example, a 2′-F or a 2′-OMe.

In some embodiments, the modified nucleotide comprises a 2′-modification. In some embodiments, the 2′-modification can be, for example, 2′-aminoethyl, 2′-fluoro, 2′-O-methyl, 2′-O-methoxyethyl, or 2′-deoxy-2′-fluoro-β-d-arabinonucleic acid. In some embodiments, the oligonucleotide is modified and includes 2′-fluoro, vinyl phosphonate uridine (VpUq), or 2′-O-methyl modifications.

In some embodiments, the oligonucleotide of the present disclosure includes at least one vinyl phosphonate uridine (VpUq) at the 5′ end of the antisense strand. In some embodiments, the oligonucleotide of the present disclosure includes at least two or more vinyl phosphonate uridine (VpUq) at the 5′ end of the antisense strand. In some embodiments, the oligonucleotide is modified and contains a vinyl phosphonate (Vp).

In some embodiments, the oligonucleotide comprises a sense strand with about 50-90% (e.g., 50%, 55%, 60%, 65%, 70%, 75%, 80%, 85%, or 90%) of the nucleotides of the sense strand comprising a 2′-OMe modification. In some embodiments, the oligonucleotide comprises a sense strand with about 50-60% of the nucleotides of the sense strand comprising a 2′-OMe modification. In some embodiments, the oligonucleotide comprises a sense strand with about 60%-70% of the nucleotides of the sense strand comprising a 2′-OMe modification. In some embodiments, the oligonucleotide comprises a sense strand with about 70%-80% of the nucleotides of the sense strand comprising a 2′-OMe modification. In some embodiments, the oligonucleotide comprises a sense strand with about 80%-90% of the nucleotides of the sense strand comprising a 2′-OMe modification. In some embodiments, the oligonucleotide comprises a sense strand with about 90%-100% of the nucleotides of the sense strand comprising a 2′-OMe modification. In some embodiments, about 78% of the nucleotides of the sense strand comprise a 2′-OMe modification. In some embodiments, about 79% of the nucleotides of the sense strand comprise a 2′-OMe modification. In some embodiments, about 80% of the nucleotides of the sense strand comprise a 2′-OMe modification. In some embodiments, about 81% of the nucleotides of the sense strand comprise a 2′-OMe modification. In some embodiments, about 84% of the nucleotides of the sense strand comprise a 2′-OMe modification.

In some embodiments, the oligonucleotide comprises an anti-sense strand with about 50-90% (e.g., 50%, 55%, 60%, 65%, 70%, 75%, 80%, 85%, or 90%) of the nucleotides of the anti-sense strand comprising a 2′-OMe modification. In some embodiments, the oligonucleotide comprises an anti-sense strand with about 50-60% of the nucleotides of the anti-sense strand comprising a 2′-OMe modification. In some embodiments, the oligonucleotide comprises an anti-sense strand with about 60%-70% of the nucleotides of the anti-sense strand comprising a 2′-OMe modification. In some embodiments, the oligonucleotide comprises an anti-sense strand with about 70%-80% of the nucleotides of the anti-sense strand comprising a 2′-OMe modification. In some embodiments, the oligonucleotide comprises an anti-sense strand with about 80%-90% of the nucleotides of the anti-sense strand comprising a 2′-OMe modification. In some embodiments, the oligonucleotide comprises an anti-sense strand with about 90%-100% of the nucleotides of the anti-sense strand comprising a 2′-OMe modification. In some embodiments, about 78% of the nucleotides of the anti-sense strand comprise a 2′-OMe modification. In some embodiments, about 79% of the nucleotides of the anti-sense strand comprise a 2′-OMe modification. In some embodiments, about 80% of the nucleotides of the anti-sense strand comprise a 2′-OMe modification. In some embodiments, about 81% of the nucleotides of the anti-sense strand comprise a 2′-OMe modification.

In some embodiments, the oligonucleotide has about 15% to about 25% (e.g., 15%, 16%, 17%, 18%, 19%, 20%, 21%, 22%, 23%, 24%, or 25%) of its nucleotides comprising a 2′-OMe modification. In some embodiments, the oligonucleotide has about 35-45% (e.g., 35%, 36%, 37%, 38%, 39%, 40%, 41%, 42%, 43%, 44% or 45%) of its nucleotides comprising a 2′-OMe modification. In some embodiments, the oligonucleotide has about 45-85% (e.g., 45%, 50%, 55%, 60%, 65%, 70%, 75%, 80%, or 85%) of its nucleotides comprising a 2′-OMe modification. In some embodiments, about 70% of the nucleotides in the oligonucleotide comprise a 2′-OMe modification. In some embodiments, about 75% of the nucleotides in the oligonucleotide comprise a 2′-OMe modification. In some embodiments, about 80% of the nucleotides in the oligonucleotide comprise a 2′-OMe modification. In some embodiments, about 85% of the nucleotides in the oligonucleotide comprise a 2′-OMe modification. In some embodiments, about 90% of the nucleotides in the oligonucleotide comprise a 2′-OMe modification. In some embodiments, about 95% of the nucleotides in the oligonucleotide comprise a 2′-OMe modification.

In some embodiments, the oligonucleotide comprises a sense strand with about 10-20% (e.g., 10%, 11%, 12%, 13%, 14%, 15%, 16%, 17%, 18%, 19% or 20%) of the nucleotides of the sense strand comprising a 2′-F modification. In some embodiments, the oligonucleotide comprises a sense strand with about 21-30% (e.g., 21%, 22%, 23%, 24%, 25%, 26%, 27%, 28%, 29%, or 30%) of the nucleotides of the sense strand comprising a 2′-F modification. In some embodiments, the oligonucleotide comprises a sense strand with about 38-43% (e.g., 38%, 39%, 40%, 41%, 42%, or 43%) of the nucleotides of the sense strand comprising a 2′-F modification. In some embodiments, the oligonucleotide comprises a sense strand with about 38-43% (e.g., 38%, 39%, 40%, 41%, 42%, or 43%) of the nucleotides of the sense strand comprising a 2′-F modification. In some embodiments, the oligonucleotide comprises a sense strand with about 15% of the nucleotides of the sense strand comprising a 2′-F modification. In some embodiments, the oligonucleotide comprises a sense strand with about 16% of the nucleotides of the sense strand comprising a 2′-F modification. In some embodiments, the oligonucleotide comprises a sense strand with about 17% of the nucleotides of the sense strand comprising a 2′-F modification. In some embodiments, the oligonucleotide comprises a sense strand with about 18% of the nucleotides of the sense strand comprising a 2′-F modification. In some embodiments, the oligonucleotide comprises a sense strand with about 19% of the nucleotides of the sense strand comprising a 2′-F modification. In some embodiments, the oligonucleotide comprises a sense strand with about 20% of the nucleotides of the sense strand comprising a 2′-F modification.

In some embodiments, the oligonucleotide comprises an anti-sense strand with about 10-20% (e.g., 10%, 11%, 12%, 13%, 14%, 15%, 16%, 17%, 18%, 19% or 20%) of the nucleotides of the anti-sense strand comprising a 2′-F modification. In some embodiments, the oligonucleotide comprises an anti-sense strand with about 21-30% (e.g., 21%, 22%, 23%, 24%, 25%, 26%, 27%, 28%, 29%, or 30%) of the nucleotides of the anti-sense strand comprising a 2′-F modification. In some embodiments, the oligonucleotide comprises an anti-sense strand with about 38-43% (e.g., 38%, 39%, 40%, 41%, 42%, or 43%) of the nucleotides of the anti-sense strand comprising a 2′-F modification. In some embodiments, the oligonucleotide comprises an anti-sense strand with about 15% of the nucleotides of the anti-sense strand comprising a 2′-F modification. In some embodiments, the oligonucleotide comprises an anti-sense strand with about 16% of the nucleotides of the anti-sense strand comprising a 2′-F modification. In some embodiments, the oligonucleotide comprises an anti-sense strand with about 17% of the nucleotides comprising a 2′-F modification. In some embodiments, the oligonucleotide comprises an anti-sense strand with about 18% of the nucleotides of the strand comprising a 2′-F modification. In some embodiments, the oligonucleotide comprises an anti-sense strand with about 19% of the nucleotides of the strand comprising a 2′-F modification. In some embodiments, the oligonucleotide comprises an anti-sense strand with about 20% of the nucleotides of the strand comprising a 2′-F modification.

In some embodiments, about 14% of the nucleotides in the oligonucleotide comprise a 2′-F modification. In some embodiments, about 15% of the nucleotides in the oligonucleotide comprise a 2′-F modification. In some embodiments, about 16% of the nucleotides in the oligonucleotide comprise a 2′-F modification. In some embodiments, about 17% of the nucleotides in the oligonucleotide comprise a 2′-F modification. In some embodiments, about 18% of the nucleotides in the oligonucleotide comprise a 2′-F modification. In some embodiments, about 19% of the nucleotides in the oligonucleotide comprise a 2′-F modification. In some embodiments, about 20% of the nucleotides in the oligonucleotide comprise a 2′-F modification. In some embodiments, about 21% of the nucleotides in the oligonucleotide comprise a 2′-F modification. In some embodiments, about 22% of the nucleotides in the oligonucleotide comprise a 2′-F modification. In some embodiments, about 23% of the nucleotides in the oligonucleotide comprise a 2′-F modification. In some embodiments, about 24% of the nucleotides in the oligonucleotide comprise a 2′-F modification. In some embodiments, about 25% of the nucleotides in the oligonucleotide comprise a 2′-F modification.

Moreover, the oligonucleotides herein can have different modification patterns. In some embodiments, the modified oligonucleotide comprises a sense strand sequence having a modification pattern as set forth in Table 4 (as well as FIG. 1A or 1B) and an antisense strand having a modification pattern as set forth in Table 4.

In some embodiments, the oligonucleotide of the present disclosure includes 2′-fluoro (2′-F) modifications at one or more nucleotides at positions 7, 8, or 9 of the sense strand. In some embodiments, the oligonucleotide is such that one or more nucleotides at positions 2, 6, 14, or 16 of the antisense strand are modified with 2′-fluoro. In some embodiments, the oligonucleotide is such that one or more nucleotides at positions 1, 2, 3, 4, 5, 6, 10, 11, 12, 13, 14, 15, 16, 17, 18, or 19 of the sense strand are modified with 2′-O-methyl (2′-OMe). In some embodiments, the oligonucleotide is such that one or more nucleotides at positions 1, 3, 4, 5, 7 to 13, 15, 17, 18, 19, 20, or 21 of the antisense strand are modified with 2′-O-methyl.

In some embodiments, the oligonucleotide of the present disclosure includes at least one modified internucleotide linkage. The internucleotide linkage can be a phosphorothioate linkage or a phosphorodithioate linkage. In some embodiments, at least two terminal nucleotides on 5′ end or 3′ end of the antisense strand or the sense strand have a phosphorothioate linkage or a phosphorodithioate linkage. In some embodiments, at least three terminal nucleotides on 5′ end or 3′ end of the antisense strand or the sense strand have phosphorothioate linkages or a phosphorodithioate linkages.

In some embodiments, the oligonucleotide is such that the sense strand has 19 nucleotides and the internucleotide linkage of nucleotides between positions 1 and 2, 2 and 3, 17 and 18, or 18 and 19 of the sense strand are modified with the phosphorothioate linkage.

In some embodiments, the oligonucleotide is such that the antisense strand has 21 nucleotides and the internucleotide linkage of one or more nucleotides between positions 1 and 2, 2 and 3, 19 and 20, or 20 and 21 of the antisense strand are modified with the phosphorothioate linkage.

In some embodiments, the oligonucleotide of the present disclosure is selected from Table 4. Table 4 shows some embodiments of the modified siRNA sequences of the present disclosure.

TABLE 4
Nav1.8 siRNAs with Modifications

| | | | | Start position of |
|---|---|---|---|---|
| | | | | target region on |
| siRNA | | | SEQ | human SCN10A |
| No. | Strand | Sequence (5′ to 3′) | ID NO. | transcript |
| 283 | S | m(U)[ps].m(U)[ps].m(A)p.m(C)p.m(G)p.m(G) | 565 | 407 |
| | | p.[fl2r](U)p.[fl2r](C)p.[fl2r](A)p.m(C)p.m(U) | | |
| | | p.m(A)p.m(U)p.m(U)p.m(U)p.m(U)p.m(G)[ps].m(G) | | |
| | | [ps].m(A) | | |
| | AS | m(U)[ps].[fl2r](C)[ps].m(C)p.m(A)p.m(A) | 566 | |
| | | p.[fl2r](A)p.m(A)p.m(U)p.m(A)p.m(G)p.m(U) | | |
| | | p.m(G)p.m(A)p.[fl2r](C)p.m(C)p.[fl2r](G) | | |
| | | p.m(U)p.m(A)p.m(A)[ps].m(U)[ps].m(U) | | |
| 284 | S | m(A)[ps].m(C)[ps].m(G)p.m(G)p.m(U)p.m(C) | 567 | 409 |
| | | p.[fl2r](A)p.[fl2r](C)p.[fl2r](U)p.m(A)p.m(U) | | |
| | | p.m(U)p.m(U)p.m(U)p.m(G)p.m(G)p.m(U)[ps].m(U) | | |
| | | [ps].m(A) | | |
| | AS | m(U)[ps].[fl2r](A)[ps].m(A)p.m(C)p.m(C) | 568 | |
| | | p.[fl2r](A)p.m(A)p.m(A)p.m(A)p.m(U)p.m(A) | | |
| | | p.m(G)p.m(U)p.[fl2r](G)p.m(A)p.[fl2r](C) | | |
| | | p.m(C)p.m(G)p.m(U)[ps].m(U)[ps].m(U) | | |
| 285 | S | m(C)[ps].m(G)[ps].m(G)p.m(U)p.m(C)p.m(A) | 569 | 410 |
| | | p.[fl2r](C)p.[fl2r](U)p.[fl2r](A)p.m(U)p.m(U) | | |
| | | p.m(U)p.m(U)p.m(G)p.m(G)p.m(U)p.m(U)[ps].m(A) | | |
| | | [ps].m(A) | | |
| | AS | m(U)[ps].[fl2r](U)[ps].m(A)p.m(A)p.m(C) | 570 | |
| | | p.[fl2r](C)p.m(A)p.m(A)p.m(A)p.m(A)p.m(U) | | |
| | | p.m(A)p.m(G)p.[fl2r](U)p.m(G)p.[fl2r](A) | | |
| | | p.m(C)p.m(C)p.m(G)[ps].m(U)[ps].m(U) | | |
| 286 | S | m(U)[ps].m(A)[ps].m(A)p.m(U)p.m(U)p.m(G) | 571 | 426 |
| | | p.[fl2r](U)p.[fl2r](G)p.[fl2r](U)p.m(G) | | |
| | | p.m(U)p.m(G)p.m(C)p.m(A)p.m(U)p.m(G) | | |
| | | p.m(A)[ps].m(C)[ps].m(A) | | |
| | AS | m(U)[ps].[fl2r](G)[ps].m(U)p.m(C)p.m(A) | 572 | |
| | | p.[fl2r](U)p.m(G)p.m(C)p.m(A)p.m(C)p.m(A) | | |
| | | p.m(C)p.m(A)p.[fl2r](C)p.m(A)p.[fl2r](A) | | |
| | | p.m(U)p.m(U)p.m(A)[ps].m(U)[ps].m(U) | | |
| 287 | S | m(A)[ps].m(A)[ps].m(U)p.m(U)p.m(G)p.m(U) | 573 | 427 |
| | | p.[fl2r](G)p.[fl2r](U)p.[fl2r](G)p.m(U)p.m(G) | | |
| | | p.m(C)p.m(A)p.m(U)p.m(G)p.m(A)p.m(C)[ps].m(C) | | |
| | | [ps].m(A) | | |
| | AS | m(U)[ps].[fl2r](G)[ps].m(G)p.m(U)p.m(C) | 574 | |
| | | p.[fl2r](A)p.m(U)p.m(G)p.m(C)p.m(A)p.m(C) | | |
| | | p.m(A)p.m(C)p.[fl2r](A)p.m(C)p.[fl2r](A) | | |
| | | p.m(A)p.m(U)p.m(U)[ps].m(U)[ps].m(U) | | |
| 288 | S | m(U)[ps].m(G)[ps].m(U)p.m(G)p.m(C)p.m(A) | 575 | 434 |
| | | p.[fl2r](U)p.[fl2r](G)p.[fl2r](A)p.m(C) | | |
| | | p.m(C)p.m(C)p.m(G)p.m(A)p.m(A)p.m(C)p.m(U) | | |
| | | [ps].m(G)[ps].m(A) | | |
| | AS | m(U)[ps].[fl2r](C)[ps].m(A)p.m(G)p.m(U) | 576 | |
| | | p.[fl2r](U)p.m(C)p.m(G)p.m(G)p.m(G)p.m(U) | | |
| | | p.m(C)p.m(A)p.[fl2r](U)p.m(G)p.[fl2r](C) | | |
| | | p.m(A)p.m(C)p.m(A)[ps].m(U)[ps].m(U) | | |
| 289 | S | m(G)[ps].m(U)[ps].m(G)p.m(C)p.m(A)p.m(U) | 577 | 435 |
| | | p.[fl2r](G)p.[fl2r](A)p.[fl2r](C)p.m(C) | | |
| | | p.m(C)p.m(G)p.m(A)p.m(A)p.m(C)p.m(U)p.m(G) | | |
| | | [ps].m(A)[ps].m(A) | | |
| | AS | m(U)[ps].[fl2r](U)[ps].m(C)p.m(A)p.m(G) | 578 | |
| | | p.[fl2r](U)p.m(U)p.m(C)p.m(G)p.m(G)p.m(G) | | |
| | | p.m(U)p.m(C)p.[fl2r](A)p.m(U)p.[fl2r](G) | | |
| | | p.m(C)p.m(A)p.m(C)[ps].m(U)[ps].m(U) | | |
| 290 | S | m(U)[ps].m(G)[ps].m(U)p.m(C)p.m(U)p.m(A) | 579 | 532 |
| | | p.[fl2r](A)p.[fl2r](A)p.[fl2r](U)p.m(G) | | |
| | | p.m(A)p.m(G)p.m(U)p.m(U)p.m(C)p.m(A)p.m(C) | | |
| | | [ps].m(G)[ps].m(A) | | |
| | AS | m(U)[ps].[fl2r](C)[ps].m(G)p.m(U)p.m(G) | 580 | |
| | | p.[fl2r](A)p.m(A)p.m(C)p.m(U)p.m(C)p.m(A) | | |
| | | p.m(U)p.m(U)p.[fl2r](U)p.m(A)p.[fl2r](G) | | |
| | | p.m(A)p.m(C)p.m(A)[ps].m(U)[ps].m(U) | | |
| 291 | S | m(C)[ps].m(U)[ps].m(A)p.m(A)p.m(A)p.m(U) | 581 | 535 |
| | | p.[fl2r](G)p.[fl2r](A)p.[fl2r](G)p.m(U) | | |
| | | p.m(U)p.m(C)p.m(A)p.m(C)p.m(G)p.m(U)p.m(A) | | |
| | | [ps].m(C)[ps].m(A) | | |
| | AS | m(U)[ps].[fl2r](G)[ps].m(U)p.m(A)p.m(C) | 582 | |
| | | p.[fl2r](G)p.m(U)p.m(G)p.m(A)p.m(A)p.m(C) | | |
| | | p.m(U)p.m(C)p.[fl2r](A)p.m(U)p.[fl2r](U) | | |
| | | p.m(U)p.m(A)p.m(G)[ps].m(U)[ps].m(U) | | |
| 292 | S | m(A)[ps].m(A)[ps].m(A)p.m(U)p.m(G)p.m(A) | 583 | 537 |
| | | p.[fl2r](G)p.[fl2r](U)p.[fl2r](U)p.m(C) | | |
| | | p.m(A)p.m(C)p.m(G)p.m(U)p.m(A)p.m(C)p.m(C) | | |
| | | [ps].m(U)[ps].m(A) | | |
| | AS | m(U)[ps].[fl2r](A)[ps].m(G)p.m(G)p.m(U) | 584 | |
| | | p.[fl2r](A)p.m(C)p.m(G)p.m(U)p.m(G)p.m(A) | | |
| | | p.m(A)p.m(C)p.[fl2r](U)p.m(C)p.[fl2r](A) | | |
| | | p.m(U)p.m(U)p.m(U)[ps].m(U)[ps].m(U) | | |
| 293 | S | m(G)[ps].m(G)[ps].m(A)p.m(U)p.m(U)p.m(U) | 585 | 576 |
| | | p.[fl2r](U)p.[fl2r](A)p.[fl2r](G)p.m(C)p.m(G) | | |
| | | p.m(U)p.m(C)p.m(A)p.m(U)p.m(U)p.m(A)[ps].m(C) | | |
| | | [ps].m(A) | | |
| | AS | m(U)[ps].[fl2r](G)[ps].m(U)p.m(A)p.m(A) | 586 | |
| | | p.[fl2r](U)p.m(G)p.m(A)p.m(C)p.m(G)p.m(C) | | |
| | | p.m(U)p.m(A)p.[fl2r](A)p.m(A)p.[fl2r](A) | | |
| | | p.m(U)p.m(C)p.m(C)[ps].m(U)[ps].m(U) | | |
| 294 | S | m(A)[ps].m(C)[ps].m(A)p.m(A)p.m(C)p.m(C) | 587 | 856 |
| | | p.[fl2r](A)p.[fl2r](A)p.[fl2r](C)p.m(U) | | |
| | | p.m(A)p.m(C)p.m(U)p.m(C)p.m(A)p.m(U)p.m(C) | | |
| | | [ps].m(U)[ps].m(A) | | |
| | AS | m(U)[ps].[fl2r](A)[ps].m(G)p.m(A)p.m(U) | 588 | |
| | | p.[fl2r](G)p.m(A)p.m(G)p.m(U)p.m(A)p.m(G) | | |
| | | p.m(U)p.m(U)p.[fl2r](G)p.m(G)p.[fl2r](U) | | |
| | | p.m(U)p.m(G)p.m(U)[ps].m(U)[ps].m(U) | | |
| 295 | S | m(A)[ps].m(A)[ps].m(C)p.m(U)p.m(U)p.m(C) | 589 | 981 |
| | | p.[fl2r](U)p.[fl2r](G)p.[fl2r](A)p.m(C) | | |
| | | p.m(A)p.m(A)p.m(C)p.m(C)p.m(C)p.m(G)p.m(G) | | |
| | | [ps].m(A)[ps].m(A) | | |
| | AS | m(U)[ps].[fl2r](U)[ps].m(C)p.m(C)p.m(G) | 590 | |
| | | p.[fl2r](G)p.m(G)p.m(U)p.m(U)p.m(G)p.m(U) | | |
| | | p.m(C)p.m(A)p.[fl2r](G)p.m(A)p.[fl2r](A) | | |
| | | p.m(G)p.m(U)p.m(U)[ps].m(U)[ps].m(U) | | |
| 296 | S | m(C)[ps].m(U)[ps].m(U)p.m(C)p.m(U)p.m(G) | 591 | 983 |
| | | p.[fl2r](A)p.[fl2r](C)p.[fl2r](A)p.m(A)p.m(C) | | |
| | | p.m(C)p.m(C)p.m(G)p.m(G)p.m(A)p.m(U)[ps].m(U) | | |
| | | [ps].m(A) | | |
| | AS | m(U)[ps].[f]2r](A)[ps].m(A)p.m(U)p.m(C) | 592 | |
| | | p.[f]2r](C)p.m(G)p.m(G)p.m(G)p.m(U)p.m(U) | | |
| | | p.m(G)p.m(U)p.[fl2r](C)p.m(A)p.[fl2r](G) | | |
| | | p.m(A)p.m(A)p.m(G)[ps].m(U)[ps].m(U) | | |
| 297 | S | m(C)[ps].m(U)[ps].m(G)p.m(A)p.m(C)p.m(A) | 593 | 986 |
| | | p.[f]2r](A)p.[fl2r](C)p.[fl2r](C)p.m(C) | | |
| | | p.m(G)p.m(G)p.m(A)p.m(U)p.m(U)p.m(U)p.m(U) | | |
| | | [ps].m(A)[ps].m(A) | | |
| | AS | m(U)[ps].[fl2r](U)[ps].m(A)p.m(A)p.m(A) | 594 | |
| | | p.[fl2r](A)p.m(U)p.m(C)p.m(C)p.m(G)p.m(G) | | |
| | | p.m(G)p.m(U)p.[fl2r](U)p.m(G)p.[fl2r](U) | | |
| | | p.m(C)p.m(A)p.m(G)[ps].m(U)[ps].m(U) | | |
| 298 | S | m(U)[ps].m(G)[ps].m(A)p.m(C)p.m(A)p.m(A) | 595 | 987 |
| | | p.[fl2r](C)p.[fl2r](C)p.[fl2r](C)p.m(G) | | |
| | | p.m(G)p.m(A)p.m(U)p.m(U)p.m(U)p.m(U)p.m(A) | | |
| | | [ps].m(A)[ps].m(A) | | |
| | AS | m(U)[ps].[fl2r](U)[ps].m(U)p.m(A)p.m(A) | 596 | |
| | | p.[fl2r](A)p.m(A)p.m(U)p.m(C)p.m(C)p.m(G) | | |
| | | p.m(G)p.m(G)p.[fl2r](U)p.m(U)p.[fl2r](G) | | |
| | | p.m(U)p.m(C)p.m(A)[ps].m(U)[ps].m(U) | | |
| 299 | S | m(A)[ps].m(C)[ps].m(A)p.m(A)p.m(C)p.m(C) | 597 | 989 |
| | | p.[fl2r](C)p.[fl2r](G)p.[fl2r](G)p.m(A) | | |
| | | p.m(U)p.m(U)p.m(U)p.m(U)p.m(A)p.m(A)p.m(C) | | |
| | | [ps].m(U)[ps].m(A) | | |
| | AS | m(U)[ps].[fl2r](A)[ps].m(G)p.m(U)p.m(U) | 598 | |
| | | p.[fl2r](A)p.m(A)p.m(A)p.m(A)p.m(U)p.m(C) | | |
| | | p.m(C)p.m(G)p.[fl2r](G)p.m(G)p.[fl2r](U) | | |
| | | p.m(U)p.m(G)p.m(U)[ps].m(U)[ps].m(U) | | |
| 300 | S | m(C)[ps].m(A)[ps].m(A)p.m(C)p.m(C)p.m(C) | 599 | 990 |
| | | p.[fl2r](G)p.[fl2r](G)p.[fl2r](A)p.m(U) | | |
| | | p.m(U)p.m(U)p.m(U)p.m(A)p.m(A)p.m(C)p.m(U) | | |
| | | [ps].m(A)[ps].m(A) | | |
| | AS | m(U)[ps].[fl2r](U)[ps].m(A)p.m(G)p.m(U) | 600 | |
| | | p.[fl2r](U)p.m(A)p.m(A)p.m(A)p.m(A)p.m(U) | | |
| | | p.m(C)p.m(C)p.[fl2r](G)p.m(G)p.[fl2r](G) | | |
| | | p.m(U)p.m(U)p.m(G)[ps].m(U)[ps].m(U) | | |
| 301 | S | m(U)[ps].m(G)[ps].m(A)p.m(U)p.m(C)p.m(U) | 601 | 1172 |
| | | p.[fl2r](U)p.[fl2r](G)p.[fl2r](G)p.m(C) | | |
| | | p.m(U)p.m(G)p.m(U)p.m(A)p.m(G)p.m(U)p.m(C) | | |
| | | [ps].m(A)[ps].m(A) | | |
| | AS | m(U)[ps].[fl2r](U)[ps].m(G)p.m(A)p.m(C) | 602 | |
| | | p.[fl2r](U)p.m(A)p.m(C)p.m(A)p.m(G)p.m(C) | | |
| | | p.m(C)p.m(A)p.[fl2r](A)p.m(G)p.[fl2r](A) | | |
| | | p.m(U)p.m(C)p.m(A)[ps].m(U)[ps].m(U) | | |
| 302 | S | m(U)[ps].m(A)[ps].m(C)p.m(C)p.m(U)p.m(U) | 603 | 2073 |
| | | p.[fl2r](C)p.[fl2r](G)p.[fl2r](A)p.m(A) | | |
| | | p.m(G)p.m(C)p.m(C)p.m(A)p.m(U)p.m(G)p.m(C) | | |
| | | [ps].m(U)[ps].m(A) | | |
| | AS | m(U)[ps].[fl2r](A)[ps].m(G)p.m(C)p.m(A) | 604 | |
| | | p.[fl2r](U)p.m(G)p.m(G)p.m(C)p.m(U)p.m(U) | | |
| | | p.m(C)p.m(G)p.[fl2r](A)p.m(A)p.[fl2r](G) | | |
| | | p.m(G)p.m(U)p.m(A)[ps].m(U)[ps].m(U) | | |
| 303 | S | m(U)[ps].m(U)[ps].m(A)p.m(C)p.m(G)p.m(G) | 565 | 407 |
| | | p.[fl2r](U)p.[fl2r](C)p.[fl2r](A)p.m(C) | | |
| | | p.m(U)p.m(A)p.m(U)p.m(U)p.m(U)p.m(U)p.m(G) | | |
| | | [ps].m(G)[ps].m(A) | | |
| | AS | [5vp_una2moe](U)[ps].[fl2r](C)[ps].m(C)p.m(A) | 605 | |
| | | p.m(A)p.[fl2r](A)p.m(A)p.m(U)p.m(A)p.m(G) | | |
| | | p.m(U)p.m(G)p.m(A)p.[fl2r](C)p.m(C)p.[fl2r](G) | | |
| | | p.m(U)p.m(A)p.m(A)[ps].m(U)[ps].m(U) | | |
| 304 | S | m(A)[ps].m(C)[ps].m(G)p.m(G)p.m(U)p.m(C) | 567 | 409 |
| | | p.[fl2r](A)p.[fl2r](C)p.[fl2r](U)p.m(A) | | |
| | | p.m(U)p.m(U)p.m(U)p.m(U)p.m(G)p.m(G)p.m(U) | | |
| | | [ps].m(U)[ps].m(A) | | |
| | AS | [5vp_una2moe](U)[ps].[fl2r](A)[ps].m(A) | 606 | |
| | | p.m(C)p.m(C)p.[fl2r](A)p.m(A)p.m(A)p.m(A) | | |
| | | p.m(U)p.m(A)p.m(G)p.m(U)p.[fl2r](G)p.m(A) | | |
| | | p.[fl2r](C)p.m(C)p.m(G)p.m(U)[ps].m(U)[ps].m(U) | | |
| 305 | S | m(C)[ps].m(G)[ps].m(G)p.m(U)p.m(C)p.m(A) | 569 | 410 |
| | | p.[fl2r](C)p.[fl2r](U)p.[fl2r](A)p.m(U)p.m | | |
| | | (U)p.m(U)p.m(U)p.m(G)p.m(G)p.m(U)p.m(U) | | |
| | | [ps].m(A)[ps].m(A) | | |
| | AS | [5vp_una2moe](U)[ps].[fl2r](U)[ps].m(A) | 607 | |
| | | p.m(A)p.m(C)p.[fl2r](C)p.m(A)p.m(A)p.m(A) | | |
| | | p.m(A)p.m(U)p.m(A)p.m(G)p.[fl2r](U)p.m(G) | | |
| | | p.[fl2r(A)p.m(C)p.m(C)p.m(G)[ps].m(U)[ps].m(U) | | |
| 306 | S | m(U)[ps].m(A)[ps].m(A)p.m(U)p.m(U)p.m(G) | 571 | 426 |
| | | p.[fl2r](U)p.[fl2r](G)p.[fl2r](U)p.m(G)p.m(U) | | |
| | | p.m(G)p.m(C)p.m(A)p.m(U)p.m(G)p.m(A)[ps].m(C) | | |
| | | [ps].m(A) | | |
| | AS | [5vp_una2moe](U)[ps].[fl2r](G)[ps].m(U) | 608 | |
| | | p.m(C)p.m(A)p.[fl2r](U)p.m(G)p.m(C)p.m(A) | | |
| | | p.m(C)p.m(A)p.m(C)p.m(A)p.[fl2r](C)p.m(A) | | |
| | | p.[fl2r](A)p.m(U)p.m(U)p.m(A)[ps].m(U)[ps].m(U) | | |
| 307 | S | m(A)[ps].m(A)[ps].m(U)p.m(U)p.m(G)p.m(U) | 573 | 427 |
| | | p.[fl2r](G)p.[fl2r](U)p.[fl2r](G)p.m(U)p.m(G) | | |
| | | p.m(C)p.m(A)p.m(U)p.m(G)p.m(A)p.m(C)[ps].m(C) | | |
| | | [ps].m(A) | | |
| | AS | [5vp_una2moe](U)[ps].[fl2r](G)[ps].m(G)p.m(U) | 609 | |
| | | p.m(C)p.[fl2r](A)p.m(U)p.m(G)p.m(C)p.m(A) | | |
| | | p.m(C)p.m(A)p.m(C)p.[fl2r](A)p.m(C)p.[fl2r](A) | | |
| | | p.m(A)p.m(U)p.m(U)[ps].m(U)[ps].m(U) | | |
| 308 | S | m(U)[ps].m(G)[ps].m(U)p.m(G)p.m(C)p.m(A) | 575 | 434 |
| | | p.[fl2r](U)p.[fl2r](G)p.[f]2r](A)p.m(C) | | |
| | | p.m(C)p.m(C)p.m(G)p.m(A)p.m(A)p.m(C)p.m(U) | | |
| | | [ps].m(G)[ps].m(A) | | |
| | AS | [5vp_una2moe](U)[ps].[fl2r](C)[ps].m(A) | 610 | |
| | | p.m(G)p.m(U)p.[fl2r](U)p.m(C)p.m(G)p.m(G) | | |
| | | p.m(G)p.m(U)p.m(C)p.m(A)p.[fl2r](U)p.m(G) | | |
| | | p.[fl2r](C)p.m(A)p.m(C)p.m(A)[ps].m(U)[ps].m(U) | | |
| 309 | S | m(G)[ps].m(U)[ps].m(G)p.m(C)p.m(A)p.m(U) | 577 | 435 |
| | | p.[fl2r](G)p.[fl2r](A)p.[fl2r](C)p.m(C)p.m(C) | | |
| | | p.m(G)p.m(A)p.m(A)p.m(C)p.m(U)p.m(G)[ps].m(A) | | |
| | | [ps].m(A) | | |
| | AS | [5vp_una2moe](U)[ps].[fl2r](U)[ps].m(C) | 611 | |
| | | p.m(A)p.m(G)p.[fl2r](U)p.m(U)p.m(C)p.m(G) | | |
| | | p.m(G)p.m(G)p.m(U)p.m(C)p.[fl2r](A)p.m(U) | | |
| | | p.[fl2rl(G)p.m(C)p.m(A)p.m(C)[ps].m(U)[ps].m(U) | | |
| 310 | S | m(U)[ps].m(G)[ps].m(U)p.m(C)p.m(U)p.m(A) | 579 | 532 |
| | | p.[fl2r](A)p.[fl2r](A)p.[fl2r](U)p.m(G)p.m(A) | | |
| | | p.m(G)p.m(U)p.m(U)p.m(C)p.m(A)p.m(C)[ps].m(G) | | |
| | | [ps].m(A) | | |
| | AS | [5vp_una2moe](U)[ps].[fl2r](C)[ps].m(G)p.m(U) | 612 | |
| | | p.m(G)p.[fl2r](A)p.m(A)p.m(C)p.m(U)p.m(C) | | |
| | | p.m(A)p.m(U)p.m(U)p.[fl2r](U)p.m(A)p.[fl2r](G) | | |
| | | p.m(A)p.m(C)p.m(A)[ps].m(U)[ps].m(U) | | |
| 311 | S | m(C)[ps].m(U)[ps].m(A)p.m(A)p.m(A)p.m(U) | 581 | 535 |
| | | p.[fl2r](G)p.[fl2r](A)p.[fl2r](G)p.m(U)p.m(U) | | |
| | | p.m(C)p.m(A)p.m(C)p.m(G)p.m(U)p.m(A)[ps].m(C) | | |
| | | [ps].m(A) | | |
| | AS | [5vp_una2moe](U)[ps].[fl2r](G)[ps].m(U) | 613 | |
| | | p.m(A)p.m(C)p.[fl2r](G)p.m(U)p.m(G)p.m(A) | | |
| | | p.m(A)p.m(C)p.m(U)p.m(C)p.[fl2r](A)p.m(U) | | |
| | | p.[fl2r](U)p.m(U)p.m(A)p.m(G)[ps].m(U)[ps].m(U) | | |
| 312 | S | m(A)[ps].m(A)[ps].m(A)p.m(U)p.m(G)p.m(A) | 583 | 537 |
| | | p.[fl2r](G)p.[fl2r](U)p.[fl2r](U)p.m(C)p.m(A) | | |
| | | p.m(C)p.m(G)p.m(U)p.m(A)p.m(C)p.m(C)[ps].m(U) | | |
| | | [ps].m(A) | | |
| | AS | [5vp_una2moe](U)[ps].[fl2r](A)[ps].m(G)p.m(G) | 614 | |
| | | p.m(U)p.[fl2r](A)p.m(C)p.m(G)p.m(U)p.m(G)p.m(A) | | |
| | | p.m(A)p.m(C)p.[fl2r](U)p.m(C)p.[fl2r](A) | | |
| | | p.m(U)p.m(U)p.m(U)[ps].m(U)[ps].m(U) | | |
| 313 | S | m(G)[ps].m(G)[ps].m(A)p.m(U)p.m(U)p.m(U) | 585 | 576 |
| | | p.[fl2r](U)p.[fl2r](A)p.[fl2r](G)p.m(C)p.m(G) | | |
| | | p.m(U)p.m(C)p.m(A)p.m(U)p.m(U)p.m(A)[ps].m(C) | | |
| | | [ps].m(A) | | |
| | AS | [5vp_una2moe](U)[ps].[fl2r](G)[ps].m(U) | 615 | |
| | | p.m(A)p.m(A)p.[fl2r](U)p.m(G)p.m(A)p.m(C) | | |
| | | p.m(G)p.m(C)p.m(U)p.m(A)p.[fl2r](A)p.m(A) | | |
| | | p.[fl2r](A)p.m(U)p.m(C)p.m(C)[ps].m(U)[ps].m(U) | | |
| 314 | S | m(A)[ps].m(C)[ps].m(A)p.m(A)p.m(C)p.m(C) | 587 | 856 |
| | | p.[fl2r](A)p.[fl2r](A)p.[fl2r](C)p.m(U)p.m(A) | | |
| | | p.m(C)p.m(U)p.m(C)p.m(A)p.m(U)p.m(C)[ps].m(U) | | |
| | | [ps].m(A) | | |
| | AS | [5vp_una2moe](U)[ps].[fl2r](A)[ps].m(G)p.m(A) | 616 | |
| | | p.m(U)p.[fl2r](G)p.m(A)p.m(G)p.m(U)p.m(A)p.m(G) | | |
| | | p.m(U)p.m(U)p.[fl2r](G)p.m(G)p.[fl2r](U)p.m(U) | | |
| | | p.m(G)p.m(U)[ps].m(U)[ps].m(U) | | |
| 315 | S | m(A)[ps].m(A)[ps].m(C)p.m(U)p.m(U)p.m(C) | 589 | 981 |
| | | p.[fl2r](U)p.[fl2r](G)p.[fl2r](A)p.m(C) | | |
| | | p.m(A)p.m(A)p.m(C)p.m(C)p.m(C)p.m(G)p.m(G) | | |
| | | [ps].m(A)[ps].m(A) | | |
| | AS | [5vp_una2moe](U)[ps].[fl2r](U)[ps].m(C) | 617 | |
| | | p.m(C)p.m(G)p.[fl2r](G)p.m(G)p.m(U)p.m(U) | | |
| | | p.m(G)p.m(U)p.m(C)p.m(A)p.[fl2r](G)p.m(A) | | |
| | | p.[fl2r](A)p.m(G)p.m(U)p.m(U)[ps].m(U)[ps].m(U) | | |
| 316 | S | m(C)[ps].m(U)[ps].m(U)p.m(C)p.m(U)p.m(G) | 591 | 983 |
| | | p.[fl2r](A)p.[fl2r](C)p.[fl2r](A)p.m(A)p.m(C) | | |
| | | p.m(C)p.m(C)p.m(G)p.m(G)p.m(A)p.m(U)[ps].m(U) | | |
| | | [ps].m(A) | | |
| | AS | [5vp_una2moe](U)[ps].[fl2r](A)[ps].m(A) | 618 | |
| | | p.m(U)p.m(C)p.[fl2r](C)p.m(G)p.m(G)p.m(G) | | |
| | | p.m(U)p.m(U)p.m(G)p.m(U)p.[fl2r](C)p.m(A) | | |
| | | p.[fl2r](G)p.m(A)p.m(A)p.m(G)[ps].m(U)[ps].m(U) | | |
| 317 | S | m(C)[ps].m(U)[ps].m(G)p.m(A)p.m(C)p.m(A) | 593 | 986 |
| | | p.[fl2r](A)p.[fl2r](C)p.[fl2r](C)p.m(C)p.m(G) | | |
| | | p.m(G)p.m(A)p.m(U)p.m(U)p.m(U)p.m(U)[ps].m(A) | | |
| | | [ps].m(A) | | |
| | AS | [5vp_una2moe](U)[ps].[fl2r](U)[ps].m(A)p.m(A) | 619 | |
| | | p.m(A)p.[fl2r](A)p.m(U)p.m(C)p.m(C)p.m(G)p.m(G) | | |
| | | p.m(G)p.m(U)p.[fl2r](U)p.m(G)p.[fl2r](U) | | |
| | | p.m(C)p.m(A)p.m(G)[ps].m(U)[ps].m(U) | | |
| 318 | S | m(U)[ps].m(G)[ps].m(A)p.m(C)p.m(A)p.m(A) | 595 | 987 |
| | | p.[fl2r](C)p.[fl2r](C)p.[fl2r](C)p.m(G) | | |
| | | p.m(G)p.m(A)p.m(U)p.m(U)p.m(U)p.m(U)p.m(A) | | |
| | | [ps].m(A)[ps].m(A) | | |
| | AS | [5vp_una2moe](U)[ps].[fl2r](U)[ps].m(U) | 620 | |
| | | p.m(A)p.m(A)p.[fl2r](A)p.m(A)p.m(U)p.m(C) | | |
| | | p.m(C)p.m(G)p.m(G)p.m(G)p.[fl2r](U)p.m(U) | | |
| | | p.[fl2r](G)p.m(U)p.m(C)p.m(A)[ps].m(U)[ps].m(U) | | |
| 319 | S | m(A)[ps].m(C)[ps].m(A)p.m(A)p.m(C)p.m(C) | 597 | 989 |
| | | p.[fl2r](C)p.[fl2r](G)p.[fl2r](G)p.m(A)p.m(U) | | |
| | | p.m(U)p.m(U)p.m(U)p.m(A)p.m(A)p.m(C)[ps].m(U) | | |
| | | [ps].m(A) | | |
| | AS | [5vp_una2moe](U)[ps].[fl2r](A)[ps].m(G) | 621 | |
| | | p.m(U)p.m(U)p.[fl2r](A)p.m(A)p.m(A)p.m(A) | | |
| | | p.m(U)p.m(C)p.m(C)p.m(G)p.[fl2r](G)p.m(G) | | |
| | | p.[fl2r](U)p.m(U)p.m(G)p.m(U)[ps].m(U)[ps].m(U) | | |
| 320 | S | m(C)[ps].m(A)[ps].m(A)p.m(C)p.m(C)p.m(C) | 599 | 990 |
| | | p.[fl2r](G)p.[fl2r](G)p.[fl2r](A)p.m(U)p.m(U) | | |
| | | p.m(U)p.m(U)p.m(A)p.m(A)p.m(C)p.m(U)[ps].m(A) | | |
| | | [ps].m(A) | | |
| | AS | [5vp_una2moe](U)[ps].[fl2r](U)[ps].m(A)p.m(G) | 622 | |
| | | p.m(U)p.[fl2r](U)p.m(A)p.m(A)p.m(A)p.m(A)p.m(U) | | |
| | | p.m(C)p.m(C)p.[fl2r](G)p.m(G)p.[fl2r](G) | | |
| | | p.m(U)p.m(U)p.m(G)[ps].m(U)[ps].m(U) | | |
| 321 | S | m(U)[ps].m(G)[ps].m(A)p.m(U)p.m(C)p.m(U) | 601 | 1172 |
| | | p.[fl2r](U)p.[fl2r](G)p.[fl2r](G)p.m(C) | | |
| | | p.m(U)p.m(G)p.m(U)p.m(A)p.m(G)p.m(U)p.m(C) | | |
| | | [ps].m(A)[ps].m(A) | | |
| | AS | [5vp_una2moe](U)[ps].[fl2r](U)[ps].m(G) | 623 | |
| | | p.m(A)p.m(C)p.[fl2r](U)p.m(A)p.m(C)p.m(A) | | |
| | | p.m(G)p.m(C)p.m(C)p.m(A)p.[fl2r](A)p.m(G) | | |
| | | p.[fl2r](A)p.m(U)p.m(C)p.m(A)[ps].m(U)[ps].m(U) | | |
| 322 | S | m(U)[ps].m(A)[ps].m(C)p.m(C)p.m(U)p.m(U) | 603 | 2073 |
| | | p.[fl2r](C)p.[fl2r](G)p.[fl2r](A)p.m(A) | | |
| | | p.m(G)p.m(C)p.m(C)p.m(A)p.m(U)p.m(G)p.m(C) | | |
| | | [ps].m(U)[ps].m(A) | | |
| | AS | [5vp_una2moe](U)[ps].[fl2r](A)[ps].m(G)p.m(C) | 624 | |
| | | p.m(A)p.[fl2r](U)p.m(G)p.m(G)p.m(C)p.m(U) | | |
| | | p.m(U)p.m(C)p.m(G)p.[fl2r](A)p.m(A)p.[fl2r](G) | | |
| | | p.m(G)p.m(U)p.m(A)[ps].m(U)[ps].m(U) | | |

Legend: (N) = 2′-OH (ribose; ex: A, G, C, or U); m(N) = 2′-O-Methylribose; p = Phosphate; [fl2r] (N) = 2′-Fluororibose; [ps] = Phosphorothioate backbone modification; 5vp_una2moe = Vinylphosphonate-2′-O-MethoxyEthylRibose-UNA-U; “S” means the sense strand; “AS” means the antisense strand. On the 5′-end of the sense strand, there can be either a free C6—NH₂ or cholesterol-conjugated to the amino handle.

The sense strand and antisense strand of oligonucleotides of the present disclosure can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.). John Wiley & Sons. Inc., New York, NY. USA), H-phosphonate, phosphotriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene)amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

In addition to the above modifications, the oligonucleotides herein also comprise one or more modified nucleobases. In some embodiments, modified nucleobases (also referred to herein as base analogs) are linked at the 1′ position of a nucleotide sugar moiety. In some embodiments, the modified nucleobase is a nitrogenous base. In other embodiments, the modified nucleobase does not contain nitrogen atom. See, e.g., US Patent Application Publication No. 2008/0274462. In certain other embodiments, the modified nucleotide is a universal base. However, in certain embodiments, the modified nucleotide does not contain a nucleobase (abasic).

With regard to universal bases, they comprise a heterocyclic moiety located at the 1′ position of a nucleotide sugar moiety in a modified nucleotide, or the equivalent position in a nucleotide sugar moiety substitution, that, when present in a duplex, is positioned opposite more than one type of base without substantially altering structure of the duplex. Moreover. and compared to a reference single strand (ss) nucleic acid (e.g., oligonucleotide) that is fully complementary to a target nucleic acid, a ss nucleic acid having a universal base forms a duplex with the target nucleic acid that has a lower melting temperature (Tₘ) than a duplex formed with the complementary nucleic acid. However, when compared to a reference ss nucleic acid in which the universal base has been replaced with a base to generate a single mismatch, the ss nucleic acid having the universal base forms a duplex with the target nucleic acid that has a higher Tₘ than a duplex formed with the nucleic acid having the mismatched base.

The oligonucleotides herein, or a pharmaceutically acceptable salt thereof (e.g., trifluroacetate salts, acetate salts, or hydrochloride salts), are incorporated into a formulation or pharmaceutical composition. Various formulations have been developed to facilitate oligonucleotide use. For example, oligonucleotides can be delivered to an individual or a cellular environment using a formulation that minimizes degradation, facilitates delivery and/or uptake, or provides another beneficial property to the oligonucleotides in the formulation. In some embodiments, the oligonucleotides are formulated in buffer solutions such as phosphate buffered saline solutions, liposomes, micellar structures, and capsids.

To improve in vivo compatibility and effectiveness, the oligonucleotides may be reacted with any of a number of inorganic and organic acids/bases to form pharmaceutically acceptable acid/base addition salts. Pharmaceutically acceptable salts and common methodologies for preparing them are well known in the art.

### Antibodies (TfR Binding Proteins) and Antibody-Oligonucleotide Conjugates

One aspect of the present disclosure is based, at least in part, on the development of transferrin receptor binding proteins (also referred to herein as anti-TfR antibodies or antigen-binding fragments thereof) that bind transferrin receptor, have low immunogenicity risk, and may be used for delivery of a molecular payload (such as a therapeutic, single stranded RNA (ssRNA), ASO, dsRNA, siRNA, or a drug) to different tissues in a subject's body, such as, muscle, pancreas, brain, central nervous system (CNS), peripheral nervous system (PNS), or to different cell types, such as, cancerous cells, or dorsal root ganglion (DRG) neurons.

In another aspect, provided herein are conjugates comprising human TfR binding proteins described herein and an oligonucleotide. In some embodiments, the oligonucleotide is a single stranded RNA (ssRNA). In some embodiments, the oligonucleotide is a double stranded RNA (dsRNA). In some embodiments, the oligonucleotide comprises a sense strand and/or an antisense stand, wherein the antisense strand is complementary to at least a portion of the SCN10A mRNA. In some embodiments, the oligonucleotide comprises a sense strand and/or an antisense stand, wherein the antisense strand is complementary to a portion of SNC10A mRNA.

In some aspects, the present disclosure provides data demonstrating that certain conjugates including the transferrin binding proteins (TBPs; also described herein as anti-TfR antibodies) described herein have superior benefit in that they have reduced immunogenicity risk as compared to other conjugates having anti-TfR antibodies. In some aspects, the present disclosure provides conjugates with TBPs that have superior benefit in that they can deliver a molecular payload to dorsal root ganglion (DRG) neurons. In some aspects, the present disclosure provides conjugates with TBPs that have superior benefit in that they can deliver a molecular payload to dorsal root ganglion (DRG) neurons with reduced immunogenicity risk as compared to other anti-TfR antibodies.

In some embodiments, transferrin receptor binding proteins (TBPs) described herein bind to human transferrin receptor with high specificity, affinity, and with reduced immunogenicity risk. In some embodiments, the TBPs provide for a range of affinities to TfR.

In some embodiments, the TBPs described herein may be used for targeting tissues and/or cells that express TfR. In some embodiments, the TBPs described herein may be used to deliver a molecular payload to a target cell or tissue (e.g., a cell or tissue that expresses transferrin receptor).

In some aspects the present disclosure is related to complexes (or conjugates) including at least one TBP of the present disclosure conjugated (e.g., covalently) to at least one molecular payload (e.g., a therapeutic agent, siRNA). In some embodiments, the TBPs of the present disclosure may be used to deliver the conjugate (which includes the TBP and a molecular payload) to a cell or a tissue that expresses TfR1 (e.g., DRG, or the brain) for treating or reducing pain; and/or treating a disease related to SCN10A.

One aspect of the present disclosure is related to a conjugate comprising the oligonucleotide of the present disclosure and an anti-transferrin receptor antibody (anti-TfR antibody) or fragment thereof conjugated to the sense strand of the oligonucleotide or the antisense strand of the oligonucleotide. In some embodiments, the anti-transferrin receptor antibody (anti-TfR antibody) or fragment thereof is conjugated to at least one sense strand of the oligonucleotide. In some embodiments, the anti-transferrin receptor antibody or antigen binding fragment thereof conjugated to at least two sense strands of at least two oligonucleotides. In some embodiments, the anti-transferrin receptor antibody (anti-TfR antibody) or fragment thereof is conjugated to at least one antisense strand of the oligonucleotide. In some embodiments, the anti-transferrin receptor antibody or antigen binding fragment thereof conjugated to at least two antisense strands of at least two oligonucleotides.

In some embodiments, provided herein are conjugates of Formula (I): P-(L-R)ₙ, wherein R is an oligonucleotide of the present disclosure comprising a sense stand and an antisense strand; wherein P is the anti-TfR antibody or antigen binding fragment thereof as described in the present disclosure; and wherein L is a linker, which can be optionally absent; wherein “n” can be 1, 2 or more. In some embodiments, n is 1. In some embodiments, n is 2.

In some embodiments, the R is selected from Table 2, 3, or 4. In some embodiments, R is selected from siRNA No. 1 to 322 disclosed in Table 2, 3, or 4. In some embodiments, R has an antisense strand that has 90% sequence similarity to any one of antisense strands disclosed in Table 2, 3, or 4. In some embodiments, R has an antisense strand that has 95% sequence similarity to any one of antisense strands disclosed in Table 2, 3, or 4. In some embodiments, R has a sense strand that has 90% sequence similarity to any one of sense strands disclosed in Table 2, 3, or 4. In some embodiments, R has a sense strand that has 95% sequence similarity to any one of sense strands disclosed in Table 2, 3, or 4. In some embodiments, the P is selected from Table 8.

In some embodiments, the oligonucleotide of the conjugate mediates RNA interference against the human Nav1.8 mRNA. In certain embodiments, the oligonucleotide of the conjugate mediates RNA interference against the human Nav1.8 mRNA preferentially in DRG cells in a human subject. In some embodiments, the conjugate of the present disclosure, which includes the oligonucleotide, mediates RNA interference against the human Nav1.8 mRNA and modulates pain in a subject. In some embodiments, the conjugate of the present disclosure, which includes the oligonucleotide, mediates RNA interference against the human Nav1.8 mRNA and treats pain in a subject. In some embodiments, the conjugate of the present disclosure, which includes the oligonucleotide, mediates RNA interference against the human Nav1.8 mRNA and reduces pain in a subject.

In some embodiments, the anti-TfR antibody or antigen binding fragment thereof comprises a heavy chain variable region (HCVR) and a light chain variable region (LCVR), wherein the HCVR comprises heavy chain complementarity determining regions HCDR1, HCDR2, and HCDR3, and the LCVR comprises light chain complementarity determining regions LCDR1, LCDR2, and LCDR3, wherein the HCDR1, HCDR2, HCDR3, LCDR1, LCDR2, and LCDR3 comprise the following sequences: HCDR1 comprises SEQ ID NO: 629, HCDR2 comprises SEQ ID NO: 630, HCDR3 comprises SEQ ID NO: 631, LCDR1 comprises SEQ ID NO: 632, LCDR2 comprises SEQ ID NO: 633, and LCDR3 comprises SEQ ID NO: 634. Some examples of HCDRs of the TBPs of the present disclosure are provided in Table 5.

TABLE 5
Examples of Transferrin Receptor Binding Protein (TBP) Heavy Chain
Complementarity-determining Regions (HCDRs)

| TBP # | HCDR1 (Kabat) | HCDR2 (Kabat) | HCDR3 (Kabat) |
|---|---|---|---|
| 1, 2, | SEQ ID NO: 629 | SEQ ID NO: 630 | SEQ ID NO: 631 |
| and 3 | SYSMN | SISSSSSYIYYADSVKG | RHGYSNSDAFDT |

Some examples of LCDRs of the TBPs of the present disclosure are provided in Table 6.

TABLE 6
Examples of Transferrin Receptor Binding Protein (TBP) Light Chain
Complementarity-determining Regions (LCDRs)

| TBP # | LCDR1 (Kabat) | LCDR2 (Kabat) | LCDR3 (Kabat) |
|---|---|---|---|
| 1, 2, | SEQ ID NO: 632 | SEQ ID NO: 633 | SEQ ID NO: 634 |
| and 3 | RASQGISHYLV | AASSLqS | LqHNSYPWT |

Some examples of HCVRs and LCVRs of the TBPs of the present disclosure are provided in Table 7.

TABLE 7
Transferrin Receptor Binding Protein (TBP) Heavy
Chain Variable Region (HCVR) and Light Chain
Variable Region (LCVR)

| TBP # | HCVR | LCVR |
|---|---|---|
| 1, 2, and 3 | SEQ ID NO: 627 | SEQ ID NO: 628 |

Some examples of HCs and LCs of the TBPs of the present disclosure are provided in Table 8. TBP2 is a heteromab and accordingly has two Heavy chains (namely, HCA and HCB). Such heteromab antibodies are described, e.g., in Example 1 of US Patent Application Publication No. 2021/0054103, which is hereby incorporated by reference in its entirety. Heterodimeric antibodies such as heteromab, orthomab, or duobody have been described in WO2014150973, WO2016118742, WO2018118616, WO2011131746, which are hereby incorporated by reference in their entireties. The nucleic acid sequences for the TBPs are provided in Table 9 below.

TABLE 8
Transferrin Receptor Binding Protein
Heavy Chain (HC) and Light Chain (LC)

| | | | HCA | HCB | |
|---|---|---|---|---|---|
| | | Conjuga- | (First | (Second | LC |
| TBP | Type/ | tion | Heavy | Heavy | (Light |
| # | Format | Type | Chain) | Chain) | Chain) |
| 1 | 211 Com29 | nCys DAR1 | SEQ ID | | SEQ ID |
| | B09 hIgG1- | | NO: 625 | | NO: 626 |
| | EN mAb | | | | |
| 2 | 211 Com29 | eCys DAR1 | SEQ ID | SEQ ID | SEQ ID |
| | B09 hIgG1- | (Heteromab) | NO: 635 | NO: 636 | NO: 626 |
| | AAS | | (124S) | (124C) | |
| | heteromab | | | | |
| | 124C | | | | |
| 3 | 211 Com29 | eCys DAR1 | SEQ ID | | SEQ ID |
| | B09 hIgG1 | | NO: 637 | | NO: 638 |
| | 124C Fab | | | | |

In Table 8 and this disclosure, the term “EN” refers to effector null mutations. In some embodiment, the term “EN” refers to mutations L234A/L235E/G237A/A330S/P331S in the Fc region of the antibody or derivatives thereof.

In Table 8 and this disclosure, the term “AAS” refers to L234A/L235A/D265S mutations. This is an example of effector null mutations in the Fc region. For more information see, e.g., Pejchal et al., “Profiling the Biophysical Developability Properties of Common IgG1 Fc Effector Silencing Variants,” Antibodies, 12, 54 (2023), which is hereby incorporated by reference in its entirety.

In Table 8 and this disclosure, the term “211” refers to an antibody or derivatives thereof that bind to only the apical domain of the TfR.

In Table 8 and this disclosure, “Com29 B09” (e.g., TBP1) refers to antibody or derivative thereof engineered for reduced affinity and reduced immunogenicity risk. In Table 8 and this disclosure, “TBP2” has, among other things, an asymmetric/single eCys site. “TBP3” is a monovalent Fab fragment of TBP2 that has, among other things, an eCys site engineered in. In some embodiments, the conjugates (or AOCs) of the present disclosure include an antibody or antigen binding fragment thereof that includes at least one heavy chain constant region comprising cysteine at residue 124 (according to the EU Index numbering). In some embodiments, the conjugates (or AOCs) of the present disclosure include an antibody or antigen binding fragment thereof that includes two heavy chain constant regions comprising cysteine at residue 124 (according to the EU Index numbering).

TABLE 9
Nucleic Acid Sequences for Transferrin Receptor Binding
Protein (TBP) Heavy Chain (HC) and Light Chain (LC)

| TBP | Heavy Chain | Heavy Chain B | Light Chain |
|---|---|---|---|
| # | (HC) DNA | (HCB) DNA | (LC) DNA |
| 1 | SEQ ID NO: 639 | Not Applicable | SEQ ID NO: 643 |
| 2 | SEQ ID NO: 640 | SEQ ID NO: 642 | SEQ ID NO: 644 |
| | (HCA) | | |
| 3 | SEQ ID NO: 641 | Not Applicable | SEQ ID NO: 645 |

In some embodiments, the anti-TfR antibody or antigen binding fragment thereof of the present disclosure include a HCVR and a LCVR, wherein the HCVR comprises a sequence having at least 95% sequence identity to SEQ ID NO: 627 and LCVR comprises a sequence having at least 95% sequence identity to SEQ ID NO: 628.

In some embodiments, the anti-TfR antibody or antigen binding fragment thereof of the present disclosure include a HCVR and a LCVR, and wherein the HCVR comprises a sequence having at least 90% sequence identity to SEQ ID NO: 627 and LCVR comprises a sequence having at least 90% sequence identity to SEQ ID NO: 628.

In some embodiments, the anti-TfR antibody or antigen binding fragment thereof of the present disclosure is a human IgG1 antibody with effector null (EN) mutations that comprises a HC and a LC, wherein the HC comprises the sequence of SEQ ID NO: 625 and the LC comprises the sequence of SEQ ID NO: 626.

In some embodiments, the anti-TfR antibody or antigen binding fragment thereof of the present disclosure is a human IgG1 antibody with effector null (EN) mutations that comprises a HC and a LC, wherein the HC comprises a sequence having at least 90% sequence identity to the sequence of SEQ ID NO: 625 and the LC comprises a sequence having at least 90% sequence identity to the sequence of SEQ ID NO: 626.

In some embodiments, the anti-TfR antibody or antigen binding fragment thereof of the present disclosure is a human IgG1 antibody with effector null (EN) mutations that comprises a HC and a LC, wherein the HC comprises a sequence having at least 95% sequence identity to the sequence of SEQ ID NO: 625 and the LC comprises a sequence having at least 95% sequence identity to the sequence of SEQ ID NO: 626.

In some embodiments, the anti-TfR antibody or antigen binding fragment thereof of the present disclosure is a human IgG1 antibody heteromab with AAS effector null mutations and the TBP comprises two variants of HC (HCA and HCB) and a LC, wherein the HCA comprises the sequence of SEQ ID NO: 635 which includes 124S, HCB comprises the sequence of SEQ ID NO: 636 which includes 124C, and the LC comprises the sequence of SEQ ID NO: 626.

In some embodiments, the anti-TfR antibody or antigen binding fragment thereof of the present disclosure is a human IgG1 antibody heteromab with AAS effector null mutations and the TBP comprises two variants of HC (HCA and HCB) and a LC, wherein the HCA comprises a sequence having at least 95% sequence identity to the sequence of SEQ ID NO: 635 which includes 124S, HCB comprises a sequence having at least 95% sequence identity to the sequence of SEQ ID NO: 636 which includes 124C, and the LC comprises a sequence having at least 95% sequence identity to the sequence of SEQ ID NO: 626.

In some embodiments, the anti-TfR antibody or antigen binding fragment thereof of the present disclosure is a human IgG1 antibody heteromab with AAS effector null mutations and the TBP comprises two variants of HC (HCA and HCB) and a LC, wherein the HCA comprises a sequence having at least 90% sequence identity to the sequence of SEQ ID NO: 635 which includes 124S, HCB comprises a sequence having at least 90% sequence identity to the sequence of SEQ ID NO: 636 which includes 124C, and the LC comprises a sequence having at least 90% sequence identity to the sequence of SEQ ID NO: 626.

In some embodiments, the anti-TfR antibody or antigen binding fragment thereof of the present disclosure is a human IgG1 Fab with a cysteine at position 124 of the HC and the Fab comprises a HC and a LC, wherein the HC comprises the sequence of SEQ ID NO: 637 and the LC comprises the sequence of SEQ ID NO: 638.

In some embodiments, the anti-TfR antibody or antigen binding fragment thereof of the present disclosure is a human IgG1 Fab with a cysteine at position 124 of the HC and the Fab comprises a HC and a LC, wherein the HC comprises a sequence having at least 90% sequence identity to the sequence of SEQ ID NO: 637 and the LC comprises a sequence having at least 90% sequence identity to the sequence of SEQ ID NO: 638.

In some embodiments, the anti-TfR antibody or antigen binding fragment thereof of the present disclosure is a human IgG1 Fab with a cysteine at position 124 of the HC and the Fab comprises a HC and a LC, wherein the HC comprises a sequence having at least 95% sequence identity to the sequence of SEQ ID NO: 637 and the LC comprises a sequence having at least 95% sequence identity to the sequence of SEQ ID NO: 638.

In some embodiments, the conjugate of the present disclosure is selected from the following Table 10. In some embodiments, the conjugate has one SMCC linker connecting the antibody to the oligonucleotide.

TABLE 10
Some embodiments of the Conjugates of the present disclosure.

| | |
|---|---|
| “P” | “R” |
| (anti-TfR Antibody) | oligonucleotide* |

TBP 1 Conjugate

| | |
|---|---|
| TBP1 | SEQ ID NOs: 565 and 605 |
| (HC SEQ ID NO: 625 | SEQ ID NOs: 567 and 606 |
| and LC 626). | SEQ ID NOs: 569 and 607 |
| In some embodiments | SEQ ID NOs: 571 and 608 |
| the “P” protein | SEQ ID NOs: 573 and 609 |
| has at least 90% or 95% | SEQ ID NOs: 575 and 610 |
| sequence identity to HC. | SEQ ID NOs: 577 and 611 |
| In some embodiments, | SEQ ID NOs: 579 and 612 |
| the “P” has at | SEQ ID NOs: 581 and 613 |
| least 90% or | SEQ ID NOs: 583 and 614 |
| 95% sequence | SEQ ID NOs: 585 and 615 |
| identity to LC. | SEQ ID NOs: 587 and 616 |
| | SEQ ID NOs: 589 and 617 |
| | SEQ ID NOs: 591 and 618 |
| | SEQ ID NOs: 593 and 619 |
| | SEQ ID NOs: 595 and 620 |
| | SEQ ID NOs: 597 and 621 |
| | SEQ ID NOs: 599 and 622 |
| | SEQ ID NOs: 601 and 623 |
| | SEQ ID NOs: 603 and 624 |

TBP 2 Conjugate

| | |
|---|---|
| TBP2 | SEQ ID NOs: 565 and 605 |
| (HCA SEQ ID NO: 635; | SEQ ID NOs: 567 and 606 |
| HCB SEQ ID NO: 636; | SEQ ID NOs: 569 and 607 |
| and LC 626) | SEQ ID NOs: 571 and 608 |
| In some embodiments | SEQ ID NOs: 573 and 609 |
| the “P” protein | SEQ ID NOs: 575 and 610 |
| has at least 90% or 95% | SEQ ID NOs: 577 and 611 |
| sequence identity to | SEQ ID NOs: 579 and 612 |
| HCA. | SEQ ID NOs: 581 and 613 |
| In some embodiments | SEQ ID NOs: 583 and 614 |
| the “P” protein | SEQ ID NOs: 585 and 615 |
| has at least 90% or 95% | SEQ ID NOs: 587 and 616 |
| sequence identity to | SEQ ID NOs: 589 and 617 |
| HCB. | SEQ ID NOs: 591 and 618 |
| In some embodiments, | SEQ ID NOs: 593 and 619 |
| the “P” has at | SEQ ID NOs: 595 and 620 |
| least 90% or 95% sequence | SEQ ID NOs: 597 and 621 |
| identity to LC. | SEQ ID NOs: 599 and 622 |
| | SEQ ID NOs: 601 and 623 |
| | SEQ ID NOs: 603 and 624 |

TBP 3 Conjugate

| | |
|---|---|
| TBP1 | SEQ ID NOs: 565 and 605 |
| (HC SEQ ID NO: 637 | SEQ ID NOs: 567 and 606 |
| and LC 638) | SEQ ID NOs: 569 and 607 |
| In some embodiments | SEQ ID NOs: 571 and 608 |
| the “P” protein | SEQ ID NOs: 573 and 609 |
| has at least 90% or 95% | SEQ ID NOs: 575 and 610 |
| sequence identity to HC. | SEQ ID NOs: 577 and 611 |
| In some embodiments, | SEQ ID NOs: 579 and 612 |
| the “P” has at | SEQ ID NOs: 581 and 613 |
| least 90% or 95% sequence | SEQ ID NOs: 583 and 614 |
| identity to LC. | SEQ ID NOs: 585 and 615 |
| | SEQ ID NOs: 587 and 616 |
| | SEQ ID NOs: 589 and 617 |
| | SEQ ID NOs: 591 and 618 |
| | SEQ ID NOs: 593 and 619 |
| | SEQ ID NOs: 595 and 620 |
| | SEQ ID NOs: 597 and 621 |
| | SEQ ID NOs: 599 and 622 |
| | SEQ ID NOs: 601 and 623 |
| | SEQ ID NOs: 603 and 624 |

*In some embodiments the “R” oligonucleotide has at least 90% or 95% sequence identity to the listed sequences.

The TfR binding proteins described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the TfR binding proteins) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell (e.g., a mammalian cell) includes cells stably or transiently transfected, transformed, transduced, or infected with one or more expression vectors expressing all or a portion of the TfR binding proteins described herein. According to some embodiments, a host cell may be stably or transiently transfected, transformed, transduced, or infected with an expression vector expressing HC polypeptides and an expression vector expressing LC polypeptides of the TfR binding proteins described herein. In some embodiments, a host cell may be stably or transiently transfected, transformed, transduced, or infected with an expression vector expressing HC and LC polypeptides of the TfR binding proteins described herein. The TfR binding proteins may be produced in mammalian cells such as CHO, NS0, HEK293 or COS cells according to techniques well known in the art.

In some embodiments, the cell growth medium, into which the TfR binding proteins have been secreted, may be purified by conventional techniques, such as mixed-mode methods of ion-exchange and hydrophobic interaction chromatography. For example, the cell growth medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

Embodiments of the present disclosure also include antibody fragments or antigen-binding fragments that, as used herein, comprise at least a portion of an antibody retaining the ability to specifically interact with an antigen or an epitope of the antigen, such as, Fab, Fab′, F(ab′)2, Fv fragments, scFv antibody fragments, scFv-Fc, diabody, scFab, scFv-CH3, Fv, scFa, disulfide-linked Fvs (sdFv), and a Fd fragment.

In some embodiments, the TfR binding protein is scFv. In some embodiments, the TfR binding protein is Fab. In some embodiments, the TfR binding protein is a Fab and a VHH linked to the Fab, wherein the VHH binds human serum albumin (HSA). In some embodiments, the TfR binding protein further comprises a heavy chain constant region comprising cysteine at residue 124 (according to the EU Index numbering). EU numbering system is used in the present disclosure for numbering the residues of antibodies or fragments thereof.

In some embodiments, the antibody or antigen binding fragment thereof is conjugated to the oligonucleotide using “nCys” method. “nCys” refers to a conjugation method where one or more native cysteine residues are used for conjugation of the antibody or antigen-binding fragment thereof to a molecular payload (e.g., ASO or siRNA). In some embodiments, the human TfR binding proteins described herein comprise one or more native cysteine residues, which can be used for conjugation. For example, in some embodiments, the human TfR binding protein described herein comprises a native cysteine at position 214 of the light chain and/or a native cysteine at positions 220, 226, and/or 229 of the heavy chain, which can be used for conjugation (all residues according to the EU Index numbering).

In some embodiments, the antibody or antigen binding fragment thereof is conjugated to the oligonucleotide using “eCys” method. This is a method where one or more engineered cysteine residues are artificially incorporated into the antibody or antigen binding fragment thereof and are used for conjugation of the antibody or antigen-binding fragment thereof to a molecular payload (e.g., siRNA). The approach of including engineered cysteines as a means for conjugation has been described in WO2018232088, which is hereby incorporated by reference in its entirety. In some embodiments, the human TfR binding proteins described herein comprise a heavy chain comprising one or more cysteines at the following residues: 124, 157, 162, 262, 373, 375, 397, 415 (all residues according to the EU Index numbering). In some embodiments, the human TfR binding proteins described herein comprise a light chain (e.g., a kappa light chain) comprising one or more cysteines at the following residues: 156, 171, 191, 193, 202, 208 (all residues according to the EU Index numbering). In some embodiments, the human TfR binding proteins described herein comprise a heavy chain constant region comprising cysteine at residue 124 (according to the EU Index numbering). In some embodiments, the human TfR binding proteins described herein comprise a light chain constant region comprising cysteine at residue 156 (according to the EU Index numbering). In some embodiments, the human TfR binding proteins described herein comprise an immunoglobulin Fc region comprising cysteine at residue 378 (according to the EU Index numbering).

In some embodiments, the antibody or the antigen-binding fragment thereof is conjugated to one payload (e.g., oligonucleotide) and has a drug to antibody ratio (DAR) of 1. In some embodiments, the antibody or the antigen-binding fragment thereof is conjugated to two payloads and has a drug to antibody ratio (DAR) of 2. In some embodiments, two variants of heavy chain (HCA and HCB) are such that one variant includes an engineered cysteine (eCys) for site-specific conjugation to, e.g., a payload. Such conjugation to one variant of the heavy or the light chain, e.g., may be done to yield a highly homogenous DART conjugate or product. In some embodiments, HCA has the eCys site for site-specific conjugation. In some embodiments, HCB has the eCys site for site-specific conjugation.

In some embodiments, the antibody or antigen-binding fragment thereof of the present disclosure further comprises a half-life extender, e.g., an immunoglobulin Fc region or a VHH that binds human serum albumin (see, e.g., WO2022169766, which is hereby incorporated by reference in its entirety). In some embodiments, the antibody or antigen-binding fragment thereof of the present disclosure comprises an immunoglobulin Fc region, e.g., a modified human IgG4 Fc region or a modified human IgG1 Fc region. In some embodiments, the antibody or antigen-binding fragment thereof of the present disclosure comprises a modified human IgG4 Fc region comprising proline at residue 228, and alanine at residues 234 and 235 (all residues are numbered according to the EU Index numbering, also called hIgG4PAA Fc region). In some embodiments, the antibody or antigen-binding fragment thereof of the present disclosure comprises a modified human IgG1 Fc region comprising alanine at residues 234, 235, and 329, serine at position 265, aspartic acid at position 436 (all residues are numbered according to the EU Index numbering, all called hIgG1 effector null or hIgG1EN Fc region).

In some embodiments, the antibody or antigen-binding fragment thereof of the present disclosure comprises a VHH that binds HSA. In some embodiments, the VHH also binds mouse, rat, and/or cynomolgus monkey albumin.

### Linkers

In some embodiments, the oligonucleotide of the present disclosure includes a linker moiety attached to one or more termini of the sense strand or the antisense strand. Such linker may be used to attach an antibody to the oligonucleotide. In some embodiments, the linker is SMCC and is optionally attached to the 5′ end of the sense strand. In some embodiments, at least one nucleotide of the oligonucleotide is conjugated to cholesterol, lipid, polypeptide, or an antibody or a fragment thereof. In some embodiments, a linker is used to conjugate the cholesterol, lipid, polypeptide, or an antibody or a fragment thereof to the oligonucleotide.

In some embodiments, the anti-TfR antibody or antigen binding fragment thereof of the present disclosure is linked to the sense strand or the antisense strand of the oligonucleotide through a linker. In some embodiments, this is an SMCC linker. In some embodiments, the linker is SMCC linker attached to i) the 5′ end of the sense strand, ii) the 5′ end of the antisense strand, iii) the 3′ end of the sense strand, or iv) the 3′ end of the antisense strand.

In some embodiments, a linker described herein is a cleavable linker or a non-cleavable linker. In some instances, the linker is a cleavable linker in other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof.

In some embodiments, the linker comprises a maleimide group. In some instances the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (me). In other instances, the maleimide group comprises a maleimidomethyl group, such as succinimidlyl-4-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates Nat. Biotechnol. 32(10):1059-1062 (2014). In some instances, the linker comprises a self-stabilizing maleimide. In some instances, the linker is a self-stabilizing maleimide.

In some embodiments, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivates thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some instances, the linker comprises a homobifunctional linker, Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (B3S), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g., 5-difluoro-2,4-dinitrobenzene or 1,3-difluoro-4,6-dinitrobenzene, 4,4-difluoro-3,3-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α-p-diaminodiphenyl, diiodo-p-xylene sulfonic acid, N,N′-ethylene-bis(iodoacetamide), or N,N′-hexamethylene-bis(iodoacetamide).

In some embodiments, the linker is a dendritic type linker. In some instances, the dendritic type linker comprises a branching, multifunctional linker moiety. In some instances, the dendritic type linker is used to increase the molar ratio of siRNA to the antibody. In some instances, the dendritic type linker comprises polyamidoamine (PAMAM) dendrimers.

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO20140802511 WO2014197854; WO2014145090; or WO2014177042.

### Expression Vectors and Cells

In some embodiments, the invention provides vectors for inhibiting the expression of the Nav1.8 gene in a cell, comprising a regulatory sequence operably linked to a nucleotide sequence that encodes at least one strand of one of the oligonucleotides of the invention.

In another embodiment, the invention provides a cell comprising a vector for inhibiting the expression of the Nav1.8 gene in a cell. The vector comprises a regulatory sequence operably linked to a nucleotide sequence that encodes at least one strand of one of the oligonucleotides of the invention.

In another aspect of the invention, Nav1.8 specific oligonucleotide molecules that modulate Nav1.8 gene expression activity are expressed from transcription units inserted into DNA or RNA vectors known in the art. These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., PNAS (1995) 92:1292).

The individual strands of an oligonucleotide can be transcribed by promoters on two separate expression vectors and co-transfected into a target cell. Alternatively, each individual strand of the oligonucleotide can be transcribed by promoters both of which are located on the same expression plasmid. The recombinant oligonucleotide expression vectors are preferably DNA plasmids or viral vectors. oligonucleotide expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. The oligonucleotide molecules can also be inserted into vectors and used as gene therapy vectors for human patients.

A person skilled in the art would be able to choose the appropriate regulatory/promoter sequence based on the intended use of the oligonucleotide transgene. In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J 8:20-24).

### Pharmaceutical Compositions

In one embodiment, the disclosure provides pharmaceutical compositions comprising an oligonucleotide, as described herein, and a pharmaceutically acceptable carrier. In another embodiment, the disclosure provides pharmaceutical compositions comprising a conjugate, as described herein, and a pharmaceutically acceptable carrier. The pharmaceutical composition comprising the oligonucleotide, or the conjugate is useful for treating a disease or disorder associated with the expression or activity of the Nav1.8 gene, such as chronic pain, including, neuropathic, inflammatory, or mixed pain.

In another embodiment, the disclosure provides pharmaceutical compositions comprising at least two oligonucleotides, designed to target different regions of the Nav1.8 gene, and a pharmaceutically acceptable carrier. In yet another embodiment, the disclosure provides pharmaceutical compositions comprising at least two conjugates, designed to target different regions of the Nav1.8 gene, and a pharmaceutically acceptable carrier. In this embodiment, one oligonucleotide can have a nucleotide sequence which is substantially complementary to at least one part of the Nav1.8 gene and an additional oligonucleotide which has a nucleotide sequence that is substantially complementary to different part of the Nav1.8 gene. The multiple oligonucleotides or conjugates may be combined in the same pharmaceutical composition or formulated separately. If formulated individually, the compositions containing the separate oligonucleotide or conjugates may comprise the same or different carriers and may be administered using the same or different routes of administration. Moreover, the pharmaceutical compositions comprising the individual oligonucleotides or conjugates may be administered substantially simultaneously, sequentially, or at preset intervals throughout the day or treatment period.

The pharmaceutical compositions of the disclosure are administered in dosages sufficient to inhibit expression of the Nav1.8 gene. In general, a suitable dose of oligonucleotide will be in the range of 0.01 to 10.0 milligrams per kilogram body weight of the recipient per day. A skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual oligonucleotides encompassed by the disclosure can be made using conventional methodologies or based on in vivo testing using an appropriate animal model.

The pharmaceutical compositions encompassed by the disclosure may be administered by any means known in the art including, but not limited to oral or parenteral routes, including intravenous, intramuscular, intraperitoneal, epidural, intrathecal, intracerebroventricular, intraparenchymal (within the peripheral or central nervous system), subcutaneous, transdermal, intranasal, airway (aerosol), rectal, vaginal, and topical (including buccal and sublingual) administration.

For intrathecal, intracerebroventricular, intramuscular, intraparenchymal (within the peripheral or central nervous system), subcutaneous and intravenous use, the pharmaceutical compositions of the disclosure will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of oligonucleotide in the cells that express the Nav1.8 gene. Such substances include, for example, micellar structures, such as liposomes or capsids, as described below.

The pharmaceutical compositions useful according to the disclosure also include encapsulated formulations to protect the oligonucleotide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein. Lipid nanoparticles (LNPs) may also be used for delivering the oligonucleotides or conjugates of the present disclosure.

In addition to their administration individually or as a plurality, as discussed above, the oligonucleotide or conjugates of the disclosure can be administered in combination with other known agents effective in treatment of pain. In any event, the administering physician can adjust the amount and timing of oligonucleotides or conjugates disclosed herein for administration on the basis of results observed using standard measures of efficacy known in the art or described herein.

In some embodiments, the composition herein comprises a carrier, which can confer to a composition improved stability, improved absorption, improved solubility and/or therapeutic enhancement of the active ingredient. In some embodiments, the carrier is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, the oligonucleotides herein are lyophilized for extending shelf-life and then made into a solution before use (e.g., administration to an individual). Accordingly, the carrier in a pharmaceutical composition including one or more of the oligonucleotides is a lyoprotectant (e.g., mannitol, lactose, polyethylene glycol, or polyvinylpyrrolidone) or a collapse temperature modifier (e.g., dextran, Ficoll™, or gelatin).

Pharmaceutical compositions are formulated to be compatible with its intended route of administration. Routes of administration include, but are not limited to, parenteral (e.g. intravenous, intramuscular, intraperitoneal, intradermal, and subcutaneous), oral (e.g., inhalation), transdermal (e.g., topical), transmucosal, and rectal administration.

### Methods of Reducing SCN10A Activity in Cells, Tissue, Organs, and Organisms

The oligonucleotides, conjugates, or pharmaceutical compositions disclosed herein are used to reduce SCN10A mRNA, Nav1.8 protein and/or Nav1.8 activity in cells, tissues, organs, or individuals. The methods comprise the steps described herein, and these may be, but not necessarily, carried out in the sequence as described. Other sequences, however, also are conceivable. Moreover, individual, or multiple steps are carried out either in parallel and/or overlapping in time and/or individually or in multiply repeated steps. Furthermore, the methods comprise additional, unspecified steps.

The methods comprise contacting or delivering to a cell, population of cells, tissues, organs, or individuals an effective amount any of the oligonucleotides, conjugates, or pharmaceutical compositions disclosed herein for reducing SCN10A expression. In some embodiments, reduced SCN10A activity is determined by measuring a reduction in the amount or level of SCN10A mRNA, Nav1.8 protein, and/or Nav1.8 protein activity in a cell.

With regard to an appropriate cell type, the cell type is any cell that expresses SCN10A mRNA (e.g., DRG neurons). In some embodiments, the cell is a primary cell obtained from an individual. In some embodiments, the cell is a primary cell obtained from the nervous system (CNS or PNS) of an individual. In some embodiments, the primary cell has undergone a limited number of passages such that the cell substantially maintains its natural phenotypic properties. In some embodiments, the cell is an ex vivo, in vivo, or in vitro cell (i.e., such that one or more of the oligonucleotides or conjugates described herein can be delivered to the cell in culture or to an organism in which the cell resides).

In some embodiments, the oligonucleotides herein are delivered to a cell or population of cells using a nucleic acid delivery method known in the art including, but not limited to, injecting a solution containing the oligonucleotides, bombarding by particles covered by the oligonucleotides, exposing the cell or population of cells to a solution containing the oligonucleotides, or electroporating cell membranes in the presence of the oligonucleotides. Other methods known in the art for delivering oligonucleotides to cells are used such as, for example, lipid-mediated carrier transport, chemical-mediated transport, and cationic liposome transfection such as calcium phosphate, and others. In some embodiments, the oligonucleotides are conjugated to anti-TfR antibodies or antigen-binding fragments thereof for transport into a cell, a population of cells, a tissue, or a subject's cells.

Reduced SCN10A activity is determined by an assay or technique that evaluates one or more molecules, properties or characteristics of a cell or population of cells associated with SCN10A gene expression (e.g., using a SCN10A expression biomarker) or by an assay or technique that evaluates molecules that are directly indicative of SCN10A activity in a cell or population of cells (e.g., SCN10A mRNA, Nav1.8 protein and/or Nav1.8 activity). In some embodiments, the extent to which the oligonucleotides reduce SCN10A activity are evaluated by comparing SCN10A activity in a cell or population of cells contacted with the oligonucleotides or conjugates to a control cell or population of cells (e.g., a cell or population of cells not contacted with the oligonucleotides or contacted with a control oligonucleotide). In some embodiments, a control amount or level of SCN10A activity in a control cell or population of cells is predetermined, such that the control amount or level need not be measured in every instance the assay or technique is performed. The predetermined level or value takes a variety of forms including, but not limited to, a single cut-off value, such as a median or mean.

Contacting or delivering the oligonucleotides, conjugates, or pharmaceutical compositions disclosed herein to a cell or a population of cells result in reduced SCN10A activity. In some embodiments, reduced SCN10A activity is relative to a control amount or level of SCN10A activity in the cell or the population of cells not contacted with, e.g., the oligonucleotides or contacted with a control oligonucleotide. In some embodiments, reduced SCN10A activity is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of SCN10A activity. In some embodiments, the control amount or level of SCN10A activity is an amount or level of SCN10A mRNA, Nav1.8 protein and/or Nav1.8 activity in the cell or the population of cells that has not been contacted with the oligonucleotides, conjugates, or pharmaceutical compositions disclosed herein. In some embodiments, the effect of delivery of the oligonucleotides, conjugates, or pharmaceutical compositions disclosed herein to the cell or the population of cells according to a method herein is assessed after any finite period or amount of time (e.g., minutes, hours, days, weeks, and/or months). For example, SCN10A activity is determined in the cell or the population of cells at least about 4 hours, about 8 hours, about 12 hours, about 18 hours, or about 24 hours. Alternatively, SCN10A activity is determined in the cell or the population of cells at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, or about 84 days or more after contacting or delivering the oligonucleotides, conjugates, or pharmaceutical compositions disclosed herein to the cell or population of cells. In other embodiments, SCN10A activity is determined in the cell or the population of cells at least about 1 month, about 2 months, about 3 months, about 4 months, about 5 months, or about 6 months or more after contacting or delivering the oligonucleotides, conjugates, or pharmaceutical compositions disclosed herein to the cell or the population of cells.

### Methods of Preventing, Reducing, or Treating:

One aspect of the present disclosure is related to a method of preventing, treating, or reducing pain in a patient in need thereof, the method comprising administering to the patient an effective amount of the oligonucleotide of the present disclosure; an effective amount of the conjugate of the present disclosure; or the pharmaceutical composition of the present disclosure. Another aspect of the present disclosure is related to use of the oligonucleotide of the present disclosure; the conjugate of the present disclosure; or the pharmaceutical composition of the present disclosure in the manufacture of a medicament for preventing, treating, or reducing pain. In yet another aspect, the oligonucleotide of the present disclosure; the conjugate of the present disclosure; or the pharmaceutical composition of the present disclosure is for use in preventing, treating, or reducing pain in a patient in need thereof. In some embodiments, the methods of treating an individual having, suspected of having, or at risk of developing a disease, disorder, or condition associated with SCN10A activity comprise administering at least one or more of the oligonucleotides or conjugates described herein to the individual.

In some embodiments, the individual is suffering from chronic pain, including, i) inflammatory pain, ii) neuropathic pain, or iii) mixed pain. In one embodiment, the oligonucleotide, the conjugate, or the pharmaceutical composition of the present disclosure is administered intravenously according to at least one of the methods described herein.

Additionally, methods of treating or attenuating an onset or progression of a disease, disorder, or condition associated with SCN10A activity in an individual comprise using one or more of oligonucleotides, pharmaceutical compositions, or conjugates described herein. Furthermore, methods of achieving one or more therapeutic benefits in an individual having a disease, disorder, or condition associated with SCN10A activity comprise providing one or more of the oligonucleotides, pharmaceutical compositions, or conjugates disclosed herein. In some embodiments, the individual can be treated by administering a therapeutically effective amount of any one or more of the oligonucleotides, pharmaceutical compositions, or conjugates herein. In some embodiments, the treatment comprises reducing SCN10A activity. In some embodiments, the individual is treated therapeutically. In some embodiments, the individual is treated prophylactically.

In some embodiments, the one or more oligonucleotides, one or more conjugates, or a pharmaceutical composition including the same, is administered to the individual having a disease, disorder, or condition associated with SCN10A activity such that SCN10A activity is reduced in the individual, thereby treating the individual. In some embodiments, an amount or level of SCN10A mRNA is reduced in the individual. In other embodiments, an amount or level of SCN10A (or Nav1.8) protein is reduced in the individual. In still other embodiments, an amount or level of SCN10A activity is reduced in the individual.

In some embodiments, SCN10A activity is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to SCN10A activity prior to administering the one or more oligonucleotides, conjugates, or pharmaceutical composition thereof. In other embodiments, SCN10A activity is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to SCN10A activity in an individual (e.g., a reference or control individual) not receiving the one or more oligonucleotides, conjugates, or pharmaceutical composition or receiving a control oligonucleotide, conjugates, pharmaceutical composition or treatment.

In certain embodiments, an amount or level of SCN10A mRNA is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of SCN10A mRNA prior to administering the one or more oligonucleotides, conjugates, or pharmaceutical composition thereof. In some embodiments, the amount or level of SCN10A mRNA is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of SCN10A mRNA in an individual (e.g., a reference or control individual) not administered the one or more oligonucleotides, conjugates, or pharmaceutical composition or administered a control oligonucleotide, pharmaceutical composition, or treatment.

In certain embodiments, an amount or level of SCN10A (or Nav1.8) protein is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of SCN10A protein prior to administering the one or more oligonucleotides, conjugates, or pharmaceutical composition thereof. In other embodiments, an amount or level of SCN10A protein is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of SCN10A protein in an individual (e.g., a reference or control individual) not administered the one or more oligonucleotides, conjugates, or pharmaceutical composition or administered a control oligonucleotide, pharmaceutical composition or treatment.

In certain embodiments, an amount or level of Nav1.8 activity is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of Nav1.8 activity prior to administering the one or more oligonucleotides, conjugates, or pharmaceutical composition thereof. In some embodiments, the amount or level of Nav1.8 activity is reduced in the individual by at least about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of Nav1.8 activity in an individual (e.g., a reference or control individual) not administered the one or more oligonucleotides or pharmaceutical composition or administered a control oligonucleotide, pharmaceutical composition or treatment.

Here, SCN10A activity, the amount or level of SCN10A mRNA, Nav1.8 protein, Nav1.8 activity, or any combination thereof, is reduced in a cell (e.g., a DRG neuron), a population or a group of cells (e.g., a nerve), a tissue, a sample, an organ, blood, or a fraction thereof, or any other biological material obtained or isolated from the individual. In some embodiments, SCN10A activity, the amount or level of SCN10A mRNA, Nav1.8 protein, Nav1.8 activity, or any combination thereof, is reduced in more than one type of cell, more than one groups of cells, more than one type of tissue, more than one type of sample, more than one organ, more than one fraction of blood obtained or isolated from the individual.

Examples of a disease, disorder, or condition associated with SCN10A activity include, but are not limited to, chronic pain, including neuropathic pain, inflammatory pain, mixed pain. Examples of diseases that may be related to activity of SCN10A activity include inflammatory CNS diseases such as multiple sclerosis, myelitis or syphilis, ischemia, hemorrhages, or arteriovenous malformations (e.g., post-stroke neuropathic pain) located in the thalamus, spinothalamic pathway or thalamocortical projections, and syringomyelia (Koltzenburg, Pain 2002—An Updated Review: Refresher Course Syllabus; IASP Press, Seattle, 2002). The diseases or disorders related to SCN10A activity also include “pain and related disorders,” the term “related disorders” refers to disorders that either cause or are associated with pain or have been shown to have similar mechanisms to pain. These disorders include addiction, seizure, stroke, ischemia, a neurodegenerative disorder, anxiety, depression, headache, asthma, rheumatic disease, osteoarthritis, retinopathy, inflammatory eye disorders, pruritis, ulcer, gastric lesions, uncontrollable urination, an inflammatory or unstable bladder disorder, inflammatory bowel disease, irritable bowel syndrome (IBS), irritable bowel disease (IBD), gastroesophageal reflux disease (GERD), functional dyspepsia, functional chest pain of presumed oesophageal origin, functional dysphagia, non-cardiac chest pain, symptomatic gastroesophageal disease, gastritis, aerophagia, functional constipation, functional diarrhea, burbulence, chronic functional abdominal pain, recurrent abdominal pain (RAP), functional abdominal bloating, functional biliary pain, functional incontinence, functional anorectal pain, chronic pelvic pain, pelvic floor dyssynergia, unspecified functional anorectal disorder, interstitial cystitis, dysmenorrhea, and dyspareunia.

Because of their high specificity, the oligonucleotides or conjugates described herein specifically target mRNAs of target genes of cells, tissues, or organs. In preventing disease, the target gene is the one that is required for initiation or maintenance of the disease or that has been identified as being associated with a higher risk of contracting the disease. In treating disease, one or more of the oligonucleotides or conjugates described herein are brought into contact with the cells, tissue or organ exhibiting or responsible for mediating the disease. For example, an oligonucleotide substantially complimentary to all or part of a wild-type (i.e., native) or mutated gene associated with a disease, disorder, or condition associated with SCN10A (or Nav1.8) activity is brought into contact with or introduced into a cell or tissue type of interest such as a DRG neuron.

In some embodiments, the target gene is from any mammal, such as a human. Any gene may be silenced according to the methods herein. Moreover, the methods herein typically involve administering to an individual a therapeutically effective amount of one or more oligonucleotides herein, that is, an amount capable of producing a desirable therapeutic result. The therapeutically acceptable amount is an amount that therapeutically treats a disease or disorder or condition. The appropriate dosage for any one individual will depend on certain factors, including the individual's size, body surface area, age, the composition to be administered, the active ingredient(s) in the composition, time and route of administration, general health, and other therapeutic agents being administered concurrently.

In the methods, the individual is administered any one of the oligonucleotides, conjugates, or compositions herein either enterally (e.g., orally, by gastric feeding tube, by duodenal feeding tube, via gastrostomy, or rectally), parenterally (e.g., subcutaneous injection, intravenous injection or infusion, intra-arterial injection or infusion, intraosseous infusion, intramuscular injection, intracerebral injection, intracerebroventricular injection, or intrathecal), topically (e.g., epicutaneous, inhalational, via eye drops, or through a mucous membrane), or by direct injection into a target organ (e.g., the liver of an individual). Typically, the oligonucleotides or compositions are administered intravenously or subcutaneously.

As a non-limiting set of examples, the oligonucleotides or compositions herein typically are administered quarterly (once every three months), bi-monthly (once every two months), monthly or weekly. For example, the oligonucleotides or compositions are administered every week or at intervals of two, or three weeks. In certain embodiments, the oligonucleotides, or compositions are administered daily. In some embodiments, an individual is administered one or more loading doses of the oligonucleotides or compositions followed by one or more maintenance doses of the oligonucleotides or compositions.

In some embodiments, the individual is a human, a NHP, or other mammal. In other embodiments, the individual is a domesticated animal such as a dog or a cat; livestock such as a horse, cattle, pig, sheep, goat, or chicken; and animals such as a mouse, rat, guinea pig or hamster.

The oligonucleotides, conjugates, or compositions disclosed herein can be used, or adapted for use, to treat an individual (e.g., a human having a disease, disorder, or condition associated with Nav1.8 activity) that would benefit from reducing SCN10A activity. In some embodiments, the oligonucleotides are provided for use, or adapted for use, to treat an individual having a disease, disorder, or condition associated with SCN10A activity. Also, the oligonucleotides are provided for use, or adaptable for use, in the manufacture of a medicament or pharmaceutical composition for treating a disease, disorder, or condition associated with SCN10A activity. In other embodiments, the oligonucleotides or conjugates are provided for use, or adaptable for use, in targeting SCN10A mRNA and reducing Nav1.8 activity (e.g., via the RNAi pathway). In other embodiments, the oligonucleotides or conjugates are provided for use, or adaptable for use, in targeting SCN10A mRNA and reducing an amount or level of SCN10A mRNA, Nav1.8 protein, and/or Nav1.8 activity.

The oligonucleotides, the conjugates, or the pharmaceutical compositions disclosed herein can be incorporated into a kit comprising one or more of the oligonucleotides, the conjugates, or the pharmaceutical compositions, and instructions for use. In some embodiments, the kit comprises one or more of the oligonucleotides, the conjugates, or the pharmaceutical compositions, and a package insert containing instructions for use of the kit and/or any component thereof. In other embodiments, the kit comprises a suitable container, one or more of the oligonucleotides, the conjugates, or the pharmaceutical compositions, one or more controls, and various buffers, reagents, enzymes, and other standard ingredients as are known in the art.

In some embodiments, the container can be at least one vial, well, test tube, flask, bottle, syringe, or other container means, into which the one or more the oligonucleotides, the conjugates, or the pharmaceutical compositions are placed, and in some embodiments, suitably aliquoted. In other embodiments, where an additional component is provided, the kit contains additional containers into which this component is placed. The kit also comprises a means for containing the one or more the oligonucleotides, the conjugates, or the pharmaceutical compositions and any other reagent in close confinement for commercial sale. Such containers include injection or blow-molded plastic containers into which the desired vials are retained. Containers and/or kits comprise labeling with instructions for use and/or warnings.

In some embodiments, the kit comprises one or more the oligonucleotides, the conjugates herein, and a pharmaceutically acceptable carrier, or a pharmaceutical composition comprising one or more of the oligonucleotides or the conjugates and instructions for treating or delaying progression of a disease, disorder, or condition associated with SCN10A activity in an individual in need thereof.

### Abbreviations and Definitions

Unless defined otherwise, all technical and scientific terms used herein have the same meaning as commonly understood by one of skill in the art to which the disclosure pertains. Moreover, reference to an element by the indefinite article “a” or “an” does not exclude the possibility that more than one element is present, unless the context clearly requires that there be one and only one element. The indefinite article “a” or “an” thus usually means “at least one.” Furthermore, use of “including,” as well as other forms, such as “include,” “includes” and “included” is not limiting.

Certain definitions used herein are defined as follows:

As used herein, “about” means within a statistically meaningful range of a value or values such as, for example, a stated concentration, length, molecular weight, pH, sequence similarity, time frame, temperature, volume, etc. Such a value or range can be within an order of magnitude typically within 20%, more typically within 10%, and even more typically within 5% of a given value or range. The allowable variation encompassed by “about” will depend upon the particular system or subject under study, and can be readily appreciated by one of skill in the art.

As used herein, “administer,” “administering,” “administration” and the like refers to providing a substance (e.g., an oligonucleotide, a conjugate, an AOC, or a composition herein) to an individual in a manner that is pharmacologically useful (e.g., to treat a disease, disorder, or condition in the individual or patient).

As used herein, “target sequence” refers to a contiguous portion of the nucleotide sequence of an mRNA molecule formed during the transcription of the Nav1.8 gene (SCN10A), including mRNA that is a product of RNA processing of a primary transcription product.

As used herein, “antisense strand” means an oligonucleotide herein that is complementary to a region of a target sequence. Likewise, and as used herein, “sense strand” means an oligonucleotide herein that is complementary to a region of an antisense strand.

As used herein, “attenuate,” “attenuating,” “attenuation” and the like refers to reducing or effectively halting. As a non-limiting example, one or more of the treatments herein may reduce or effectively halt the onset or progression of pain, as well as related diseases, disorders, and conditions in an individual such as, for example, chronic pain. This attenuation may be exemplified by, for example, a decrease in one or more aspects (e.g., symptoms, tissue characteristics, and cellular, inflammatory, or immunological activity, etc.), as well as related diseases, disorders, and conditions in an individual.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides. This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but preferably not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the antisense of a ssRNA and target sequence, between the sense strand and the antisense strand of a dsRNA, or between the antisense strand of a dsRNA and a target sequence, as will be understood from the context of their use.

As used herein, a polynucleotide which is “substantially complementary to at least part of a messenger RNA (mRNA) refers to a polynucleotide which is substantially complementary to a contiguous portion of the mRNA of interest (e.g., encoding Nav1.8). For example, a polynucleotide is complementary to at least a part of a Nav1.8 mRNA if the sequence is substantially complementary to a non-interrupted portion of an mRNA encoding Nav1.8.

As used herein, “contact,” “contacting” and the like means directly or indirectly introducing or delivering an oligonucleotide such as an oligonucleotide or an AOC into, for example, a cell by facilitating or effecting uptake or absorption into the cell.

“Introducing into a cell”, when referring to a ssRNA or dsRNA, means facilitating uptake or absorption into the cell, as is understood by those skilled in the art. Absorption or uptake of ssRNA or dsRNA can occur through unaided diffusive or active cellular processes, or by auxiliary agents or devices. The meaning of this term is not limited to cells in vitro; a ssRNA or a dsRNA may also be “introduced into a cell”, wherein the cell is part of a living organism. In such instance, introduction into the cell will include the delivery to the organism.

For example, for in vivo delivery, ssRNA or dsRNA can be injected into a tissue site or administered systemically. In vitro introduction into a cell includes methods known in the art such as electroporation and lipofection.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than at the 2′ position, including modifications or substitutions in or of the nucleobase, sugar, or phosphate group.

The term “double-stranded RNA” or “dsRNA”, as used herein, refers to a ribonucleic acid molecule, or complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the dsRNA. In addition to the duplex structure, a dsRNA may comprise one or more nucleotide overhangs.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double stranded over its entire length, i.e., no nucleotide overhang at either end of the molecule.

As used herein, “duplex,” in reference to nucleic acids (e.g., oligonucleotides), means a structure formed through complementary base pairing of two antiparallel sequences of nucleotides.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4). In some embodiments, the antibody of the present disclosure is an IgG1 antibody.

An immunoglobulin G (IgG) type antibody is comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH (also referred to herein as HCVR) and VL (also referred to herein as LCVR) regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, MD. (1991), which is hereby incorporated by reference in its entirety), Chothia (Chothia et al., “Canonical Structures for the Hypervariable Regions of Immunoglobulins,” J. Mol. Biol., 196, 901-917 (1987); Al-Lazikani et al., “Standard Conformations for the Canonical Structures of Immunoglobulins,” J Mol. Biol., 273, 927-948 (1997), which are hereby incorporated by reference in their entirety), North (North et al., “A New Clustering of Antibody CDR Loop Conformations,” J Mol. Biol., 406, 228-256 (2011)), or IMGT® (the International ImMunoGeneTics database available on at imgt.org; see Lefranc et al., Nucleic Acids Res. 27:209-212 (1999), which are hereby incorporated by reference in their entireties).

The term “antigen-binding fragments”, as used herein, refers to a portion of an antibody that binds an antigen or an epitope of the antigen. For example. “TfR binding protein” refers to a portion of an antibody or antibody fragment that binds TfR or an epitope of TfR.

As referred to herein, the term “epitope” refers to the amino acid residues, of an antigen, that are bound by an antibody. An epitope can be a linear epitope, a conformational epitope, or a hybrid epitope. The term “epitope” may be used in reference to a structural epitope. A structural epitope, according to some embodiments, may be used to describe the region of an antigen which is covered by an antibody or antigen binding protein. In some embodiments, a structural epitope may describe the amino acid residues of the antigen that are within a specified proximity (e.g., within a specified number of angstroms) of an amino acid residue of the antibody or antigen binding protein. The term “epitope” may also be used in reference to a functional epitope. A functional epitope, according to some embodiments, may be used to describe amino acid residues of the antigen that interact with amino acid residues of the antibody or antigen binding protein in a manner contributing to the binding energy between the antigen and the antibody or antigen binding protein.

An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene after treatment of a reagent.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

As used herein, “TfR” refers to a transferrin receptor protein or polypeptide, e.g., a human transferrin receptor protein or polypeptide. The amino acid sequence of the human transferrin receptor protein (hTfR) can be found at NCBI Reference Sequence: NP_001121620.1, which is hereby incorporated by reference in its entirety.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or talc. If desired, the tablets may be coated with a material such as glyceryl monostearate or glyceryl distearate, to delay absorption in the gastrointestinal tract.

As used herein, “individual” means any mammal, including cats, dogs, mice, rats, and primates, especially humans. Moreover, “subject” or “patient” may be used interchangeably with “individual.”

As used herein, “labile linker” means a linker that can be cleaved (e.g., by acidic pH). Likewise, “fairly stable linker” means a linker that cannot be cleaved.

As used herein, “modified internucleotide linkage” means an internucleotide linkage having one or more chemical modifications when compared with a reference internucleotide linkage having a phosphodiester bond. A modified nucleotide can be a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. A modified nucleotide can be a non-naturally occurring nucleotide. A modified nucleotide can have, for example, one or more chemical modifications in its sugar, nucleobase and/or phosphate group. Additionally, or alternatively, a modified nucleotide can have one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nucleoside” means a nucleobase-sugar combination, where the nucleobase portion is normally a heterocyclic base. The two most common classes of such heterocyclic bases are purines and pyrimidines. The sugar is normally a pentose sugar such as a ribose or a deoxyribose (e.g., 2′-deoxyribose).

As used herein, “nucleotide” means an organic molecule having a nucleoside (a nucleobase such as, for example, adenine, cytosine, guanine, thymine, or uracil; and a pentose sugar such as, e.g., ribose or 2′-deoxyribose) and a phosphate group, which can serve as a monomeric unit of nucleic acid polymers such as deoxyribonucleic acid (DNA) and ribonucleic acid (RNA).

As used herein, “oligonucleotide” means a short nucleic acid molecule (e.g., less than about 100 nucleotides in length). An oligonucleotide may be single-stranded (ss) or double-stranded (ds). An oligonucleotide may or may not have duplex regions. As a set of non-limiting examples, an oligonucleotide may be, but is not limited to, a small interfering RNA (siRNA), microRNA (miRNA), short hairpin RNA (shRNA), Dicer substrate interfering RNA (DsiRNA), antisense oligonucleotide (ASO), short siRNA or ss siRNA. In some instances, the oligonucleotide is a phosphorodiamidate morpholino oligomers (PMO), which are short single-stranded oligonucleotide analogs that are built upon a backbone of morpholine rings connected by phosphorodiamidate linkages. An oligonucleotide is a single stranded (ss) oligonucleotide (e.g., ASO) or a double stranded (ds) oligonucleotide (e.g., siRNA).

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include, but are not limited to, 5′ phosphonates, such as 5′ methylenephosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nucleic Acids Res. 43:2993-3011).

As used herein, “reduced expression” or “reduced activity,” and with respect to a gene (e.g., SCN10A), means a decrease in the amount or level of RNA transcript (e.g., SCN10A mRNA) or protein (e.g., Nav1.8 protein) encoded by the gene and/or a decrease in the amount or level of activity of the gene or protein in a cell, a population of cells, a sample or a subject, when compared to an appropriate reference (e.g., a reference cell, population of cells, sample or individual).

The terms “silence” and “inhibit the expression of”, in as far as they refer to the Nav1.8 gene, herein refer to the at least partial suppression of the expression of the Nav1.8 gene, as manifested by a reduction of the amount of mRNA transcribed from the Nav1.8 gene which may be isolated from a first cell or group of cells in which the Nav1.8 gene is transcribed and which has or have been treated such that the expression of the Nav1.8 gene is inhibited, as compared to a second cell or group of cells substantially identical to the first cell or group of cells but which has or have not been so treated (control cells).

Alternatively, the degree of inhibition may be given in terms of a reduction of a parameter that is functionally linked to Nav1.8 gene transcription, e.g., the amount of protein encoded by the Nav1.8 gene which is secreted by a cell, or the number of cells displaying a certain phenotype, e.g., apoptosis. In principle, Nav1.8 gene silencing may be determined in any cell expressing the target, either constitutively or by genomic engineering, and by any appropriate assay. However, when a reference is needed in order to determine whether a given siRNA inhibits the expression of the Nav1.8 gene by a certain degree and therefore is encompassed by the instant invention.

As used herein, “region of complementarity” means a sequence of nucleotides of a nucleic acid (e.g., a double stranded oligonucleotide) that is sufficiently complementary to an antiparallel sequence of nucleotides to permit hybridization between the two sequences of nucleotides under appropriate hybridization conditions (e.g., in a phosphate buffer, in a cell, etc.). An oligonucleotide herein includes a targeting sequence having a region of complementary to a mRNA target sequence.

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the nucleobase, sugar, or phosphate group.

As used herein, siRNA includes a double stranded oligonucleotide having a sense strand and antisense strand, in which the antisense strand or part of the antisense strand is used by the Argonaute 2 (Ago2) endonuclease of the RNA induced silencing complex (RISC) in the cleavage of a target mRNA.

The term a “single stranded oligonucleotide” as used herein includes an antisense strand (or part of that antisense strand) that in combination with the ribonuclease H (RNase H) endonuclease mediates the cleavage of a target mRNA.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “synthetic” refers to a nucleic acid or other molecule that is artificially synthesized (e.g., using a machine such as, for example, a solid-state nucleic acid synthesizer) or that is otherwise not derived from a natural source (e.g., a cell or organism) that normally produces the nucleic acid or other molecule.

As used herein, “treat” or “treating” means an act of providing care to an individual in need thereof, for example, by administering a therapeutic agent (e.g., an oligonucleotide herein) to the individual for purposes of improving the health and/or well-being of the individual with respect to an existing a disease, disorder, or condition, or to prevent or decrease the likelihood of the occurrence of a disease, disorder, or condition. Treating also can involve reducing the frequency or severity of at least one sign, symptom or contributing factor of a disease, disorder, or condition experienced by the individual. The term “treat” or “treating” also includes relief from or alleviation of the perception of pain, including the relief from or alleviation of the intensity and/or duration of a pain (e.g., burning sensation, tingling, electric-shock-like feelings, etc.) experienced by a subject in response to a given stimulus (e.g., pressure, tissue injury, cold temperature, etc.). Relief from or alleviation of the perception of pain can be any detectable decrease in the intensity or duration of pain. Treatment can occur in a subject (e.g., a human or companion animal) suffering from a pain condition or having one or more symptoms of a pain-related disorder that can be treated according to the present disclosure, or in an animal model of pain.

By “Nav1.8” as used herein is meant, any Nav1.8 protein, peptide, or polypeptide associated with the development or maintenance of an ion channel. The terms “Nav1.8” also refer to nucleic acid sequences encoding any Nav1.8 protein, peptide, or polypeptide. The gene encoding for Nav1.8 is referred to as SCN10A.

As used herein, a “pharmaceutical composition” comprises a pharmacologically effective amount of an oligonucleotide or conjugate and a pharmaceutically acceptable carrier. As used herein, “pharmacologically effective amount,” “therapeutically effective amount” or simply “effective amount” refers to that amount of an RNA effective to produce the intended pharmacological, therapeutic, or preventive result. For example, if a given clinical treatment is considered effective when there is at least a 25% reduction in a measurable parameter associated with a disease or disorder, a therapeutically effective amount of a drug for the treatment of that disease or disorder is the amount necessary to affect at least a 25% reduction in that parameter.

As used herein, the phrases “therapeutically effective amount” and “prophylactically effective amount” refer to an amount that provides a therapeutic benefit in the treatment, prevention, or management of pain or an overt symptom of pain. The specific amount that is therapeutically effective can be readily determined by ordinary medical practitioner and may vary depending on factors known in the art, such as, e.g., the type of pain, the patient's history and age, the stage of pain, and the administration of other anti-pain agents.

As used herein, a “transformed cell” is a cell into which a vector has been introduced from which a dsRNA molecule may be expressed.

Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The following non-limiting examples are offered for purposes of illustration, not limitation.

### Example 1: Bioinformatics Based Testing/Selection of siRNAs

Design and In-Silico Selection of siRNAs for Targeting of Human SCN10A Messenger RNA

An in-silico triage was conducted to select siRNA sequences targeting SCN10A (sodium voltage-gated channel alpha subunit 10, Gene ID:6336, Alias:NAv1.8) for in vitro testing. All possible 5856 19-mer siRNA sequences for the SCN10A human messenger RNA (mRNA) were created in-silico using the SCN10A reference sequence NM_006514.3 (SEQ ID NO: 648), which is hereby incorporated by reference in its entirety. For bioinformatics analysis, all transcript RNA sequences were obtained from NCBI RefSeq DB release 95 (July 2019) and Ensembl DB release 97 (July 2019). Table 11 shows the reference sequences used for in-silico screening and selection. The selection criteria used to select the initial round of siRNAs were:
a. Perfect match (19-mer) with sequence of human SCN10A and cynomolgus/rhesus macaques SCN10A was required, and cross-reactivity with mouse or rat were analyzed but not required.
b. No sequences with perfect matches or one mismatch against non-SCN10A genes within positions 2-18 (5′-3′) of the antisense strand were included.
c. Three or more mismatches in antisense strand were required for a set of other sodium channel sequences, including SCN10A (Nav1.4, Gene ID 6329); SCN5A (Nav1.5, Gene ID 6331); SCN9A (Nav1.7, Gene ID 6335), and SCN11A (Nav1.9, Gene ID 11280).
d. Sequences with identical seed region of known miRNAs, based on miRBase R22 (human, rhesus, mouse, rat, March 2018), were excluded.
e. siRNA sequences targeting regions with known SNPs of >1% minor allele frequency (MAF) based on human SNP database (NCBI dbSNP Build 151, October 2017) were excluded.

TABLE 11
Transcript Sequences Used for in-silico
Nav1.8 siRNA Design and Triage.

| Species | Transcript (Accession/ID) |
|---|---|
| cyno | XM_005546684.2 Mfa |
| cyno | XM_015445464.1 Mfa |
| cyno | XM_015445465.1 Mfa |
| cyno | ENSMFAT00000023288.1 |
| human | NM_001293306.2 |
| human | NM_001293307.2 |
| human | NM_006514.3 |
| human | XM_005265371.3 |
| human | XM_011533993.2 |
| human | XM_011533994.2 |
| human | ENST00000643924.1 |
| human | ENST00000449082.3 |
| human | ENST00000643108.1 |
| human | ENST00000644826.1 |
| human | ENST00000655275.1 |
| mouse | NM_001205321.1 |
| mouse | NM_009134.3 |
| mouse | XM_017313223.1 |
| mouse | ENSMUST00000084787.5 |
| mouse | ENSMUST00000213392.1 |
| mouse | ENSMUST00000214408.1 |
| mouse | XM_017313221.1 |
| mouse | XM_017313222.1 |
| mouse | ENSMUST00000216583.1 |
| mouse | ENSMUST00000213401.1 |
| rat | NM_017247.1 |
| rat | XM_017595503.1 |
| rat | XM_017595504.1 |
| rat | XM_017595505.1 |
| rat | ENSRNOT00000046864.4 |
| rhesus | XM_015131314.2 |
| rhesus | XM_015131316.2 |
| rhesus | XM_015131317.2 |
| rhesus | ENSMMUT00000022236.3 |

With the exclusion criteria for selecting Nav1.8 siRNA, the in-silico screen identified a total of 141 siRNA sequences (see Table 2 above), approximately 2.4% of the total possible 19-mer sequences. Each selected siRNA matched the desired selection criteria of human and cyno/rhesus species predicted cross-reactivity (Table 12) with low predicted off-target effect and miRNA activity.

TABLE 12
Cross-reactivity of the 19-mer siRNAs from the
in-silico Screen (“X” indicates the siRNA
species showed in-silico predicted cross-reactivity).

| | | | | Start position |
|---|---|---|---|---|
| | | | | of target |
| siRNA | NHP | Mouse | Rat | region on human |
| NO. | (predicted) | (predicted) | (predicted) | SCN10A transcript |
| 1 | X | | | 407 |
| 2 | X | | | 1172 |
| 3 | X | | | 426 |
| 4 | X | | | 410 |
| 5 | X | | | 349 |
| 6 | X | | | 537 |
| 7 | X | X | X | 990 |
| 8 | X | X | X | 986 |
| 9 | X | | | 538 |
| 10 | X | X | X | 989 |
| 11 | X | | | 361 |
| 12 | X | X | | 983 |
| 13 | X | | | 532 |
| 14 | X | | | 1794 |
| 15 | X | | | 2073 |
| 16 | X | X | X | 435 |
| 17 | X | X | X | 434 |
| 18 | X | X | X | 1442 |
| 19 | X | | | 2071 |
| 20 | X | | | 584 |
| 21 | X | | | 427 |
| 22 | X | | | 576 |
| 23 | X | | | 1790 |
| 24 | X | | | 2063 |
| 25 | X | X | X | 987 |
| 26 | X | | | 535 |
| 27 | X | | | 1787 |
| 28 | X | | | 858 |
| 29 | X | | | 43 |
| 30 | X | | | 856 |
| 31 | X | | | 3958 |
| 32 | X | | | 546 |
| 33 | X | | | 5331 |
| 34 | X | | | 1952 |
| 35 | X | | | 398 |
| 36 | X | | | 396 |
| 37 | X | | | 859 |
| 38 | X | | | 350 |
| 39 | X | | | 1125 |
| 40 | X | | | 4 |
| 41 | X | | | 597 |
| 42 | X | | | 5780 |
| 43 | X | | | 587 |
| 44 | X | | | 1830 |
| 45 | X | | | 5803 |
| 46 | X | | | 1437 |
| 47 | X | | | 400 |
| 48 | X | | | 360 |
| 49 | X | X | X | 988 |
| 50 | X | | | 5809 |
| 51 | X | | | 619 |
| 52 | X | | | 306 |
| 53 | X | | | 2599 |
| 54 | X | | | 5337 |
| 55 | X | | | 3583 |
| 56 | X | | | 3346 |
| 57 | X | | | 5770 |
| 58 | X | | | 1124 |
| 59 | X | | | 3967 |
| 60 | X | X | | 1825 |
| 61 | X | | | 1127 |
| 62 | X | | | 1759 |
| 63 | X | | | 2445 |
| 64 | X | | | 409 |
| 65 | X | | | 3352 |
| 66 | X | X | | 1826 |
| 67 | X | | | 5787 |
| 68 | X | | | 1439 |
| 69 | X | | | 3960 |
| 70 | X | | | 3959 |
| 71 | X | | | 1869 |
| 72 | X | | | 1842 |
| 73 | X | X | | 1824 |
| 74 | X | | | 1829 |
| 75 | X | | | 3355 |
| 76 | X | | | 1123 |
| 77 | X | | | 2952 |
| 78 | X | X | | 5484 |
| 79 | X | | | 2016 |
| 80 | X | | | 1974 |
| 81 | X | | | 1028 |
| 82 | X | | | 5807 |
| 83 | X | | | 1837 |
| 84 | X | | | 50 |
| 85 | X | | | 2753 |
| 86 | X | X | X | 433 |
| 87 | X | | | 2744 |
| 88 | X | | | 1799 |
| 89 | X | | | 4147 |
| 90 | X | | | 5775 |
| 91 | X | | | 5342 |
| 92 | X | | | 5558 |
| 93 | X | | | 1849 |
| 94 | X | X | | 5485 |
| 95 | X | X | | 5514 |
| 96 | X | | | 860 |
| 97 | X | | | 2652 |
| 98 | X | | | 2602 |
| 99 | X | | | 5583 |
| 100 | X | | | 5423 |
| 101 | X | X | X | 253 |
| 102 | X | | | 397 |
| 103 | X | | | 5508 |
| 104 | X | | | 2752 |
| 105 | X | | | 260 |
| 106 | X | | | 4413 |
| 107 | X | | | 4060 |
| 108 | X | | | 2603 |
| 109 | X | | | 5343 |
| 110 | X | | | 1760 |
| 111 | X | | | 5575 |
| 112 | X | | | 4561 |
| 113 | X | | | 5591 |
| 114 | X | | | 209 |
| 115 | X | | | 263 |
| 116 | X | | | 5768 |
| 117 | X | | | 3968 |
| 118 | X | X | | 981 |
| 119 | X | | | 5592 |
| 120 | X | | | 3985 |
| 121 | X | | | 3964 |
| 122 | X | | | 309 |
| 123 | X | X | X | 1440 |
| 124 | X | | | 5336 |
| 125 | X | | | 432 |
| 126 | X | | | 2655 |
| 127 | X | X | X | 1441 |
| 128 | X | | | 2458 |
| 129 | X | | | 2605 |
| 130 | X | | | 5771 |
| 131 | X | | | 4562 |
| 132 | X | X | X | 985 |
| 133 | X | | | 1843 |
| 134 | X | | | 598 |
| 135 | X | | | 919 |
| 136 | X | X | X | 255 |
| 137 | X | | | 2449 |
| 138 | X | | | 783 |
| 139 | X | | | 3993 |
| 140 | X | | | 40 |
| 141 | X | X | X | 5509 |

Of the 5856 possible 19-mer siRNAs sequences targeting the human Nav1.8 transcript (reference sequence NM_006514.3), 141 sequences matched the selected criteria such as low potential off-target toxicity to other NAV channels and genes as well as perfect 19-mer matching to known cynomolgus and rhesus SCN10A mRNA transcripts. These 141 siRNAs were then selected to be synthesized for in vitro screening.

### Example 2: Methods of Making of siRNA Molecules and Description of RNA Modifications

The Nav1.8 21-mer duplexes with 19 bases of complementarity and 3′ dinucleotide overhangs on the guide strand targeting the human SCN10A mRNA (also called Nav1.8 mRNA) were synthesized (see Table 3). RNA modifications were used to optimize the potency of the siRNA duplex and reduce immunogenicity (as shown in FIG. 1A or 1B; with or without the amine or cholesterol handle at the 5′-end of the passenger strand).

All siRNA single strands were fully assembled on solid phase using standard phosphoramidite chemistry and purified over HPLC. Purified single strands were duplexed to get the double stranded 21-mer/19-mer siRNAs (Malecova et al., “Targeted Tissue Delivery of RNA Therapeutics using Antibody—oligonucleotide Conjugates (AOCs),” Nucleic Acids Res. Volume 51, Issue 12, Pages 5901-5910 (2023)).

A non-target control siRNA sequence (siNTC), a published non-targeting control sequence, having a 21-mer duplex with 19 bases of complementarity and 3′ dinucleotide overhangs on the guide strand sequence was synthesized (Burke et al., “siRNA-Mediated Knockdown of P450 Oxidoreductase in Rats: A Tool to Reduce Metabolism by CYPs and Increase Exposure of High Clearance Compounds,” Pharm. Res. 31, 3445-3460 (2014)). The sequence for the siNTC is shown in Table 13.

TABLE 13
Non-target Control siRNA Sequence.

| siRNA | Sense Strand | Antisense Strand |
|---|---|---|
| ID | (5′ to 3′) | (5′ to 3′) |
| siNTC | CUAGCUGGACACGUCGAUA | UAUCGACGUGUCCAGCUAGUU |
| | (SEQ ID NO: 646) | (SEQ ID NO: 647) |

In some embodiments, the siRNA included vinylphosphonate-modified nucleotide (VpUq) in the antisense strand as illustrated in FIG. 1B and shown in Table 4 (5vp_una2moe). VpUq is a vinylphosphonate-2′-O-methoxyethylribose unlocked nucleic acid with a uridine base. It was synthesized according to procedures found in U.S. Pat. No. 11,110,180, which is hereby incorporated by reference in its entirety. The chemical structure of the VpUq is shown below:

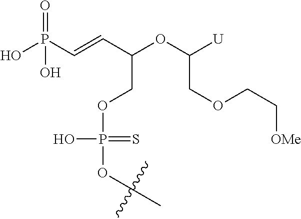

### Example 3: Effect of VpUq Guide Strand Modification in Nav1.8 siRNAs

The siRNAs with modification pattern with VpUq are described in Table 4 and illustrated in FIG. 1B. To evaluate the activity and potency of the VpUq modification in the siRNAs, Nav1.8 siRNA sequences with and without the VpUq modification were tested in human primary neurons. Modified siRNA 410 without VpUq (i.e., siRNA No. 285 in Table 4) and modified siRNA 410 with VpUq (i.e., siRNA No. 305 in Table 4) and modified siRNA 535 without VpUq (i.e., siRNA No. 291 in Table 4) and modified siRNA 535 with VpUq (i.e., siRNA No. 311 in Table 4) were tested. All tested siRNAs were enclosed in lipid nanoparticles as described below.

Human dorsal root ganglia neurons (DRGs) were used as a source of primary cells expressing Nav1.8 mRNA. Fresh primary human DRGs were purchased from AnaBios (San Diego, CA) and maintained in serum-free NbActive4 neural maintenance medium (Axol, Easter Brush, United Kingdom) with 25 ng/mL of recombinant human nerve growth factor (NGF, Axol). Primary DRGs were treated with siRNA encapsulated in Lipid Nano Particles (LNP, Precision Nanosystems, Vancouver, Canada) following the manufacturer's recommendation. LNP-siRNAs (with VpUq) modifications were introduced to growth medium at 150-0.008 nM for 3 days as-is, with no transfection reagent.

Treated human DRGs were kept at 37° C. in 5% CO₂ for 3 days, followed by a wash in phosphate-buffer saline (PBS, ThermoFisher™), and stored in 300 μL of TRIzol™ (ThermoFisher™) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml, STMN2: Hs00975900_ml, and SNAP25: Hs00938957_ml). The relative reduction in Nav1.8 transcripts was determined by normalizing the expression of siRNA treated DRGs to the mock-transfected group using the DDCt method using the following formula: [% hNav1.8 mRNA=100*2^{-DCt(siRNA treated cells)-DCt(mock-transfected cells)}] with DCt determined by the difference in Ct values between SCN10A and the housekeeping genes STMN2 and SNAP25 {avg DCt=[((2*Ct(SCN10A))−Ct(STMN2)−Ct(SNAP25))/2]}(Green M R, Sambrook J. “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)).

The IC50s were calculated by fitting the siRNA concentration-response data into a 3-parameter non-linear regression fitting model [log(concentration of siRNA) vs. % hNav1.8 mRNA] using the GraphPad Prism software (GraphPad Software, LLC).

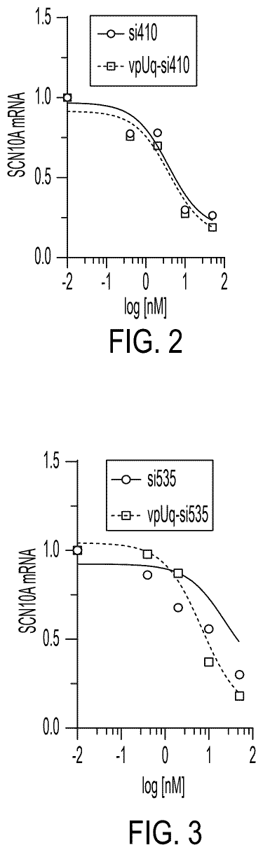

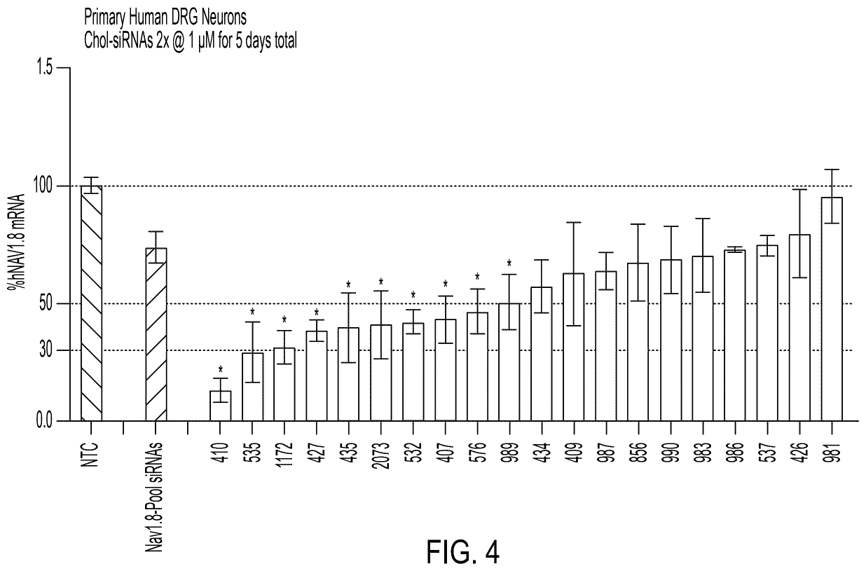

The dose response curves are shown in FIGS. 2 and 3. The addition of the modified nucleotide VpUq to the siRNA had minimal effects in the maximum levels of mRNA reduction. However, the presence of VpUq at the 5′-end of the guide strand affected the siRNA IC50 value in a sequence specific manner. For the siRNA No. 305 (start position of 410), minor differences in the IC50 values were calculated whereas for siRNA No. 311 (start position of 535), the addition of VpUq in lowered the siRNA IC50 value greater than 3-fold from 23.4 nM to 6.9 nM as shown in Table 14.

TABLE 14
VpUq Modification Pattern Effects

| | | | Start position of |
|---|---|---|---|
| | | | target region on |
| siRNA | VpUq | | human SCN10A |
| No. | modification | IC50 (pM) | transcript |
| 285 | not present | 3.787 | 410 |
| 305 | present | 3.931 | 410 |
| 291 | not present | 23.41 | 535 |
| 311 | present | 6.929 | 535 |

In primary human DRGs, incorporation of the VpUq modified nucleotide had a beneficial effect in a sequence-specific manner. The data showed that the IC50 value of the Nav1.8 siRNA sequence siRNA No. 311 was improved by greater than −3-fold. However, minor differences were observed Nav1.8 siRNA sequence siRNA No. 305. Small changes in the maximum mRNA reduction were observed with the incorporation of the modified nucleotide VpUq to the siRNA.

### Example 4: Nav1.8 siRNAs Activity in HEK293 Cell In Vitro

HEK293 cells expressing the human Nav1.8 transcript (HEK293-hNav1.8) were used to determine the in vitro activity of the siRNA sequences. The cells were grown in Dulbecco's Modified Eagle Medium (DMEM; Gibco, Billings, MT) supplemented with 10% of heat-inactivated fetal bovine serum (FBS; Corning, Corning, NY), 1% Pen/Strep (Gibco), 0.1 mg/mL of Zeocin™ (Gibco), and 0.5 μg/mL of Puromycin (ThermoFisher, Waltham, MA). For transfections, HEK293-hNav1.8 cells were seeded at 15,000 cells/well in 100 μL in 96-well tissue culture plates and transfected within 24 hours post-seeding. The siRNAs were gently mixed with Lipofectamine™ RNAiMAX transfection reagent (ThermoFisher) added to the wells to a final concentration of 10 nM or 1 nM according to manufacturer's recommendation.

Treated cells were kept at 37° C. in 5% CO₂ for 72 hours, followed by a wash in phosphate-buffer saline (PBS, ThermoFisher), and stored in 300 μL of TRIzol™ (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml and PPIB: Hs00168719_ml). The relative reduction in Nav1.8 transcripts was determined by normalizing the expression of siRNA treated HEK-hNav1.8 cells to the mock-transfected group using the DDCt method and the following formula: [% hNav1.8 mRNA=100*2^{-DCt (siRNA treated cells)-DCt (mock-transfected cells)}] with DCt determined by the difference in Ct values between SCN10A and the housekeeping gene PPIB [DCt=Ct(SCN10A)−Ct(PPIB)](Green M R, Sambrook J. “Quantification of RNA by Real-Time Reverse Transcription-Polymerase Chain Reaction (RT-PCR),” Cold Spring Harb. Protoc., 2018(10): pdb.prot095042; Livak K J, Schmittgen T D, “Analysis of Relative Gene Expression Data Using Real-Time Quantitative PCR and the 2-ΔΔCT Method,” Methods, 25(4), 402-8 (2001)).

Table 15 summarizes the screening results, showing the Nav1.8 targeting siRNA sequences ranked based on the in vitro activity at the 10 nM concentration of siRNA. The % hNav1.8 mRNA represents the relative expression of human NAv1.8 mRNA remaining after siRNA treatment compared to mock-transfected control cells. Accompanying the siRNA number in the table is the starting siRNA sequence position based on the SCN10A reference transcript sequence (NM_006514.3).

A non-target human siRNA sequence (siNTC, Table 13) was used as a transfection control to account for unspecific mRNA reduction. The control results are also shown in Table 15. Significant mRNA reduction cut-off was determined using the combined average of the siNTC group (10 nM and 1 nM siRNA concentrations), subtracted by 3 standard deviations (std.dev) (Average siNTC=95.6%; std.dev=14.8%).

TABLE 15
In vitro Activity at Two siRNA Concentrations in HEK-
293 hNaᵥ1.8 Cells. The siRNAs are modified as shown
in FIG. 1A but without the amino/cholesterol handle.

| | | | Start position |
|---|---|---|---|
| | | | of target |
| | | | region on |
| | % hNaᵥ1.8 | % hNaᵥ1.8 | human |
| | mRNA at | mRNA at | SCN10A |
| siRNA No. | 10 nM | 1 nM | transcript |
| 142 | 17.84 | 32.98 | 407 |
| 143 | 19.55 | 32.77 | 1172 |
| 144 | 22.59 | 48.88 | 426 |
| 145 | 25.71 | 30.56 | 410 |
| 146 | 27.11 | 65.56 | 349 |
| 147 | 27.32 | 31.37 | 537 |
| 148 | 30.89 | 37.77 | 990 |
| 149 | 31.82 | 37.30 | 986 |
| 150 | 32.46 | 51.47 | 538 |
| 151 | 33.14 | 46.34 | 989 |
| 152 | 33.18 | 46.49 | 361 |
| 153 | 33.58 | 38.86 | 983 |
| 154 | 34.14 | 45.02 | 532 |
| 155 | 34.28 | 51.22 | 1794 |
| 156 | 34.63 | 57.53 | 2073 |
| 157 | 35.08 | 45.33 | 435 |
| 158 | 35.45 | 43.51 | 434 |
| 159 | 36.02 | 55.63 | 1442 |
| 160 | 36.09 | 55.46 | 2071 |
| 161 | 36.48 | 58.97 | 584 |
| 162 | 36.55 | 58.42 | 427 |
| 163 | 36.76 | 56.63 | 576 |
| 164 | 36.99 | 40.63 | 1790 |
| 165 | 37.04 | 40.15 | 2063 |
| 166 | 37.64 | 43.22 | 987 |
| 167 | 38.29 | 51.17 | 535 |
| 168 | 39.65 | 37.18 | 1787 |
| 169 | 39.81 | 41.64 | 858 |
| 170 | 41.35 | 56.28 | 43 |
| 171 | 41.94 | 38.18 | 856 |
| 172 | 42.34 | 55.14 | 3958 |
| 173 | 42.72 | 66.55 | 546 |
| 174 | 42.74 | 53.86 | 5331 |
| 175 | 43.14 | 54.38 | 1952 |
| 176 | 43.22 | 71.53 | 398 |
| 177 | 43.39 | 71.96 | 396 |
| 178 | 44.20 | 49.82 | 859 |
| 179 | 44.20 | 52.20 | 350 |
| 180 | 44.67 | 57.52 | 1125 |
| 181 | 45.74 | 76.21 | 4 |
| 182 | 46.05 | 66.08 | 597 |
| 183 | 46.29 | 45.37 | 5780 |
| 184 | 46.40 | 46.51 | 587 |
| 185 | 47.40 | 43.23 | 1830 |
| 186 | 48.41 | 66.25 | 5803 |
| 187 | 48.42 | 63.52 | 1437 |
| 188 | 48.57 | 69.65 | 400 |
| 189 | 48.91 | 76.47 | 360 |
| 190 | 49.51 | 56.68 | 988 |
| 191 | 49.78 | 49.95 | 5809 |
| 192 | 51.04 | 81.72 | 619 |
| 193 | 51.24 | 72.83 | 306 |
| 194 | 51.73 | 64.94 | 2599 |
| 195 | 53.33 | 55.92 | 5337 |
| 196 | 54.79 | 75.38 | 3583 |
| 197 | 54.99 | 61.97 | 3346 |
| 198 | 55.20 | 68.33 | 5770 |
| 199 | 55.39 | 56.55 | 1124 |
| 200 | 55.62 | 67.05 | 3967 |
| 201 | 55.64 | 57.15 | 1825 |
| 202 | 56.04 | 53.50 | 1127 |
| 203 | 56.16 | 65.78 | 1759 |
| 204 | 56.48 | 68.04 | 2445 |
| 205 | 56.76 | 57.52 | 409 |
| 206 | 56.85 | 67.37 | 3352 |
| 207 | 57.02 | 55.99 | 1826 |
| 208 | 57.34 | 72.13 | 5787 |
| 209 | 57.48 | 63.17 | 1439 |
| 210 | 57.99 | 69.56 | 3960 |
| 211 | 58.31 | 72.54 | 3959 |
| 212 | 58.46 | 73.96 | 1869 |
| 213 | 58.58 | 69.56 | 1842 |
| 214 | 58.62 | 66.49 | 1824 |
| 215 | 58.68 | 58.20 | 1829 |
| 216 | 58.87 | 69.06 | 3355 |
| 217 | 59.51 | 65.56 | 1123 |
| 218 | 59.64 | 75.02 | 2952 |
| 219 | 60.10 | 62.27 | 5484 |
| 220 | 60.42 | 69.49 | 2016 |
| 221 | 60.57 | 73.50 | 1974 |
| 222 | 60.76 | 60.02 | 1028 |
| 223 | 62.60 | 65.58 | 5807 |
| 224 | 62.71 | 57.12 | 1837 |
| 225 | 63.15 | 77.17 | 50 |
| 226 | 63.97 | 66.33 | 2753 |
| 227 | 64.20 | 73.24 | 433 |
| 228 | 64.75 | 57.20 | 2744 |
| 229 | 64.93 | 71.93 | 1799 |
| 230 | 65.36 | 67.42 | 4147 |
| 231 | 65.76 | 84.60 | 5775 |
| 232 | 66.11 | 59.35 | 5342 |
| 233 | 66.22 | 72.64 | 5558 |
| 234 | 66.30 | 86.94 | 1849 |
| 235 | 66.54 | 62.96 | 5485 |
| 236 | 66.88 | 65.58 | 5514 |
| 237 | 67.14 | 37.56 | 860 |
| 238 | 67.34 | 70.56 | 2652 |
| 239 | 67.95 | 53.73 | 2602 |
| 240 | 69.16 | 82.02 | 5583 |
| 241 | 69.66 | 84.60 | 5423 |
| 242 | 69.76 | 75.41 | 253 |
| 243 | 70.94 | 81.22 | 397 |
| 244 | 70.96 | 78.95 | 5508 |
| 245 | 71.16 | 66.87 | 2752 |
| 246 | 74.64 | 74.06 | 260 |
| 247 | 75.13 | 54.74 | 4413 |
| 248 | 76.07 | 63.95 | 4060 |
| 249 | 76.39 | 63.98 | 2603 |
| 250 | 77.23 | 84.47 | 5343 |
| 251 | 78.37 | 90.37 | 1760 |
| 252 | 78.69 | 96.77 | 5575 |
| 253 | 79.20 | 63.59 | 4561 |
| 254 | 79.60 | 75.48 | 5591 |
| 255 | 79.95 | 81.01 | 209 |
| 256 | 80.80 | 84.54 | 263 |
| 257 | 82.25 | 79.49 | 5768 |
| 258 | 82.38 | 63.61 | 3968 |
| 259 | 82.79 | 86.62 | 981 |
| 260 | 82.92 | 102.21 | 5592 |
| 261 | 83.74 | 74.75 | 3985 |
| 262 | 85.50 | 68.20 | 3964 |
| 263 | 85.51 | 74.33 | 309 |
| 264 | 87.06 | 109.87 | 1440 |
| 265 | 87.57 | 65.61 | 5336 |
| 266 | 87.89 | 83.12 | 432 |
| 267 | 89.31 | 74.03 | 2655 |
| 268 | 89.52 | 82.54 | 1441 |
| 269 | 90.37 | 91.60 | 2458 |
| 270 | 91.65 | 70.58 | 2605 |
| 271 | 92.03 | 103.69 | 5771 |
| 272 | 94.04 | 67.76 | 4562 |
| 273 | 97.08 | 99.70 | 985 |
| 274 | 98.19 | 87.94 | 1843 |
| 275 | 99.42 | 78.43 | 598 |
| 276 | 99.94 | 106.80 | 919 |
| 277 | 101.80 | 92.03 | 255 |
| 278 | 107.04 | 61.69 | 2449 |
| 279 | 110.97 | 85.12 | 783 |
| 280 | 112.95 | 77.68 | 3993 |
| 281 | 113.99 | 85.41 | 40 |
| 282 | 241.69 | 259.71 | 5509 |
| siNTC* | 107.21 | 84.35 | not applicable |

*indicates the non-target control siRNA (siNTC) sequence.

The selected 141 siRNA sequences from the in-silico screen were synthesized and were further evaluated in vitro in HEK293 cells expressing the human Nav1.8. The HEK293-hNav1.8 cells were transfected with Nav1.8 siRNA sequences using lipofectamine. Of the 141 siRNA sequences tested in HEK293-hNav1.8 cells, 52 siRNA sequences induced greater than 50% reduction in Nav1.8 mRNA levels (% hNav1.8 mRNA<51.2%) at 10 nM or 1 nM (see Table 15). The siRNA sequence siRNA No. 142 (start position of target region on human SCN10A transcript at 407) had the best activity in decreasing Nav1.8 mRNA levels with more than 85% decrease in Nav1.8 mRNA levels at 10 nM (% hNav1.8 mRNA=17.8%).

In the HEK293-hNav1.8 cells line, it was shown that Nav1.8 mRNA levels can be down regulated by Nav1.8 siRNA sequences. Although 141 19-mer siRNA sequences were identified the in-silico screen, 52 of the tested Nav1.8 siRNA sequences showed greater than 50% inhibitory activity at 10 nM or 1 nM (% hNav1.8 mRNA<51.2%).

### Example 5: Nav1.8 siRNAs Concentration-Dependent Activity in HEK293 Expressing Human Nav1.8

HEK293 cells expressing the human Nav1.8 transcript (HEK293-hNav1.8) were used to determine the in vitro hNav1.8 mRNA reduction and the half-maximal inhibitory concentration (IC50) of the selected lead siRNA sequences. The cells were grown in DMEM (Gibco, Billings, MT) supplemented with 10% of heat-inactivated fetal bovine serum (FBS; Corning, Corning, NY), 1% Pen/Strep (Gibco), 0.1 mg/mL of Zeocin™ (Gibco), and 0.5 μg/mL of Puromycin (ThermoFisher, Waltham, MA). For transfections, HEK293-hNav1.8 cells were seeded at 15,000 cells/well in 100 μL in 96-well tissue culture plates and transfected within 24 hours post-seeding. The siRNAs were gently mixed with Lipofectamine™ RNAiMAX transfection reagent (ThermoFisher) added to the wells to a final concentration ranging from 67-0.27 nM according to manufacturer's recommendation.

Treated cells were kept at 37° C. in 5% CO₂ for 72 hours, washed in phosphate buffer saline (PBS, ThermoFisher), and stored in 300 μL of TRIzol™ (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by RT-qPCR using commercially available TaqMan probes (Life Technologies; SCN10A: Hs01045150_ml and PPIB: Hs00168719_ml). The relative reduction in Nav1.8 transcripts was determined by normalizing the expression of siRNA treated HEK-hNav1.8 cells to the mock-transfected group using the DDCt method with the following formula: [% hNav1.8 mRNA=100*2^{-DCt (siRNA treated cells)-DCt (mock-transfected cells)}] with DCt determined by the difference in Ct values between SCN10A and the housekeeping gene PPIB [DCt=Ct(SCN10A)-Ct(PPIB)](Green M R, Sambrook J. “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)).

Table 16 summarizes the screening results, showing the Nav1.8 targeting siRNA sequences ordered based on the siRNA gene starting position of the SCN10A reference transcript sequence (NM 006514.3). The IC50 and percentage of maximum hNav1.8 knockdown (% maxKD) target mRNA were calculated by fitting the siRNA concentration-response data into a 3-parameter non-linear regression fitting model [log(concentration of siRNA) vs. % hNav1.8 mRNA] using the GraphPad Prism software (GraphPad Software, LLC). The maximum knockdown (maxKD) was calculated by the following formula: % maxKD=100-hNav1.8 eMax, with eMax representing the predicted lowest value of the fitted concentration-response curve (Bottom-value of the 3-parameter non-linear regression curve). The in-silico predictions of siRNAs with expected activity against mouse and or rat Nav1.8 mRNA based upon an analysis of the in-silico predictions are also included with the in vitro results.

TABLE 16
Na_{V}1.8 siRNAs Summary of the Concentration-dependent Response
Assays in HEK293-hNa_{V}1.8 Cells. The siRNAs are modified as
shown in FIG. 1A but without the amino/cholesterol handle.

| | | | | | | Start position |
|---|---|---|---|---|---|---|
| | | | | | in- | of target |
| | | | | | silico | region on |
| | | % maxKD (100- | | | predicted | human |
| siRNA | % hNa_{V}1.8 | hNav1.8 mRNA | IC50 | | rodent | SCN10A |
| No. | mRNA eMax | eMax) | (nM) | R² | active | transcript |
| 170 | 36.23 | 63.77 | 0.56 | 0.92 | X | 43 |
| 146 | 41.18 | 58.82 | 1.36 | 0.93 | | 349 |
| 152 | 33.96 | 66.04 | 0.28 | 0.97 | | 361 |
| 142 | 24.85 | 75.15 | 0.13 | 0.98 | | 407 |
| 145 | 20.27 | 79.73 | 0.2 | 0.98 | | 410 |
| 144 | 31.68 | 68.32 | 0.37 | 0.92 | | 426 |
| 162 | 38.46 | 61.54 | 0.22 | 0.92 | | 427 |
| 158 | 29.01 | 70.99 | 1.01 | 0.96 | X | 434 |
| 157 | 38.71 | 61.29 | 0.66 | 0.81 | X | 435 |
| 154 | 30.76 | 69.24 | 0.18 | 0.97 | | 532 |
| 167 | 32.96 | 67.04 | 0.28 | 0.98 | | 535 |
| 147 | 28.17 | 71.83 | 0.26 | 0.96 | | 537 |
| 150 | 34.4 | 65.6 | 0.35 | 0.97 | | 538 |
| 163 | 34.67 | 65.33 | 0.25 | 0.97 | | 576 |
| 161 | 41.09 | 58.91 | 0.66 | 0.97 | | 584 |
| 171 | 36.4 | 63.6 | 0.26 | 0.97 | | 856 |
| 169 | 42.56 | 57.44 | 1.07 | 0.94 | | 858 |
| 237 | 37.78 | 62.22 | 0.19 | 0.97 | | 860 |
| 153 | 29.41 | 70.59 | 0.73 | 0.98 | X | 983 |
| 149 | 28.78 | 71.22 | 0.37 | 0.97 | X | 986 |
| 166 | 41.74 | 58.26 | 0.71 | 0.94 | X | 987 |
| 151 | 40.52 | 59.48 | 0.46 | 0.94 | X | 989 |
| 148 | 38.88 | 61.12 | 0.23 | 0.92 | X | 990 |
| 143 | 28.8 | 71.2 | 0.7 | 0.85 | | 1172 |
| 159 | 54.55 | 45.45 | 0.63 | 0.81 | | 1442 |
| 168 | 43.09 | 56.91 | 0.47 | 0.73 | | 1787 |
| 164 | 43.01 | 56.99 | 0.68 | 0.75 | | 1790 |
| 155 | 52.93 | 47.07 | 1.49 | 0.72 | | 1794 |
| 165 | 36.8 | 63.2 | 0.33 | 0.82 | | 2063 |
| 160 | 35.01 | 64.99 | 1.24 | 0.8 | | 2071 |
| 156 | 37.01 | 62.99 | 0.57 | 0.93 | | 2073 |
| siNTC | 93.54 | 6.46 | ND | <0.1 | | |

Legend: ND means not determined; X indicates expected activity against mouse and or rat Na_{V}1.8 mRNA based on the in-silico analysis.

Based on the activities of the siRNA sequences from the in vitro screening in HEK cells as shown in Table 15, the 31 siRNA sequences with the highest inhibitory activity (lowest % hNav1.8 mRNA) at 10 nM or 1 nM were further assayed in concentration-dependent response assays in the HEK293-hNav1.8 cell line (top 30 rows from Table 15 plus siRNA No. 237 sequence (start position of 860)) to determine the maximum Nav1.8 transcript reduction and the siRNA half-maximal inhibitory concentration.

As shown in Table 16, most siRNA sequences induced greater than 50% decrease in Nav1.8 mRNA levels. The Nav1.8 siRNA No. 145 sequence (start position of 410) had the highest maxKD of 80%, and the Nav1.8 siRNA No. 142 sequence (start position of 407) had the lowest IC50 with 0.13. Overall, the 31 Nav1.8 siRNA sequences had a maxKD average of 63.8% (std.dev=7.3%) and an average IC50 of 0.54 nM (std.dev=0.37 nM). As expected, the siNTC negative control had a negligible effect, with a poor non-linear regression R-square (R2<0.1). A summary of the 3-parameter non-linear regression is also shown in Table 16.

The selected siRNA sequences showed in vitro activities and potencies in the HEK293-hNav1.8 cell line, with 7 siRNA sequences reducing Nav1.8 transcript expression levels by more than 70%. Regarding the IC50s, 26 of the 31 Nav1.8 siRNAs had activities in the subnanomolar range (<1 nM), with the remaining siRNA sequences in the low single-digit nanomolar range (<2 nM).

### Example 6: Evaluation of Off Target Effects of Nav1.8 siRNAs

Bulk RNA-seq analysis was performed on human skeletal myotubes (Institute of Myology, France), grown, and differentiated using respective medias from Promocell (Heidelberg, Germany). After differentiation, cells were treated with 50 nM LNP-siRNA negative control (siNTC) or Nav1.8 siRNAs. The tested siRNAs are: siRNA No. 283 of Table 4 (start position of 407), siRNA No. 285 of Table 4 (start position of 410), siRNA No. 291 of Table 4 (start position of 535) and siRNA No. 301 of Table 4 (start position of 1172)) for 24 hours (n=4, per LNP-siRNA). The modification pattern of the siRNA sequences is described in Table 4.

Total RNA was isolated using TRIzol™ (ThermoFisher™) followed by chloroform extraction and isopropanol precipitation for the QIAGEN RNeasy® mini kit following the manufacturer's recommendations (QIAGEN, Hilden Germany). Isolated RNA was DNAseI (QIAGEN) treated on-column and quantified via Nanodrop (ThermoFisher) after resuspension, using the 260/280 nm ratio. cDNA libraries were prepared with Poly(A)+selection. Sequencing reads were normalized to Transcripts Per Kilobase Million (TPM) for downstream analyses.

The siRNA sequences siRNA No. 283, siRNA No. 285, siRNA No. 291 and siRNA No. 301 were selected for off-target analysis in human skeletal myotube cells. Bulk RNA-seq analysis was performed and differentially expressed (DE) genes were identified using the DESeq2 package for R and a P-Adjusted value<0.05 (Love M I, et al., “Moderated Estimation of Fold Change and Dispersion for RNA-seq Data with DESeq2,” Genome Biol. 15(12):550 (2014)). The siNTC treated cells were used as the baseline comparison condition. The number of DE genes with a log 2 fold-change (FC) greater than 2 are reported in Table 17.

Lipid nanoparticle delivery of the 4 Nav1.8 siRNA sequences in vitro to human skeletal myotubes showed a negligible to small downregulation of SCN10A non-related genes. The siRNA No. 291 (start position of 535) showed the highest undesired off-target profile (total of 14 DE genes, with a log 2 FC over siNTC greater than 2) while the siRNA No. 285 (start position of 410) sequence showed the lowest off target effects (no DE genes with a log 2 FC over siNTC greater than 2).

TABLE 17
Off-target Analysis of Human Skeletal
Myotubes Treated with Nav1.8 siRNAs.

| | | | | Start position of |
|---|---|---|---|---|
| | Nav1.8 | Number of | Number of | target region on |
| Cell | siRNA | upregulated | downregulated | human SCN10A |
| type | No. | genes | genes | transcript |
| Human | 283 | 0 | 1 | 407 |
| skeletal | 285 | 0 | 0 | 410 |
| myotubes | 291 | 0 | 14 | 535 |
| | 301 | 0 | 8 | 1172 |

Overall, all lead Nav1.8 siRNAs showed little to negligible off-target profile, with sequence siRNA No. 285 (start position of 410) showing the best overall performance in vitro, with a negligible off-target profile after 24 hours LNP-siRNA treatment.

### Example 7: Activity of Lead Nav1.8-siRNAs in DRG Neurons

Human neurons expressing functional Nav1.8 mRNA were used to confirm the activity of lead Nav1.8 siRNAs. The top 14 Nav1.8 siRNA sequences with the highest % maxKD and/or the lowest IC50s were selected for further evaluation. An additional 5 siRNA sequences were selected based on the in-silico predicted species cross-reactivity to mouse Nav1.8 mRNA. An extra siRNA sequence, siRNA No. 205 sequence of Table 3 (start position of 409), was selected based on its high sequence similarity to the siRNA No. 142 of Table 3 (start position of 407) and siRNA No. 145 of Table 3 (start position of 410). Thus, a total of 20 siRNA sequences were prioritized for synthesis, using the modification pattern as illustrated in FIG. 1A, with a 5′-end cholesterol handle in the passenger strand to allow for efficient transfection in primary human neuron cells without the need of a lipofectamine reagent.

Human dorsal root ganglia neurons (DRGs) were used as a source of primary cells expressing functional Nav1.8 mRNA and protein. Fresh primary human DRGs were purchased from AnaBios (San Diego, CA) and maintained in serum-free NbActive4 neural maintenance medium (Axol, Easter Brush, United Kingdom) with 25 ng/mL of recombinant human NGF (Axol). Cholesterol-conjugated siRNAs (Chol-siRNA) were introduced to growth medium at 1 μM twice (day 0 and day 3, for a total of 5 days) as-is, with no transfection reagent.

Treated human DRGs were kept at 37° C. in 5% CO₂ for 5 days, followed by a wash in phosphate-buffer saline (PBS, ThermoFisher), and stored in 300 μL of TRIzol™ (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml, STMN2: Hs00975900_ml, and SNAP25: Hs00938957_ml). The relative reduction in Nav1.8 transcripts was determined by normalizing the expression of siRNA treated DRGs to the mock-transfected group using the DDCt method using the following formula: [% hNAv1.8 mRNA=₁₀₀*2-DCt (siRNA treated cells)-DCt (mock-transfected cells)] with DCt determined by the difference in Ct values between SCN10A and the housekeeping genes STMN2 and SNAP25 {avg DCt=[((2*Ct(SCN10A))-Ct(STMN2)-Ct(SNAP25))/2]}(Green M R, Sambrook J. “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)).

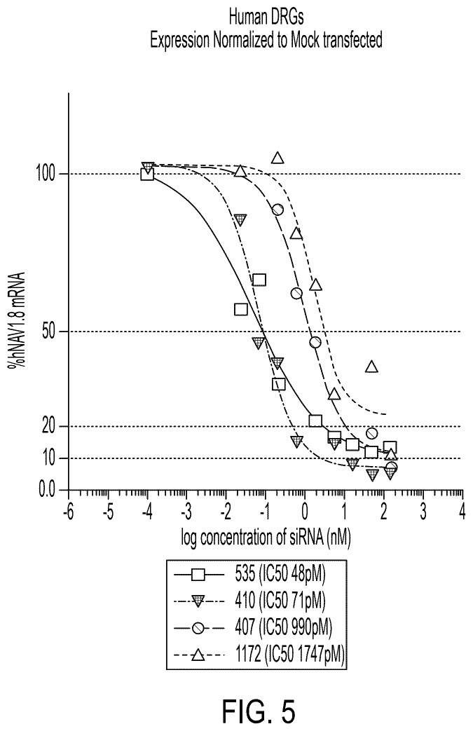

FIG. 4 summarizes the results in human DRGs, showing the Nav1.8 targeting cholesterol modified siRNA sequences ranked based on the in vitro activity at the 1 μM concentration of siRNA. The % hNav1.8 mRNA represents the relative expression of human Nav1.8 mRNA remaining after siRNA treatment compared to mock-transfected control cells. The number below each bar on the bar graph indicates the starting siRNA sequence position based on the SCN10A reference transcript sequence (GenBank: NM_006514.3). The non-target cholesterol modified siRNA sequence (siNTC) was used as a transfection control to account for unspecific mRNA reduction. A commercially available Accell cholesterol modified Nav1.8 siRNA pool was used as positive control (Horizon Discovery Ltd, Waterbeach, UK). An ordinary one-way ANOVA with Dunnett's multiple comparison test was used to determine the adjusted P-values, using the software tool GraphPad Prism (GraphPad Software, LLC).

From the in vitro assay with the HEK293-hNav1.8 cells, 14 Nav1.8 siRNA sequences with the highest % maxKD and or lowest IC50 values and 6 additional siRNA sequences from in the in-silico screen were further selected for synthesis and modified with cholesterol. These modified cholesterol siRNA sequences were further assayed in in vitro using human neurons expressing functional Nav1.8 mRNA transfected with cholesterol modified Nav1.8 siRNA sequences. As shown in FIG. 4, the selected Nav1.8 cholesterol modified siRNA sequences decreased Nav1.8 mRNA levels in human DRGs at the siRNA concentration of 1 μM. Out of the 20 siRNA sequences tested in primary human DRGs, 10 siRNA sequences had at least 50% reduction in the levels of NAv1.8 transcripts in human primary DRGs when compared to the siNTC sequence (P-adjusted value<0.05). The siRNA No. 285 sequence (start position of 410) had the best activity in decreasing Nav1.8 mRNA levels in in human primary DRGs.

Out of the 18 siRNA sequences identified in the preliminary screen in the HEK293-hNav1.8 cell line (Table 14), 10 showed significant Nav1.8 transcript reduction in primary human DRG cell cultures with cholesterol-modified siRNAs. Sequences siRNA No. 285 (start position of 410), siRNA No. 291 (start position of 535) and siRNA No. 301 (start position of 1172) showed consistent high inhibitory activity in vitro, with an overall reduction of Nav1.8 mRNA equal or greater than 70%.

### Example 8: Concentration-Response of Selected Lead Nav1.8 siRNAs in Human DRGs Show IC50 in the Subnanomolar Range

To further characterize the activity and potency of the top 3 Nav1.8 siRNAs in human DRGs, a concentration-response assay was performed in vitro. The 3 siRNA sequences that showed the highest activity in primary neurons were tested. The tested siRNAs are: siRNA No. 285 of Table 4 (start position of 410), siRNA No. 291 of Table 4 (start position of 535), siRNA No. 301 of Table 4 (start position of 1172), and siRNA No. 283 of Table 4 (start position of 407). The siRNAs were encapsulated in Lipid Nano Particles.

Human dorsal root ganglia neurons (DRGs) were used as a source of primary cells expressing Nav1.8 mRNA. Fresh primary human DRGs obtained were purchased from AnaBios (San Diego, CA) and maintained in serum-free NbActive4 neural maintenance medium (Axol, Easter Brush, United Kingdom) with 25 ng/mL of recombinant human NGF (Axol). Primary DRGs were treated with siRNA encapsulated in Lipid Nano Particles (LNP, Precision Nanosystems, Vancouver, Canada) following manufacturer's recommendation.

LNP-siRNAs were introduced to growth medium at 150-0.008 nM for 3 days as-is, with no transfection reagent. The modification patterns of the siRNA sequences are described in Table 4.

Treated human DRGs were kept at 37° C. in 5% CO₂ for 3 days, followed by a wash in phosphate-buffer saline (PBS, ThermoFisher), and stored in 300 μL of TRIzol™ (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml, STMN2: Hs00975900_ml, and SNAP25: Hs00938957_ml). The relative reduction in Nav1.8 transcripts was determined by normalizing the expression of siRNA treated DRGs to the mock-transfected group using the DDCt method using the following formula: [% hNav1.8 mRNA=100*2^{-DCt (siRNA treated cells)-DCt (mock-transfected cells)}] with DCt determined by the difference in Ct values between SCN10A and the housekeeping genes STMN2 and SNAP25 {avg DCt=[((2*Ct(SCN10A))-Ct(STMN2)-Ct(SNAP25))/2]}(Green M R, Sambrook J. “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)).

The IC50 and percentage of maximum hNav1.8 knockdown (% maxKD) target mRNA were calculated by fitting the siRNA concentration-response data into a 3-parameter non-linear regression fitting model [log(concentration of siRNA) vs. % hNav1.8 mRNA] using the GraphPad Prism software (GraphPad Software, LLC). The maximum knockdown (maxKD) was calculated by the following formula: % maxKD=100-hNav1.8 eMax, with eMax representing the predicted lowest value of the fitted concentration-response curve (Bottom-value of the 3-parameter non-linear regression curve).

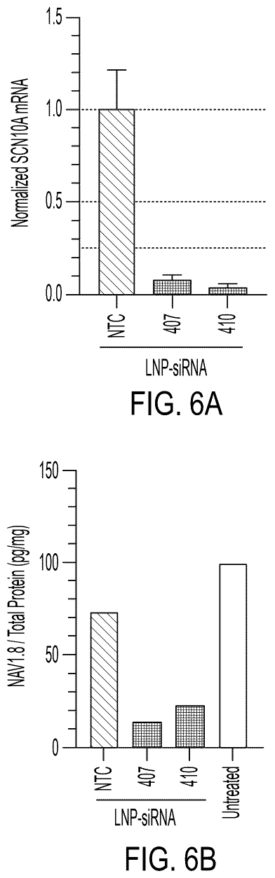

As shown in FIG. 5, the concentration-response curves of the lead top 3 siRNA sequences and siRNA No. 283 showed similar maximum Nav1.8 mRNA reduction in human DRGs, with greater than 75% target transcript reduction for all tested sequences in the LNP format. Nav1.8 siRNA IC50s were in the subnanomolar (<1 nM) range to 3 out of the 4 sequences tested (siRNA No.'s 285, 291 and 283), with siRNA No. 301 in the single digit nanomolar range (<2 nM) (see Table 18).

TABLE 18
Summary Results of the Concentration-
response Assay in Primary Human DRGs.

| | % | | | Start position of |
|---|---|---|---|---|
| | hNav1.8 | % maxKD (100 − | | target region on |
| siRNA | mRNA | hNav1.8 mRNA | IC50 | human SCN10A |
| No. | eMax | eMax) | (pM) | transcript |
| 285 | 7.15 | 92.85 | 71 | 410 |
| 291 | 10.03 | 89.97 | 48 | 535 |
| 283 | 10.81 | 89.19 | 990 | 407 |
| 301 | 23.33 | 76.67 | 1747 | 1172 |

All tested Nav1.8 siRNA sequences significantly reduced Nav1.8 mRNA in human primary DRGs by more than 70%. The siRNA No. 285 showed the highest activity, with a transcript reduction greater than 90% and an IC50 of 71 μM.

### Example 9: Downregulation of SCN10A Transcripts Using siRNA-LNP Leads to Reduction in Nav1.8 Protein Expression in Human Primary Neurons

Test siRNA sequences (i.e., siRNA No. 283 of Table 4 (start position of 407) and siRNA No. 285 of Table 4 (start position of 410)) were tested for their capability to reduce the expression of Nav1.8 protein in human primary neurons. The modification patterns of the siRNA sequences are described in Table 4.

Human dorsal root ganglia neurons (DRGs) were used as a source of primary cells expressing Nav1.8 mRNA. Fresh primary human DRGs were purchased from AnaBios (San Diego, CA) and maintained in serum-free NbActive4 neural maintenance medium (Axol, Easter Brush, United Kingdom) with 25 ng/mL of recombinant human NGF (Axol). Primary DRGs were treated with the test siRNA encapsulated in Lipid Nano Particles (LNP, Precision Nanosystems, Vancouver, Canada) following the manufacturer's recommendation. LNP-siRNAs were introduced to growth medium at 0.5 μg/mL twice for 6 days, with no transfection reagent.

Treated human DRGs were kept at 37° C. in 5% CO₂ for 6 days, followed by a wash in phosphate-buffer saline (PBS, ThermoFisher), and stored in 300 μL of TRIzol (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml, STMN2: Hs00975900_ml, and SNAP25: Hs00938957_ml). The relative reduction in Nav1.8 transcripts was determined by normalizing the expression of siRNA treated DRGs to the mock-transfected group using the DDCt method using the following formula: [% hNav1.8 mRNA=100*2^{-DCt (siRNA treated cells)-DCt (mock-transfected cells)}], with DCt determined by the difference in Ct values between SCN10A and the housekeeping genes STMN2 and SNAP25 {avg DCt=((2*Ct(SCN10A))−Ct(STMN2)−Ct(SNAP25))/2}(Green M R, Sambrook J. “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)).

ELISA was used to measure the Nav1.8 protein concentration in cultured human DRG neuron protein lysate. Growth media was aspirated off and the cultured human DRG neurons were washed once in Dulbecco's phosphate buffered saline (DPBS; Gibco, Cat #14190-136). Whole cell lysates were prepared by suspending cultured human DRG neurons in 100 μL/well of the 1× lysis Buffer (CST, Cat #9803) along with Halt (Protease and Phosphatase Inhibitor (TFS, Cat #78440) diluted to 2× and PMSF (Sigma, #78830-25G, (17.4 mg in 1 mL isopropanol, diluted to 1×). Lysis was incubated on ice for five minutes, then centrifuged at 4° C. at 800×g for 20 minutes. Supernatant was collected and frozen at −80° C. for analysis the next day. Protein concentration was measured with the Pierce™ BCA Protein Assay Kit (Cat #23227) by following the “Microplate Procedure” described in the manufacturer's manual. Samples were further diluted 1:3 in cell lysis buffer before plating. The Meso Scale Discovery (MSD)S-Plex platform was used to detect Nav1.8 protein levels in the cultured human DRG neuron protein lysate. The custom developed assay utilized S-PLEX Development Pack B, SECTOR (25 Plate) (Cat #K15601S-4) kit. Capture and detection antibodies were tagged by MSD. Capture antibody (NeuroMab, Cat #75-166) was diluted to 0.5 μg/mL and a detection antibody was diluted to 0.2 μg/mL. Calibrator was a recombinant C-terminal fragment of human Nav1.8. Sample lysates were diluted 4-fold in assay buffer. Assay buffer base was Diluent 39 (MSD, Cat #R5ABB2) with 100 μL each of the components included in the MSD® Inhibitor Pack (Cat #R70AA-1). Washing steps utilized MSD Tris Wash Buffer (MSD, Cat #R61TX-1) diluted to 1× in double-distilled water (ddH₂O). Plates were read using an MSD plate reader MESO QuickPlex SQ 120MM with MSD Methodical Mind software. Data was analyzed using Discovery Workbench v4. Data was normalized by dividing the Nav1.8 protein concentration by the total protein concentration for each lysate sample. Statistical significance was determined by One-way ANOVA.

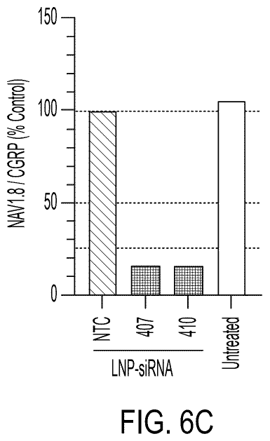

Results: The siRNA sequences siRNA No. 283 and siRNA No. 285 having a minimal off-target profile were further assayed in vitro in human DRGs transfected as the Lipid Nano Particle (LNP) siRNA sequences. Nav1.8 mRNA levels were determined by qPCR (FIG. 6A) and Nav1.8 protein levels were determined by ELISA (FIG. 6B). Negligible transcript regulation was observed with the LNP-siNTC negative control sequence, with an approximately 30% reduction in Nav1.8 protein expression when the assay is normalized by the total protein amount. No difference between the untreated and siNTC group was observed when Nav1.8 protein expression was corrected by CGRP protein expression (human DRG specific protein). The Nav1.8 LNP-siRNAs downregulated SCN10A transcripts by more than 90%. More importantly, reduction in Nav1.8 mRNA levels was correlated by more than 80% inhibition in protein expression levels, independent of the normalization method (total protein loading or human DRGs normalization).

Nav1.8 mRNA and protein expression are highly correlated inhuman primary DRGs in vitro. Lead siRNA sequences siRNA No. 283 and siRNA No. 285 showed great mRNA and protein reduction in vitro following treatment with LNP-Nav1.8 siRNAs. Nav1.8 protein reduction in the Nav1.8 siRNA treated groups, was independent of the protein normalization method. Confirming the linked association between Nav1.8 mRNA regulation and protein translation in human DRGs in vitro.

### Example 10: Expression and Purification of Transferrin Receptor Antibody (TfR)

The exemplified anti-TfR antibodies and antibody fragments of the present disclosure were expressed and purified as follows. Antibodies TBP1, TBP2, and TBP3 (as shown in Table 8 above) are expressed in an appropriate host cell, such CHO cells, either transiently or stably transfected with an expression system for secreting the TBP1 antibody or TBP3 antibody using an optimal predetermined HC:LC vector ratio or a single vector system encoding both HC and LC.

For TBP2 antibody, an optimal predetermined HCA:HCB:LC vector ratio or a single vector system encoding HCA, HCB, and LC is used. The expression plasmids contain cDNA versions of the LC and HC genes for antibody TBP1, TBP2, or TBP3; and are expressed from a commonly used and suitable construct for this purpose, such as one based on human cytomegalovirus major immediate early promoters.

Medium, into which the antibody was secreted, may be purified by conventional techniques, such as mixed-mode methods of ion-exchange and hydrophobic interaction chromatography. For example, the medium containing TBP1 antibody or TBP2 antibody may be applied to and eluted from a Protein A column (Cytiva) using conventional methods. Medium containing TBP3 antibody may be applied to and eluted from a CaptureSelect™ CH1-XL column (Thermo Scientific™) using conventional methods.

Soluble aggregate, multimers, and fragments may be effectively removed by cation exchange chromatography using a POROS™ HS 50 column (Thermo Scientific™) using conventional methods. The product may be immediately frozen, for example at −70° C., refrigerated, or may be lyophilized. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994). Antibodies TBP1, TBP2 or TBP3 may be immediately frozen at −70° C. or stored at 2-8° C. for several months, or may be lyophilized, or preserved in 4° C. for immediate use. Amino acid sequences for the antibodies of the present disclosure are shown in Tables 5-8. The nucleic acid sequences for the antibodies are provided in Table 9.

### Example 11: Method of Making Native Cysteine (nCys) Conjugated Antibody Oligonucleotide Conjugates (AOCs)

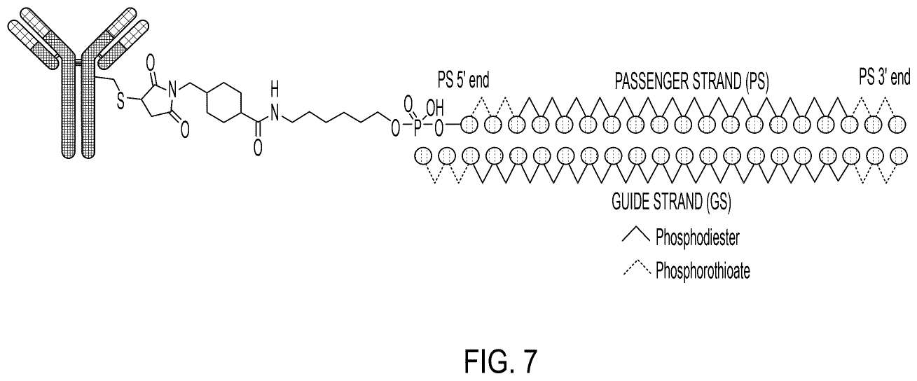

Nav1.8-AOC (FIG. 7) is an antibody-siRNA conjugate (also referred to herein as antibody oligonucleotide conjugate (AOC)) formed by the conjugation of a human IgG1 antibody specific to the human transferrin receptor 1 (anti-TFR1 antibody) to a double stranded siRNA oligonucleotide (Nav1.8 siRNA) targeting Nav1.8 mRNA. Any of the antibodies provided in Tables 5 to 8 may be used in the AOCs and conjugated to any one of the siRNAs provided in Tables 2 to 4. The conjugation may be done via a linker, e.g., a SMCC linker.

A succinimidyl 4-(N-maleimidomethyl)cyclohexane-1-carboxylate (SMCC) maleimide linker is located on 5′ end of the passenger strand and it is conjugated to the antibody through one of the cysteines in the antibody amino acid sequence. The conjugate binds human transferrin receptor on the cell surface, internalizes into the cell and delivers the siRNA oligonucleotide to the intracellular compartment. Upon uptake by the cells, the siRNA loads into the RNA-induced silencing complex (RISC) and hydrolyses the intracellular Nav1.8 mRNA.

Step 1: Antibody Interchain Disulfide Reduction with TCEP

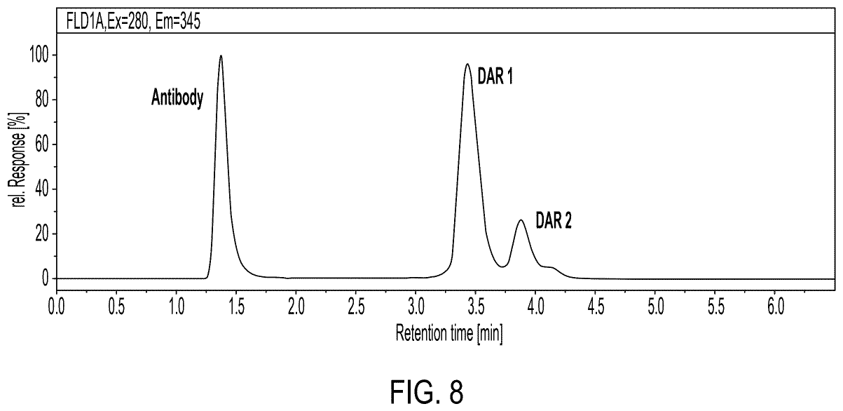

Antibody (anti-human transferrin receptor 1, TBP1) was formulated in phosphate buffered saline (PBS) pH 7.4 and adjusted to 2 mM ethylenediamine tetra acetic acid (EDTA). To this solution, 2 equivalents (EQ) of tris(2-carboxyethyl)phosphine (TCEP) in water was added and rotated for 4 hours at room temperature (RT). To the resultant reaction mixture was added a solution of 4-(N-maleimidomethyl)cyclohexane-1-carboxylic acid (MCC)-siRNA (0.9 EQ) in pH 6 10 mM sodium acetate at RT and rotated for 1 hour. Analysis of the reaction mixture by analytical strong anion exchange (SAX) column chromatography showed antibody siRNA conjugate along with unreacted antibody and siRNA (FIG. 8). The reaction mixture was treated with 10 EQ of N-ethylmaleimide (in dimethyl sulfoxide (DMSO) at 10 mg/mL) to cap any remaining free cysteine residues.

### Step 2: Purification

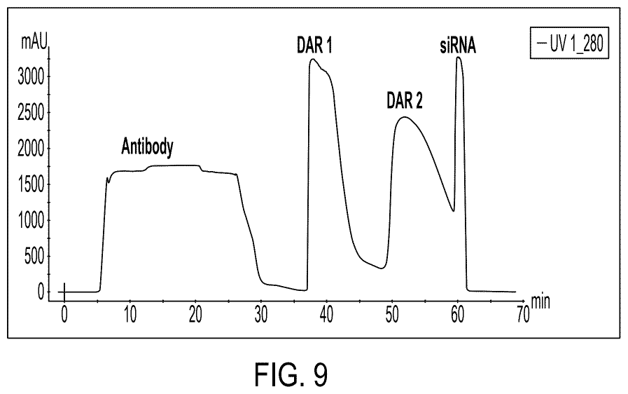

The crude reaction mixture was purified by AKTA explorer fast purification liquid chromatography (FPLC) using anion exchange chromatography (FIG. 9). Fractions containing DAR1 siRNA conjugates were separated, concentrated, and formulated with pH 7.4 PBS.

### FPLC Method 1 Details

Column: HiScale™ 50/20 column, TSKgel® SuperQ-5PW resin
Solvent A: 20 mM phosphate buffer, pH 7.2
Solvent B: 20 mM phosphate buffer, 1.5M NaCl, pH 7.2
Gradients used (% A/% B): 90:10; 81:19; 68:32
Volumes used: 0.5, 0.2, 6

### Step 3: Analysis of the Purified Conjugate

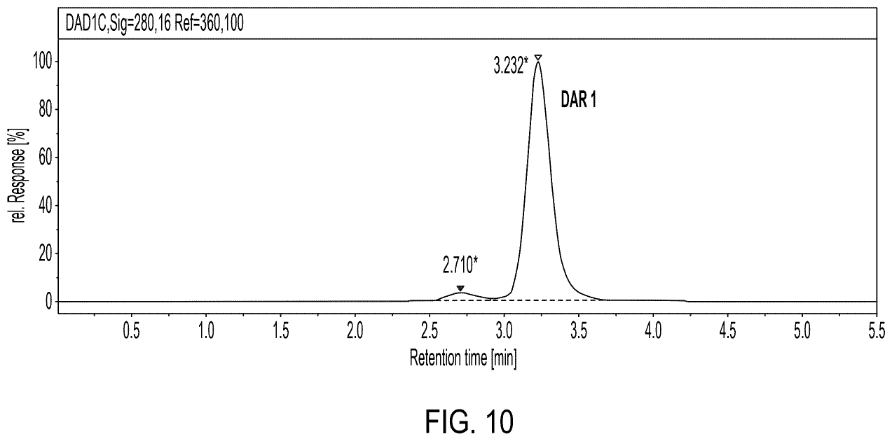

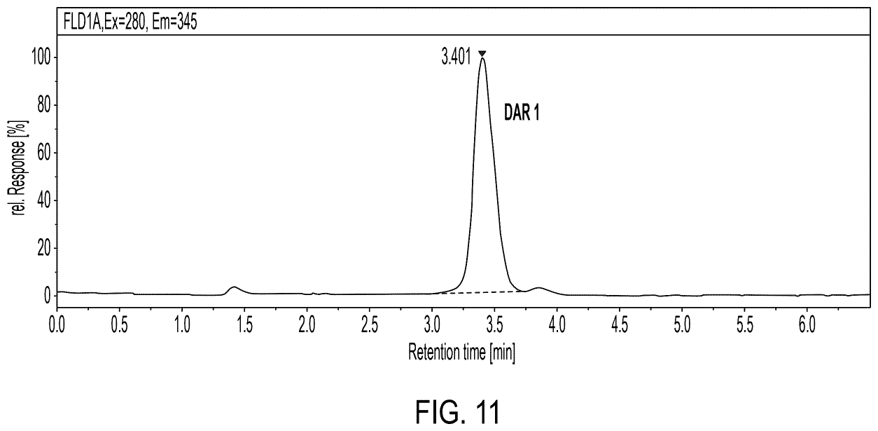

The isolated conjugates were characterized by size exclusion (SEC) and SAX chromatography. The purity of the conjugate was assessed by analytical high pressure liquid chromatography (HPLC) with isolated DAR1 conjugates being greater than 90% pure (FIGS. 10 and 11).

### SAX Method 1 Details

Column: Thermo Scientific, PropPac™ SAX-10, Bio LC, 4×250 mM
Solvent A: 80% 10 mM TRIS pH 8, 20% ethanol
Solvent B: Solvent B: 80% 10 mM TRIS pH 8, 20% ethanol, 1.5 M NaCl
Flow Rate: 0.9 mL/minute
Gradients (% A/% B): 84:16; 60:40; 46.5:53.5; 40:60; 84:16; 84:16
Time associated to gradients: 0, 2, 4, 4.5, 5, 6.5

### SEC Method 1 Details

Column: Waters™ Bioresolve SEC mAb, 2.5 μm, 4.6×150 mm
Solvent A: 55 mM 1 KH₂PO₄, 62 mM Na₂HPO₄·7H₂O, 100 mM Na₂SO₄, 0.05% w/v NaN₃,
pH 6.7
Flow Rate: 0.4 mL/minute
Isocratic (% A): 100
Time associated to isocratic: 0, 5.5

### Example 12: Method of Generating of Engineered Cysteine (eCys) Conjugated AOCs

Site-specific native or engineered cysteine amino acid residues in the antibody molecule were used to conjugate dsRNA (siRNA). Cysteines can be engineered into the primary amino acid sequence of the TfR binding proteins (TBPs) disclosed herein. The approach of introducing cysteines as a means for conjugation has been described in WO 2018/232088, which is both incorporated by reference in its entirety and incorporated specifically in relation to conjugation via cysteine residues. For engineered cysteine conjugation, the TfR binding proteins were first reduced with 10 molar equivalents reducing agent tris(2-carboxyethyl)phosphine (TCEP) at room temperature for two hours, followed by buffer exchange by, for example, tangential flow filtration (TFF) to remove reduction reagent. For TBP2, this is followed by re-oxidation of the TfR binding protein to reform the interchain disulfides with 20 molar equivalent dehydroascorbic acid (DHAA) incubation at room temperature for two hours. Buffer exchange was performed again to remove oxidizing agent. For TBP3, the reoxidation step was omitted as scaffold does not contain interchain disulfide bonds.

Conjugation of dsRNA onto TfR binding proteins were done using the SMCC-functionalized dsRNA. The prepared TBP above was incubated with 0.9-1.0 molar equivalents of the SMCC-dsRNA for conjugation for 1-2 hours at room temperature. Conjugation was monitored using analytical anion exchange chromatography (AEX). A ProPac™ SAX-10 HPLC Column, 10 μm particle, 4 mm diameter, 50 mm length (Thermo Scientific™) was utilized with the following method. Flow rate of 1 mL/min, Buffer A: 20 mM TRIS pH 7.0, Buffer B: 20 mM TRIS pH 7.0+1 M NaCl, at 30° C. The mobile phase gradient is shown in Table 19. Drug antibody/protein ratio (DAR) was calculated based on peak area % from the analytical anion exchange (aAEX) chromatogram.

TABLE 19
HPLC Gradient used to Determine dsRNA
Conjugation to TfR Binding Protein

| Time [min] | A [%] | B [%] |
|---|---|---|
| 0.00 | 85.0 | 15.0 |
| 8.00 | 0.0 | 100.0 |
| 9.00 | 0.0 | 100.0 |
| 9.10 | 85.0 | 15.0 |
| 10.00 | 85.0 | 15.0 |

### Example 13: In Vitro Stability of Antibody Oligonucleotide Conjugates (AOCs)

Stability of the native cysteine conjugated bivalent mAb (TBP1), the engineered cysteine conjugated bivalent mAb (TBP2), and engineered cysteine conjugated monovalent Fab (TBP3) DAR1 AOCs were assessed in vitro. The siRNA portion of the AOC was modified as shown in FIG. 1B with the amine handle.

Samples were prepared at 1 mg/mL protein concentration in phosphate buffered saline (PBS) pH 7.2, then held at 4° C. and 40° C. for 2 weeks. Samples were analyzed by analytical size exclusion chromatography (SEC), AEX (as described in Example 12), and reduced and non-reduced CE-SDS. SEC was performed using an Acquity UPLC Protein BEH SEC 200 Å 1.7 μM particle size, 4.6 mm diameter, 150 mm length column (Waters™). Mobile phase was 50 mM sodium phosphate, 0.3 M NaCl, 0.005% sodium azide, pH 6.8, and elution was isocratic at 0.3 mL/min for an 8-minute run time. Reduced CE-SDS was performed using a Maurice (BioTechne) following the manufacturer's protocol. Non-reduced CE-SDS was performed with a Labchip® GXII (PerkinElmer) following the manufacturer's protocol.

Results are shown in Table 20. Across all assays, the native cysteine conjugated TBP1-410 AOC showed decreased stability relative to the engineered cysteine conjugated TBP2-410 AOC. The monovalent Fab engineered cysteine AOC (TBP3-410 AOC) shows comparable stability relative to the bivalent mAb engineered cysteine AOC.

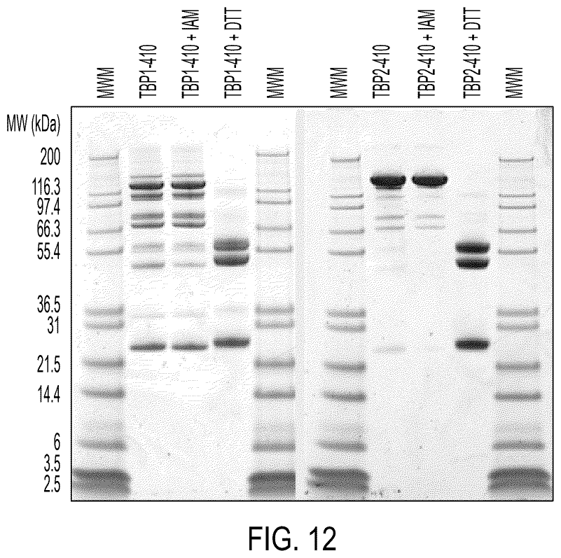

AOCs were analyzed by non-reduced and reduced SDS-PAGE to assess covalent assembly (FIG. 12). NuPAGE™ 4-12% Bis-Tris gels, LDS sample buffer, and MES SDS running buffer were used (ThermoFisher Scientific). Mark12™ molecular weight marker (MWM) was used as a standard. Non-reduced samples were analyzed with and without alkylation by iodoacetamide (JAM) to confirm that the apparent fragmentation under denatured conditions was not a result of artifact reduction during SDS-PAGE sample preparation.

Multiple fragments are observed for the TBP1-410 AOC with and without JAM alkylation corresponding to light chain, heavy chain, half-antibody, heavy chain dimer, heavy chain dimer+light chain, and intact antibody are observed for the TBP1-410 while the TBP2-410 AOC is observed as majority intact antibody with only minor fragment species observed. In the reduced analysis, samples are comparable showing roughly equal intensity bands for heavy chain and heavy chain+RNA with little to no observed light chain+RNA. The fragmentation observed in the TBP1-410 AOC but not in the TBP2-410 AOC is an expected result from the conjugation approaches and may be causing the observed stability differences. Together, these results demonstrate that the engineered cysteine conjugation approach provides a more homogenous, more stable AOC compared to the native cysteine conjugation approach.

TABLE 20
In vitro Stability Results Showing the Change in Main Peak at 40°
C. Relative to 4 ° C. Following 2-week Incubation.

| | | | | CE-SDS |
|---|---|---|---|---|
| | | | CE-SDS | Non- |
| | SEC | AEX | Reduced | Reduced |
| AOC | Δ% Main | Δ% Main | Δ% Main | Δ% Main |
| TBP1-410 | −33.0 | −37 | −8.6 | N/A^{a} |
| TBP2-410 | −1.7 | −7 | 0 | −0.4 |
| TBP3-410 | −2.7 | −0.9 | 0^{b} | 0^{b} |

^{a}Non-reduced CE-SDS analysis could not be performed for the TBP1-410 due to significant peak heterogeneity.
^{b}No % change in peak area was observed, however main peak was shifted due to maleimide ring hydrolysis.

### Example 14: In Vitro Evaluation of the TfR1-Nav1.8 siRNA AOC in Human DRGs

To confirm that the siRNA sequence can be delivered to human DRGs by an anti-transferrin antibody (anti-TfR1) of the present disclosure, an anti-TfR1 antibody conjugated to the siRNA 305 of Table 4 (TBP1-si410 AOC) was synthesized and evaluated in an in vitro assay using primary human DRGs. The siRNA sequence used in the AOC was siRNA 305 as shown in Table 4 and the siRNA was conjugated to the antibody via SMCC linker as shown in FIG. 7. The antibody used in the AOC is TBP1 as shown in Table 8.

Human dorsal root ganglia neurons (DRGs) were used as a source of primary cells expressing Nav1.8 mRNA. Fresh primary human DRGs were purchased from AnaBios (San Diego, CA) and maintained in serum-free NbActive4 neural maintenance medium (Axol, Easter Brush, United Kingdom) with 25 ng/mL of recombinant human NGF (Axol). Primary DRGs were treated with AOCs at 100 nM, twice, for 5 days.

Treated human DRGs were kept at 37° C. in 5% CO₂ for 5 days, followed by a wash in phosphate-buffer saline (PBS, ThermoFisher), and stored in 300 μL of TRIzol (ThermoFisher) at −80° C. until RNA extraction. The total mRNA was isolated using the ZYMO 96-well RNA kit (Zymo Research, Irvine, CA), and 100-250 ng of purified mRNA was used to generate the cDNA with the iScript cDNA synthesis kit (BioRad, Hercules, CA).

RNA expression levels were determined by quantitative reverse transcription polymerase chain reaction (RT-qPCR) using commercially available TaqMan probes (Life Technologies; SCN10A: Hs01045150_ml, STMN2: Hs00975900_ml, and SNAP25: Hs00938957_ml). The relative reduction in Nav1.8 transcripts was determined by normalizing the expression of TBP1-si410 AOC treated DRGs to the siNTC group using the DDCt method and the following formula: [% hNav1.8 mRNA=100*2^{-DCt (si410 treated cells)-DCt (siNTC-treated cells)}] with the DCt determined by the difference in Ct values between SCN10A and the housekeeping genes STMN2 and SNAP25 {avg DCt=((2*Ct(SCN10A))-Ct(STMN2)-Ct(SNAP25))/2}(Green M R, Sambrook J., “Quantification of RNA by Real-Time Reverse Transcription-Polymerase Chain Reaction (RT-PCR),” Cold Spring Harb Protoc. 2018 October, 2018(10): pdb.prot095042; and Livak K J, Schmittgen T D, “Analysis of Relative Gene Expression Data Using Real-Time Quantitative PCR and the 2-ΔΔCT Method,” Methods, 25(4), 402-8 (2001)). A parametric unpaired t-test was performed using the software tool GraphPad Prism (GraphPad Software, LLC).

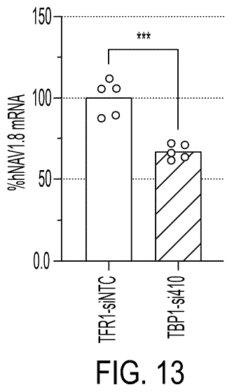

As illustrated in FIG. 13, the tested AOC significantly reduced Nav1.8 mRNA levels (by approximately 30%) when compared to the negative control treated cells (TFR1-siNTC AOC) in primary human DRGs. In primary human DRGs, it was demonstrated that an AOC can deliver aNav1.8 specific siRNA. AOC treatment results in receptor mediated uptake of the Nav1.8 siRNA, as human DRGs express TfR1 on their surface. The AOC uptake resulted in siRNA mediated knockdown of the Nav1.8 mRNA.

### Example 15: In Vivo Activity of OX26-410 and OX26-535 Tool AOCs in Humanized Nav1.8 Rats

Humanized Nav 1.8 rats, in which the rat SCN10A gene was knocked out and replaced with the human SCN10A gene, were developed to allow testing of oligonucleotide therapeutics that only had predicted activity on human SCN10A transcript.

Test siRNAs-410 and siRNA-535 were conjugated to a commercial rat transferrin antibody (OX26 clone, BioXcell) using native cysteine conjugation and purified to a drug-antibody ratio of 1.0. Both the test siRNAs had the modifications as shown in FIG. 1B without the amine or cholesterol handle.

TABLE 21
Description of the AOCs

| Test AOC Name | Description |
|---|---|
| OX26-siRNA410 | OX26 antibody linked to 410 siRNA via |
| | SMCC linker using nCys sites. |
| OX26-siRNA535 | OX26 antibody linked to 535 siRNA via |
| | SMCC linker using nCys sites. |

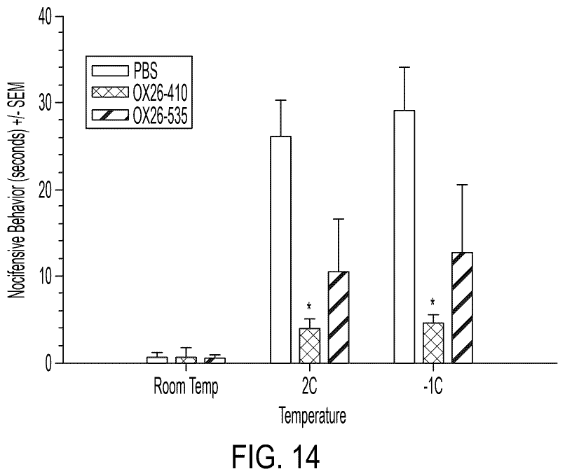

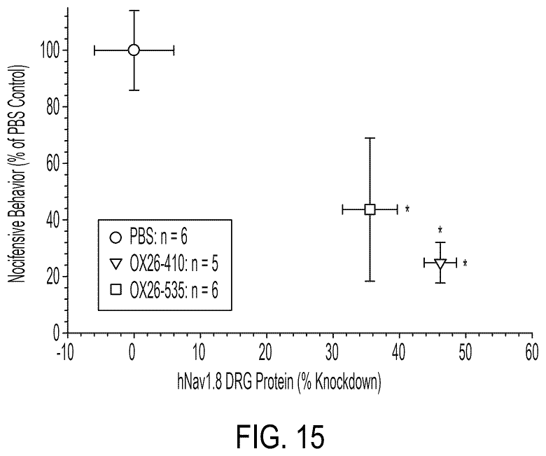

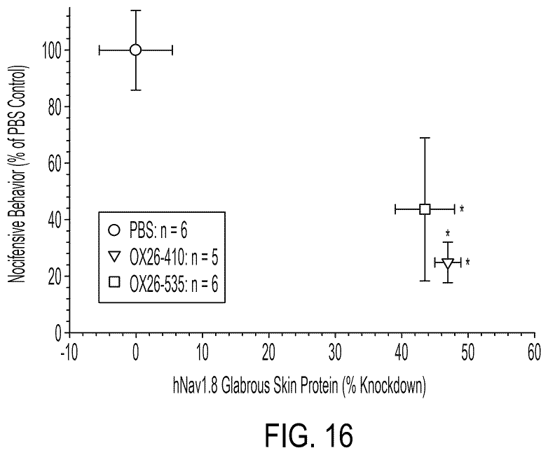

Female rats, approximately 4 weeks old, received an intravenous (IV) administration of either vehicle (PBS), OX26-siRNA410 (6 mg/kg oligo) or OX26-siRNA535 (6 mg/kg oligo). Thirteen days after administration, rats received 3 test sessions in the cold plate system. Rats first received a 5-minute session at room temperature (23° C.) followed by 10 minutes in the home cage. Half of the rats then received a 4-minute test session at 2° C. and the other half received a test session at −1° C. After approximately 2 hours, rats received a test session at the other temperature. No effects of test order were observed. Nocifensive behavior was defined as the time spent licking either rear paw or time spent guarding the rear paws (paw lifted into the body). This time was summed into a Nocifensive Behavior score reported on the y-axis of the FIGS. 14, 15 and 16. Statistical analysis was performed in JMP version 15 via ANOVA of each temperature session, and in the case of the knockdown study, ANOVAs with p-values less than 0.05 were followed with a Dunnet's post-hoc analysis with the PBS control as the comparator. An alpha level of 0.05 was used to determine statistical significance in these studies. Following the collection of behavioral data, rats were humanely euthanized and dorsal root ganglia and glabrous skin samples were collected and assayed for Nav1.8 protein levels.

Nav1.8 expression in glabrous skin is due completely to Nav1.8 in sensory nerve terminals, so assay of both DRG soma and skin allows confirmation of Nav1.8 protein knockdown throughout the entire dorsal root ganglia neuron. To assay Nav1.8 protein concentration, tissue lysates were prepared by suspending tissue in 200 μL/sample of the 1× lysis Buffer (CST, Cat #9803) along with Halt protease and phosphatase inhibitor (TFS, Cat #78440) diluted to 2× and phenylmethanesulfonyl fluoride (PMSF; Sigma, #78830-25G, (17.4 mg in 1 mL isopropanol, diluted to 1×) and two (2) 5 mm Tissuelyser beads (QIAGEN). Tissue tube was run in the Tissuelyser (QIAGEN) for 3 minutes at 30 1/s, then centrifuged at 4° C. at 5000×g for 20 minutes. Supernatant was collected and frozen at −80° C. for analysis the next day. Protein concentration was measured with the Pierce™ BCA Protein Assay Kit (Cat #23227) by following the “Microplate Procedure” described in the manufacturer's manual. Samples were further diluted 1:3 in cell lysis buffer before plating.

The Meso Scale Discovery (MSD)S-plex platform was used to detect Nav1.8 protein levels. The custom developed assay utilized S-PLEX Development Pack B, SECTOR (25 Plate) (Cat #K15601S-4) kit. Capture and detection antibodies were tagged by MSD. Capture antibody (NeuroMab, Cat #75-166) was diluted to 0.5 μg/mL and the detection antibody (Eli Lilly) was diluted to 0.2 μg/mL. Calibrator was a recombinant C-terminal fragment of human Nav1.8 generated in-house. Sample lysates were diluted 4-fold in assay buffer. Assay buffer base was Diluent 39 (MSD, Cat #R5ABB2) with 100 μL each of the components included in the MSD® Inhibitor Pack (Cat #R70AA-1). Washing steps utilized MSD Tris Wash Buffer (MSD, Cat #R61TX-1) diluted to 1× in double-distilled water (ddH₂O). Plates were read using an MSD plate reader MESO QuickPlex SQ 120 with MSD Methodical Mind software. Data was analyzed using Discovery Workbench v4. Data was normalized by dividing the Nav1.8 protein concentration by the total protein concentration for each lysate sample. Statistical significance was determined by One-way ANOVA, p<0.05.

Effects on cold pressor have been previously reported with a selective Nav1.8 small molecule in early clinical testing (Hijma, et al., “A Phase 1, Randomized, Double-Blind, Placebo-Controlled, Crossover Study to Evaluate the Pharmacodynamic Effects of VX-150, a Highly Selective Nav1.8 Inhibitor, in Healthy Male Adults,” Pain Medicine, 22 (8), pages 1814-26, (2021)). Similar effects in rodent cold behavior were observed following IV administration of AOCs targeting Nav1.8. Dosing with AOCs OX26-siRNA410 and OX26-siRNA535 resulted in a decrease in cold-induced nocifensive behavior, however only animals receiving OX26-siRNA410 showed a statistically significant effect (p<0.05, one-way ANOVA, Dunnet's post-hoc). This finding was consistent at both temperatures assayed (2° C. and −1° C.; FIG. 14). Administration of OX26-siRNA410 and OX26-siRNA535 AOCs also resulted in a significant decrease in Nav1.8 protein in both DRG soma and glabrous paw (the latter finding confirming Nav1.8 protein knockdown in sensory nerve terminals) for both treatment groups (p<0.05, one-way ANOVA).

Further, these data suggest a good correlation between the degree of Nav1.8 protein knockdown and the reversal of cold-induced behavior. These AOCs employed the same OX26 delivery antibody and only differed in the siRNA conjugated. The AOC containing siRNA410 displays superior in vivo efficacy as reflected by both a greater reversal of nocifensive cold behavior and a larger degree of Nav1.8 protein knockdown versus the AOC containing the siRNA535 and PBS controls.

### Example 16: In Vivo Activity of TfR-410siRNA AOCs in Non-Human Primates

The objective of this study was to determine the pharmacodynamic effects of anti-TfR antibody-siRNA410 AOC (TBP1 anti-TfR antibody conjugated to 410 siRNA via SMCC linker) when given by intravenous bolus injection in cynomolgus monkeys. Groups included PBS control, TBP1-410 nCys mAb, TBP2-410 eCys mAb, and TBP3-410 eCys Fab AOCs, N=4/group. The siRNA molecules were modified as shown in FIG. 1B but without the amine or cholesterol handle.

TABLE 22
Description of Test AOCs

| Test AOC | |
|---|---|
| Name | Description |
| TBP1-410 | Anti-TfR antibody (TBP1) linked to modified 410 |
| nCys mAb | siRNA via SMCC linker using nCys linking site. |
| TBP2-410 | Anti-TfR antibody (TBP2) linked to a modified 410 |
| eCys mAb | siRNA via SMCC linker using eCys linking site. |
| TBP3-410 | Fab Anti-TfR antibody (TBP3) linked to modified |
| eCys Fab | 410 siRNA via SMCC linker using eCys linking |
| | site. |

Female cynomolgus monkeys (age range, 2.4-4.0 years, and weight range 2.4-4.0 kgs) were dosed with a single IV dose of PBS control, or 1.0 or 6.0 mg/kg of the three test AOCs (by oligo weight). In addition, a single group was dosed twice with TBP1-siRNA410 nCys mAb at 3.0 mg/kg, by oligo weight, on day 1 and day 8. Humane euthanasia occurred on day 29, and glabrous paw skin was collected to assay for Nav1.8 protein levels. Nav1.8 protein in glabrous paw skin is indicative of protein expression in sensory nerve terminals. NHP skin lysates were prepared by suspending tissue in 150 μL of the 1× lysis Buffer (CST, Cat #9803) along with Halt (Protease and Phosphatase Inhibitor (TFS, Cat #78440) diluted to 2× and PMSF (Sigma, #78830-25G, (17.4 mg in 1 mL isopropanol, diluted to 1×) and two (2) 5 mm Tissuelyser Beads (QIAGEN). Tissue tube was run in the Tissuelyser (QIAGEN) for 3 minutes at 30 1/s, then centrifuged at 4° C. at 5000×g for 20 minutes. Supernatant was collected and frozen at −80° C. for analysis the next day. Protein concentration was measured with the Pierce™ BCA Protein Assay Kit (Cat #23227) by following the “Microplate Procedure” described in the manufacturer's manual. Samples were further diluted 1:3 in cell lysis buffer before plating. The Meso Scale Discovery (MSD)S-Plex platform was used to detect Nav1.8 protein levels in the cultured human DRG neuron protein lysate. The custom developed assay utilized S-PLEX Development Pack B, SECTOR (25 Plate) (Cat #K1560IS-4) kit. Capture and detection antibodies were tagged by MSD. Capture antibody (NeuroMab, Cat #75-166) was diluted to 0.5 μg/mL and the detection antibody was diluted to 0.2 μg/mL. Calibrator was a recombinant C-terminal fragment of human Nav1.8. Sample lysates were diluted 4-fold in assay buffer. Assay buffer base was Diluent 39 (MSD, Cat #R5ABB2) with 100 μL each of the components included in the MSD® Inhibitor Pack (Cat #R70AA-1). Washing steps utilized MSD Tris Wash Buffer (MSD, Cat #R61TX-1) diluted to 1× in ddH₂O. Plates were read using an MSD plate reader MESO QuickPlex SQ 120 with MSD Methodical Mind software. Data was analyzed using Discovery Workbench v4. Data was normalized by dividing the Nav1.8 protein concentration by the total protein concentration for each lysate sample. Statistical significance was determined by one-way ANOVA.

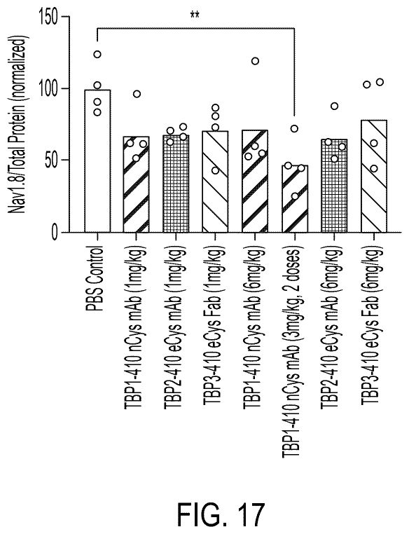

All groups administered a single dose of test AOC showed a consistent, but modest decrease in Nav1.8 protein levels when compared to PBS controls (FIG. 17), but none of these reductions reached statistical significance. However, the treatment group that received two doses of 3.0 mg/kg (by oligo weight) of TBP1-siRNA410 nCys mAb showed a significant reduction of Nav1.8 protein (47.3% of the PBS control group, p<0.05, one-way ANOVA). While the effects of cold-induced behavior were not tested in these animals, the previous testing in humanized Nav1.8 rats suggests that this level of Nav1.8 protein knockdown in skin would be expected to have a significant effect and suggests that this is a physiologically relevant reduction in Nav1.8 protein.

### Example 17: Dose-Response Study of TBP2-410 eCys in Non-Human Primates

The objective of this study was to determine the pharmacodynamic effects of TBP2-410 eCys mAb DAR1 (referred to hereafter as TBP2-410 eCys) when given by intravenous bolus injection in cynomolgus monkeys.

TABLE 23
Description of AOC

| AOC Name | Description |
|---|---|
| TBP2-410 eCys | Anti-TfR antibody (TBP2) as shown in Table |
| mAb DAR1 | 8 linked to a modified 410 siRNA via SMCC |
| | linker using eCys linking site having DAR |
| | of 1. |
| | The modifications of the siRNA are |
| | described in Table 4 for siRNA No. 305 |
| | (which has a start position of target |
| | region on human SCN10A transcript at 410) |

Female cynomolgus monkeys were dosed with four weekly IV doses of PBS control or 3 dose groups of TBP2-410 eCys at 0.3, 3.0, and 30.0 mg/kg (by oligo weight), N=4/group for the PBS control and two low dose groups, and N=2/group for high dose group. Humane euthanasia occurred on day 49 (27 days following the final dose), and glabrous paw skin was collected to assay for Nav1.8 protein levels. Nav1.8 protein in glabrous paw skin is indicative of protein expression in sensory nerve terminals.

NHP skin lysates were prepared by suspending tissue in 150 μl of the 1× lysis Buffer (CST, Cat #9803) along with Halt (Protease and Phosphatase Inhibitor (TFS, Cat #78440) diluted to 2× and PMSF (Sigma, #78830-25G, (17.4 mg in 1 mL isopropanol, diluted to 1×) and two (2) 5 mm Tissuelyser Beads (QIAGEN). Tissue tube was run in the Tissuelyser (QIAGEN) for 3 minutes at 30 1/s, then centrifuged at 4° C. at 5000×g for 20 minutes. Supernatant was collected and frozen at −80° C. for analysis the next day. Protein concentration was measured with the Pierce™ BCA Protein Assay Kit (Catalog #23227) by following the “Microplate Procedure” described in the manufacturer's manual.

Samples were further diluted 1:3 in cell lysis buffer before plating. The Meso Scale Discovery (MSD)S-Plex platform was used to detect Nav1.8 protein levels in the cultured human DRG neuron protein lysate. The custom developed assay utilized S-PLEX Development Pack B, SECTOR (25 Plate) (Cat #K15601S-4) kit. Capture and detection antibodies were tagged by MSD. Capture antibody (NeuroMab, Cat #75-166) was diluted to 0.5 μg/mL and the detection antibody (6B2, produced onsite at Lilly Research Laboratories) was diluted to 0.2 μg/mL. Calibrator was a recombinant C-terminal fragment of human Nav1.8 generated by Lilly at Lilly Research Laboratories. Sample lysates were diluted 4-fold in assay buffer. Assay buffer base was Diluent 39 (MSD, Cat #R5ABB2) with 100 μL each of the components included in the MSD® Inhibitor Pack (Cat #R70AA-1). Washing steps utilized MSD Tris Wash Buffer (MSD, Cat #R61TX-1) diluted to 1× in ddH₂O. Plates were read using an MSD plate reader MESO QuickPlex SQ 120 with MSD Methodical Mind software. Data was analyzed using Discovery Workbench v4. Data was normalized by dividing the Nav1.8 protein concentration by the total protein concentration for each lysate sample. Statistical significance was determined by one-way ANOVA.

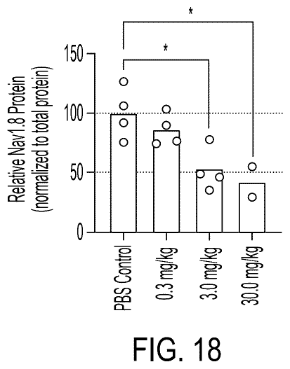

As shown in FIG. 18, administration of the middle and high doses of TBP2-410 eCys (3.0 and 30.0 mg/kg by oligo weight) resulted in significant reduction in Nav1.8 protein in glabrous paw skin (52.3% and 42.2% of the PBS control group respectively, *p<0.05, one-way ANOVA). While the effects of cold-induced behavior were not tested in these animals, the previous testing in humanized Nav1.8 rats suggests that this level of Nav1.8 protein knockdown in skin would be expected to have a significant effect and suggests that this is a physiologically relevant reduction in Nav1.8 protein.

### SEQUENCES

The following nucleic and/or amino acid sequences of the antibodies of the present disclosure are provided below for reference.

TBP1; 211 Com29 B09 hIgG1-EN mAb:
Heavy Chain (HC) (SEQ ID NO: 625)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCD
KTHTCPPCPAPEAEGAPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPSSIE
KTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSP
GK
Light Chain (LC) (SEQ ID NO: 626)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Heavy Chain Variable Region (HCVR) (SEQ ID NO: 627)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSS
Light Chain Variable Region (LCVR) (SEQ ID NO: 628)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIK
HCDR1 (SEQ ID NO: 629)
SYSMN
HCDR2 (SEQ ID NO: 630)
SISSSSSYIYYADSVKG
HCDR3 (SEQ ID NO: 631)
RHGYSNSDAFDT
LCDR1 (SEQ ID NO: 632)
RASQGISHYLV
LCDR2 (SEQ ID NO: 633)
AASSLQS
LCDR3 (SEQ ID NO: 634)
LQHNSYPWT
TBP2; 211 Com29 B09 hIgG1-AAS Heteromab 124C Antibody (for eCys DAR1
conjugation):
Heavy Chain A (HCA) (SEQ ID NO: 635)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCD
KTHTCPPCPAPEAAGGPSVFLFPPKPKDTLMISRTPEVTCVVVSVSHEDPEVKFNWY
VDGVEVHNAKTKPREEQYNSTYRVVSVLTVLHQDWLNGKEYKCKVSNKALPAPIE
KTISKAKGQPRRPRVYTLPPSREEMTKNQVSLVCLVKGFYPSDIAVEWESNGQPEN
NYKTTPPVLDSDGSFFLYSVLTVDKSRWQQGNVFSCSVMHEALHNHYTQKSLSLSP
GK
Heavy Chain B (HCB) (SEQ ID NO: 636)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSSASTKGPCVFPLAPSSKSTSGGTAALGCLVKDYFPEPVTVSWNSGAL
TSGVHTFPAVLQSSGLYSLSSVVTVPSSSLGTQTYICNVNHKPSNTKVDKRVEPKSC
DKTHTCPPCPAPEAAGGPSVFLFPPKPKDTLMISRTPEVTCVVVSVSHEDPEVKFNW
YVDGVEVHNAKTKPREEQYNSTYRVVSVLTVLHQDWLNGKEYKCKVSNKALPAP
IEKTISKAKGQPREPQVYTLPPSREEMTDNQVSLTCLVKGFYPSDIAVEWESNGQPE
NNYKTTPPVLMSDGSFFLASKLTVDKSRWQQGNVFSCSVMHEALHNHYTQKSLSL
SPGK
Light Chain (LC) (SEQ ID NO: 626)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
HCVR (SEQ ID NO: 627)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSS
LCVR (SEQ ID NO: 628)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIK
HCDR1 (SEQ ID NO: 629)
SYSMN
HCDR2 (SEQ ID NO: 630)
SISSSSSYIYYADSVKG
HCDR3 (SEQ ID NO: 631)
RHGYSNSDAFDT
LCDR1 (SEQ ID NO: 632)
RASQGISHYLV
LCDR2 (SEQ ID NO: 633)
AASSLQS
LCDR3 (SEQ ID NO: 634)
LQHNSYPWT
TBP3; 211 Com29 B09 hIgG1 124C Fab:
Heavy Chain (HC) (SEQ ID NO: 637)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSSASTKGPCVFPLAPSSKSTSGGTAALGCLVKDYFPEPVTVSWNSGAL
TSGVHTFPAVLQSSGLYSLSSVVTVPSSSLGTQTYICNVNHKPSNTKVDKRV
Light Chain (LC) (SEQ ID NO: 638)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGE
HCVR (SEQ ID NO: 627)
EVQLVESGGGLVKPGGSLRLSCVASGFTFSSYSMNWVRQAPGKGLEWVSSISSSSS
YIYYADSVKGRFTISRDNAKNSLYLQMNSLRAEDTAVYYCARRHGYSNSDAFDTW
GQGTLVTVSS
LCVR (SEQ ID NO: 628)
DIQMTQSPSAMSASVGDRVTITCRASQGISHYLVWFQQKPGKVPKRLIYAASSLQSG
VPSRFSGSGSGTEFTLTISSLQPEDFATYYCLQHNSYPWTFGQGTKVEIK
HCDR1 (SEQ ID NO: 629)
SYSMN
HCDR2 (SEQ ID NO: 630)
SISSSSSYIYYADSVKG
HCDR3 (SEQ ID NO: 631)
RHGYSNSDAFDT
LCDR1 (SEQ ID NO: 632)
RASQGISHYLV
LCDR2 (SEQ ID NO: 633)
AASSLQS
LCDR3 (SEQ ID NO: 634)
LQHNSYPWT
TBP1; Heavy Chain (SEQ ID NO: 639)
GAGGTGCAGCTGGTGGAGTCTGGGGGAGGCCTGGTCAAGCCTGGGGGGTCCCT
GAGACTCTCCTGTGTAGCCTCTGGATTCACCTTCAGTAGCTATAGCATGAACTG
GGTCCGCCAGGCTCCAGGGAAGGGGCTGGAGTGGGTCTCATCCATTAGTTCCAG
TAGTAGTTACATATATTATGCAGACTCAGTGAAGGGCCGATTCACCATCTCCAG
AGACAACGCCAAGAACTCACTGTATCTGCAAATGAACAGCCTGAGAGCCGAGG
ACACGGCTGTGTATTACTGTGCGAGAAGGCATGGCTACAGTAACTCCGATGCTT
TTGATACTTGGGGCCAAGGGACATTGGTCACCGTCTCTTCAGCCTCCACCAAGG
GCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGT
GGAACTCAGGCGCACTGACCAGCGGCGTGCACACCTTCCCGGCTGTCCTACAGT
CCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTG
GACAAGAGAGTTGAGCCCAAATCTTGTGACAAAACTCACACATGCCCACCGTGC
CCAGCACCTGAAGCCGAGGGGGCACCGTCAGTCTTCCTCTTCCCCCCAAAACCC
AAGGACACCCTCATGATCTCCCGGACCCCTGAGGTCACATGCGTGGTGGTGGAC
GTGAGCCACGAAGACCCTGAGGTCAAGTTCAACTGGTATGTGGACGGCGTGGA
GGTGCATAATGCCAAGACAAAGCCGCGGGAGGAGCAGTACAACAGCACGTACC
GTGTGGTCAGCGTCCTCACCGTCCTGCACCAAGACTGGCTGAATGGCAAGGAGT
ACAAGTGCAAGGTCTCCAACAAAGCCCTCCCATCCTCCATCGAGAAAACCATCT
CCAAAGCCAAAGGGCAGCCCCGAGAACCACAGGTGTACACCCTGCCCCCATCC
CGGGAGGAGATGACCAAGAACCAAGTCAGCCTGACCTGCCTGGTCAAAGGCTT
CTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATTC
CAAGCTCACCGTGGACAAGAGCAGGTGGCAGCAGGGGAACGTCTTCTCATGCT
CCGTGATGCATGAGGCTCTGCACAACCACTACACGCAGAAGAGCCTCTCCCTGT
CTCCGGGCAAA
TBP2; Heavy Chain A (SEQ ID NO: 640)
GAGGTGCAGCTGGTGGAGTCTGGGGGAGGCCTGGTCAAGCCTGGGGGGTCCCT
GAGACTCTCCTGTGTAGCCTCTGGATTCACCTTCAGTAGCTATAGCATGAACTG
GGTCCGCCAGGCTCCAGGGAAGGGGCTGGAGTGGGTCTCATCCATTAGTTCCAG
TAGTAGTTACATATATTATGCAGACTCAGTGAAGGGCCGATTCACCATCTCCAG
AGACAACGCCAAGAACTCACTGTATCTGCAAATGAACAGCCTGAGAGCCGAGG
ACACGGCTGTGTATTACTGTGCGAGAAGGCATGGCTACAGTAACTCCGATGCTT
TTGATACTTGGGGCCAAGGGACATTGGTCACCGTCTCTTCAGCCTCCACCAAGG
GCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGT
GGAACTCAGGCGCGCTGACCAGCGGCGTGCACACCTTCCCGGCTGTCCTACAGT
CCTCAGGACTCTACTCCCTCAGCAGCGTGGTGACCGTGCCCTCCAGCAGCTTGG
GCACCCAGACCTACATCTGCAACGTGAATCACAAGCCCAGCAACACCAAGGTG
GACAAGAGAGTTGAGCCCAAATCTTGTGACAAAACTCACACATGCCCACCGTGC
CCAGCACCTGAAGCCGCCGGGGGACCGTCAGTCTTCCTCTTCCCCCCAAAACCC
AAGGACACCCTCATGATCTCCCGGACCCCTGAGGTCACATGCGTGGTGGTGTCC
GTGAGCCACGAAGACCCTGAGGTCAAGTTCAACTGGTATGTGGACGGCGTGGA
GGTGCATAATGCCAAGACAAAGCCGCGGGAGGAGCAGTACAACAGCACGTACC
GTGTGGTCAGCGTCCTCACCGTCCTGCACCAAGACTGGCTGAATGGCAAGGAGT
ACAAGTGCAAGGTCTCCAACAAAGCCCTCCCAGCCCCCATCGAGAAAACCATCT
CCAAAGCCAAAGGGCAGCCCCGAAGACCACGGGTGTACACCCTGCCCCCATCC
CGGGAGGAGATGACCAAGAACCAAGTCAGCCTGGTCTGCCTGGTCAAAGGCTT
CTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTATTC
CGTGCTCACCGTGGACAAGAGCAGGTGGCAGCAGGGGAACGTCTTCTCATGCTC
CGTGATGCATGAGGCTCTGCACAACCACTACACGCAGAAGAGCCTCTCCCTGTC
TCCGGGCAAA
TBP3; Heavy Chain (SEQ ID NO: 641)
GAAGTGCAGCTGGTCGAGTCAGGAGGCGGCCTCGTGAAACCGGGTGGTAGCCT
GAGGCTGTCCTGCGTGGCATCAGGGTTCACCTTCTCGTCCTACTCCATGAACTGG
GTCAGACAGGCCCCCGGAAAGGGACTTGAATGGGTGTCCAGCATCAGCAGCTC
CTCCTCGTACATCTACTACGCCGATTCCGTGAAGGGCCGGTTCACCATTAGCCG
CGACAATGCCAAGAACTCGCTGTATTTGCAAATGAACTCTCTGCGCGCCGAGGA
CACTGCTGTGTACTACTGTGCGCGGAGACACGGGTACTCCAACTCCGATGCCTT
TGACACCTGGGGCCAGGGAACTCTCGTGACCGTGTCGTCCGCCAGCACCAAGGG
CCCATGCGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTG
GAACTCAGGCGCACTGACCAGCGGCGTGCACACCTTCCCGGCTGTCCTACAGTC
CTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGG
ACAAGAGAGTT
TBP2; Heavy Chain B (SEQ ID NO: 642)
GAAGTGCAGCTGGTCGAGTCAGGAGGCGGCCTCGTGAAACCGGGTGGTAGCCT
GAGGCTGTCCTGCGTGGCATCAGGGTTCACCTTCTCGTCCTACTCCATGAACTGG
GTCAGACAGGCCCCCGGAAAGGGACTTGAATGGGTGTCCAGCATCAGCAGCTC
CTCCTCGTACATCTACTACGCCGATTCCGTGAAGGGCCGGTTCACCATTAGCCG
CGACAATGCCAAGAACTCGCTGTATTTGCAAATGAACTCTCTGCGCGCCGAGGA
CACTGCTGTGTACTACTGTGCGCGGAGACACGGGTACTCCAACTCCGATGCCTT
TGACACCTGGGGCCAGGGAACTCTCGTGACCGTGTCGTCCGCCAGCACCAAGGG
CCCATGCGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTG
GAACTCAGGCGCGCTGACCAGCGGCGTGCACACCTTCCCGGCTGTCCTACAGTC
CTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGG
ACAAGAGAGTTGAGCCCAAATCTTGTGACAAAACTCACACATGCCCACCGTGCC
CAGCACCTGAAGCCGCCGGGGGACCGTCAGTCTTCCTCTTCCCCCCAAAACCCA
AGGACACCCTCATGATCTCCCGGACCCCTGAGGTCACATGCGTGGTGGTGTCCG
TGAGCCACGAAGACCCTGAGGTCAAGTTCAACTGGTATGTGGACGGCGTGGAG
GTGCATAATGCCAAGACAAAGCCGCGGGAGGAGCAGTACAACAGCACGTACCG
TGTGGTCAGCGTCCTCACCGTCCTGCACCAAGACTGGCTGAATGGCAAGGAGTA
CAAGTGCAAGGTCTCCAACAAAGCCCTCCCAGCCCCCATCGAGAAAACCATCTC
CAAAGCCAAAGGGCAGCCCCGAGAACCACAGGTGTACACCCTGCCCCCATCCC
GGGAGGAGATGACCGACAACCAAGTCAGCCTGACCTGCCTGGTCAAAGGCTTC
TATCCCAGCGACATCGCCGTGGAGTGGGAGAGCAATGGGCAGCCGGAGAACAA
CTACAAGACCACGCCTCCCGTGCTGATGTCCGACGGCTCCTTCTTCCTCGCCTCC
AAGCTCACCGTGGACAAGAGCAGGTGGCAGCAGGGGAACGTCTTCTCATGCTC
CGTGATGCATGAGGCTCTGCACAACCACTACACGCAGAAGAGCCTCTCCCTGTC
TCCGGGCAAA
TBP1; Light Chain (SEQ ID NO: 643)
GACATCCAGATGACCCAGTCTCCATCTGCCATGTCTGCATCTGTAGGAGACAGA
GTCACCATCACTTGTCGGGCGAGTCAGGGCATTAGCCACTATTTAGTGTGGTTTC
AGCAGAAACCAGGGAAAGTCCCTAAGCGCCTGATCTATGCTGCATCCAGTTTAC
AAAGTGGGGTCCCATCAAGGTTCAGCGGCAGTGGATCTGGGACAGAATTCACTC
TCACAATCAGCAGCCTGCAGCCTGAAGATTTTGCAACTTATTACTGTCTACAGC
ATAATAGTTACCCTTGGACGTTCGGCCAAGGGACCAAGGTGGAAATCAAACGA
ACCGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGC
TBP2; Light Chain (SEQ ID NO: 644)
GACATCCAGATGACCCAGTCTCCATCTGCCATGTCTGCATCTGTAGGAGACAGA
GTCACCATCACTTGTCGGGCGAGTCAGGGCATTAGCCACTATTTAGTGTGGTTTC
AGCAGAAACCAGGGAAAGTCCCTAAGCGCCTGATCTATGCTGCATCCAGTTTAC
AAAGTGGGGTCCCATCAAGGTTCAGCGGCAGTGGATCTGGGACAGAATTCACTC
TCACAATCAGCAGCCTGCAGCCTGAAGATTTTGCAACTTATTACTGTCTACAGC
ATAATAGTTACCCTTGGACGTTCGGCCAAGGGACCAAGGTGGAAATCAAACGA
ACCGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAGTGC
TBP3; Light Chain (SEQ ID NO: 645)
GACATCCAGATGACCCAGTCTCCATCTGCCATGTCTGCATCTGTAGGAGACAGA
GTCACCATCACTTGTCGGGCGAGTCAGGGCATTAGCCACTATTTAGTGTGGTTTC
AGCAGAAACCAGGGAAAGTCCCTAAGCGCCTGATCTATGCTGCATCCAGTTTAC
AAAGTGGGGTCCCATCAAGGTTCAGCGGCAGTGGATCTGGGACAGAATTCACTC
TCACAATCAGCAGCCTGCAGCCTGAAGATTTTGCAACTTATTACTGTCTACAGC
ATAATAGTTACCCTTGGACGTTCGGCCAAGGGACCAAGGTGGAAATCAAACGA
ACCGTGGCTGCACCATCTGTCTTCATCTTCCCGCCATCTGATGAGCAGTTGAAAT
CTGGAACTGCCTCTGTTGTGTGCCTGCTGAATAACTTCTATCCCAGAGAGGCCA
AAGTACAGTGGAAGGTGGATAACGCCCTCCAATCGGGTAACTCCCAGGAGAGT
GTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCC
ATCAGGGCCTGAGCTCGCCCGTCACAAAGAGCTTCAACAGGGGAGAG
siNTC (SEQ ID NO: 646)
CUAGCUGGACACGUCGAUA
siNTC (SEQ ID NO: 647)
UAUCGACGUGUCCAGCUAGUU
NM_006514.3 Homo sapiens Sodium Voltage-gated Channel Alpha Subunit 10
(SCN10A), Transcript Variant 1, mRNA (SEQ ID NO: 648)
ATGGAATTCCCCATTGGATCCCTCGAAACTAACAACTTCCGTCGCTTTACTCCGG
AGTCACTGGTGGAGATAGAGAAGCAAATTGCTGCCAAGCAGGGAACAAAGAAA
GCCAGAGAGAAGCATAGGGAGCAGAAGGACCAAGAAGAGAAGCCTCGGCCCC
AGCTGGACTTGAAAGCCTGCAACCAGCTGCCCAAGTTCTATGGTGAGCTCCCAG
CAGAACTGATCGGGGAGCCCCTGGAGGATCTAGATCCGTTCTACAGCACACACC
GGACATTTATGGTGCTGAACAAAGGGAGGACCATTTCCCGGTTTAGTGCCACTC
GGGCCCTGTGGCTATTCAGTCCTTTCAACCTGATCAGAAGAACGGCCATCAAAG
TGTCTGTCCACTCGTGGTTCAGTTTATTTATTACGGTCACTATTTTGGTTAATTGT
GTGTGCATGACCCGAACTGACCTTCCAGAGAAAATTGAATATGTCTTCACTGTC
ATTTACACCTTTGAAGCCTTGATAAAGATACTGGCAAGAGGATTTTGTCTAAAT
GAGTTCACGTACCTGAGAGATCCTTGGAACTGGCTGGATTTTAGCGTCATTACC
CTGGCATATGTTGGCACAGCAATAGATCTCCGTGGGATCTCAGGCCTGCGGACA
TTCAGAGTTCTTAGAGCATTAAAAACAGTTTCTGTGATCCCAGGCCTGAAGGTC
ATTGTGGGGGCCCTGATTCACTCAGTGAAGAAACTGGCTGATGTGACCATCCTC
ACCATCTTCTGCCTAAGTGTTTTTGCCTTGGTGGGGCTGCAACTCTTCAAGGGCA
ACCTCAAAAATAAATGTGTCAAGAATGACATGGCTGTCAATGAGACAACCAAC
TACTCATCTCACAGAAAACCAGATATCTACATAAATAAGCGAGGCACTTCTGAC
CCCTTACTGTGTGGCAATGGATCTGACTCAGGCCACTGCCCTGATGGTTATATCT
GCCTTAAAACTTCTGACAACCCGGATTTTAACTACACCAGCTTTGATTCCTTTGC
TTGGGCTTTCCTCTCACTGTTCCGCCTCATGACACAGGATTCCTGGGAACGCCTC
TACCAGCAGACCCTGAGGACTTCTGGGAAAATCTATATGATCTTTTTTGTGCTCG
TAATCTTCCTGGGATCTTTCTACCTGGTCAACTTGATCTTGGCTGTAGTCACCAT
GGCGTATGAGGAGCAGAACCAGGCAACCACTGATGAAATTGAAGCAAAGGAGA
AGAAGTTCCAGGAGGCCCTCGAGATGCTCCGGAAGGAGCAGGAGGTGCTAGCA
GCACTAGGGATTGACACAACCTCTCTCCACTCCCACAATGGATCACCTTTAACC
TCCAAAAATGCCAGTGAGAGAAGGCATAGAATAAAGCCAAGAGTGTCAGAGGG
CTCCACAGAAGACAACAAATCACCCCGCTCTGATCCTTACAACCAGCGCAGGAT
GTCTTTTCTAGGCCTCGCCTCTGGAAAACGCCGGGCTAGTCATGGCAGTGTGTTC
CATTTCCGGTCCCCTGGCCGAGATATCTCACTCCCTGAGGGAGTCACAGATGAT
GGAGTCTTTCCTGGAGACCACGAAAGCCATCGGGGCTCTCTGCTGCTGGGTGGG
GGTGCTGGCCAGCAAGGCCCCCTCCCTAGAAGCCCTCTTCCTCAACCCAGCAAC
CCTGACTCCAGGCATGGAGAAGATGAACACCAACCGCCGCCCACTAGTGAGCTT
GCCCCTGGAGCTGTCGATGTCTCGGCATTCGATGCAGGACAAAAGAAGACTTTC
TTGTCAGCAGAATACTTAGATGAACCTTTCCGGGCCCAAAGGGCAATGAGTGTT
GTCAGTATCATAACCTCCGTCCTTGAGGAACTCGAGGAGTCTGAACAGAAGTGC
CCACCCTGCTTGACCAGCTTGTCTCAGAAGTATCTGATCTGGGATTGCTGCCCCA
TGTGGGTGAAGCTCAAGACAATTCTCTTTGGGCTTGTGACGGATCCCTTTGCAG
AGCTCACCATCACCTTGTGCATCGTGGTGAACACCATCTTCATGGCCATGGAGC
ACCATGGCATGAGCCCTACCTTCGAAGCCATGCTCCAGATAGGCAACATCGTCT
TTACCATATTTTTTACTGCTGAAATGGTCTTCAAAATCATTGCCTTCGACCCATA
CTATTATTTCCAGAAGAAGTGGAATATCTTTGACTGCATCATCGTCACTGTGAGT
CTGCTAGAGCTGGGCGTGGCCAAGAAGGGAAGCCTGTCTGTGCTGCGGAGCTTC
CGCTTGCTGCGCGTATTCAAGCTGGCCAAATCCTGGCCCACCTTAAACACACTC
ATCAAGATCATCGGAAACTCAGTGGGGGCACTGGGGAACCTCACCATCATCCTG
GCCATCATTGTCTTTGTCTTTGCTCTGGTTGGCAAGCAGCTCCTAGGGGAAAACT
ACCGTAACAACCGAAAAAATATCTCCGCGCCCCATGAAGACTGGCCCCGCTGGC
ACATGCACGACTTCTTCCACTCTTTCCTCATTGTCTTCCGTATCCTCTGTGGAGA
GTGGATTGAGAACATGTGGGCCTGCATGGAAGTTGGCCAAAAATCCATATGCCT
CATCCTTTTCTTGACGGTGATGGTGCTAGGGAACCTGGTGGTGCTTAACCTGTTC
ATCGCCCTGCTATTGAACTCTTTCAGTGCTGACAACCTCACAGCCCCGGAGGAC
GATGGGGAGGTGAACAACCTGCAGGTGGCCCTGGCACGGATCCAGGTCTTTGG
CCATCGTACCAAACAGGCTCTTTGCAGCTTCTTCAGCAGGTCCTGCCCATTCCCC
CAGCCCAAGGCAGAGCCTGAGCTGGTGGTGAAACTCCCACTCTCCAGCTCCAAG
GCTGAGAACCACATTGCTGCCAACACTGCCAGGGGGAGCTCTGGAGGGCTCCA
AGCTCCCAGAGGCCCCAGGGATGAGCACAGTGACTTCATCGCTAATCCGACTGT
GTGGGTCTCTGTGCCCATTGCTGAGGGTGAATCTGATCTTGATGACTTGGAGGA
TGATGGTGGGGAAGATGCTCAGAGCTTCCAGCAGGAAGTGATCCCCAAAGGAC
AGCAGGAGCAGCTGCAGCAAGTCGAGAGGTGTGGGGACCACCTGACACCCAGG
AGCCCAGGCACTGGAACATCTTCTGAGGACCTGGCTCCATCCCTGGGTGAGACG
TGGAAAGATGAGTCTGTTCCTCAGGTCCCTGCTGAGGGAGTGGACGACACAAGC
TCCTCTGAGGGCAGCACGGTGGACTGCCTAGATCCTGAGGAAATCCTGAGGAA
GATCCCTGAGCTGGCAGATGACCTGGAAGAACCAGATGACTGCTTCACAGAAG
GATGCATTCGCCACTGTCCCTGCTGCAAACTGGATACCACCAAGAGTCCATGGG
ATGTGGGCTGGCAGGTGCGCAAGACTTGCTACCGTATCGTGGAGCACAGCTGGT
TTGAGAGCTTCATCATCTTCATGATCCTGCTCAGCAGTGGATCTCTGGCCTTTGA
AGACTATTACCTGGACCAGAAGCCCACGGTGAAAGCTTTGCTGGAGTACACTGA
CAGGGTCTTCACCTTTATCTTTGTGTTCGAGATGCTGCTTAAGTGGGTGGCCTAT
GGCTTCAAAAAGTACTTCACCAATGCCTGGTGCTGGCTGGACTTCCTCATTGTG
AATATCTCACTGATAAGTCTCACAGCGAAGATTCTGGAATATTCTGAAGTGGCT
CCCATCAAAGCCCTTCGAACCCTTCGCGCTCTGCGGCCACTGCGGGCTCTTTCTC
GATTTGAAGGCATGCGGGTGGTGGTGGATGCCCTGGTGGGCGCCATCCCATCCA
TCATGAATGTCCTCCTCGTCTGCCTCATCTTCTGGCTCATCTTCAGCATCATGGG
TGTGAACCTCTTCGCAGGGAAGTTTTGGAGGTGCATCAACTATACCGATGGAGA
GTTTTCCCTTGTACCTTTGTCGATTGTGAATAACAAGTCTGACTGCAAGATTCAA
AACTCCACTGGCAGCTTCTTCTGGGTCAATGTGAAAGTCAACTTTGATAATGTTG
CAATGGGTTACCTTGCACTTCTGCAGGTGGCAACCTTTAAAGGCTGGATGGACA
TTATGTATGCAGCTGTTGATTCCCGGGAGGTCAACATGCAACCCAAGTGGGAGG
ACAACGTGTACATGTATTTGTACTTTGTCATCTTCATCATTTTTGGAGGCTTCTTC
ACACTGAATCTCTTTGTTGGGGTCATAATTGACAACTTCAATCAACAGAAAAAA
AAGTTAGGGGGCCAGGACATCTTCATGACAGAGGAGCAGAAGAAATACTACAA
TGCCATGAAGAAGTTGGGCTCCAAGAAGCCCCAGAAGCCCATCCCACGGCCCCT
GAACAAGTTCCAGGGTTTTGTCTTTGACATCGTGACCAGACAAGCTTTTGACAT
CACCATCATGGTCCTCATCTGCCTCAACATGATCACCATGATGGTGGAGACTGA
TGACCAAAGTGAAGAAAAGACGAAAATTCTGGGCAAAATCAACCAGTTCTTTG
TGGCCGTCTTCACAGGCGAATGTGTCATGAAGATGTTCGCTTTGAGGCAGTACT
ACTTCACAAATGGCTGGAATGTGTTTGACTTCATTGTGGTGGTTCTCTCCATTGC
GAGCCTGATTTTTTCTGCAATTCTTAAGTCACTTCAAAGTTACTTCTCCCCAACG
CTCTTCAGAGTCATCCGCCTGGCCCGAATTGGCCGCATCCTCAGACTGATCCGA
GCGGCCAAGGGGATCCGCACACTGCTCTTTGCCCTCATGATGTCCCTGCCTGCC
CTCTTCAACATCGGGCTGTTGCTATTCCTTGTCATGTTCATCTACTCTATCTTCGG
TATGTCCAGCTTTCCCCATGTGAGGTGGGAGGCTGGCATCGACGACATGTTCAA
CTTCCAGACCTTCGCCAACAGCATGCTGTGCCTCTTCCAGATTACCACGTCGGCC
GGCTGGGATGGCCTCCTCAGCCCCATCCTCAACACAGGGCCCCCCTACTGTGAC
CCCAATCTGCCCAACAGCAATGGCACCAGAGGGGACTGTGGGAGCCCAGCCGT
AGGCATCATCTTCTTCACCACCTACATCATCATCTCCTTCCTCATCGTGGTCAAC
ATGTACATTGCAGTGATTCTGGAGAACTTCAATGTGGCCACGGAGGAGAGCACT
GAGCCCCTGAGTGAGGACGACTTTGACATGTTCTATGAGACCTGGGAGAAGTTT
GACCCAGAGGCCACTCAGTTTATTACCTTTTCTGCTCTCTCGGACTTTGCAGACA
CTCTCTCTGGTCCCCTGAGAATCCCAAAACCCAATCGAAATATACTGATCCAGA
TGGACCTGCCTTTGGTCCCTGGAGATAAGATCCACTGCTTGGACATCCTTTTTGC
TTTCACCAAGAATGTCCTAGGAGAATCCGGGGAGTTGGATTCTCTGAAGGCAAA
TATGGAGGAGAAGTTTATGGCAACTAATCTTTCAAAATCATCCTATGAACCAAT
AGCAACCACTCTCCGATGGAAGCAAGAAGACATTTCAGCCACTGTCATTCAAAA
GGCCTATCGGAGCTATGTGCTGCACCGCTCCATGGCACTCTCTAACACCCCATG
TGTGCCCAGAGCTGAGGAGGAGGCTGCATCACTCCCAGATGAAGGTTTTGTTGC
ATTCACAGCAAATGAAAATTGTGTACTCCCAGACAAATCTGAAACTGCTTCTGC
CACATCATTCCCACCGTCCTATGAGAGTGTCACTAGAGGCCTTAGTGATAGAGT
CAACATGAGGACATCTAGCTCAATACAAAATGAAGATGAAGCCACCAGTATGG
AGCTGATTGCCCCTGGGCCCTAGTGA

What is claimed is:

1. An oligonucleotide for inhibiting the expression of a human Nav1.8 gene (SCN10A) in a cell comprising a single stranded oligonucleotide or double stranded oligonucleotide that comprises a region of complementarity to Nav1.8 mRNA.

2. The oligonucleotide of claim 1, wherein the double stranded oligonucleotide comprises a sense strand and an antisense strand, wherein the sense strand and the antisense strand form a duplex region and the antisense strand comprises a region of complementarity to Nav1.8 mRNA, wherein said region of complementarity is less than 30 nucleotides in length.

3. The oligonucleotide of claim 2, wherein the region of complementarity of the antisense strand to the Nav1.8 mRNA target sequence is at least 15 contiguous nucleotides in length, at least 18 contiguous nucleotides in length, at least 19 contiguous nucleotides in length, at least 20 contiguous nucleotides in length, or at least 21 contiguous nucleotides in length.

4. The oligonucleotide of claim 2, wherein the sense strand is 15 to 30 nucleotides in length, 18 to 25 nucleotides in length, or 19 nucleotides in length.

5. The oligonucleotide of claim 2, wherein the antisense strand is 15 to 30 nucleotides in length, 18 to 25 nucleotides in length, 19 nucleotides in length, or 21 nucleotides in length.

6. The oligonucleotide of claim 2, wherein the sense strand is 19 nucleotides in length and the antisense strand is 21 nucleotides in length.

7. The oligonucleotide of claim 2, wherein the antisense strand comprises a region of complementarity to a target sequence of any one of SEQ ID NOs: 1 to 141.

8. The oligonucleotide of claim 2, wherein the region of complementarity is at least 15 contiguous nucleotides in length, at least 16 contiguous nucleotides in length, at least 17 contiguous nucleotides in length, at least 18 contiguous nucleotides in length, at least 19 contiguous nucleotides in length, or at least 20 contiguous nucleotides in length.

9. The oligonucleotide of claim 2, wherein the antisense strand comprises a 3′ overhang sequence of 1 or more nucleotides in length.

10. The oligonucleotide of claim 9, wherein the 3′ overhang sequence is 2 nucleotides in length, and optionally wherein the 3′ overhang sequence is UU.

11. The oligonucleotide of claim 2 comprising at least 1 modified nucleotide.

12. The oligonucleotide of claim 11, wherein all nucleotides of the oligonucleotide are modified.

13. The oligonucleotide of claim 11, wherein the modified nucleotide comprises a 2′-modification on the sugar.

14. The oligonucleotide of claim 13, wherein the 2′-modification is 2′-fluoro, 2′-O-methyl, or 2′-O-methoxyethyl.

15. The oligonucleotide of claim 12, wherein the modification is 2′-fluoro, vinyl phosphonate uridine (VpUq), or 2′-O-methyl.

16. The oligonucleotide of claim 11, wherein the 5′ end of the antisense strand comprises at least one vinyl phosphonate uridine (VpUq).

17. The oligonucleotide of claim 11, wherein one or more nucleotides at positions 7, 8, or 9 of the sense strand are modified with 2′-fluoro.

18. The oligonucleotide of claim 11, wherein one or more nucleotides at positions 2, 6, 14, or 16 of the antisense strand are modified with 2′-fluoro.

19. The oligonucleotide of claim 11, wherein one or more nucleotides at positions 1, 2, 3, 4, 5, 6, 10, 11, 12, 13, 14, 15, 16, 17, 18, or 19 of the sense strand are modified with 2′-O-methyl.

20. The oligonucleotide of claim 11, wherein one or more nucleotides at positions 1, 3, 4, 5, 7 to 13, 15, 17, 18, 19, 20, or 21 of the antisense strand are modified with 2′-O-methyl.

21. The oligonucleotide of claim 2 comprising at least one modified internucleotide linkage.

22. The oligonucleotide of claim 21, wherein at least one modified internucleotide linkage is a phosphorothioate linkage or a phosphorodithioate linkage.

23. The oligonucleotide of claim 22, wherein at least two or three terminal nucleotides on 5′ end or 3′ end of the antisense strand or the sense strand have a phosphorothioate linkage or a phosphorodithioate linkage.

24. The oligonucleotide of claim 21, wherein the sense strand has 19 nucleotides and the internucleotide linkage of nucleotides between positions 1 and 2, 2 and 3, 17 and 18, or 18 and 19 of the sense strand are modified with the phosphorothioate linkage.

25. The oligonucleotide of claim 21, wherein the antisense strand has 21 nucleotides and the internucleotide linkage of one or more nucleotides between positions 1 and 2, 2 and 3, 19 and 20, or 20 and 21 of the antisense strand are modified with the phosphorothioate linkage.

26. The oligonucleotide of claim 2, wherein a linker moiety is attached to one or more termini of the sense strand or the antisense strand.

27. The oligonucleotide of claim 27, wherein a linker is SMCC, optionally attached to the 5′ end of the sense strand.

28. The oligonucleotide of claim 2, wherein the oligonucleotide has a modification pattern as shown in FIG. 1A, optionally without the amine or cholesterol handle.

29. The oligonucleotide of claim 2, wherein the oligonucleotide has a modification pattern as shown in FIG. 1B, optionally without the amine or cholesterol handle.

30. The oligonucleotide of claim 2, wherein at least one nucleotide of the oligonucleotide is conjugated to cholesterol, lipid, polypeptide, or an antibody or an antigen binding fragment thereof.

31. The oligonucleotide of claim 2, wherein the sense strand has the sequence of anyone of the even numbers of SEQ ID NOs: 1 to 422.

32. The oligonucleotide of claim 2, wherein the antisense strand has the sequence of i) anyone of the odd numbers of SEQ ID NOs: 143 to 423 or ii) anyone of SEQ ID Nos: 424 to 564.

33. The oligonucleotide of claim 2, wherein the sense strand and the antisense strands comprise nucleotide sequences selected from the group consisting of:
(a) SEQ ID NOs: 565 and 566, respectively;
(b) SEQ ID NOs: 567 and 568, respectively;
(c) SEQ ID NOs: 569 and 570, respectively;
(d) SEQ ID NOs: 571 and 572, respectively;
(e) SEQ ID NOs: 573 and 574, respectively;
(f) SEQ ID NOs: 575 and 576, respectively;
(g) SEQ ID NOs: 577 and 578 respectively;
(h) SEQ ID NOs: 579 and 580, respectively;
(i) SEQ ID NOs: 581 and 582, respectively;
(j) SEQ ID NOs: 583 and 584, respectively;
(k) SEQ ID NOs: 585 and 586, respectively;
(l) SEQ ID NOs: 587 and 588, respectively;
(m) SEQ ID NOs: 589 and 590 respectively;
(n) SEQ ID NOs: 591 and 592, respectively;
(o) SEQ ID NOs: 593 and 594, respectively;
(p) SEQ ID NOs: 595 and 596, respectively;
(q) SEQ ID NOs: 597 and 598, respectively;
(r) SEQ ID NOs: 599 and 600, respectively;
(s) SEQ ID NOs: 601 and 602 respectively; and
(t) SEQ ID NOs: 603 and 604 respectively.

34. The oligonucleotide of claim 2, wherein the sense strand and the antisense strands comprise nucleotide sequences selected from the group consisting of:
(a) SEQ ID NOs: 565 and 605, respectively;
(b) SEQ ID NOs: 567 and 606, respectively;
(c) SEQ ID NOs: 569 and 607, respectively;
(d) SEQ ID NOs: 571 and 608, respectively;
(e) SEQ ID NOs: 573 and 609, respectively;
(f) SEQ ID NOs: 575 and 610, respectively;
(g) SEQ ID NOs: 577 and 611 respectively;
(h) SEQ ID NOs: 579 and 612, respectively;
(i) SEQ ID NOs: 581 and 613, respectively;
(j) SEQ ID NOs: 583 and 614, respectively;
(k) SEQ ID NOs: 585 and 615, respectively;
(l) SEQ ID NOs: 587 and 616, respectively;
(m) SEQ ID NOs: 589 and 617 respectively;
(n) SEQ ID NOs: 591 and 618, respectively;
(o) SEQ ID NOs: 593 and 619, respectively;
(p) SEQ ID NOs: 595 and 620, respectively;
(q) SEQ ID NOs: 597 and 621, respectively;
(r) SEQ ID NOs: 599 and 622, respectively;
(s) SEQ ID NOs: 601 and 623 respectively; and
(t) SEQ ID NOs: 603 and 624 respectively.

35. The oligonucleotide of claim 2, wherein
i) the sense strand has a sequence as set forth in Table 2 and antisense strand has a sequence as set forth in Table 2;
ii) the sense strand has a sequence set forth in Table 3 and the antisense strand has a sequence set forth in Table 3;
iii) the oligonucleotide has a sequence set forth by any one of siRNA No. 1 to 141;
iv) the oligonucleotide has a sequence set forth by any one of siRNA No. 142 to 282; or
v) the oligonucleotide has a sequence set forth by any one of siRNA No. 283 to 322.

36. The oligonucleotide of claim 1, wherein the single stranded oligonucleotide comprises an antisense sense strand that includes a region of complementarity to Nav1.8 mRNA, wherein said region of complementarity is less than 30 nucleotides in length.

37. The oligonucleotide of claim 36, wherein the antisense strand is 15 to 30 nucleotides in length, 18 to 25 nucleotides in length, 19 nucleotides in length, or 21 nucleotides in length.

38. The oligonucleotide of claim 36, wherein the antisense strand comprises a region of complementarity to a target sequence of any one of SEQ ID NOs: 1 to 141.

39. The oligonucleotide of claim 36, wherein the region of complementarity is at least 15 contiguous nucleotides in length, at least 16 contiguous nucleotides in length, at least 17 contiguous nucleotides in length, at least 18 contiguous nucleotides in length, at least 19 contiguous nucleotides in length, or at least 20 contiguous nucleotides in length.

40. The oligonucleotide of claim 36, wherein the antisense strand comprises a 3′ overhang sequence of 1 or more nucleotides in length.

41. The oligonucleotide of claim 36 comprising at least 1 modified nucleotide.

42. The oligonucleotide of claim 41, wherein all nucleotides of the oligonucleotide are modified.

43. The oligonucleotide of claim 41, wherein the modified nucleotide comprises a 2′-modification on the sugar.

44. The oligonucleotide of claim 43, wherein the 2′-modification is 2′-fluoro, 2′-O-methyl, or 2′-O-methoxyethyl.

45. The oligonucleotide of claim 41, wherein the modification is 2′-fluoro, vinyl phosphonate uridine (VpUq), or 2′-O-methyl.

46. The oligonucleotide of claim 41, wherein the 5′ end of the antisense strand comprises at least one vinyl phosphonate uridine (VpUq).

47. The oligonucleotide of claim 41, wherein one or more nucleotides at positions 1-21 of the antisense strand are modified with 2′-fluoro.

48. The oligonucleotide of claim 41, wherein one or more nucleotides at positions 1-21 of the antisense strand are modified with 2′-O-methyl.

49. The oligonucleotide of claim 36 comprising at least one modified internucleotide linkage.

50. The oligonucleotide of claim 49, wherein at least one modified internucleotide linkage is a phosphorothioate linkage or a phosphorodithioate linkage.

51. The oligonucleotide of claim 50, wherein at least two terminal nucleotides on 5′ end or 3′ end of the antisense strand have a phosphorothioate linkage or a phosphorodithioate linkage.

52. The oligonucleotide of claim 50, wherein at least three terminal nucleotides on 5′ end or 3′ end of the antisense strand have phosphorothioate linkages or a phosphorodithioate linkages.

53. The oligonucleotide of claim 36, wherein a linker moiety is attached to one or more termini of the antisense strand.

54. The oligonucleotide of claim 53, wherein a linker is SMCC.

55. The oligonucleotide of claim 36, wherein at least one nucleotide of the oligonucleotide is conjugated to cholesterol, lipid, polypeptide, or an antibody or an antigen binding fragment thereof.

56. The oligonucleotide of claim 36, wherein the antisense strand has the sequence of i) anyone of the odd numbers of SEQ ID NOs: 143 to 423 or ii) anyone of SEQ ID Nos: 424 to 564.

57. The oligonucleotide of claim 36, wherein (i) the antisense strand has a sequence as set forth in Table 2 or (ii) the antisense strand has a sequence as set forth in Table 3.

58. A cell comprising an oligonucleotide:
i) wherein the oligonucleotide is a double stranded oligonucleotide comprising a sense strand and an antisense strand, wherein the sense strand and the antisense strand form a duplex region and the antisense strand comprises a region of complementarity to Nav1.8 mRNA, wherein said region of complementarity is less than 30 nucleotides in length, or
ii) wherein the oligonucleotide is a single stranded oligonucleotide comprising an antisense sense strand that includes a region of complementarity to Nav1.8 mRNA, wherein said region of complementarity is less than 30 nucleotides in length.

59. A vector comprising an oligonucleotide:
i) wherein the oligonucleotide is a double stranded oligonucleotide comprising a sense strand and an antisense strand, wherein the sense strand and the antisense strand form a duplex region and the antisense strand comprises a region of complementarity to Nav1.8 mRNA, wherein said region of complementarity is less than 30 nucleotides in length, or
ii) wherein the oligonucleotide is a single stranded oligonucleotide comprising an antisense sense strand that includes a region of complementarity to Nav1.8 mRNA, wherein said region of complementarity is less than 30 nucleotides in length.

60. The vector of claim 59, wherein the oligonucleotide sequence is operably linked to a regulatory sequence.

61. A pharmaceutical composition comprising a pharmaceutically acceptable carrier and an oligonucleotide:
i) wherein the oligonucleotide is a double stranded oligonucleotide comprising a sense strand and an antisense strand, wherein the sense strand and the antisense strand form a duplex region and the antisense strand comprises a region of complementarity to Nav1.8 mRNA, wherein said region of complementarity is less than 30 nucleotides in length, or
ii) wherein the oligonucleotide is a single stranded oligonucleotide comprising an antisense sense strand that includes a region of complementarity to Nav1.8 mRNA, wherein said region of complementarity is less than 30 nucleotides in length.

62. A conjugate comprising an oligonucleotide of claim 2 and an anti-transferrin receptor antibody or antigen binding fragment thereof conjugated to the sense strand of the oligonucleotide or the antisense strand of the oligonucleotide.

63. The conjugate of claim 62, wherein the antibody or antigen binding fragment thereof is linked to the sense strand or the antisense strand through a linker.

64. The conjugate of claim 63, wherein the linker is an SMCC linker.

65. The conjugate of claim 64, wherein the linker is SMCC linker attached to: i) the 5′ end of the sense strand, ii) the 5′ end of the antisense strand, iii) the 3′ end of the sense strand, or iv) the 3′ end of the antisense strand.

66. The conjugate of claim 62, wherein the oligonucleotide mediates RNA interference against the human Nav1.8 mRNA preferentially in DRG cells in a human subject.

67. The conjugate of claim 66, wherein the mediation of RNA interference against the human Nav1.8 mRNA modulates pain in a subject.

68. The conjugate of claim 66, wherein the medication of RNA interference against the human Nav1.8 mRNA reduces pain in a subject.

69. The conjugate of claim 62, wherein the antibody or antigen binding fragment thereof comprises a heavy chain variable region (HCVR) and a light chain variable region (LCVR), wherein the HCVR comprises heavy chain complementarity determining regions HCDR1, HCDR2, and HCDR3, and the LCVR comprises light chain complementarity determining regions LCDR1, LCDR2, and LCDR3, wherein the HCDR1, HCDR2, HCDR3, LCDR1, LCDR2, and LCDR3 comprise the following sequences: HCDR1 comprises SEQ ID NO: 629, HCDR2 comprises SEQ ID NO: 630, HCDR3 comprises SEQ ID NO: 631, LCDR1 comprises SEQ ID NO: 632, LCDR2 comprises SEQ ID NO: 633, and LCDR3 comprises SEQ ID NO: 634.

70. The conjugate of claim 62, wherein the antibody or antigen binding fragment thereof comprises a HCVR of SEQ ID NO: 627 and a LCVR of SEQ ID NO: 628.

71. The conjugate of claim 62, wherein the antibody comprises a heavy chain (HC) and a light chain (LC) of following sequences:
(a) HC of SEQ ID NO: 625 and LC of SEQ ID NO: 626;
(b) HCA (first heavy chain) of SEQ ID NO: 635, HCB (second heavy chain) of SEQ ID NO: 636, and a LC of SEQ ID NO: 626; or
(c) HC of SEQ ID NO: 637 and LC of SEQ ID NO: 638.

72. The conjugate of claim 62, wherein the antibody fragment is a Fab, scFv, Fv, or scFab.

73. The conjugate of claim 62, wherein the antibody or antigen binding fragment thereof comprises at least one heavy chain constant region comprising cysteine at residue 124 (according to the EU Index numbering).

74. The conjugate of claim 62, wherein the antibody or antigen binding fragment thereof comprises two heavy chain constant regions comprising cysteine at residue 124 (according to the EU Index numbering).

75. The conjugate of claim 62, wherein the antibody or antigen binding fragment thereof further comprises a half-life extender.

76. The conjugate of claim 75, wherein the half-life extender is a VHH that binds human serum albumin (HSA).

77. A pharmaceutical composition comprising the conjugate of claim 63 and a pharmaceutically acceptable carrier.

78. A method of preventing, treating, or reducing pain in a patient in need thereof, the method comprising administering to the patient an effective amount of:
i) a double stranded oligonucleotide comprising a sense strand and an antisense strand, wherein the sense strand and the antisense strand form a duplex region and the antisense strand comprises a region of complementarity to Nav1.8 mRNA, wherein said region of complementarity is less than 30 nucleotides in length,
ii) a single stranded oligonucleotide comprising an antisense sense strand that includes a region of complementarity to Nav1.8 mRNA, wherein said region of complementarity is less than 30 nucleotides in length, or
iii) a conjugate comprising the double stranded oligonucleotide, and an anti-transferrin receptor antibody or antigen binding fragment thereof conjugated to the sense strand of the oligonucleotide or the antisense strand of the oligonucleotide.

79. The method of claim 78, wherein the pain is chronic pain.

80. The method of claim 78, wherein the pain is: i) inflammatory pain, ii) neuropathic pain, or iii) mixed pain.

81. The method of claim 78, wherein the double stranded oligonucleotide, the single stranded oligonucleotide, or the conjugate is administered intravenously or subcutaneously.


SEQUENCE LISTING (648 sequences)

SEQ ID NO: 1 - RNA, 18 nt, synthetic construct
  source 1..18: mol_type = other RNA
ttacggtcactattttgg

SEQ ID NO: 2 - RNA, 18 nt, synthetic construct
  source 1..18: mol_type = other RNA
tgatcttggctgtagtca

SEQ ID NO: 3 - RNA, 18 nt, synthetic construct
  source 1..18: mol_type = other RNA
taattgtgtgtgcatgac

SEQ ID NO: 4 - RNA, 18 nt, synthetic construct
  source 1..18: mol_type = other RNA
cggtcactattttggtta

SEQ ID NO: 5 - RNA, 18 nt, synthetic construct
  source 1..18: mol_type = other RNA
aacctgatcagaagaacg

SEQ ID NO: 6 - RNA, 18 nt, synthetic construct
  source 1..18: mol_type = other RNA
aaatgagttcacgtacct

SEQ ID NO: 7 - RNA, 18 nt, synthetic construct
  source 1..18: mol_type = other RNA
caacccggattttaacta

SEQ ID NO: 8 - RNA, 18 nt, synthetic construct
  source 1..18: mol_type = other RNA
ctgacaacccggatttta

SEQ ID NO: 9 - RNA, 18 nt, synthetic construct
  source 1..18: mol_type = other RNA
aatgagttcacgtacctg

SEQ ID NO: 10 - RNA, 18 nt, synthetic construct
  source 1..18: mol_type = other RNA
acaacccggattttaact

SEQ ID NO: 11 - RNA, 18 nt, synthetic construct
  source 1..18: mol_type = other RNA
agaacggccatcaaagtg

SEQ ID NO: 12 - RNA, 18 nt, synthetic construct
  source 1..18: mol_type = other RNA
cttctgacaacccggatt

SEQ ID NO: 13 - RNA, 18 nt, synthetic construct
  source 1..18: mol_type = other RNA
tgtctaaatgagttcacg

SEQ ID NO: 14 - RNA, 18 nt, synthetic construct
  source 1..18: mol_type = other RNA
agaatacttagatgaacc

SEQ ID NO: 15 - RNA, 18 nt, synthetic construct
  source 1..18: mol_type = other RNA
taccttcgaagccatgct

SEQ ID NO: 16 - RNA, 18 nt, synthetic construct
  source 1..18: mol_type = other RNA
gtgcatgacccgaactga

SEQ ID NO: 17 - RNA, 18 nt, synthetic construct
  source 1..18: mol_type = other RNA
tgtgcatgacccgaactg

SEQ ID NO: 18 - RNA, 18 nt, synthetic construct
  source 1..18: mol_type = other RNA
cttacaaccagcgcagga

SEQ ID NO: 19 - RNA, 18 nt, synthetic construct
  source 1..18: mol_type = other RNA
cctaccttcgaagccatg

SEQ ID NO: 20 - RNA, 18 nt, synthetic construct
  source 1..18: mol_type = other RNA
gcgtcattaccctggcat

SEQ ID NO: 21 - RNA, 18 nt, synthetic construct
  source 1..18: mol_type = other RNA
aattgtgtgtgcatgacc

SEQ ID NO: 22 - RNA, 18 nt, synthetic construct
  source 1..18: mol_type = other RNA
ggattttagcgtcattac

SEQ ID NO: 23 - RNA, 18 nt, synthetic construct
  source 1..18: mol_type = other RNA
cagcagaatacttagatg

SEQ ID NO: 24 - RNA, 18 nt, synthetic construct
  source 1..18: mol_type = other RNA
gcatgagccctaccttcg

SEQ ID NO: 25 - RNA, 18 nt, synthetic construct
  source 1..18: mol_type = other RNA
tgacaacccggattttaa

SEQ ID NO: 26 - RNA, 18 nt, synthetic construct
  source 1..18: mol_type = other RNA
ctaaatgagttcacgtac

SEQ ID NO: 27 - RNA, 18 nt, synthetic construct
  source 1..18: mol_type = other RNA
tgtcagcagaatacttag

SEQ ID NO: 28 - RNA, 18 nt, synthetic construct
  source 1..18: mol_type = other RNA
aaccaactactcatctca

SEQ ID NO: 29 - RNA, 18 nt, synthetic construct
  source 1..18: mol_type = other RNA
cgctttactccggagtca

SEQ ID NO: 30 - RNA, 18 nt, synthetic construct
  source 1..18: mol_type = other RNA
acaaccaactactcatct

SEQ ID NO: 31 - RNA, 18 nt, synthetic construct
  source 1..18: mol_type = other RNA
cttgtacctttgtcgatt

SEQ ID NO: 32 - RNA, 18 nt, synthetic construct
  source 1..18: mol_type = other RNA
cacgtacctgagagatcc

SEQ ID NO: 33 - RNA, 18 nt, synthetic construct
  source 1..18: mol_type = other RNA
cccaaaacccaatcgaaa

SEQ ID NO: 34 - RNA, 18 nt, synthetic construct
  source 1..18: mol_type = other RNA
gggtgaagctcaagacaa

SEQ ID NO: 35 - RNA, 18 nt, synthetic construct
  source 1..18: mol_type = other RNA
gtttatttattacggtca

SEQ ID NO: 36 - RNA, 18 nt, synthetic construct
  source 1..18: mol_type = other RNA
cagtttatttattacggt

SEQ ID NO: 37 - RNA, 18 nt, synthetic construct
  source 1..18: mol_type = other RNA
accaactactcatctcac

SEQ ID NO: 38 - RNA, 18 nt, synthetic construct
  source 1..18: mol_type = other RNA
acctgatcagaagaacgg

SEQ ID NO: 39 - RNA, 18 nt, synthetic construct
  source 1..18: mol_type = other RNA
cttttttgtgctcgtaat

SEQ ID NO: 40 - RNA, 18 nt, synthetic construct
  source 1..18: mol_type = other RNA
gaattccccattggatcc

SEQ ID NO: 41 - RNA, 18 nt, synthetic construct
  source 1..18: mol_type = other RNA
ggcatatgttggcacagc

SEQ ID NO: 42 - RNA, 18 nt, synthetic construct
  source 1..18: mol_type = other RNA
gccttagtgatagagtca

SEQ ID NO: 43 - RNA, 18 nt, synthetic construct
  source 1..18: mol_type = other RNA
tcattaccctggcatatg

SEQ ID NO: 44 - RNA, 18 nt, synthetic construct
  source 1..18: mol_type = other RNA
aatgagtgttgtcagtat

SEQ ID NO: 45 - RNA, 18 nt, synthetic construct
  source 1..18: mol_type = other RNA
aggacatctagctcaata

SEQ ID NO: 46 - RNA, 18 nt, synthetic construct
  source 1..18: mol_type = other RNA
tgatccttacaaccagcg

SEQ ID NO: 47 - RNA, 18 nt, synthetic construct
  source 1..18: mol_type = other RNA
ttatttattacggtcact

SEQ ID NO: 48 - RNA, 18 nt, synthetic construct
  source 1..18: mol_type = other RNA
aagaacggccatcaaagt

SEQ ID NO: 49 - RNA, 18 nt, synthetic construct
  source 1..18: mol_type = other RNA
gacaacccggattttaac

SEQ ID NO: 50 - RNA, 18 nt, synthetic construct
  source 1..18: mol_type = other RNA
tctagctcaatacaaaat

SEQ ID NO: 51 - RNA, 18 nt, synthetic construct
  source 1..18: mol_type = other RNA
gatctccgtgggatctca

SEQ ID NO: 52 - RNA, 18 nt, synthetic construct
  source 1..18: mol_type = other RNA
ccggtttagtgccactcg

SEQ ID NO: 53 - RNA, 18 nt, synthetic construct
  source 1..18: mol_type = other RNA
ctcatccttttcttgacg

SEQ ID NO: 54 - RNA, 18 nt, synthetic construct
  source 1..18: mol_type = other RNA
acccaatcgaaatatact

SEQ ID NO: 55 - RNA, 18 nt, synthetic construct
  source 1..18: mol_type = other RNA
tttatctttgtgttcgag

SEQ ID NO: 56 - RNA, 18 nt, synthetic construct
  source 1..18: mol_type = other RNA
acagaaggatgcattcgc

SEQ ID NO: 57 - RNA, 18 nt, synthetic construct
  source 1..18: mol_type = other RNA
gtcactagaggccttagt

SEQ ID NO: 58 - RNA, 18 nt, synthetic construct
  source 1..18: mol_type = other RNA
tcttttttgtgctcgtaa

SEQ ID NO: 59 - RNA, 18 nt, synthetic construct
  source 1..18: mol_type = other RNA
ttgtcgattgtgaataac

SEQ ID NO: 60 - RNA, 18 nt, synthetic construct
  source 1..18: mol_type = other RNA
agggcaatgagtgttgtc

SEQ ID NO: 61 - RNA, 18 nt, synthetic construct
  source 1..18: mol_type = other RNA
tttttgtgctcgtaatct

SEQ ID NO: 62 - RNA, 18 nt, synthetic construct
  source 1..18: mol_type = other RNA
ttcgatgcaggacaaaag

SEQ ID NO: 63 - RNA, 18 nt, synthetic construct
  source 1..18: mol_type = other RNA
caaccgaaaaaatatctc

SEQ ID NO: 64 - RNA, 18 nt, synthetic construct
  source 1..18: mol_type = other RNA
acggtcactattttggtt

SEQ ID NO: 65 - RNA, 18 nt, synthetic construct
  source 1..18: mol_type = other RNA
ggatgcattcgccactgt

SEQ ID NO: 66 - RNA, 18 nt, synthetic construct
  source 1..18: mol_type = other RNA
gggcaatgagtgttgtca

SEQ ID NO: 67 - RNA, 18 nt, synthetic construct
  source 1..18: mol_type = other RNA
tgatagagtcaacatgag

SEQ ID NO: 68 - RNA, 18 nt, synthetic construct
  source 1..18: mol_type = other RNA
atccttacaaccagcgca

SEQ ID NO: 69 - RNA, 18 nt, synthetic construct
  source 1..18: mol_type = other RNA
tgtacctttgtcgattgt

SEQ ID NO: 70 - RNA, 18 nt, synthetic construct
  source 1..18: mol_type = other RNA
ttgtacctttgtcgattg

SEQ ID NO: 71 - RNA, 18 nt, synthetic construct
  source 1..18: mol_type = other RNA
actcgaggagtctgaaca

SEQ ID NO: 72 - RNA, 18 nt, synthetic construct
  source 1..18: mol_type = other RNA
cagtatcataacctccgt

SEQ ID NO: 73 - RNA, 18 nt, synthetic construct
  source 1..18: mol_type = other RNA
aagggcaatgagtgttgt

SEQ ID NO: 74 - RNA, 18 nt, synthetic construct
  source 1..18: mol_type = other RNA
caatgagtgttgtcagta

SEQ ID NO: 75 - RNA, 18 nt, synthetic construct
  source 1..18: mol_type = other RNA
tgcattcgccactgtccc

SEQ ID NO: 76 - RNA, 18 nt, synthetic construct
  source 1..18: mol_type = other RNA
atcttttttgtgctcgta

SEQ ID NO: 77 - RNA, 18 nt, synthetic construct
  source 1..18: mol_type = other RNA
cagtgacttcatcgctaa

SEQ ID NO: 78 - RNA, 18 nt, synthetic construct
  source 1..18: mol_type = other RNA
gtttatggcaactaatct

SEQ ID NO: 79 - RNA, 18 nt, synthetic construct
  source 1..18: mol_type = other RNA
cttgtgcatcgtggtgaa

SEQ ID NO: 80 - RNA, 18 nt, synthetic construct
  source 1..18: mol_type = other RNA
ctttgggcttgtgacgga

SEQ ID NO: 81 - RNA, 18 nt, synthetic construct
  source 1..18: mol_type = other RNA
cttgggctttcctctcac

SEQ ID NO: 82 - RNA, 18 nt, synthetic construct
  source 1..18: mol_type = other RNA
catctagctcaatacaaa

SEQ ID NO: 83 - RNA, 18 nt, synthetic construct
  source 1..18: mol_type = other RNA
gttgtcagtatcataacc

SEQ ID NO: 84 - RNA, 18 nt, synthetic construct
  source 1..18: mol_type = other RNA
ctccggagtcactggtgg

SEQ ID NO: 85 - RNA, 18 nt, synthetic construct
  source 1..18: mol_type = other RNA
aggtctttggccatcgta

SEQ ID NO: 86 - RNA, 18 nt, synthetic construct
  source 1..18: mol_type = other RNA
gtgtgcatgacccgaact

SEQ ID NO: 87 - RNA, 18 nt, synthetic construct
  source 1..18: mol_type = other RNA
cacggatccaggtctttg

SEQ ID NO: 88 - RNA, 18 nt, synthetic construct
  source 1..18: mol_type = other RNA
acttagatgaacctttcc

SEQ ID NO: 89 - RNA, 18 nt, synthetic construct
  source 1..18: mol_type = other RNA
aacatgcaacccaagtgg

SEQ ID NO: 90 - RNA, 18 nt, synthetic construct
  source 1..18: mol_type = other RNA
tagaggccttagtgatag

SEQ ID NO: 91 - RNA, 18 nt, synthetic construct
  source 1..18: mol_type = other RNA
atcgaaatatactgatcc

SEQ ID NO: 92 - RNA, 18 nt, synthetic construct
  source 1..18: mol_type = other RNA
tttcagccactgtcattc

SEQ ID NO: 93 - RNA, 18 nt, synthetic construct
  source 1..18: mol_type = other RNA
ataacctccgtccttgag

SEQ ID NO: 94 - RNA, 18 nt, synthetic construct
  source 1..18: mol_type = other RNA
tttatggcaactaatctt

SEQ ID NO: 95 - RNA, 18 nt, synthetic construct
  source 1..18: mol_type = other RNA
ctatgaaccaatagcaac

SEQ ID NO: 96 - RNA, 18 nt, synthetic construct
  source 1..18: mol_type = other RNA
ccaactactcatctcaca

SEQ ID NO: 97 - RNA, 18 nt, synthetic construct
  source 1..18: mol_type = other RNA
gttcatcgccctgctatt

SEQ ID NO: 98 - RNA, 18 nt, synthetic construct
  source 1..18: mol_type = other RNA
atccttttcttgacggtg

SEQ ID NO: 99 - RNA, 18 nt, synthetic construct
  source 1..18: mol_type = other RNA
ctatcggagctatgtgct

SEQ ID NO: 100 - RNA, 18 nt, synthetic construct
  source 1..18: mol_type = other RNA
ccaagaatgtcctaggag

SEQ ID NO: 101 - RNA, 18 nt, synthetic construct
  source 1..18: mol_type = other RNA
ttctacagcacacaccgg

SEQ ID NO: 102 - RNA, 18 nt, synthetic construct
  source 1..18: mol_type = other RNA
agtttatttattacggtc

SEQ ID NO: 103 - RNA, 18 nt, synthetic construct
  source 1..18: mol_type = other RNA
atcatcctatgaaccaat

SEQ ID NO: 104 - RNA, 18 nt, synthetic construct
  source 1..18: mol_type = other RNA
caggtctttggccatcgt

SEQ ID NO: 105 - RNA, 18 nt, synthetic construct
  source 1..18: mol_type = other RNA
gcacacaccggacattta

SEQ ID NO: 106 - RNA, 18 nt, synthetic construct
  source 1..18: mol_type = other RNA
catcgtgaccagacaagc

SEQ ID NO: 107 - RNA, 18 nt, synthetic construct
  source 1..18: mol_type = other RNA
gcaatgggttaccttgca

SEQ ID NO: 108 - RNA, 18 nt, synthetic construct
  source 1..18: mol_type = other RNA
tccttttcttgacggtga

SEQ ID NO: 109 - RNA, 18 nt, synthetic construct
  source 1..18: mol_type = other RNA
tcgaaatatactgatcca

SEQ ID NO: 110 - RNA, 18 nt, synthetic construct
  source 1..18: mol_type = other RNA
tcgatgcaggacaaaaga

SEQ ID NO: 111 - RNA, 18 nt, synthetic construct
  source 1..18: mol_type = other RNA
caaaaggcctatcggagc

SEQ ID NO: 112 - RNA, 18 nt, synthetic construct
  source 1..18: mol_type = other RNA
ggcgaatgtgtcatgaag

SEQ ID NO: 113 - RNA, 18 nt, synthetic construct
  source 1..18: mol_type = other RNA
gctatgtgctgcaccgct

SEQ ID NO: 114 - RNA, 18 nt, synthetic construct
  source 1..18: mol_type = other RNA
tcccagcagaactgatcg

SEQ ID NO: 115 - RNA, 18 nt, synthetic construct
  source 1..18: mol_type = other RNA
cacaccggacatttatgg

SEQ ID NO: 116 - RNA, 18 nt, synthetic construct
  source 1..18: mol_type = other RNA
gtgtcactagaggcctta

SEQ ID NO: 117 - RNA, 18 nt, synthetic construct
  source 1..18: mol_type = other RNA
tgtcgattgtgaataaca

SEQ ID NO: 118 - RNA, 18 nt, synthetic construct
  source 1..18: mol_type = other RNA
aacttctgacaacccgga

SEQ ID NO: 119 - RNA, 18 nt, synthetic construct
  source 1..18: mol_type = other RNA
ctatgtgctgcaccgctc

SEQ ID NO: 120 - RNA, 18 nt, synthetic construct
  source 1..18: mol_type = other RNA
aagtctgactgcaagatt

SEQ ID NO: 121 - RNA, 18 nt, synthetic construct
  source 1..18: mol_type = other RNA
cctttgtcgattgtgaat

SEQ ID NO: 122 - RNA, 18 nt, synthetic construct
  source 1..18: mol_type = other RNA
gtttagtgccactcgggc

SEQ ID NO: 123 - RNA, 18 nt, synthetic construct
  source 1..18: mol_type = other RNA
tccttacaaccagcgcag

SEQ ID NO: 124 - RNA, 18 nt, synthetic construct
  source 1..18: mol_type = other RNA
aacccaatcgaaatatac

SEQ ID NO: 125 - RNA, 18 nt, synthetic construct
  source 1..18: mol_type = other RNA
tgtgtgcatgacccgaac

SEQ ID NO: 126 - RNA, 18 nt, synthetic construct
  source 1..18: mol_type = other RNA
catcgccctgctattgaa

SEQ ID NO: 127 - RNA, 18 nt, synthetic construct
  source 1..18: mol_type = other RNA
ccttacaaccagcgcagg

SEQ ID NO: 128 - RNA, 18 nt, synthetic construct
  source 1..18: mol_type = other RNA
atctccgcgccccatgaa

SEQ ID NO: 129 - RNA, 18 nt, synthetic construct
  source 1..18: mol_type = other RNA
cttttcttgacggtgatg

SEQ ID NO: 130 - RNA, 18 nt, synthetic construct
  source 1..18: mol_type = other RNA
tcactagaggccttagtg

SEQ ID NO: 131 - RNA, 18 nt, synthetic construct
  source 1..18: mol_type = other RNA
gcgaatgtgtcatgaaga

SEQ ID NO: 132 - RNA, 18 nt, synthetic construct
  source 1..18: mol_type = other RNA
tctgacaacccggatttt

SEQ ID NO: 133 - RNA, 18 nt, synthetic construct
  source 1..18: mol_type = other RNA
agtatcataacctccgtc

SEQ ID NO: 134 - RNA, 18 nt, synthetic construct
  source 1..18: mol_type = other RNA
gcatatgttggcacagca

SEQ ID NO: 135 - RNA, 18 nt, synthetic construct
  source 1..18: mol_type = other RNA
cccttactgtgtggcaat

SEQ ID NO: 136 - RNA, 18 nt, synthetic construct
  source 1..18: mol_type = other RNA
ctacagcacacaccggac

SEQ ID NO: 137 - RNA, 18 nt, synthetic construct
  source 1..18: mol_type = other RNA
cgaaaaaatatctccgcg

SEQ ID NO: 138 - RNA, 18 nt, synthetic construct
  source 1..18: mol_type = other RNA
cttggtggggctgcaact

SEQ ID NO: 139 - RNA, 18 nt, synthetic construct
  source 1..18: mol_type = other RNA
ctgcaagattcaaaactc

SEQ ID NO: 140 - RNA, 18 nt, synthetic construct
  source 1..18: mol_type = other RNA
cgtcgctttactccggag

SEQ ID NO: 141 - RNA, 18 nt, synthetic construct
  source 1..18: mol_type = other RNA
tcatcctatgaaccaata

SEQ ID NO: 142 - RNA, 19 nt, synthetic construct
  source 1..19: mol_type = other RNA
ttacggtcactattttgga

SEQ ID NO: 143 - RNA, 19 nt, synthetic construct
  source 1..19: mol_type = other RNA
tccaaaatagtgaccgtaa

SEQ ID NO: 144 - RNA, 19 nt, synthetic construct
  source 1..19: mol_type = other RNA
tgatcttggctgtagtcaa

SEQ ID NO: 145 - RNA, 19 nt, synthetic construct
  source 1..19: mol_type = other RNA
ttgactacagccaagatca

SEQ ID NO: 146 - RNA, 19 nt, synthetic construct
  source 1..19: mol_type = other RNA
taattgtgtgtgcatgaca

SEQ ID NO: 147 - RNA, 19 nt, synthetic construct
  source 1..19: mol_type = other RNA
tgtcatgcacacacaatta

SEQ ID NO: 148 - RNA, 19 nt, synthetic construct
  source 1..19: mol_type = other RNA
cggtcactattttggttaa

SEQ ID NO: 149 - RNA, 19 nt, synthetic construct
  source 1..19: mol_type = other RNA
ttaaccaaaatagtgaccg

SEQ ID NO: 150 - RNA, 19 nt, synthetic construct
  source 1..19: mol_type = other RNA
aacctgatcagaagaacga

SEQ ID NO: 151 - RNA, 19 nt, synthetic construct
  source 1..19: mol_type = other RNA
tcgttcttctgatcaggtt

SEQ ID NO: 152 - RNA, 19 nt, synthetic construct
  source 1..19: mol_type = other RNA
aaatgagttcacgtaccta

SEQ ID NO: 153 - RNA, 19 nt, synthetic construct
  source 1..19: mol_type = other RNA
taggtacgtgaactcattt

SEQ ID NO: 154 - RNA, 19 nt, synthetic construct
  source 1..19: mol_type = other RNA
caacccggattttaactaa

SEQ ID NO: 155 - RNA, 19 nt, synthetic construct
  source 1..19: mol_type = other RNA
ttagttaaaatccgggttg

SEQ ID NO: 156 - RNA, 19 nt, synthetic construct
  source 1..19: mol_type = other RNA
ctgacaacccggattttaa

SEQ ID NO: 157 - RNA, 19 nt, synthetic construct
  source 1..19: mol_type = other RNA
ttaaaatccgggttgtcag

SEQ ID NO: 158 - RNA, 19 nt, synthetic construct
  source 1..19: mol_type = other RNA
aatgagttcacgtacctga

SEQ ID NO: 159 - RNA, 19 nt, synthetic construct
  source 1..19: mol_type = other RNA
tcaggtacgtgaactcatt

SEQ ID NO: 160 - RNA, 19 nt, synthetic construct
  source 1..19: mol_type = other RNA
acaacccggattttaacta

SEQ ID NO: 161 - RNA, 19 nt, synthetic construct
  source 1..19: mol_type = other RNA
tagttaaaatccgggttgt

SEQ ID NO: 162 - RNA, 19 nt, synthetic construct
  source 1..19: mol_type = other RNA
agaacggccatcaaagtga

SEQ ID NO: 163 - RNA, 19 nt, synthetic construct
  source 1..19: mol_type = other RNA
tcactttgatggccgttct

SEQ ID NO: 164 - RNA, 19 nt, synthetic construct
  source 1..19: mol_type = other RNA
cttctgacaacccggatta

SEQ ID NO: 165 - RNA, 19 nt, synthetic construct
  source 1..19: mol_type = other RNA
taatccgggttgtcagaag

SEQ ID NO: 166 - RNA, 19 nt, synthetic construct
  source 1..19: mol_type = other RNA
tgtctaaatgagttcacga

SEQ ID NO: 167 - RNA, 19 nt, synthetic construct
  source 1..19: mol_type = other RNA
tcgtgaactcatttagaca

SEQ ID NO: 168 - RNA, 19 nt, synthetic construct
  source 1..19: mol_type = other RNA
agaatacttagatgaacca

SEQ ID NO: 169 - RNA, 19 nt, synthetic construct
  source 1..19: mol_type = other RNA
tggttcatctaagtattct

SEQ ID NO: 170 - RNA, 19 nt, synthetic construct
  source 1..19: mol_type = other RNA
taccttcgaagccatgcta

SEQ ID NO: 171 - RNA, 19 nt, synthetic construct
  source 1..19: mol_type = other RNA
tagcatggcttcgaaggta

SEQ ID NO: 172 - RNA, 19 nt, synthetic construct
  source 1..19: mol_type = other RNA
gtgcatgacccgaactgaa

SEQ ID NO: 173 - RNA, 19 nt, synthetic construct
  source 1..19: mol_type = other RNA
ttcagttcgggtcatgcac

SEQ ID NO: 174 - RNA, 19 nt, synthetic construct
  source 1..19: mol_type = other RNA
tgtgcatgacccgaactga

SEQ ID NO: 175 - RNA, 19 nt, synthetic construct
  source 1..19: mol_type = other RNA
tcagttcgggtcatgcaca

SEQ ID NO: 176 - RNA, 19 nt, synthetic construct
  source 1..19: mol_type = other RNA
cttacaaccagcgcaggaa

SEQ ID NO: 177 - RNA, 19 nt, synthetic construct
  source 1..19: mol_type = other RNA
ttcctgcgctggttgtaag

SEQ ID NO: 178 - RNA, 19 nt, synthetic construct
  source 1..19: mol_type = other RNA
cctaccttcgaagccatga

SEQ ID NO: 179 - RNA, 19 nt, synthetic construct
  source 1..19: mol_type = other RNA
tcatggcttcgaaggtagg

SEQ ID NO: 180 - RNA, 19 nt, synthetic construct
  source 1..19: mol_type = other RNA
gcgtcattaccctggcata

SEQ ID NO: 181 - RNA, 19 nt, synthetic construct
  source 1..19: mol_type = other RNA
tatgccagggtaatgacgc

SEQ ID NO: 182 - RNA, 19 nt, synthetic construct
  source 1..19: mol_type = other RNA
aattgtgtgtgcatgacca

SEQ ID NO: 183 - RNA, 19 nt, synthetic construct
  source 1..19: mol_type = other RNA
tggtcatgcacacacaatt

SEQ ID NO: 184 - RNA, 19 nt, synthetic construct
  source 1..19: mol_type = other RNA
ggattttagcgtcattaca

SEQ ID NO: 185 - RNA, 19 nt, synthetic construct
  source 1..19: mol_type = other RNA
tgtaatgacgctaaaatcc

SEQ ID NO: 186 - RNA, 19 nt, synthetic construct
  source 1..19: mol_type = other RNA
cagcagaatacttagatga

SEQ ID NO: 187 - RNA, 19 nt, synthetic construct
  source 1..19: mol_type = other RNA
tcatctaagtattctgctg

SEQ ID NO: 188 - RNA, 19 nt, synthetic construct
  source 1..19: mol_type = other RNA
gcatgagccctaccttcga

SEQ ID NO: 189 - RNA, 19 nt, synthetic construct
  source 1..19: mol_type = other RNA
tcgaaggtagggctcatgc

SEQ ID NO: 190 - RNA, 19 nt, synthetic construct
  source 1..19: mol_type = other RNA
tgacaacccggattttaaa

SEQ ID NO: 191 - RNA, 19 nt, synthetic construct
  source 1..19: mol_type = other RNA
tttaaaatccgggttgtca

SEQ ID NO: 192 - RNA, 19 nt, synthetic construct
  source 1..19: mol_type = other RNA
ctaaatgagttcacgtaca

SEQ ID NO: 193 - RNA, 19 nt, synthetic construct
  source 1..19: mol_type = other RNA
tgtacgtgaactcatttag

SEQ ID NO: 194 - RNA, 19 nt, synthetic construct
  source 1..19: mol_type = other RNA
tgtcagcagaatacttaga

SEQ ID NO: 195 - RNA, 19 nt, synthetic construct
  source 1..19: mol_type = other RNA
tctaagtattctgctgaca

SEQ ID NO: 196 - RNA, 19 nt, synthetic construct
  source 1..19: mol_type = other RNA
aaccaactactcatctcaa

SEQ ID NO: 197 - RNA, 19 nt, synthetic construct
  source 1..19: mol_type = other RNA
ttgagatgagtagttggtt

SEQ ID NO: 198 - RNA, 19 nt, synthetic construct
  source 1..19: mol_type = other RNA
cgctttactccggagtcaa

SEQ ID NO: 199 - RNA, 19 nt, synthetic construct
  source 1..19: mol_type = other RNA
ttgactccggagtaaagcg

SEQ ID NO: 200 - RNA, 19 nt, synthetic construct
  source 1..19: mol_type = other RNA
acaaccaactactcatcta

SEQ ID NO: 201 - RNA, 19 nt, synthetic construct
  source 1..19: mol_type = other RNA
tagatgagtagttggttgt

SEQ ID NO: 202 - RNA, 19 nt, synthetic construct
  source 1..19: mol_type = other RNA
cttgtacctttgtcgatta

SEQ ID NO: 203 - RNA, 19 nt, synthetic construct
  source 1..19: mol_type = other RNA
taatcgacaaaggtacaag

SEQ ID NO: 204 - RNA, 19 nt, synthetic construct
  source 1..19: mol_type = other RNA
cacgtacctgagagatcca

SEQ ID NO: 205 - RNA, 19 nt, synthetic construct
  source 1..19: mol_type = other RNA
tggatctctcaggtacgtg

SEQ ID NO: 206 - RNA, 19 nt, synthetic construct
  source 1..19: mol_type = other RNA
cccaaaacccaatcgaaaa

SEQ ID NO: 207 - RNA, 19 nt, synthetic construct
  source 1..19: mol_type = other RNA
ttttcgattgggttttggg

SEQ ID NO: 208 - RNA, 19 nt, synthetic construct
  source 1..19: mol_type = other RNA
gggtgaagctcaagacaaa

SEQ ID NO: 209 - RNA, 19 nt, synthetic construct
  source 1..19: mol_type = other RNA
tttgtcttgagcttcaccc

SEQ ID NO: 210 - RNA, 19 nt, synthetic construct
  source 1..19: mol_type = other RNA
gtttatttattacggtcaa

SEQ ID NO: 211 - RNA, 19 nt, synthetic construct
  source 1..19: mol_type = other RNA
ttgaccgtaataaataaac

SEQ ID NO: 212 - RNA, 19 nt, synthetic construct
  source 1..19: mol_type = other RNA
cagtttatttattacggta

SEQ ID NO: 213 - RNA, 19 nt, synthetic construct
  source 1..19: mol_type = other RNA
taccgtaataaataaactg

SEQ ID NO: 214 - RNA, 19 nt, synthetic construct
  source 1..19: mol_type = other RNA
accaactactcatctcaca

SEQ ID NO: 215 - RNA, 19 nt, synthetic construct
  source 1..19: mol_type = other RNA
tgtgagatgagtagttggt

SEQ ID NO: 216 - RNA, 19 nt, synthetic construct
  source 1..19: mol_type = other RNA
acctgatcagaagaacgga

SEQ ID NO: 217 - RNA, 19 nt, synthetic construct
  source 1..19: mol_type = other RNA
tccgttcttctgatcaggt

SEQ ID NO: 218 - RNA, 19 nt, synthetic construct
  source 1..19: mol_type = other RNA
cttttttgtgctcgtaata

SEQ ID NO: 219 - RNA, 19 nt, synthetic construct
  source 1..19: mol_type = other RNA
tattacgagcacaaaaaag

SEQ ID NO: 220 - RNA, 19 nt, synthetic construct
  source 1..19: mol_type = other RNA
gaattccccattggatcca

SEQ ID NO: 221 - RNA, 19 nt, synthetic construct
  source 1..19: mol_type = other RNA
tggatccaatggggaattc

SEQ ID NO: 222 - RNA, 19 nt, synthetic construct
  source 1..19: mol_type = other RNA
ggcatatgttggcacagca

SEQ ID NO: 223 - RNA, 19 nt, synthetic construct
  source 1..19: mol_type = other RNA
tgctgtgccaacatatgcc

SEQ ID NO: 224 - RNA, 19 nt, synthetic construct
  source 1..19: mol_type = other RNA
gccttagtgatagagtcaa

SEQ ID NO: 225 - RNA, 19 nt, synthetic construct
  source 1..19: mol_type = other RNA
ttgactctatcactaaggc

SEQ ID NO: 226 - RNA, 19 nt, synthetic construct
  source 1..19: mol_type = other RNA
tcattaccctggcatatga

SEQ ID NO: 227 - RNA, 19 nt, synthetic construct
  source 1..19: mol_type = other RNA
tcatatgccagggtaatga

SEQ ID NO: 228 - RNA, 19 nt, synthetic construct
  source 1..19: mol_type = other RNA
aatgagtgttgtcagtata

SEQ ID NO: 229 - RNA, 19 nt, synthetic construct
  source 1..19: mol_type = other RNA
tatactgacaacactcatt

SEQ ID NO: 230 - RNA, 19 nt, synthetic construct
  source 1..19: mol_type = other RNA
aggacatctagctcaataa

SEQ ID NO: 231 - RNA, 19 nt, synthetic construct
  source 1..19: mol_type = other RNA
ttattgagctagatgtcct

SEQ ID NO: 232 - RNA, 19 nt, synthetic construct
  source 1..19: mol_type = other RNA
tgatccttacaaccagcga

SEQ ID NO: 233 - RNA, 19 nt, synthetic construct
  source 1..19: mol_type = other RNA
tcgctggttgtaaggatca

SEQ ID NO: 234 - RNA, 19 nt, synthetic construct
  source 1..19: mol_type = other RNA
ttatttattacggtcacta

SEQ ID NO: 235 - RNA, 19 nt, synthetic construct
  source 1..19: mol_type = other RNA
tagtgaccgtaataaataa

SEQ ID NO: 236 - RNA, 19 nt, synthetic construct
  source 1..19: mol_type = other RNA
aagaacggccatcaaagta

SEQ ID NO: 237 - RNA, 19 nt, synthetic construct
  source 1..19: mol_type = other RNA
tactttgatggccgttctt

SEQ ID NO: 238 - RNA, 19 nt, synthetic construct
  source 1..19: mol_type = other RNA
gacaacccggattttaaca

SEQ ID NO: 239 - RNA, 19 nt, synthetic construct
  source 1..19: mol_type = other RNA
tgttaaaatccgggttgtc

SEQ ID NO: 240 - RNA, 19 nt, synthetic construct
  source 1..19: mol_type = other RNA
tctagctcaatacaaaata

SEQ ID NO: 241 - RNA, 19 nt, synthetic construct
  source 1..19: mol_type = other RNA
tattttgtattgagctaga

SEQ ID NO: 242 - RNA, 19 nt, synthetic construct
  source 1..19: mol_type = other RNA
gatctccgtgggatctcaa

SEQ ID NO: 243 - RNA, 19 nt, synthetic construct
  source 1..19: mol_type = other RNA
ttgagatcccacggagatc

SEQ ID NO: 244 - RNA, 19 nt, synthetic construct
  source 1..19: mol_type = other RNA
ccggtttagtgccactcga

SEQ ID NO: 245 - RNA, 19 nt, synthetic construct
  source 1..19: mol_type = other RNA
tcgagtggcactaaaccgg

SEQ ID NO: 246 - RNA, 19 nt, synthetic construct
  source 1..19: mol_type = other RNA
ctcatccttttcttgacga

SEQ ID NO: 247 - RNA, 19 nt, synthetic construct
  source 1..19: mol_type = other RNA
tcgtcaagaaaaggatgag

SEQ ID NO: 248 - RNA, 19 nt, synthetic construct
  source 1..19: mol_type = other RNA
acccaatcgaaatatacta

SEQ ID NO: 249 - RNA, 19 nt, synthetic construct
  source 1..19: mol_type = other RNA
tagtatatttcgattgggt

SEQ ID NO: 250 - RNA, 19 nt, synthetic construct
  source 1..19: mol_type = other RNA
tttatctttgtgttcgaga

SEQ ID NO: 251 - RNA, 19 nt, synthetic construct
  source 1..19: mol_type = other RNA
tctcgaacacaaagataaa

SEQ ID NO: 252 - RNA, 19 nt, synthetic construct
  source 1..19: mol_type = other RNA
acagaaggatgcattcgca

SEQ ID NO: 253 - RNA, 19 nt, synthetic construct
  source 1..19: mol_type = other RNA
tgcgaatgcatccttctgt

SEQ ID NO: 254 - RNA, 19 nt, synthetic construct
  source 1..19: mol_type = other RNA
gtcactagaggccttagta

SEQ ID NO: 255 - RNA, 19 nt, synthetic construct
  source 1..19: mol_type = other RNA
tactaaggcctctagtgac

SEQ ID NO: 256 - RNA, 19 nt, synthetic construct
  source 1..19: mol_type = other RNA
tcttttttgtgctcgtaaa

SEQ ID NO: 257 - RNA, 19 nt, synthetic construct
  source 1..19: mol_type = other RNA
tttacgagcacaaaaaaga

SEQ ID NO: 258 - RNA, 19 nt, synthetic construct
  source 1..19: mol_type = other RNA
ttgtcgattgtgaataaca

SEQ ID NO: 259 - RNA, 19 nt, synthetic construct
  source 1..19: mol_type = other RNA
tgttattcacaatcgacaa

SEQ ID NO: 260 - RNA, 19 nt, synthetic construct
  source 1..19: mol_type = other RNA
agggcaatgagtgttgtca

SEQ ID NO: 261 - RNA, 19 nt, synthetic construct
  source 1..19: mol_type = other RNA
tgacaacactcattgccct

SEQ ID NO: 262 - RNA, 19 nt, synthetic construct
  source 1..19: mol_type = other RNA
tttttgtgctcgtaatcta

SEQ ID NO: 263 - RNA, 19 nt, synthetic construct
  source 1..19: mol_type = other RNA
tagattacgagcacaaaaa

SEQ ID NO: 264 - RNA, 19 nt, synthetic construct
  source 1..19: mol_type = other RNA
ttcgatgcaggacaaaaga

SEQ ID NO: 265 - RNA, 19 nt, synthetic construct
  source 1..19: mol_type = other RNA
tcttttgtcctgcatcgaa

SEQ ID NO: 266 - RNA, 19 nt, synthetic construct
  source 1..19: mol_type = other RNA
caaccgaaaaaatatctca

SEQ ID NO: 267 - RNA, 19 nt, synthetic construct
  source 1..19: mol_type = other RNA
tgagatattttttcggttg

SEQ ID NO: 268 - RNA, 19 nt, synthetic construct
  source 1..19: mol_type = other RNA
acggtcactattttggtta

SEQ ID NO: 269 - RNA, 19 nt, synthetic construct
  source 1..19: mol_type = other RNA
taaccaaaatagtgaccgt

SEQ ID NO: 270 - RNA, 19 nt, synthetic construct
  source 1..19: mol_type = other RNA
ggatgcattcgccactgta

SEQ ID NO: 271 - RNA, 19 nt, synthetic construct
  source 1..19: mol_type = other RNA
tacagtggcgaatgcatcc

SEQ ID NO: 272 - RNA, 19 nt, synthetic construct
  source 1..19: mol_type = other RNA
gggcaatgagtgttgtcaa

SEQ ID NO: 273 - RNA, 19 nt, synthetic construct
  source 1..19: mol_type = other RNA
ttgacaacactcattgccc

SEQ ID NO: 274 - RNA, 19 nt, synthetic construct
  source 1..19: mol_type = other RNA
tgatagagtcaacatgaga

SEQ ID NO: 275 - RNA, 19 nt, synthetic construct
  source 1..19: mol_type = other RNA
tctcatgttgactctatca

SEQ ID NO: 276 - RNA, 19 nt, synthetic construct
  source 1..19: mol_type = other RNA
atccttacaaccagcgcaa

SEQ ID NO: 277 - RNA, 19 nt, synthetic construct
  source 1..19: mol_type = other RNA
ttgcgctggttgtaaggat

SEQ ID NO: 278 - RNA, 19 nt, synthetic construct
  source 1..19: mol_type = other RNA
tgtacctttgtcgattgta

SEQ ID NO: 279 - RNA, 19 nt, synthetic construct
  source 1..19: mol_type = other RNA
tacaatcgacaaaggtaca

SEQ ID NO: 280 - RNA, 19 nt, synthetic construct
  source 1..19: mol_type = other RNA
ttgtacctttgtcgattga

SEQ ID NO: 281 - RNA, 19 nt, synthetic construct
  source 1..19: mol_type = other RNA
tcaatcgacaaaggtacaa

SEQ ID NO: 282 - RNA, 19 nt, synthetic construct
  source 1..19: mol_type = other RNA
actcgaggagtctgaacaa

SEQ ID NO: 283 - RNA, 19 nt, synthetic construct
  source 1..19: mol_type = other RNA
ttgttcagactcctcgagt

SEQ ID NO: 284 - RNA, 19 nt, synthetic construct
  source 1..19: mol_type = other RNA
cagtatcataacctccgta

SEQ ID NO: 285 - RNA, 19 nt, synthetic construct
  source 1..19: mol_type = other RNA
tacggaggttatgatactg

SEQ ID NO: 286 - RNA, 19 nt, synthetic construct
  source 1..19: mol_type = other RNA
aagggcaatgagtgttgta

SEQ ID NO: 287 - RNA, 19 nt, synthetic construct
  source 1..19: mol_type = other RNA
tacaacactcattgccctt

SEQ ID NO: 288 - RNA, 19 nt, synthetic construct
  source 1..19: mol_type = other RNA
caatgagtgttgtcagtaa

SEQ ID NO: 289 - RNA, 19 nt, synthetic construct
  source 1..19: mol_type = other RNA
ttactgacaacactcattg

SEQ ID NO: 290 - RNA, 19 nt, synthetic construct
  source 1..19: mol_type = other RNA
tgcattcgccactgtccca

SEQ ID NO: 291 - RNA, 19 nt, synthetic construct
  source 1..19: mol_type = other RNA
tgggacagtggcgaatgca

SEQ ID NO: 292 - RNA, 19 nt, synthetic construct
  source 1..19: mol_type = other RNA
atcttttttgtgctcgtaa

SEQ ID NO: 293 - RNA, 19 nt, synthetic construct
  source 1..19: mol_type = other RNA
ttacgagcacaaaaaagat

SEQ ID NO: 294 - RNA, 19 nt, synthetic construct
  source 1..19: mol_type = other RNA
cagtgacttcatcgctaaa

SEQ ID NO: 295 - RNA, 19 nt, synthetic construct
  source 1..19: mol_type = other RNA
tttagcgatgaagtcactg

SEQ ID NO: 296 - RNA, 19 nt, synthetic construct
  source 1..19: mol_type = other RNA
gtttatggcaactaatcta

SEQ ID NO: 297 - RNA, 19 nt, synthetic construct
  source 1..19: mol_type = other RNA
tagattagttgccataaac

SEQ ID NO: 298 - RNA, 19 nt, synthetic construct
  source 1..19: mol_type = other RNA
cttgtgcatcgtggtgaaa

SEQ ID NO: 299 - RNA, 19 nt, synthetic construct
  source 1..19: mol_type = other RNA
tttcaccacgatgcacaag

SEQ ID NO: 300 - RNA, 19 nt, synthetic construct
  source 1..19: mol_type = other RNA
ctttgggcttgtgacggaa

SEQ ID NO: 301 - RNA, 19 nt, synthetic construct
  source 1..19: mol_type = other RNA
ttccgtcacaagcccaaag

SEQ ID NO: 302 - RNA, 19 nt, synthetic construct
  source 1..19: mol_type = other RNA
cttgggctttcctctcaca

SEQ ID NO: 303 - RNA, 19 nt, synthetic construct
  source 1..19: mol_type = other RNA
tgtgagaggaaagcccaag

SEQ ID NO: 304 - RNA, 19 nt, synthetic construct
  source 1..19: mol_type = other RNA
catctagctcaatacaaaa

SEQ ID NO: 305 - RNA, 19 nt, synthetic construct
  source 1..19: mol_type = other RNA
ttttgtattgagctagatg

SEQ ID NO: 306 - RNA, 19 nt, synthetic construct
  source 1..19: mol_type = other RNA
gttgtcagtatcataacca

SEQ ID NO: 307 - RNA, 19 nt, synthetic construct
  source 1..19: mol_type = other RNA
tggttatgatactgacaac

SEQ ID NO: 308 - RNA, 19 nt, synthetic construct
  source 1..19: mol_type = other RNA
ctccggagtcactggtgga

SEQ ID NO: 309 - RNA, 19 nt, synthetic construct
  source 1..19: mol_type = other RNA
tccaccagtgactccggag

SEQ ID NO: 310 - RNA, 19 nt, synthetic construct
  source 1..19: mol_type = other RNA
aggtctttggccatcgtaa

SEQ ID NO: 311 - RNA, 19 nt, synthetic construct
  source 1..19: mol_type = other RNA
ttacgatggccaaagacct

SEQ ID NO: 312 - RNA, 19 nt, synthetic construct
  source 1..19: mol_type = other RNA
gtgtgcatgacccgaacta

SEQ ID NO: 313 - RNA, 19 nt, synthetic construct
  source 1..19: mol_type = other RNA
tagttcgggtcatgcacac

SEQ ID NO: 314 - RNA, 19 nt, synthetic construct
  source 1..19: mol_type = other RNA
cacggatccaggtctttga

SEQ ID NO: 315 - RNA, 19 nt, synthetic construct
  source 1..19: mol_type = other RNA
tcaaagacctggatccgtg

SEQ ID NO: 316 - RNA, 19 nt, synthetic construct
  source 1..19: mol_type = other RNA
acttagatgaacctttcca

SEQ ID NO: 317 - RNA, 19 nt, synthetic construct
  source 1..19: mol_type = other RNA
tggaaaggttcatctaagt

SEQ ID NO: 318 - RNA, 19 nt, synthetic construct
  source 1..19: mol_type = other RNA
aacatgcaacccaagtgga

SEQ ID NO: 319 - RNA, 19 nt, synthetic construct
  source 1..19: mol_type = other RNA
tccacttgggttgcatgtt

SEQ ID NO: 320 - RNA, 19 nt, synthetic construct
  source 1..19: mol_type = other RNA
tagaggccttagtgataga

SEQ ID NO: 321 - RNA, 19 nt, synthetic construct
  source 1..19: mol_type = other RNA
tctatcactaaggcctcta

SEQ ID NO: 322 - RNA, 19 nt, synthetic construct
  source 1..19: mol_type = other RNA
atcgaaatatactgatcca

SEQ ID NO: 323 - RNA, 19 nt, synthetic construct
  source 1..19: mol_type = other RNA
tggatcagtatatttcgat

SEQ ID NO: 324 - RNA, 19 nt, synthetic construct
  source 1..19: mol_type = other RNA
tttcagccactgtcattca

SEQ ID NO: 325 - RNA, 19 nt, synthetic construct
  source 1..19: mol_type = other RNA
tgaatgacagtggctgaaa

SEQ ID NO: 326 - RNA, 19 nt, synthetic construct
  source 1..19: mol_type = other RNA
ataacctccgtccttgaga

SEQ ID NO: 327 - RNA, 19 nt, synthetic construct
  source 1..19: mol_type = other RNA
tctcaaggacggaggttat

SEQ ID NO: 328 - RNA, 19 nt, synthetic construct
  source 1..19: mol_type = other RNA
tttatggcaactaatctta

SEQ ID NO: 329 - RNA, 19 nt, synthetic construct
  source 1..19: mol_type = other RNA
taagattagttgccataaa

SEQ ID NO: 330 - RNA, 19 nt, synthetic construct
  source 1..19: mol_type = other RNA
ctatgaaccaatagcaaca

SEQ ID NO: 331 - RNA, 19 nt, synthetic construct
  source 1..19: mol_type = other RNA
tgttgctattggttcatag

SEQ ID NO: 332 - RNA, 19 nt, synthetic construct
  source 1..19: mol_type = other RNA
ccaactactcatctcacaa

SEQ ID NO: 333 - RNA, 19 nt, synthetic construct
  source 1..19: mol_type = other RNA
ttgtgagatgagtagttgg

SEQ ID NO: 334 - RNA, 19 nt, synthetic construct
  source 1..19: mol_type = other RNA
gttcatcgccctgctatta

SEQ ID NO: 335 - RNA, 19 nt, synthetic construct
  source 1..19: mol_type = other RNA
taatagcagggcgatgaac

SEQ ID NO: 336 - RNA, 19 nt, synthetic construct
  source 1..19: mol_type = other RNA
atccttttcttgacggtga

SEQ ID NO: 337 - RNA, 19 nt, synthetic construct
  source 1..19: mol_type = other RNA
tcaccgtcaagaaaaggat

SEQ ID NO: 338 - RNA, 19 nt, synthetic construct
  source 1..19: mol_type = other RNA
ctatcggagctatgtgcta

SEQ ID NO: 339 - RNA, 19 nt, synthetic construct
  source 1..19: mol_type = other RNA
tagcacatagctccgatag

SEQ ID NO: 340 - RNA, 19 nt, synthetic construct
  source 1..19: mol_type = other RNA
ccaagaatgtcctaggaga

SEQ ID NO: 341 - RNA, 19 nt, synthetic construct
  source 1..19: mol_type = other RNA
tctcctaggacattcttgg

SEQ ID NO: 342 - RNA, 19 nt, synthetic construct
  source 1..19: mol_type = other RNA
ttctacagcacacaccgga

SEQ ID NO: 343 - RNA, 19 nt, synthetic construct
  source 1..19: mol_type = other RNA
tccggtgtgtgctgtagaa

SEQ ID NO: 344 - RNA, 19 nt, synthetic construct
  source 1..19: mol_type = other RNA
agtttatttattacggtca

SEQ ID NO: 345 - RNA, 19 nt, synthetic construct
  source 1..19: mol_type = other RNA
tgaccgtaataaataaact

SEQ ID NO: 346 - RNA, 19 nt, synthetic construct
  source 1..19: mol_type = other RNA
atcatcctatgaaccaata

SEQ ID NO: 347 - RNA, 19 nt, synthetic construct
  source 1..19: mol_type = other RNA
tattggttcataggatgat

SEQ ID NO: 348 - RNA, 19 nt, synthetic construct
  source 1..19: mol_type = other RNA
caggtctttggccatcgta

SEQ ID NO: 349 - RNA, 19 nt, synthetic construct
  source 1..19: mol_type = other RNA
tacgatggccaaagacctg

SEQ ID NO: 350 - RNA, 19 nt, synthetic construct
  source 1..19: mol_type = other RNA
gcacacaccggacatttaa

SEQ ID NO: 351 - RNA, 19 nt, synthetic construct
  source 1..19: mol_type = other RNA
ttaaatgtccggtgtgtgc

SEQ ID NO: 352 - RNA, 19 nt, synthetic construct
  source 1..19: mol_type = other RNA
catcgtgaccagacaagca

SEQ ID NO: 353 - RNA, 19 nt, synthetic construct
  source 1..19: mol_type = other RNA
tgcttgtctggtcacgatg

SEQ ID NO: 354 - RNA, 19 nt, synthetic construct
  source 1..19: mol_type = other RNA
gcaatgggttaccttgcaa

SEQ ID NO: 355 - RNA, 19 nt, synthetic construct
  source 1..19: mol_type = other RNA
ttgcaaggtaacccattgc

SEQ ID NO: 356 - RNA, 19 nt, synthetic construct
  source 1..19: mol_type = other RNA
tccttttcttgacggtgaa

SEQ ID NO: 357 - RNA, 19 nt, synthetic construct
  source 1..19: mol_type = other RNA
ttcaccgtcaagaaaagga

SEQ ID NO: 358 - RNA, 19 nt, synthetic construct
  source 1..19: mol_type = other RNA
tcgaaatatactgatccaa

SEQ ID NO: 359 - RNA, 19 nt, synthetic construct
  source 1..19: mol_type = other RNA
ttggatcagtatatttcga

SEQ ID NO: 360 - RNA, 19 nt, synthetic construct
  source 1..19: mol_type = other RNA
tcgatgcaggacaaaagaa

SEQ ID NO: 361 - RNA, 19 nt, synthetic construct
  source 1..19: mol_type = other RNA
ttcttttgtcctgcatcga

SEQ ID NO: 362 - RNA, 19 nt, synthetic construct
  source 1..19: mol_type = other RNA
caaaaggcctatcggagca

SEQ ID NO: 363 - RNA, 19 nt, synthetic construct
  source 1..19: mol_type = other RNA
tgctccgataggccttttg

SEQ ID NO: 364 - RNA, 19 nt, synthetic construct
  source 1..19: mol_type = other RNA
ggcgaatgtgtcatgaaga

SEQ ID NO: 365 - RNA, 19 nt, synthetic construct
  source 1..19: mol_type = other RNA
tcttcatgacacattcgcc

SEQ ID NO: 366 - RNA, 19 nt, synthetic construct
  source 1..19: mol_type = other RNA
gctatgtgctgcaccgcta

SEQ ID NO: 367 - RNA, 19 nt, synthetic construct
  source 1..19: mol_type = other RNA
tagcggtgcagcacatagc

SEQ ID NO: 368 - RNA, 19 nt, synthetic construct
  source 1..19: mol_type = other RNA
tcccagcagaactgatcga

SEQ ID NO: 369 - RNA, 19 nt, synthetic construct
  source 1..19: mol_type = other RNA
tcgatcagttctgctggga

SEQ ID NO: 370 - RNA, 19 nt, synthetic construct
  source 1..19: mol_type = other RNA
cacaccggacatttatgga

SEQ ID NO: 371 - RNA, 19 nt, synthetic construct
  source 1..19: mol_type = other RNA
tccataaatgtccggtgtg

SEQ ID NO: 372 - RNA, 19 nt, synthetic construct
  source 1..19: mol_type = other RNA
gtgtcactagaggccttaa

SEQ ID NO: 373 - RNA, 19 nt, synthetic construct
  source 1..19: mol_type = other RNA
ttaaggcctctagtgacac

SEQ ID NO: 374 - RNA, 19 nt, synthetic construct
  source 1..19: mol_type = other RNA
tgtcgattgtgaataacaa

SEQ ID NO: 375 - RNA, 19 nt, synthetic construct
  source 1..19: mol_type = other RNA
ttgttattcacaatcgaca

SEQ ID NO: 376 - RNA, 19 nt, synthetic construct
  source 1..19: mol_type = other RNA
aacttctgacaacccggaa

SEQ ID NO: 377 - RNA, 19 nt, synthetic construct
  source 1..19: mol_type = other RNA
ttccgggttgtcagaagtt

SEQ ID NO: 378 - RNA, 19 nt, synthetic construct
  source 1..19: mol_type = other RNA
ctatgtgctgcaccgctca

SEQ ID NO: 379 - RNA, 19 nt, synthetic construct
  source 1..19: mol_type = other RNA
tgagcggtgcagcacatag

SEQ ID NO: 380 - RNA, 19 nt, synthetic construct
  source 1..19: mol_type = other RNA
aagtctgactgcaagatta

SEQ ID NO: 381 - RNA, 19 nt, synthetic construct
  source 1..19: mol_type = other RNA
taatcttgcagtcagactt

SEQ ID NO: 382 - RNA, 19 nt, synthetic construct
  source 1..19: mol_type = other RNA
cctttgtcgattgtgaata

SEQ ID NO: 383 - RNA, 19 nt, synthetic construct
  source 1..19: mol_type = other RNA
tattcacaatcgacaaagg

SEQ ID NO: 384 - RNA, 19 nt, synthetic construct
  source 1..19: mol_type = other RNA
gtttagtgccactcgggca

SEQ ID NO: 385 - RNA, 19 nt, synthetic construct
  source 1..19: mol_type = other RNA
tgcccgagtggcactaaac

SEQ ID NO: 386 - RNA, 19 nt, synthetic construct
  source 1..19: mol_type = other RNA
tccttacaaccagcgcaga

SEQ ID NO: 387 - RNA, 19 nt, synthetic construct
  source 1..19: mol_type = other RNA
tctgcgctggttgtaagga

SEQ ID NO: 388 - RNA, 19 nt, synthetic construct
  source 1..19: mol_type = other RNA
aacccaatcgaaatataca

SEQ ID NO: 389 - RNA, 19 nt, synthetic construct
  source 1..19: mol_type = other RNA
tgtatatttcgattgggtt

SEQ ID NO: 390 - RNA, 19 nt, synthetic construct
  source 1..19: mol_type = other RNA
tgtgtgcatgacccgaaca

SEQ ID NO: 391 - RNA, 19 nt, synthetic construct
  source 1..19: mol_type = other RNA
tgttcgggtcatgcacaca

SEQ ID NO: 392 - RNA, 19 nt, synthetic construct
  source 1..19: mol_type = other RNA
catcgccctgctattgaaa

SEQ ID NO: 393 - RNA, 19 nt, synthetic construct
  source 1..19: mol_type = other RNA
tttcaatagcagggcgatg

SEQ ID NO: 394 - RNA, 19 nt, synthetic construct
  source 1..19: mol_type = other RNA
ccttacaaccagcgcagga

SEQ ID NO: 395 - RNA, 19 nt, synthetic construct
  source 1..19: mol_type = other RNA
tcctgcgctggttgtaagg

SEQ ID NO: 396 - RNA, 19 nt, synthetic construct
  source 1..19: mol_type = other RNA
atctccgcgccccatgaaa

SEQ ID NO: 397 - RNA, 19 nt, synthetic construct
  source 1..19: mol_type = other RNA
tttcatggggcgcggagat

SEQ ID NO: 398 - RNA, 19 nt, synthetic construct
  source 1..19: mol_type = other RNA
cttttcttgacggtgatga

SEQ ID NO: 399 - RNA, 19 nt, synthetic construct
  source 1..19: mol_type = other RNA
tcatcaccgtcaagaaaag

SEQ ID NO: 400 - RNA, 19 nt, synthetic construct
  source 1..19: mol_type = other RNA
tcactagaggccttagtga

SEQ ID NO: 401 - RNA, 19 nt, synthetic construct
  source 1..19: mol_type = other RNA
tcactaaggcctctagtga

SEQ ID NO: 402 - RNA, 19 nt, synthetic construct
  source 1..19: mol_type = other RNA
gcgaatgtgtcatgaagaa

SEQ ID NO: 403 - RNA, 19 nt, synthetic construct
  source 1..19: mol_type = other RNA
ttcttcatgacacattcgc

SEQ ID NO: 404 - RNA, 19 nt, synthetic construct
  source 1..19: mol_type = other RNA
tctgacaacccggatttta

SEQ ID NO: 405 - RNA, 19 nt, synthetic construct
  source 1..19: mol_type = other RNA
taaaatccgggttgtcaga

SEQ ID NO: 406 - RNA, 19 nt, synthetic construct
  source 1..19: mol_type = other RNA
agtatcataacctccgtca

SEQ ID NO: 407 - RNA, 19 nt, synthetic construct
  source 1..19: mol_type = other RNA
tgacggaggttatgatact

SEQ ID NO: 408 - RNA, 19 nt, synthetic construct
  source 1..19: mol_type = other RNA
gcatatgttggcacagcaa

SEQ ID NO: 409 - RNA, 19 nt, synthetic construct
  source 1..19: mol_type = other RNA
ttgctgtgccaacatatgc

SEQ ID NO: 410 - RNA, 19 nt, synthetic construct
  source 1..19: mol_type = other RNA
cccttactgtgtggcaata

SEQ ID NO: 411 - RNA, 19 nt, synthetic construct
  source 1..19: mol_type = other RNA
tattgccacacagtaaggg

SEQ ID NO: 412 - RNA, 19 nt, synthetic construct
  source 1..19: mol_type = other RNA
ctacagcacacaccggaca

SEQ ID NO: 413 - RNA, 19 nt, synthetic construct
  source 1..19: mol_type = other RNA
tgtccggtgtgtgctgtag

SEQ ID NO: 414 - RNA, 19 nt, synthetic construct
  source 1..19: mol_type = other RNA
cgaaaaaatatctccgcga

SEQ ID NO: 415 - RNA, 19 nt, synthetic construct
  source 1..19: mol_type = other RNA
tcgcggagatattttttcg

SEQ ID NO: 416 - RNA, 19 nt, synthetic construct
  source 1..19: mol_type = other RNA
cttggtggggctgcaacta

SEQ ID NO: 417 - RNA, 19 nt, synthetic construct
  source 1..19: mol_type = other RNA
tagttgcagccccaccaag

SEQ ID NO: 418 - RNA, 19 nt, synthetic construct
  source 1..19: mol_type = other RNA
ctgcaagattcaaaactca

SEQ ID NO: 419 - RNA, 19 nt, synthetic construct
  source 1..19: mol_type = other RNA
tgagttttgaatcttgcag

SEQ ID NO: 420 - RNA, 19 nt, synthetic construct
  source 1..19: mol_type = other RNA
cgtcgctttactccggaga

SEQ ID NO: 421 - RNA, 19 nt, synthetic construct
  source 1..19: mol_type = other RNA
tctccggagtaaagcgacg

SEQ ID NO: 422 - RNA, 19 nt, synthetic construct
  source 1..19: mol_type = other RNA
tcatcctatgaaccaataa

SEQ ID NO: 423 - RNA, 19 nt, synthetic construct
  source 1..19: mol_type = other RNA
ttattggttcataggatga

SEQ ID NO: 424 - RNA, 21 nt, synthetic construct
  source 1..21: mol_type = other RNA
tccaaaatagtgaccgtaatt

SEQ ID NO: 425 - RNA, 21 nt, synthetic construct
  source 1..21: mol_type = other RNA
ttgactacagccaagatcatt

SEQ ID NO: 426 - RNA, 21 nt, synthetic construct
  source 1..21: mol_type = other RNA
tgtcatgcacacacaattatt

SEQ ID NO: 427 - RNA, 21 nt, synthetic construct
  source 1..21: mol_type = other RNA
ttaaccaaaatagtgaccgtt

SEQ ID NO: 428 - RNA, 21 nt, synthetic construct
  source 1..21: mol_type = other RNA
tcgttcttctgatcaggtttt

SEQ ID NO: 429 - RNA, 21 nt, synthetic construct
  source 1..21: mol_type = other RNA
taggtacgtgaactcattttt

SEQ ID NO: 430 - RNA, 21 nt, synthetic construct
  source 1..21: mol_type = other RNA
ttagttaaaatccgggttgtt

SEQ ID NO: 431 - RNA, 21 nt, synthetic construct
  source 1..21: mol_type = other RNA
ttaaaatccgggttgtcagtt

SEQ ID NO: 432 - RNA, 21 nt, synthetic construct
  source 1..21: mol_type = other RNA
tcaggtacgtgaactcatttt

SEQ ID NO: 433 - RNA, 21 nt, synthetic construct
  source 1..21: mol_type = other RNA
tagttaaaatccgggttgttt

SEQ ID NO: 434 - RNA, 21 nt, synthetic construct
  source 1..21: mol_type = other RNA
tcactttgatggccgttcttt

SEQ ID NO: 435 - RNA, 21 nt, synthetic construct
  source 1..21: mol_type = other RNA
taatccgggttgtcagaagtt

SEQ ID NO: 436 - RNA, 21 nt, synthetic construct
  source 1..21: mol_type = other RNA
tcgtgaactcatttagacatt

SEQ ID NO: 437 - RNA, 21 nt, synthetic construct
  source 1..21: mol_type = other RNA
tggttcatctaagtattcttt

SEQ ID NO: 438 - RNA, 21 nt, synthetic construct
  source 1..21: mol_type = other RNA
tagcatggcttcgaaggtatt

SEQ ID NO: 439 - RNA, 21 nt, synthetic construct
  source 1..21: mol_type = other RNA
ttcagttcgggtcatgcactt

SEQ ID NO: 440 - RNA, 21 nt, synthetic construct
  source 1..21: mol_type = other RNA
tcagttcgggtcatgcacatt

SEQ ID NO: 441 - RNA, 21 nt, synthetic construct
  source 1..21: mol_type = other RNA
ttcctgcgctggttgtaagtt

SEQ ID NO: 442 - RNA, 21 nt, synthetic construct
  source 1..21: mol_type = other RNA
tcatggcttcgaaggtaggtt

SEQ ID NO: 443 - RNA, 21 nt, synthetic construct
  source 1..21: mol_type = other RNA
tatgccagggtaatgacgctt

SEQ ID NO: 444 - RNA, 21 nt, synthetic construct
  source 1..21: mol_type = other RNA
tggtcatgcacacacaatttt

SEQ ID NO: 445 - RNA, 21 nt, synthetic construct
  source 1..21: mol_type = other RNA
tgtaatgacgctaaaatcctt

SEQ ID NO: 446 - RNA, 21 nt, synthetic construct
  source 1..21: mol_type = other RNA
tcatctaagtattctgctgtt

SEQ ID NO: 447 - RNA, 21 nt, synthetic construct
  source 1..21: mol_type = other RNA
tcgaaggtagggctcatgctt

SEQ ID NO: 448 - RNA, 21 nt, synthetic construct
  source 1..21: mol_type = other RNA
tttaaaatccgggttgtcatt

SEQ ID NO: 449 - RNA, 21 nt, synthetic construct
  source 1..21: mol_type = other RNA
tgtacgtgaactcatttagtt

SEQ ID NO: 450 - RNA, 21 nt, synthetic construct
  source 1..21: mol_type = other RNA
tctaagtattctgctgacatt

SEQ ID NO: 451 - RNA, 21 nt, synthetic construct
  source 1..21: mol_type = other RNA
ttgagatgagtagttggtttt

SEQ ID NO: 452 - RNA, 21 nt, synthetic construct
  source 1..21: mol_type = other RNA
ttgactccggagtaaagcgtt

SEQ ID NO: 453 - RNA, 21 nt, synthetic construct
  source 1..21: mol_type = other RNA
tagatgagtagttggttgttt

SEQ ID NO: 454 - RNA, 21 nt, synthetic construct
  source 1..21: mol_type = other RNA
taatcgacaaaggtacaagtt

SEQ ID NO: 455 - RNA, 21 nt, synthetic construct
  source 1..21: mol_type = other RNA
tggatctctcaggtacgtgtt

SEQ ID NO: 456 - RNA, 21 nt, synthetic construct
  source 1..21: mol_type = other RNA
ttttcgattgggttttgggtt

SEQ ID NO: 457 - RNA, 21 nt, synthetic construct
  source 1..21: mol_type = other RNA
tttgtcttgagcttcaccctt

SEQ ID NO: 458 - RNA, 21 nt, synthetic construct
  source 1..21: mol_type = other RNA
ttgaccgtaataaataaactt

SEQ ID NO: 459 - RNA, 21 nt, synthetic construct
  source 1..21: mol_type = other RNA
taccgtaataaataaactgtt

SEQ ID NO: 460 - RNA, 21 nt, synthetic construct
  source 1..21: mol_type = other RNA
tgtgagatgagtagttggttt

SEQ ID NO: 461 - RNA, 21 nt, synthetic construct
  source 1..21: mol_type = other RNA
tccgttcttctgatcaggttt

SEQ ID NO: 462 - RNA, 21 nt, synthetic construct
  source 1..21: mol_type = other RNA
tattacgagcacaaaaaagtt

SEQ ID NO: 463 - RNA, 21 nt, synthetic construct
  source 1..21: mol_type = other RNA
tggatccaatggggaattctt

SEQ ID NO: 464 - RNA, 21 nt, synthetic construct
  source 1..21: mol_type = other RNA
tgctgtgccaacatatgcctt

SEQ ID NO: 465 - RNA, 21 nt, synthetic construct
  source 1..21: mol_type = other RNA
ttgactctatcactaaggctt

SEQ ID NO: 466 - RNA, 21 nt, synthetic construct
  source 1..21: mol_type = other RNA
tcatatgccagggtaatgatt

SEQ ID NO: 467 - RNA, 21 nt, synthetic construct
  source 1..21: mol_type = other RNA
tatactgacaacactcatttt

SEQ ID NO: 468 - RNA, 21 nt, synthetic construct
  source 1..21: mol_type = other RNA
ttattgagctagatgtccttt

SEQ ID NO: 469 - RNA, 21 nt, synthetic construct
  source 1..21: mol_type = other RNA
tcgctggttgtaaggatcatt

SEQ ID NO: 470 - RNA, 21 nt, synthetic construct
  source 1..21: mol_type = other RNA
tagtgaccgtaataaataatt

SEQ ID NO: 471 - RNA, 21 nt, synthetic construct
  source 1..21: mol_type = other RNA
tactttgatggccgttctttt

SEQ ID NO: 472 - RNA, 21 nt, synthetic construct
  source 1..21: mol_type = other RNA
tgttaaaatccgggttgtctt

SEQ ID NO: 473 - RNA, 21 nt, synthetic construct
  source 1..21: mol_type = other RNA
tattttgtattgagctagatt

SEQ ID NO: 474 - RNA, 21 nt, synthetic construct
  source 1..21: mol_type = other RNA
ttgagatcccacggagatctt

SEQ ID NO: 475 - RNA, 21 nt, synthetic construct
  source 1..21: mol_type = other RNA
tcgagtggcactaaaccggtt

SEQ ID NO: 476 - RNA, 21 nt, synthetic construct
  source 1..21: mol_type = other RNA
tcgtcaagaaaaggatgagtt

SEQ ID NO: 477 - RNA, 21 nt, synthetic construct
  source 1..21: mol_type = other RNA
tagtatatttcgattgggttt

SEQ ID NO: 478 - RNA, 21 nt, synthetic construct
  source 1..21: mol_type = other RNA
tctcgaacacaaagataaatt

SEQ ID NO: 479 - RNA, 21 nt, synthetic construct
  source 1..21: mol_type = other RNA
tgcgaatgcatccttctgttt

SEQ ID NO: 480 - RNA, 21 nt, synthetic construct
  source 1..21: mol_type = other RNA
tactaaggcctctagtgactt

SEQ ID NO: 481 - RNA, 21 nt, synthetic construct
  source 1..21: mol_type = other RNA
tttacgagcacaaaaaagatt

SEQ ID NO: 482 - RNA, 21 nt, synthetic construct
  source 1..21: mol_type = other RNA
tgttattcacaatcgacaatt

SEQ ID NO: 483 - RNA, 21 nt, synthetic construct
  source 1..21: mol_type = other RNA
tgacaacactcattgcccttt

SEQ ID NO: 484 - RNA, 21 nt, synthetic construct
  source 1..21: mol_type = other RNA
tagattacgagcacaaaaatt

SEQ ID NO: 485 - RNA, 21 nt, synthetic construct
  source 1..21: mol_type = other RNA
tcttttgtcctgcatcgaatt

SEQ ID NO: 486 - RNA, 21 nt, synthetic construct
  source 1..21: mol_type = other RNA
tgagatattttttcggttgtt

SEQ ID NO: 487 - RNA, 21 nt, synthetic construct
  source 1..21: mol_type = other RNA
taaccaaaatagtgaccgttt

SEQ ID NO: 488 - RNA, 21 nt, synthetic construct
  source 1..21: mol_type = other RNA
tacagtggcgaatgcatcctt

SEQ ID NO: 489 - RNA, 21 nt, synthetic construct
  source 1..21: mol_type = other RNA
ttgacaacactcattgccctt

SEQ ID NO: 490 - RNA, 21 nt, synthetic construct
  source 1..21: mol_type = other RNA
tctcatgttgactctatcatt

SEQ ID NO: 491 - RNA, 21 nt, synthetic construct
  source 1..21: mol_type = other RNA
ttgcgctggttgtaaggattt

SEQ ID NO: 492 - RNA, 21 nt, synthetic construct
  source 1..21: mol_type = other RNA
tacaatcgacaaaggtacatt

SEQ ID NO: 493 - RNA, 21 nt, synthetic construct
  source 1..21: mol_type = other RNA
tcaatcgacaaaggtacaatt

SEQ ID NO: 494 - RNA, 21 nt, synthetic construct
  source 1..21: mol_type = other RNA
ttgttcagactcctcgagttt

SEQ ID NO: 495 - RNA, 21 nt, synthetic construct
  source 1..21: mol_type = other RNA
tacggaggttatgatactgtt

SEQ ID NO: 496 - RNA, 21 nt, synthetic construct
  source 1..21: mol_type = other RNA
tacaacactcattgccctttt

SEQ ID NO: 497 - RNA, 21 nt, synthetic construct
  source 1..21: mol_type = other RNA
ttactgacaacactcattgtt

SEQ ID NO: 498 - RNA, 21 nt, synthetic construct
  source 1..21: mol_type = other RNA
tgggacagtggcgaatgcatt

SEQ ID NO: 499 - RNA, 21 nt, synthetic construct
  source 1..21: mol_type = other RNA
ttacgagcacaaaaaagattt

SEQ ID NO: 500 - RNA, 21 nt, synthetic construct
  source 1..21: mol_type = other RNA
tttagcgatgaagtcactgtt

SEQ ID NO: 501 - RNA, 21 nt, synthetic construct
  source 1..21: mol_type = other RNA
tagattagttgccataaactt

SEQ ID NO: 502 - RNA, 21 nt, synthetic construct
  source 1..21: mol_type = other RNA
tttcaccacgatgcacaagtt

SEQ ID NO: 503 - RNA, 21 nt, synthetic construct
  source 1..21: mol_type = other RNA
ttccgtcacaagcccaaagtt

SEQ ID NO: 504 - RNA, 21 nt, synthetic construct
  source 1..21: mol_type = other RNA
tgtgagaggaaagcccaagtt

SEQ ID NO: 505 - RNA, 21 nt, synthetic construct
  source 1..21: mol_type = other RNA
ttttgtattgagctagatgtt

SEQ ID NO: 506 - RNA, 21 nt, synthetic construct
  source 1..21: mol_type = other RNA
tggttatgatactgacaactt

SEQ ID NO: 507 - RNA, 21 nt, synthetic construct
  source 1..21: mol_type = other RNA
tccaccagtgactccggagtt

SEQ ID NO: 508 - RNA, 21 nt, synthetic construct
  source 1..21: mol_type = other RNA
ttacgatggccaaagaccttt

SEQ ID NO: 509 - RNA, 21 nt, synthetic construct
  source 1..21: mol_type = other RNA
tagttcgggtcatgcacactt

SEQ ID NO: 510 - RNA, 21 nt, synthetic construct
  source 1..21: mol_type = other RNA
tcaaagacctggatccgtgtt

SEQ ID NO: 511 - RNA, 21 nt, synthetic construct
  source 1..21: mol_type = other RNA
tggaaaggttcatctaagttt

SEQ ID NO: 512 - RNA, 21 nt, synthetic construct
  source 1..21: mol_type = other RNA
tccacttgggttgcatgtttt

SEQ ID NO: 513 - RNA, 21 nt, synthetic construct
  source 1..21: mol_type = other RNA
tctatcactaaggcctctatt

SEQ ID NO: 514 - RNA, 21 nt, synthetic construct
  source 1..21: mol_type = other RNA
tggatcagtatatttcgattt

SEQ ID NO: 515 - RNA, 21 nt, synthetic construct
  source 1..21: mol_type = other RNA
tgaatgacagtggctgaaatt

SEQ ID NO: 516 - RNA, 21 nt, synthetic construct
  source 1..21: mol_type = other RNA
tctcaaggacggaggttattt

SEQ ID NO: 517 - RNA, 21 nt, synthetic construct
  source 1..21: mol_type = other RNA
taagattagttgccataaatt

SEQ ID NO: 518 - RNA, 21 nt, synthetic construct
  source 1..21: mol_type = other RNA
tgttgctattggttcatagtt

SEQ ID NO: 519 - RNA, 21 nt, synthetic construct
  source 1..21: mol_type = other RNA
ttgtgagatgagtagttggtt

SEQ ID NO: 520 - RNA, 21 nt, synthetic construct
  source 1..21: mol_type = other RNA
taatagcagggcgatgaactt

SEQ ID NO: 521 - RNA, 21 nt, synthetic construct
  source 1..21: mol_type = other RNA
tcaccgtcaagaaaaggattt

SEQ ID NO: 522 - RNA, 21 nt, synthetic construct
  source 1..21: mol_type = other RNA
tagcacatagctccgatagtt

SEQ ID NO: 523 - RNA, 21 nt, synthetic construct
  source 1..21: mol_type = other RNA
tctcctaggacattcttggtt

SEQ ID NO: 524 - RNA, 21 nt, synthetic construct
  source 1..21: mol_type = other RNA
tccggtgtgtgctgtagaatt

SEQ ID NO: 525 - RNA, 21 nt, synthetic construct
  source 1..21: mol_type = other RNA
tgaccgtaataaataaacttt

SEQ ID NO: 526 - RNA, 21 nt, synthetic construct
  source 1..21: mol_type = other RNA
tattggttcataggatgattt

SEQ ID NO: 527 - RNA, 21 nt, synthetic construct
  source 1..21: mol_type = other RNA
tacgatggccaaagacctgtt

SEQ ID NO: 528 - RNA, 21 nt, synthetic construct
  source 1..21: mol_type = other RNA
ttaaatgtccggtgtgtgctt

SEQ ID NO: 529 - RNA, 21 nt, synthetic construct
  source 1..21: mol_type = other RNA
tgcttgtctggtcacgatgtt

SEQ ID NO: 530 - RNA, 21 nt, synthetic construct
  source 1..21: mol_type = other RNA
ttgcaaggtaacccattgctt

SEQ ID NO: 531 - RNA, 21 nt, synthetic construct
  source 1..21: mol_type = other RNA
ttcaccgtcaagaaaaggatt

SEQ ID NO: 532 - RNA, 21 nt, synthetic construct
  source 1..21: mol_type = other RNA
ttggatcagtatatttcgatt

SEQ ID NO: 533 - RNA, 21 nt, synthetic construct
  source 1..21: mol_type = other RNA
ttcttttgtcctgcatcgatt

SEQ ID NO: 534 - RNA, 21 nt, synthetic construct
  source 1..21: mol_type = other RNA
tgctccgataggccttttgtt

SEQ ID NO: 535 - RNA, 21 nt, synthetic construct
  source 1..21: mol_type = other RNA
tcttcatgacacattcgcctt

SEQ ID NO: 536 - RNA, 21 nt, synthetic construct
  source 1..21: mol_type = other RNA
tagcggtgcagcacatagctt

SEQ ID NO: 537 - RNA, 21 nt, synthetic construct
  source 1..21: mol_type = other RNA
tcgatcagttctgctgggatt

SEQ ID NO: 538 - RNA, 21 nt, synthetic construct
  source 1..21: mol_type = other RNA
tccataaatgtccggtgtgtt

SEQ ID NO: 539 - RNA, 21 nt, synthetic construct
  source 1..21: mol_type = other RNA
ttaaggcctctagtgacactt

SEQ ID NO: 540 - RNA, 21 nt, synthetic construct
  source 1..21: mol_type = other RNA
ttgttattcacaatcgacatt

SEQ ID NO: 541 - RNA, 21 nt, synthetic construct
  source 1..21: mol_type = other RNA
ttccgggttgtcagaagtttt

SEQ ID NO: 542 - RNA, 21 nt, synthetic construct
  source 1..21: mol_type = other RNA
tgagcggtgcagcacatagtt

SEQ ID NO: 543 - RNA, 21 nt, synthetic construct
  source 1..21: mol_type = other RNA
taatcttgcagtcagactttt

SEQ ID NO: 544 - RNA, 21 nt, synthetic construct
  source 1..21: mol_type = other RNA
tattcacaatcgacaaaggtt

SEQ ID NO: 545 - RNA, 21 nt, synthetic construct
  source 1..21: mol_type = other RNA
tgcccgagtggcactaaactt

SEQ ID NO: 546 - RNA, 21 nt, synthetic construct
  source 1..21: mol_type = other RNA
tctgcgctggttgtaaggatt

SEQ ID NO: 547 - RNA, 21 nt, synthetic construct
  source 1..21: mol_type = other RNA
tgtatatttcgattgggtttt

SEQ ID NO: 548 - RNA, 21 nt, synthetic construct
  source 1..21: mol_type = other RNA
tgttcgggtcatgcacacatt

SEQ ID NO: 549 - RNA, 21 nt, synthetic construct
  source 1..21: mol_type = other RNA
tttcaatagcagggcgatgtt

SEQ ID NO: 550 - RNA, 21 nt, synthetic construct
  source 1..21: mol_type = other RNA
tcctgcgctggttgtaaggtt

SEQ ID NO: 551 - RNA, 21 nt, synthetic construct
  source 1..21: mol_type = other RNA
tttcatggggcgcggagattt

SEQ ID NO: 552 - RNA, 21 nt, synthetic construct
  source 1..21: mol_type = other RNA
tcatcaccgtcaagaaaagtt

SEQ ID NO: 553 - RNA, 21 nt, synthetic construct
  source 1..21: mol_type = other RNA
tcactaaggcctctagtgatt

SEQ ID NO: 554 - RNA, 21 nt, synthetic construct
  source 1..21: mol_type = other RNA
ttcttcatgacacattcgctt

SEQ ID NO: 555 - RNA, 21 nt, synthetic construct
  source 1..21: mol_type = other RNA
taaaatccgggttgtcagatt

SEQ ID NO: 556 - RNA, 21 nt, synthetic construct
  source 1..21: mol_type = other RNA
tgacggaggttatgatacttt

SEQ ID NO: 557 - RNA, 21 nt, synthetic construct
  source 1..21: mol_type = other RNA
ttgctgtgccaacatatgctt

SEQ ID NO: 558 - RNA, 21 nt, synthetic construct
  source 1..21: mol_type = other RNA
tattgccacacagtaagggtt

SEQ ID NO: 559 - RNA, 21 nt, synthetic construct
  source 1..21: mol_type = other RNA
tgtccggtgtgtgctgtagtt

SEQ ID NO: 560 - RNA, 21 nt, synthetic construct
  source 1..21: mol_type = other RNA
tcgcggagatattttttcgtt

SEQ ID NO: 561 - RNA, 21 nt, synthetic construct
  source 1..21: mol_type = other RNA
tagttgcagccccaccaagtt

SEQ ID NO: 562 - RNA, 21 nt, synthetic construct
  source 1..21: mol_type = other RNA
tgagttttgaatcttgcagtt

SEQ ID NO: 563 - RNA, 21 nt, synthetic construct
  source 1..21: mol_type = other RNA
tctccggagtaaagcgacgtt

SEQ ID NO: 564 - RNA, 21 nt, synthetic construct
  source 1..21: mol_type = other RNA
ttattggttcataggatgatt

SEQ ID NO: 565 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = cm
  modified_base 5: mod_base = gm
  modified_base 6: mod_base = gm
  modified_base 7: mod_base = OTHER; note = 2'-fluorouridine
  modified_base 8: mod_base = OTHER; note = 2'-fluorocytidine
  modified_base 9: mod_base = OTHER; note = 2'-fluoroadenosine
  modified_base 10: mod_base = cm
  modified_base 11: mod_base = um
  modified_base 12: mod_base = OTHER; note = 2'-O-methyladenosine
  modified_base 13: mod_base = um
  modified_base 14: mod_base = um
  modified_base 15: mod_base = um
  modified_base 16: mod_base = um
  modified_base 17: mod_base = OTHER; note = 2'-O-methylguanosine with a 3'-phosphorothioate linkage
  modified_base 18: mod_base = OTHER; note = 2'-O-methylguanosine with a 3'-phosphorothioate linkage
  modified_base 19: mod_base = OTHER; note = 2'-O-methyladenosine
ttacggtcactattttgga

SEQ ID NO: 566 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tccaaaatagtgaccgtaatt

SEQ ID NO: 567 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = gm
  modified_base 4: mod_base = gm
  modified_base 5: mod_base = um
  modified_base 6: mod_base = cm
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluorouridine
  modified_base 10: mod_base = OTHER; note = 2'-O-methyladenosine
  modified_base 11: mod_base = um
  modified_base 12: mod_base = um
  modified_base 13: mod_base = um
  modified_base 14: mod_base = um
  modified_base 15: mod_base = gm
  modified_base 16: mod_base = gm
  modified_base 17: mod_base = OTHER; note = 2'-O-methyluridine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
acggtcactattttggtta

SEQ ID NO: 568 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = cm
  modified_base 5: mod_base = cm
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = um
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluorocytidine
  modified_base 17: mod_base = cm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
taaccaaaatagtgaccgttt

SEQ ID NO: 569 - RNA, 19 nt, synthetic construct
  source 1..19: mol_type = other RNA
  modified_base 1: mod_base = OTHER; note = 2'-O-methylcytidine with a 3'-phosphorothioate linkage
  modified_base 2: mod_base = OTHER; note = 2'-O-methylguanosine with a 3'-phosphorothioate linkage
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-fluorouridine
  modified_base 9: mod_base = OTHER; note = 2'-fluoroadenosine
  modified_base 10: mod_base = um
  modified_base 11: mod_base = um
  modified_base 12: mod_base = um
  modified_base 13: mod_base = um
  modified_base 14: mod_base = gm
  modified_base 15: mod_base = gm
  modified_base 16: mod_base = um
  modified_base 17: mod_base = OTHER; note = 2'-O-methyluridine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
cggtcactattttggttaa

SEQ ID NO: 570 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttaaccaaaatagtgaccgtt

SEQ ID NO: 571 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = gm
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluorouridine
  modified_base 10: mod_base = gm
  modified_base 11: mod_base = um
  modified_base 12: mod_base = gm
  modified_base 13: mod_base = cm
  modified_base 14: mod_base = OTHER; note = 2'-O-methyladenosine
  modified_base 15: mod_base = um
  modified_base 16: mod_base = gm
  modified_base 17: mod_base = OTHER; note = 2'-O-methyladenosine with a 3'-phosphorothioate linkage
  modified_base 18: mod_base = OTHER; note = 2'-O-methylcytidine with a 3'-phosphorothioate linkage
  modified_base 19: mod_base = OTHER; note = 2'-O-methyladenosine
taattgtgtgtgcatgaca

SEQ ID NO: 572 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = cm
  modified_base 9: mod_base = OTHER; note = 2'-O-methyladenosine
  modified_base 10: mod_base = cm
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tgtcatgcacacacaattatt

SEQ ID NO: 573 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = um
  modified_base 4: mod_base = um
  modified_base 5: mod_base = gm
  modified_base 6: mod_base = um
  modified_base 7: mod_base = OTHER; note = 2'-fluoroguanosine
  modified_base 8: mod_base = OTHER; note = 2'-fluorouridine
  modified_base 9: mod_base = OTHER; note = 2'-fluoroguanosine
  modified_base 10: mod_base = um
  modified_base 11: mod_base = gm
  modified_base 12: mod_base = cm
  modified_base 13: mod_base = OTHER; note = 2'-O-methyladenosine
  modified_base 14: mod_base = um
  modified_base 15: mod_base = gm
  modified_base 16: mod_base = OTHER; note = 2'-O-methyladenosine
  modified_base 17: mod_base = OTHER; note = 2'-O-methylcytidine with a 3'-phosphorothioate linkage
  modified_base 18: mod_base = OTHER; note = 2'-O-methylcytidine with a 3'-phosphorothioate linkage
  modified_base 19: mod_base = OTHER; note = 2'-O-methyladenosine
aattgtgtgtgcatgacca

SEQ ID NO: 574 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = gm
  modified_base 4: mod_base = um
  modified_base 5: mod_base = cm
  modified_base 6: mod_base = OTHER; note = 2'-fluoroadenosine
  modified_base 7: mod_base = um
  modified_base 8: mod_base = gm
  modified_base 9: mod_base = cm
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tggtcatgcacacacaatttt

SEQ ID NO: 575 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = cm
  modified_base 6: mod_base = OTHER; note = 2'-O-methyladenosine
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluoroadenosine
  modified_base 10: mod_base = cm
  modified_base 11: mod_base = cm
  modified_base 12: mod_base = cm
  modified_base 13: mod_base = gm
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = OTHER; note = 2'-O-methyluridine with a 3'-phosphorothioate linkage
  modified_base 18: mod_base = OTHER; note = 2'-O-methylguanosine with a 3'-phosphorothioate linkage
  modified_base 19: mod_base = OTHER; note = 2'-O-methyladenosine
tgtgcatgacccgaactga

SEQ ID NO: 576 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluorouridine
  modified_base 7: mod_base = cm
  modified_base 8: mod_base = gm
  modified_base 9: mod_base = gm
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tcagttcgggtcatgcacatt

SEQ ID NO: 577 - RNA, 19 nt, synthetic construct
  source 1..19: mol_type = other RNA
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = um
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluorocytidine
  modified_base 10: mod_base = cm
  modified_base 11: mod_base = cm
  modified_base 12: mod_base = gm
  modified_base 13: mod_base = OTHER; note = 2'-O-methyladenosine
  modified_base 14: mod_base = OTHER; note = 2'-O-methyladenosine
  modified_base 15: mod_base = cm
  modified_base 16: mod_base = um
  modified_base 17: mod_base = OTHER; note = 2'-O-methylguanosine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
gtgcatgacccgaactgaa

SEQ ID NO: 578 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = gm
  modified_base 6: mod_base = OTHER; note = 2'-fluorouridine
  modified_base 7: mod_base = um
  modified_base 8: mod_base = cm
  modified_base 9: mod_base = gm
  modified_base 10: mod_base = gm
  modified_base 11: mod_base = gm
  modified_base 12: mod_base = um
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluoroguanosine
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = um
ttcagttcgggtcatgcactt

SEQ ID NO: 579 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-fluoroadenosine
  modified_base 9: mod_base = OTHER; note = 2'-fluorouridine
  modified_base 10: mod_base = gm
  modified_base 11: mod_base = OTHER; note = 2'-O-methyladenosine
  modified_base 12: mod_base = gm
  modified_base 13: mod_base = um
  modified_base 14: mod_base = um
  modified_base 15: mod_base = cm
  modified_base 16: mod_base = OTHER; note = 2'-O-methyladenosine
  modified_base 17: mod_base = OTHER; note = 2'-O-methylcytidine with a 3'-phosphorothioate linkage
  modified_base 18: mod_base = OTHER; note = 2'-O-methylguanosine with a 3'-phosphorothioate linkage
  modified_base 19: mod_base = OTHER; note = 2'-O-methyladenosine
tgtctaaatgagttcacga

SEQ ID NO: 580 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tcgtgaactcatttagacatt

SEQ ID NO: 581 - RNA, 19 nt, synthetic construct
  source 1..19: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = um
  modified_base 7: mod_base = OTHER; note = 2'-fluoroguanosine
  modified_base 8: mod_base = OTHER; note = 2'-fluoroadenosine
  modified_base 9: mod_base = OTHER; note = 2'-fluoroguanosine
  modified_base 10: mod_base = um
  modified_base 11: mod_base = um
  modified_base 12: mod_base = cm
  modified_base 13: mod_base = OTHER; note = 2'-O-methyladenosine
  modified_base 14: mod_base = cm
  modified_base 15: mod_base = gm
  modified_base 16: mod_base = um
  modified_base 17: mod_base = OTHER; note = 2'-O-methyladenosine with a 3'-phosphorothioate linkage
  modified_base 18: mod_base = OTHER; note = 2'-O-methylcytidine with a 3'-phosphorothioate linkage
  modified_base 19: mod_base = OTHER; note = 2'-O-methyladenosine
ctaaatgagttcacgtaca

SEQ ID NO: 582 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-fluoroguanosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = um
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
tgtacgtgaactcatttagtt

SEQ ID NO: 583 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = um
  modified_base 5: mod_base = gm
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-fluorouridine
  modified_base 9: mod_base = OTHER; note = 2'-fluorouridine
  modified_base 10: mod_base = cm
  modified_base 11: mod_base = OTHER; note = 2'-O-methyladenosine
  modified_base 12: mod_base = cm
  modified_base 13: mod_base = gm
  modified_base 14: mod_base = um
  modified_base 15: mod_base = OTHER; note = 2'-O-methyladenosine
  modified_base 16: mod_base = cm
  modified_base 17: mod_base = OTHER; note = 2'-O-methylcytidine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
aaatgagttcacgtaccta

SEQ ID NO: 584 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = gm
  modified_base 5: mod_base = um
  modified_base 6: mod_base = OTHER; note = 2'-fluoroadenosine
  modified_base 7: mod_base = cm
  modified_base 8: mod_base = gm
  modified_base 9: mod_base = um
  modified_base 10: mod_base = gm
  modified_base 11: mod_base = OTHER; note = 2'-O-methyladenosine
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
taggtacgtgaactcattttt

SEQ ID NO: 585 - RNA, 19 nt, synthetic construct
  source 1..19: mol_type = other RNA
  modified_base 1: mod_base = OTHER; note = 2'-O-methylguanosine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = um
  modified_base 5: mod_base = um
  modified_base 6: mod_base = um
  modified_base 7: mod_base = OTHER; note = 2'-fluorouridine
  modified_base 8: mod_base = OTHER; note = 2'-fluoroadenosine
  modified_base 9: mod_base = OTHER; note = 2'-fluoroguanosine
  modified_base 10: mod_base = cm
  modified_base 11: mod_base = gm
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine with a 3'-phosphorothioate linkage
  modified_base 18: mod_base = OTHER; note = 2'-O-methylcytidine with a 3'-phosphorothioate linkage
  modified_base 19: mod_base = OTHER; note = 2'-O-methyladenosine
ggattttagcgtcattaca

SEQ ID NO: 586 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = cm
  modified_base 10: mod_base = gm
  modified_base 11: mod_base = cm
  modified_base 12: mod_base = um
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = um
tgtaatgacgctaaaatcctt

SEQ ID NO: 587 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluoroadenosine
  modified_base 8: mod_base = OTHER; note = 2'-fluoroadenosine
  modified_base 9: mod_base = OTHER; note = 2'-fluorocytidine
  modified_base 10: mod_base = um
  modified_base 11: mod_base = OTHER; note = 2'-O-methyladenosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = um
  modified_base 17: mod_base = OTHER; note = 2'-O-methylcytidine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
acaaccaactactcatcta

SEQ ID NO: 588 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = um
  modified_base 13: mod_base = um
  modified_base 14: mod_base = OTHER; note = 2'-fluoroguanosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tagatgagtagttggttgttt

SEQ ID NO: 589 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = um
  modified_base 5: mod_base = um
  modified_base 6: mod_base = cm
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluoroadenosine
  modified_base 10: mod_base = cm
  modified_base 11: mod_base = OTHER; note = 2'-O-methyladenosine
  modified_base 12: mod_base = OTHER; note = 2'-O-methyladenosine
  modified_base 13: mod_base = cm
  modified_base 14: mod_base = cm
  modified_base 15: mod_base = cm
  modified_base 16: mod_base = gm
  modified_base 17: mod_base = OTHER; note = 2'-O-methylguanosine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
aacttctgacaacccggaa

SEQ ID NO: 590 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = cm
  modified_base 4: mod_base = cm
  modified_base 5: mod_base = gm
  modified_base 6: mod_base = OTHER; note = 2'-fluoroguanosine
  modified_base 7: mod_base = gm
  modified_base 8: mod_base = um
  modified_base 9: mod_base = um
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroadenosine
  modified_base 17: mod_base = gm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
ttccgggttgtcagaagtttt

SEQ ID NO: 591 - RNA, 19 nt, synthetic construct
  source 1..19: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = um
  modified_base 4: mod_base = cm
  modified_base 5: mod_base = um
  modified_base 6: mod_base = gm
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluoroadenosine
  modified_base 10: mod_base = OTHER; note = 2'-O-methyladenosine
  modified_base 11: mod_base = cm
  modified_base 12: mod_base = cm
  modified_base 13: mod_base = cm
  modified_base 14: mod_base = gm
  modified_base 15: mod_base = gm
  modified_base 16: mod_base = OTHER; note = 2'-O-methyladenosine
  modified_base 17: mod_base = OTHER; note = 2'-O-methyluridine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
cttctgacaacccggatta

SEQ ID NO: 592 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = OTHER; note = 2'-fluorocytidine
  modified_base 7: mod_base = gm
  modified_base 8: mod_base = gm
  modified_base 9: mod_base = gm
  modified_base 10: mod_base = um
  modified_base 11: mod_base = um
  modified_base 12: mod_base = gm
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
taatccgggttgtcagaagtt

SEQ ID NO: 593 - RNA, 19 nt, synthetic construct
  source 1..19: mol_type = other RNA
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-fluoroadenosine
  modified_base 8: mod_base = OTHER; note = 2'-fluorocytidine
  modified_base 9: mod_base = OTHER; note = 2'-fluorocytidine
  modified_base 10: mod_base = cm
  modified_base 11: mod_base = gm
  modified_base 12: mod_base = gm
  modified_base 13: mod_base = OTHER; note = 2'-O-methyladenosine
  modified_base 14: mod_base = um
  modified_base 15: mod_base = um
  modified_base 16: mod_base = um
  modified_base 17: mod_base = OTHER; note = 2'-O-methyluridine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
ctgacaacccggattttaa

SEQ ID NO: 594 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = cm
  modified_base 9: mod_base = cm
  modified_base 10: mod_base = gm
  modified_base 11: mod_base = gm
  modified_base 12: mod_base = gm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttaaaatccgggttgtcagtt

SEQ ID NO: 595 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorocytidine
  modified_base 8: mod_base = OTHER; note = 2'-fluorocytidine
  modified_base 9: mod_base = OTHER; note = 2'-fluorocytidine
  modified_base 10: mod_base = gm
  modified_base 11: mod_base = gm
  modified_base 12: mod_base = OTHER; note = 2'-O-methyladenosine
  modified_base 13: mod_base = um
  modified_base 14: mod_base = um
  modified_base 15: mod_base = um
  modified_base 16: mod_base = um
  modified_base 17: mod_base = OTHER; note = 2'-O-methyladenosine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
tgacaacccggattttaaa

SEQ ID NO: 596 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = cm
  modified_base 10: mod_base = cm
  modified_base 11: mod_base = gm
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tttaaaatccgggttgtcatt

SEQ ID NO: 597 - RNA, 19 nt, synthetic construct
  source 1..19: mol_type = other RNA
  modified_base 1: mod_base = OTHER; note = 2'-O-methyladenosine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = cm
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-O-methyladenosine
  modified_base 11: mod_base = um
  modified_base 12: mod_base = um
  modified_base 13: mod_base = um
  modified_base 14: mod_base = um
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methylcytidine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
acaacccggattttaacta

SEQ ID NO: 598 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = um
  modified_base 11: mod_base = cm
  modified_base 12: mod_base = cm
  modified_base 13: mod_base = gm
  modified_base 14: mod_base = OTHER; note = 2'-fluoroguanosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tagttaaaatccgggttgttt

SEQ ID NO: 599 - RNA, 19 nt, synthetic construct
  source 1..19: mol_type = other RNA
  modified_base 1: mod_base = OTHER; note = 2'-O-methylcytidine with a 3'-phosphorothioate linkage
  modified_base 2: mod_base = OTHER; note = 2'-O-methyladenosine with a 3'-phosphorothioate linkage
  modified_base 3: mod_base = OTHER; note = 2'-O-methyladenosine
  modified_base 4: mod_base = cm
  modified_base 5: mod_base = cm
  modified_base 6: mod_base = cm
  modified_base 7: mod_base = OTHER; note = 2'-fluoroguanosine
  modified_base 8: mod_base = OTHER; note = 2'-fluoroguanosine
  modified_base 9: mod_base = OTHER; note = 2'-fluoroadenosine
  modified_base 10: mod_base = um
  modified_base 11: mod_base = um
  modified_base 12: mod_base = um
  modified_base 13: mod_base = um
  modified_base 14: mod_base = OTHER; note = 2'-O-methyladenosine
  modified_base 15: mod_base = OTHER; note = 2'-O-methyladenosine
  modified_base 16: mod_base = cm
  modified_base 17: mod_base = OTHER; note = 2'-O-methyluridine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
caacccggattttaactaa

SEQ ID NO: 600 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = cm
  modified_base 13: mod_base = cm
  modified_base 14: mod_base = OTHER; note = 2'-fluoroguanosine
  modified_base 15: mod_base = gm
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttagttaaaatccgggttgtt

SEQ ID NO: 601 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = um
  modified_base 7: mod_base = OTHER; note = 2'-fluorouridine
  modified_base 8: mod_base = OTHER; note = 2'-fluoroguanosine
  modified_base 9: mod_base = OTHER; note = 2'-fluoroguanosine
  modified_base 10: mod_base = cm
  modified_base 11: mod_base = um
  modified_base 12: mod_base = gm
  modified_base 13: mod_base = um
  modified_base 14: mod_base = OTHER; note = 2'-O-methyladenosine
  modified_base 15: mod_base = gm
  modified_base 16: mod_base = um
  modified_base 17: mod_base = OTHER; note = 2'-O-methylcytidine with a 3'-phosphorothioate linkage
  modified_base 18: mod_base = OTHER; note = 2'-O-methyladenosine with a 3'-phosphorothioate linkage
  modified_base 19: mod_base = OTHER; note = 2'-O-methyladenosine
tgatcttggctgtagtcaa

SEQ ID NO: 602 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluorouridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-fluorouridine
  modified_base 7: mod_base = OTHER; note = 2'-O-methyladenosine
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
ttgactacagccaagatcatt

SEQ ID NO: 603 - RNA, 19 nt, synthetic construct
  source 1..19: mol_type = other RNA
  modified_base 1: mod_base = OTHER; note = 2'-O-methyluridine with a 3'-phosphorothioate linkage
  modified_base 2: mod_base = OTHER; note = 2'-O-methyladenosine with a 3'-phosphorothioate linkage
  modified_base 3: mod_base = cm
  modified_base 4: mod_base = cm
  modified_base 5: mod_base = um
  modified_base 6: mod_base = um
  modified_base 7: mod_base = OTHER; note = 2'-fluorocytidine
  modified_base 8: mod_base = OTHER; note = 2'-fluoroguanosine
  modified_base 9: mod_base = OTHER; note = 2'-fluoroadenosine
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-O-methyladenosine
  modified_base 15: mod_base = um
  modified_base 16: mod_base = gm
  modified_base 17: mod_base = OTHER; note = 2'-O-methylcytidine with a 3'-phosphorothioate linkage
  modified_base 18: mod_base = OTHER; note = 2'-O-methyluridine with a 3'-phosphorothioate linkage
  modified_base 19: mod_base = OTHER; note = 2'-O-methyladenosine
taccttcgaagccatgcta

SEQ ID NO: 604 - RNA, 21 nt, synthetic construct
  source 1..21: mol_type = other RNA
  modified_base 1: mod_base = OTHER; note = 2'-O-methyluridine with a 3'-phosphorothioate linkage
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = gm
  modified_base 9: mod_base = cm
  modified_base 10: mod_base = um
  modified_base 11: mod_base = um
  modified_base 12: mod_base = cm
  modified_base 13: mod_base = gm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tagcatggcttcgaaggtatt

SEQ ID NO: 605 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = OTHER; note = 2'-O-methyladenosine
  modified_base 14: mod_base = OTHER; note = 2'-fluorocytidine
  modified_base 15: mod_base = cm
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tccaaaatagtgaccgtaatt

SEQ ID NO: 606 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = cm
  modified_base 5: mod_base = cm
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = um
  modified_base 11: mod_base = OTHER; note = 2'-O-methyladenosine
  modified_base 12: mod_base = gm
  modified_base 13: mod_base = um
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluorocytidine
  modified_base 17: mod_base = cm
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
taaccaaaatagtgaccgttt

SEQ ID NO: 607 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = cm
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = OTHER; note = 2'-O-methyladenosine
  modified_base 13: mod_base = gm
  modified_base 14: mod_base = OTHER; note = 2'-fluorouridine
  modified_base 15: mod_base = gm
  modified_base 16: mod_base = OTHER; note = 2'-fluoroadenosine
  modified_base 17: mod_base = cm
  modified_base 18: mod_base = cm
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttaaccaaaatagtgaccgtt

SEQ ID NO: 608 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = cm
  modified_base 9: mod_base = OTHER; note = 2'-O-methyladenosine
  modified_base 10: mod_base = cm
  modified_base 11: mod_base = OTHER; note = 2'-O-methyladenosine
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tgtcatgcacacacaattatt

SEQ ID NO: 609 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = um
  modified_base 5: mod_base = cm
  modified_base 6: mod_base = OTHER; note = 2'-fluoroadenosine
  modified_base 7: mod_base = um
  modified_base 8: mod_base = gm
  modified_base 9: mod_base = cm
  modified_base 10: mod_base = OTHER; note = 2'-O-methyladenosine
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = OTHER; note = 2'-O-methyladenosine
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tggtcatgcacacacaatttt

SEQ ID NO: 610 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluorouridine
  modified_base 7: mod_base = cm
  modified_base 8: mod_base = gm
  modified_base 9: mod_base = gm
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluorouridine
  modified_base 15: mod_base = gm
  modified_base 16: mod_base = OTHER; note = 2'-fluorocytidine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tcagttcgggtcatgcacatt

SEQ ID NO: 611 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = OTHER; note = 2'-O-methyladenosine
  modified_base 5: mod_base = gm
  modified_base 6: mod_base = OTHER; note = 2'-fluorouridine
  modified_base 7: mod_base = um
  modified_base 8: mod_base = cm
  modified_base 9: mod_base = gm
  modified_base 10: mod_base = gm
  modified_base 11: mod_base = gm
  modified_base 12: mod_base = um
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluoroguanosine
  modified_base 17: mod_base = cm
  modified_base 18: mod_base = OTHER; note = 2'-O-methyladenosine
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = um
ttcagttcgggtcatgcactt

SEQ ID NO: 612 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = gm
  modified_base 4: mod_base = um
  modified_base 5: mod_base = gm
  modified_base 6: mod_base = OTHER; note = 2'-fluoroadenosine
  modified_base 7: mod_base = OTHER; note = 2'-O-methyladenosine
  modified_base 8: mod_base = cm
  modified_base 9: mod_base = um
  modified_base 10: mod_base = cm
  modified_base 11: mod_base = OTHER; note = 2'-O-methyladenosine
  modified_base 12: mod_base = um
  modified_base 13: mod_base = um
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tcgtgaactcatttagacatt

SEQ ID NO: 613 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-fluoroguanosine
  modified_base 7: mod_base = um
  modified_base 8: mod_base = gm
  modified_base 9: mod_base = OTHER; note = 2'-O-methyladenosine
  modified_base 10: mod_base = OTHER; note = 2'-O-methyladenosine
  modified_base 11: mod_base = cm
  modified_base 12: mod_base = um
  modified_base 13: mod_base = cm
  modified_base 14: mod_base = OTHER; note = 2'-fluoroadenosine
  modified_base 15: mod_base = um
  modified_base 16: mod_base = OTHER; note = 2'-fluorouridine
  modified_base 17: mod_base = um
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
tgtacgtgaactcatttagtt

SEQ ID NO: 614 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = gm
  modified_base 5: mod_base = um
  modified_base 6: mod_base = OTHER; note = 2'-fluoroadenosine
  modified_base 7: mod_base = cm
  modified_base 8: mod_base = gm
  modified_base 9: mod_base = um
  modified_base 10: mod_base = gm
  modified_base 11: mod_base = OTHER; note = 2'-O-methyladenosine
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = cm
  modified_base 16: mod_base = OTHER; note = 2'-fluoroadenosine
  modified_base 17: mod_base = um
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
taggtacgtgaactcattttt

SEQ ID NO: 615 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = OTHER; note = 2'-O-methyladenosine
  modified_base 9: mod_base = cm
  modified_base 10: mod_base = gm
  modified_base 11: mod_base = cm
  modified_base 12: mod_base = um
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = um
tgtaatgacgctaaaatcctt

SEQ ID NO: 616 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = um
  modified_base 6: mod_base = OTHER; note = 2'-fluoroguanosine
  modified_base 7: mod_base = OTHER; note = 2'-O-methyladenosine
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = um
  modified_base 13: mod_base = um
  modified_base 14: mod_base = OTHER; note = 2'-fluoroguanosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tagatgagtagttggttgttt

SEQ ID NO: 617 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = cm
  modified_base 4: mod_base = cm
  modified_base 5: mod_base = gm
  modified_base 6: mod_base = OTHER; note = 2'-fluoroguanosine
  modified_base 7: mod_base = gm
  modified_base 8: mod_base = um
  modified_base 9: mod_base = um
  modified_base 10: mod_base = gm
  modified_base 11: mod_base = um
  modified_base 12: mod_base = cm
  modified_base 13: mod_base = OTHER; note = 2'-O-methyladenosine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroadenosine
  modified_base 17: mod_base = gm
  modified_base 18: mod_base = um
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
ttccgggttgtcagaagtttt

SEQ ID NO: 618 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = OTHER; note = 2'-O-methyladenosine
  modified_base 4: mod_base = um
  modified_base 5: mod_base = cm
  modified_base 6: mod_base = OTHER; note = 2'-fluorocytidine
  modified_base 7: mod_base = gm
  modified_base 8: mod_base = gm
  modified_base 9: mod_base = gm
  modified_base 10: mod_base = um
  modified_base 11: mod_base = um
  modified_base 12: mod_base = gm
  modified_base 13: mod_base = um
  modified_base 14: mod_base = OTHER; note = 2'-fluorocytidine
  modified_base 15: mod_base = OTHER; note = 2'-O-methyladenosine
  modified_base 16: mod_base = OTHER; note = 2'-fluoroguanosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
taatccgggttgtcagaagtt

SEQ ID NO: 619 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = cm
  modified_base 9: mod_base = cm
  modified_base 10: mod_base = gm
  modified_base 11: mod_base = gm
  modified_base 12: mod_base = gm
  modified_base 13: mod_base = um
  modified_base 14: mod_base = OTHER; note = 2'-fluorouridine
  modified_base 15: mod_base = gm
  modified_base 16: mod_base = OTHER; note = 2'-fluorouridine
  modified_base 17: mod_base = cm
  modified_base 18: mod_base = OTHER; note = 2'-O-methyladenosine
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttaaaatccgggttgtcagtt

SEQ ID NO: 620 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = cm
  modified_base 10: mod_base = cm
  modified_base 11: mod_base = gm
  modified_base 12: mod_base = gm
  modified_base 13: mod_base = gm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tttaaaatccgggttgtcatt

SEQ ID NO: 621 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = um
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = um
  modified_base 11: mod_base = cm
  modified_base 12: mod_base = cm
  modified_base 13: mod_base = gm
  modified_base 14: mod_base = OTHER; note = 2'-fluoroguanosine
  modified_base 15: mod_base = gm
  modified_base 16: mod_base = OTHER; note = 2'-fluorouridine
  modified_base 17: mod_base = um
  modified_base 18: mod_base = gm
  modified_base 19: mod_base = OTHER; note = 2'-O-methyluridine with a 3'-phosphorothioate linkage
  modified_base 20: mod_base = OTHER; note = 2'-O-methyluridine with a 3'-phosphorothioate linkage
  modified_base 21: mod_base = um
tagttaaaatccgggttgttt

SEQ ID NO: 622 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = um
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = um
  modified_base 12: mod_base = cm
  modified_base 13: mod_base = cm
  modified_base 14: mod_base = OTHER; note = 2'-fluoroguanosine
  modified_base 15: mod_base = gm
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methyluridine with a 3'-phosphorothioate linkage
  modified_base 21: mod_base = um
ttagttaaaatccgggttgtt

SEQ ID NO: 623 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = OTHER; note = 2'-fluorouridine
  modified_base 7: mod_base = OTHER; note = 2'-O-methyladenosine
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = OTHER; note = 2'-O-methyladenosine
  modified_base 14: mod_base = OTHER; note = 2'-fluoroadenosine
  modified_base 15: mod_base = gm
  modified_base 16: mod_base = OTHER; note = 2'-fluoroadenosine
  modified_base 17: mod_base = um
  modified_base 18: mod_base = cm
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
ttgactacagccaagatcatt

SEQ ID NO: 624 - RNA, 21 nt, synthetic construct
  source 1..21: mol_type = other RNA
  modified_base 1: mod_base = OTHER; note = 2'-O-methoxyethylribose-unlocked-uridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-fluorouridine
  modified_base 7: mod_base = gm
  modified_base 8: mod_base = gm
  modified_base 9: mod_base = cm
  modified_base 10: mod_base = um
  modified_base 11: mod_base = um
  modified_base 12: mod_base = cm
  modified_base 13: mod_base = gm
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = gm
  modified_base 18: mod_base = um
  modified_base 19: mod_base = OTHER; note = 2'-O-methyladenosine with a 3'-phosphorothioate linkage
  modified_base 20: mod_base = OTHER; note = 2'-O-methyluridine with a 3'-phosphorothioate linkage
  modified_base 21: mod_base = um
tagcatggcttcgaaggtatt

SEQ ID NO: 625 - protein, 451 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 626 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSS

SEQ ID NO: 628 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 629 - protein, 5 aa, synthetic construct
SYSMN

SEQ ID NO: 630 - protein, 17 aa, synthetic construct
SISSSSSYIYYADSVKG

SEQ ID NO: 631 - protein, 12 aa, synthetic construct
RHGYSNSDAFDT

SEQ ID NO: 632 - protein, 11 aa, synthetic construct
RASQGISHYLV

SEQ ID NO: 633 - protein, 7 aa, synthetic construct
AASSLQS

SEQ ID NO: 634 - protein, 9 aa, synthetic construct
LQHNSYPWT

SEQ ID NO: 635 - protein, 451 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTKNQVSLVCLVKGFYPSDIAVEWESNGQPENNYKTTPPVLDSDGSFFLYSVLTVDKSRWQQGNVFSCSVMHEALHNHYTQKSLSLSPGK

SEQ ID NO: 636 - protein, 451 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTDNQVSLTCLVKGFYPSDIAVEWESNGQPENNYKTTPPVLMSDGSFFLASKLTVDKSRWQQGNVFSCSVMHEALHNHYTQKSLSLSPGK

SEQ ID NO: 637 - protein, 219 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDTWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 638 - protein, 213 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 639 - DNA, 1353 bp, synthetic construct
  source 1..1353: mol_type = other DNA
gaggtgcagctggtggagtctgggggaggcctggtcaagcctggggggtccctgagactctcctgtgtagcctctggattcaccttcagtagctatagcatgaactgggtccgccaggctccagggaaggggctggagtgggtctcatccattagttccagtagtagttacatatattatgcagactcagtgaagggccgattcaccatctccagagacaacgccaagaactcactgtatctgcaaatgaacagcctgagagccgaggacacggctgtgtattactgtgcgagaaggcatggctacagtaactccgatgcttttgatacttggggccaagggacattggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 640 - DNA, 1353 bp, synthetic construct
  source 1..1353: mol_type = other DNA
gaggtgcagctggtggagtctgggggaggcctggtcaagcctggggggtccctgagactctcctgtgtagcctctggattcaccttcagtagctatagcatgaactgggtccgccaggctccagggaaggggctggagtgggtctcatccattagttccagtagtagttacatatattatgcagactcagtgaagggccgattcaccatctccagagacaacgccaagaactcactgtatctgcaaatgaacagcctgagagccgaggacacggctgtgtattactgtgcgagaaggcatggctacagtaactccgatgcttttgatacttggggccaagggacattggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgaagaccacgggtgtacaccctgcccccatcccgggaggagatgaccaagaaccaagtcagcctggtctgcctggtcaaaggcttctatcccagcgacatcgccgtggagtgggagagcaatgggcagccggagaacaactacaagaccacgcctcccgtgctggactccgacggctccttcttcctctattccgtgctcaccgtggacaagagcaggtggcagcaggggaacgtcttctcatgctccgtgatgcatgaggctctgcacaaccactacacgcagaagagcctctccctgtctccgggcaaa

SEQ ID NO: 641 - DNA, 657 bp, synthetic construct
  source 1..657: mol_type = other DNA
gaagtgcagctggtcgagtcaggaggcggcctcgtgaaaccgggtggtagcctgaggctgtcctgcgtggcatcagggttcaccttctcgtcctactccatgaactgggtcagacaggcccccggaaagggacttgaatgggtgtccagcatcagcagctcctcctcgtacatctactacgccgattccgtgaagggccggttcaccattagccgcgacaatgccaagaactcgctgtatttgcaaatgaactctctgcgcgccgaggacactgctgtgtactactgtgcgcggagacacgggtactccaactccgatgcctttgacacctggggccagggaactctcgtgaccgtgtcgtccgccagcaccaagggcccatgcgtcttccccctggcaccctcctccaagagcacctctgggggcacagcggccctgggctgcctggtcaaggactacttccccgaaccggtgacggtgtcgtggaactcaggcgcactgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagtt

SEQ ID NO: 642 - DNA, 1353 bp, synthetic construct
  source 1..1353: mol_type = other DNA
gaagtgcagctggtcgagtcaggaggcggcctcgtgaaaccgggtggtagcctgaggctgtcctgcgtggcatcagggttcaccttctcgtcctactccatgaactgggtcagacaggcccccggaaagggacttgaatgggtgtccagcatcagcagctcctcctcgtacatctactacgccgattccgtgaagggccggttcaccattagccgcgacaatgccaagaactcgctgtatttgcaaatgaactctctgcgcgccgaggacactgctgtgtactactgtgcgcggagacacgggtactccaactccgatgcctttgacacctggggccagggaactctcgtgaccgtgtcgtccgccagcaccaagggcccatgcgtcttccccctggcaccctcctccaagagcacctctgggggcacagcggccctgggctgcctggtcaaggactacttccccgaaccggtgacggtgtcgtggaactcaggcgcgctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgtccgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccgacaaccaagtcagcctgacctgcctggtcaaaggcttctatcccagcgacatcgccgtggagtgggagagcaatgggcagccggagaacaactacaagaccacgcctcccgtgctgatgtccgacggctccttcttcctcgcctccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 643 - DNA, 642 bp, synthetic construct
  source 1..642: mol_type = other DNA
gacatccagatgacccagtctccatctgccatgtctgcatctgtaggagacagagtcaccatcacttgtcgggcgagtcagggcattagccactatttagtgtggtttcagcagaaaccagggaaagtccctaagcgcctgatctatgctgcatccagtttacaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaacgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 644 - DNA, 642 bp, synthetic construct; identical to SEQ ID NO: 643
  source 1..642: mol_type = other DNA

SEQ ID NO: 645 - DNA, 639 bp, synthetic construct
  source 1..639: mol_type = other DNA
gacatccagatgacccagtctccatctgccatgtctgcatctgtaggagacagagtcaccatcacttgtcgggcgagtcagggcattagccactatttagtgtggtttcagcagaaaccagggaaagtccctaagcgcctgatctatgctgcatccagtttacaaagtggggtcccatcaaggttcagcggcagtggatctgggacagaattcactctcacaatcagcagcctgcagcctgaagattttgcaacttattactgtctacagcataatagttacccttggacgttcggccaagggaccaaggtggaaatcaaacgaaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagag

SEQ ID NO: 646 - RNA, 19 nt, synthetic construct
  source 1..19: mol_type = other RNA
ctagctggacacgtcgata

SEQ ID NO: 647 - RNA, 21 nt, synthetic construct
  source 1..21: mol_type = other RNA
tatcgacgtgtccagctagtt

SEQ ID NO: 648 - RNA, 5874 nt, Homo sapiens
  source 1..5874: mol_type = mRNA
atggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcgtggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctagtga